(19) United States
(12) Patent Application Publication
(10) Pub. No.: US 2018/0370927 A1
(43) Pub. Date: Dec. 27, 2018

(54) MICROBIOCIDAL OXADIAZOLE DERIVATIVES

(71) Applicant: SYNGENTA PARTICIPATIONS AG, Basel (CH)

(72) Inventors: Thomas James Hoffman, Stein (CH); Daniel Stierli, Stein (CH); Renaud Beaudegnies, Stein (CH); Martin Pouliot, Stein (CH)

(73) Assignee: SYNGENTA PARTICIPATIONS AG, Basel (CH)

(21) Appl. No.: 16/064,876

(22) PCT Filed: Dec. 22, 2016
(86) PCT No.: PCT/EP2016/082327

§ 371 Date: Jun. 21, 2018

(30) Foreign Application Priority Data
Dec. 22, 2015 (EP) 15202187.9
Feb. 2, 2016 (EP) 16153938.2

(51) Int. Cl. C07D 271/06; C07D 413/04; C07D 413/10; C07D 413/14; A01N 43/82

(52) U.S. Cl. CPC C07D 271/06; C07D 413/04; A01N 43/82; C07D 413/14; C07D 413/10

(57) ABSTRACT

Compounds of the formula (I); wherein the substituents are as defined in claim 1, useful as a pesticides, especially as fungicides.

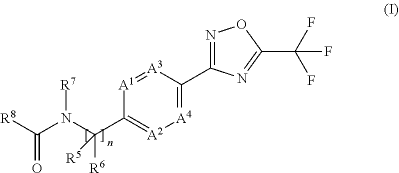

The present invention relates to microbiocidal oxadiazole derivatives, eg, as active ingredients, which have microbiocidal activity, in particular, fungicidal activity. The invention also relates to agrochemical compositions which comprise at least one of the oxadiazole derivatives, to processes of preparation of these compounds and to uses of the oxadiazole derivatives or compositions in agriculture or horticulture for controlling or preventing infestation of plants, harvested food crops, seeds or non-living materials by phytopathogenic microorganisms, preferably fungi.

Phenyl oxadiazole derivatives are known as pharmaceutically-active agents from, eg, WO 2013/066835.

According to the present invention, there is provided a compound of formula (I):

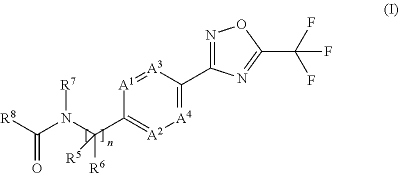

wherein

n represents 0, 1 or 2;

A¹ represents N or CR¹, wherein R¹ represents hydrogen, halogen, methyl, ethyl, difluoromethyl, trifluoromethyl, methoxy, ethoxy, or difluoromethoxy;

A² represents N or CR², wherein R² represents hydrogen, halogen, methyl, ethyl, difluoromethyl, trifluoromethyl, methoxy, ethoxy, or difluoromethoxy;

A³ represents N or CR³, wherein R³ represents hydrogen or halogen;

A⁴ represents N or CR⁴, wherein R⁴ represents hydrogen or halogen; and wherein no more than two of A¹ to A⁴ are N; R⁵ and R⁶ are independently selected from hydrogen, C₁₋₄alkyl, halogen, cyano, trifluoromethyl and difluoromethyl, or R⁵ and R⁶ together with the carbon atom they share form a cyclopropyl;

R⁷ represents hydrogen, hydroxy, C₁₋₄alkyl, C₁₋₄haloalkyl, C₁₋₄alkoxy, C₁₋₄haloalkoxy, hydroxyC₁₋₄alkyl, C₁₋₂alkoxyC₁₋₄alkyl, C₁₋₂haloalkoxyC₁₋₄alkyl, cyanoC₁₋₄alkyl, C₃₋₆alkenyl, C₃₋₆alkynyl, C₃₋₆alkenyloxy, C₃₋₆alkynyloxy, C₃₋₆haloalkenyl, C₃₋₆haloalkenyloxy, or

R⁷ represents C₃₋₆cycloalkyl, C₃₋₆cycloalkylC₁₋₂alkyl, C₃₋₆cycloalkylC₁₋₂alkoxy, phenyl, phenylC₁₋₂alkyl, phenylC₁₋₂alkoxy, heteroaryl, heteroarylC₁₋₂alkyl, heteroarylC₁₋₂alkoxy, heterocyclyl, heterocyclylC₁₋₂alkyl, or heterocyclylC₁₋₂alkoxy,

wherein the heteroaryl moiety is a 5- or 6-membered monocyclic aromatic ring which comprises 1, 2, 3 or 4 heteroatoms individually selected from N, O and S, the heterocyclyl moiety is a 4- to 6-membered non-aromatic ring which comprises 1 or 2 heteroatoms individually selected from N, O and S, and wherein any of cycloalkyl, phenyl, heteroaryl and heterocyclyl moieties are optionally substituted by 1 or 2 substituents selected from cyano, fluoro, chloro, bromo, methyl, difluoromethyl, trifluoromethyl, methoxy, ethoxy, or difluoromethoxy;

R⁸ represents —C(═R⁹)—R¹⁰, wherein R⁹ represents O or N—C₁₋₄alkoxy;

R¹⁰ represents hydrogen, cyano, C₁₋₆alkyl, C₂₋₆alkenyl, C₃₋₆alkenyloxy, C₂₋₆alkynyl, cyanoC₁₋₆alkyl, C₁₋₆haloalkyl, C₃₋₆haloalkenyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkylcarbonylC₂₋₆alkenyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₆alkylsulfanylC₁₋₆alkyl, C₁₋₆alkylsulfonylC₁₋₆alkyl, C₁₋₆alkylsulfonylaminoC₁₋₆alkyl, or

R¹⁰ represents C₃₋₈cycloalkyl, C₃₋₈cycloalkylC₁₋₆alkyl, C₃₋₈cycloalkylC₁₋₆alkoxy wherein the cycloalkyl moiety is optionally partially unsaturated, phenyl, phenylC₁₋₆alkyl, phenylC₁₋₆alkoxy, heteroaryl, heteroarylC₁₋₆alkyl, heteroarylC₁₋₆alkoxy, wherein the heteroaryl moiety is a 5- or 6-membered monocyclic aromatic ring which comprises 1, 2, 3 or 4 heteroatoms individually selected from N, O and S, heterocyclyl, heterocyclylC₁₋₆alkyl, heterocyclylC₁₋₆alkoxy wherein the heterocyclyl moiety is a 4- to 6-membered non-aromatic ring which comprises 1, 2 or 3 heteroatoms individually selected from N, O and S;

wherein for R¹⁰, any C₃₋₈cycloalkyl, phenyl, heteroaryl, or heterocyclyl is optionally substituted by 1, 2 or 3 substituents, which may be the same or different, selected from R¹¹;

R¹¹ represents cyano, halogen, hydroxy, C₁₋₄alkyl, C₂₋₄alkenyl, C₂₋₄alkynyl, C₁₋₄haloalkyl, C₂₋₄haloalkenyl, C₁₋₄alkoxy, C₁₋₄haloalkoxy, C₃₋₄alkenyloxy, C₃₋₄alkynyloxy, N—C₁₋₄alkylamino, N,N-diC₁₋₄alkylamino, C₁₋₄alkylcarbonyl, C₁₋₄alkoxycarbonyl, carbonylamino, N—C₁₋₄alkylaminocarbonyl, N,N-diC₁₋₄alkylaminocarbonyl or C₁₋₄alkoxycarbonylamino;

and wherein when R¹⁰ is substituted C₃₋₈cycloalkyl, C₃₋₈cycloalkylC₁₋₆alkyl, C₃₋₈cycloalkylC₁₋₆alkoxy, heterocyclyl, heterocyclylC₁₋₆alkyl or heterocyclylC₁₋₆alkoxy, R¹¹ may also represent oxo on the C₃₋₈cycloalkyl or heterocyclyl moiety; or

R⁸ represents —C(═O)—OR¹²;

R¹² represents hydrogen, C₁₋₄alkyl, C₃₋₆alkenyl, C₃₋₆alkynyl, cyanoC₁₋₆alkyl, C₁₋₆haloalkyl, C₃₋₆haloalkenyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkylcarbonylC₂₋₆alkenyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₄alkylsulfanylC₁₋₆alkyl, C₁₋₆alkylsulfonylC₁₋₆alkyl, C₁₋₆alkylsulfonylaminoC₁₋₆alkyl, or

R¹² represents C₃₋₈cycloalkyl or C₃₋₈cycloalkylC₁₋₆alkyl wherein the cycloalkyl moiety is optionally partially unsaturated, phenyl, phenylC₁₋₆alkyl, heteroaryl or heteroarylC₁₋₆alkyl, wherein the heteroaryl moiety is a 5- or 6-membered monocyclic aromatic ring which comprises 1, 2, 3 or 4 heteroatoms individually selected from N, O and S, heterocyclyl or heterocyclylC₁₋₆-alkyl wherein the heterocyclyl moiety is a 4- to 6-membered non-aromatic ring which comprises 1, 2 or 3 heteroatoms individually selected from N, O and S,

wherein for R¹², any C₃₋₈cycloalkyl, phenyl, heteroaryl or heterocyclyl is optionally substituted by 1, 2 or 3 substituents, which may be the same or different, selected from R¹³; wherein

R¹³ represents cyano, halogen, hydroxy, C₁₋₄alkyl, C₂₋₄alkenyl, C₂₋₄alkynyl, C₁₋₄haloalkyl, C₂₋₄haloalkenyl, C₁₋₄alkoxy, C₁₋₄haloalkoxy, C₃₋₄alkenyloxy, C₃₋₄alkynyloxy, N—C₁₋₄alkylamino, N,N-diC₁₋₄alkylamino, C₁₋₄alkylcarbonyl, C₁₋₄alkoxycarbonyl, carbonylamino, N—C₁₋₄alkylaminocarbonyl, N,N-diC₁₋₄alkylaminocarbonyl or C₁₋₄alkoxycarbonylamino;

and wherein when R¹² is substituted C₃₋₈cycloalkyl, C₃₋₈cycloalkylC₁₋₆alkyl, heterocyclyl or heterocyclylC₁₋₆alkyl, R¹³ may also represent oxo on the C₃₋₈cycloalkyl or heterocyclyl moiety; or

R⁸ represents —C(═O)—NR¹⁴R¹⁵; wherein

R¹⁴ represents hydrogen, amino, cyano, N,N-diC₁₋₄alkylamino, C₁₋₆alkyl, C₁₋₆alkoxy, C₂₋₆alkenyl, C₂₋₆alkynyl, cyanoC₁₋₆alkyl, C₁₋₆haloalkyl, C₃₋₆haloalkenyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkylcarbonylC₂₋₆alkenyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₄alkylsulfanylC₁₋₆alkyl, C₁₋₆alkylsulfonylC₁₋₆alkyl, C₁₋₆alkylsulfonylaminoC₁₋₆alkyl, or

R¹⁴ represents C₃₋₈cycloalkyl, C₃₋₈cycloalkylC₁₋₆alkyl, C₃₋₈cycloalkylC₁₋₆alkoxy wherein the cycloalkyl moiety is optionally partially unsaturated, phenyl, phenylC₁₋₆alkyl, phenylC₁₋₆alkoxy, heteroaryl, heteroarylC₁₋₆alkyl, heteroarylC₁₋₆alkoxy, wherein the heteroaryl moiety is a 5- or 6-membered monocyclic aromatic ring which comprises 1, 2, 3 or 4 heteroatoms individually selected from N, O and S, heterocyclyl, heterocyclylC₁₋₆alkyl, heterocyclylC₁₋₆alkoxy wherein the heterocyclyl moiety is a 4- to 6-membered non-aromatic ring which comprises 1, 2 or 3 heteroatoms individually selected from N, O and S;

wherein for R¹⁴, any C₃₋₈cycloalkyl, phenyl, heteroaryl or heterocyclyl is optionally substituted by 1, 2 or 3 substituents, which may be the same or different, selected from R¹⁶;

R¹⁶ represents cyano, halogen, hydroxy, C₁₋₄alkyl, C₂₋₄alkenyl, C₂₋₄alkynyl, C₁₋₄haloalkyl, C₂₋₄haloalkenyl, C₁₋₄alkoxy, C₁₋₄haloalkoxy, C₃₋₄alkenyloxy, C₃₋₄alkynyloxy, N—C₁₋₄alkylamino, N,N-diC₁₋₄alkylamino, C₁₋₄alkylcarbonyl, C₁₋₄alkoxycarbonyl, carbonylamino, N—C₁₋₄alkylaminocarbonyl, N,N-diC₁₋₄alkylaminocarbonyl or C₁₋₄alkoxycarbonylamino;

and wherein when R¹⁴ is substituted C₃₋₈cycloalkyl, C₃₋₈cycloalkylC₁₋₆alkyl, C₃₋₈cycloalkylC₁₋₆alkoxy, heterocyclyl, heterocyclylC₁₋₆alkyl or heterocyclylC₁₋₆alkoxy, R¹⁶ may also represent oxo on the C₃₋₈cycloalkyl or heterocyclyl moiety;

R¹⁵ is hydrogen, C₁₋₄alkyl, C₁₋₄alkoxyC₁₋₄alkyl, cyanoC₁₋₄alkyl, N-diC₁₋₄alkylamino; or

R¹⁴ and R¹⁵ together with the nitrogen atom to which they are bonded form a 4-, 5- or 6-membered cycle optionally containing a further heteroatom or group selected from O, S, S(O)₂, C(O) or NR¹⁷; and

R¹⁷ is hydrogen, methyl, methoxy, formyl or acyl; or

a salt or an N-oxide thereof.

Surprisingly, it has been found that the novel compounds of formula (I) have, for practical purposes, a very advantageous level of biological activity for protecting plants against diseases that are caused by fungi.

According to a second aspect of the invention, there is provided an agrochemical composition comprising a fungicidally effective amount of a compound of formula (I). Such an agricultural composition may further comprise at least one additional active ingredient and/or an agrochemically-acceptable diluent or carrier.

According to a third aspect of the invention, there is provided a method of controlling or preventing infestation of useful plants by phytopathogenic microorganisms, wherein a fungicidally effective amount of a compound of formula (I), or a composition comprising this compound as active ingredient, is applied to the plants, to parts thereof or the locus thereof.

According to a fourth aspect of the invention, there is provided the use of a compound of formula (I) as a fungicide. According to this particular aspect of the invention, the use may exclude methods for the treatment of the human or animal body by surgery or therapy.

As used herein, the term “halogen” or “halo” refers to fluorine, chlorine, bromine or iodine, preferably fluorine, chlorine or bromine.

As used herein, the term “C₁₋₆alkyl” refers to a straight or branched hydrocarbon chain radical consisting solely of carbon and hydrogen atoms, containing no unsaturation, having from one to six carbon atoms, and which is attached to the rest of the molecule by a single bond. C₁₋₄alkyl is to be construed accordingly. Examples of C₁₋₆ alkyl include, but are not limited to, methyl, ethyl, n-propyl, 1-methylethyl (iso-propyl), n-butyl, and 1-dimethylethyl (t-butyl). A “C₁₋₆alkylene” group refers to the corresponding definition of C₁₋₆alkyl, except that such radical is attached to the rest of the molecule by two single bonds. Examples of C₁₋₆alkylene, include, but are not limited to, —CH₂—, —CH₂CH₂— and —(CH₂)₃—.

As used herein, cyano means a —CN group.

As used herein, hydroxy means an —OH group.

As used herein, “formyl” means a —C(O)H group and “acyl” means a —C(O)CH₃ group.

As used herein, the term “C₁₋₆alkoxy” refers to a radical of the formula —ORₐ where Rₐ is a C₁₋₆ alkyl radical as generally defined above. C₁₋₂alkoxy and C₁₋₄alkoxy are to be construed accordingly. Examples of C₁₋₆alkoxy include, but are not limited to, methoxy, ethoxy, propoxy, iso-propoxy, butoxy.

As used herein, the term “C₁₋₆haloalkyl” refers to a C₁₋₆ alkyl radical as generally defined above substituted by one or more of the same or different halogen atoms. C₁₋₄haloalkyl is to be construed accordingly. Examples of C₁₋₆haloalkyl include, but are not limited to fluoromethyl, fluoroethyl, difluoromethyl, trifluoromethyl, 2,2,2-trifluoroethyl.

As used herein, the term “C₂₋₆alkenyl” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule by a single bond. C₃₋₄alkenyl and C₃₋₆alkenyl are to be construed accordingly. Examples of C₂₋₆ alkenyl include, but are not limited to, ethenyl, prop-1-enyl, but-1-enyl.

As used herein, the term “C₂₋₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. The term “C₃₋₆alkynyl” is to be construed accordingly. Examples of C₂₋₆alkynyl include, but are not limited to, ethynyl, prop-1-ynyl, but-1-ynyl.

As used herein, the term “C₁₋₄haloalkoxy” refers to a C₁₋₄alkoxy group as defined above substituted by one or more of the same or different halogen atoms. C₁₋₂haloalkoxy is to be construed accordingly. Examples of C₁₋₄haloalkoxy include, but are not limited to, fluoromethoxy, difluoromethoxy, fluoroethoxy, trifluoromethoxy, trifluoroethoxy.

As used herein, the term “C₁₋₄alkoxyC₁₋₆alkyl” refers to radical of the formula R_{b}—O—Rₐ— where R_{b} is a C₁₋₄alkoxy radical as generally defined above, and Rₐ is a C₁₋₆alkyl radical as generally defined above. C₁₋₄alkoxyC₁₋₄alkyl and C₁₋₂alkoxyC₁₋₄alkyl is to be construed accordingly.

As used herein, the term “C₁₋₄haloalkoxyC₁₋₆alkyl” refers to radical of the formula R_{b}—O—Rₐ— where R_{b} is a C₁₋₄haloalkoxy radical as generally defined above, and Rₐ is a C₁₋₆alkyl radical as generally defined above. C₁₋₂haloalkoxyC₁₋₄alkyl is to be construed accordingly.

As used herein, the term “C₂₋₆haloalkenyl” refers to a C₂₋₆ alkenyl radical as generally defined above substituted by one or more of the same or different halogen atoms. C₂₋₄haloalkenyl and C₃₋₆haloalkenyl are to be construed accordingly.

As used herein, the term “hydroxyC₁₋₆alkyl” refers to a C₁₋₆alkyl radical as generally defined above substituted by one or more hydroxyl groups. HydroxyC₁₋₄alkyl is to be construed accordingly.

As used herein, the term “aminoC₁₋₆alkyl” refers to a C₁₋₆alkyl radical as generally defined above substituted by one or more amino (—NH₂) groups. AminoC₁₋₆alkyl is to be construed accordingly.

As used herein, the term “C₁₋₄alkoxyC₁₋₄alkoxyC₁₋₆alkyl” refers to radical of the formula R_{b}—O—Rₐ— where R_{b} is a C₁₋₄alkoxy radical as generally defined above, and Rₐ is a C₁₋₄alkoxyC₁₋₆alkyl radical as generally defined above.

As used herein, the term “C₁₋₆alkylcarbonyl” refers to a radical of the formula —C(O)Rₐ where Rₐ is a C₁₋₆alkyl radical as generally defined above. C₁₋₄alkylcarbonyl is to be construed accordingly.

As used herein, the term “C₃₋₆alkenyloxy” refers to a radical of the formula —ORₐ where Rₐ is a C₃₋₆alkenyl radical as generally defined above. C₃₋₄alkenyloxy is to be construed accordingly.

As used herein, the term “C₃₋₆alkynyloxy” refers to a radical of the formula —ORₐ where Rₐ is a C₃₋₆alkynyl radical as generally defined above. C₃₋₄alkynyloxy is to be construed accordingly.

As used herein, the term “C₃₋₆haloalkenyloxy” refers to radical of the formula —ORₐ where Rₐ is a C₃₋₆haloalkenyl radical as generally defined above. C₃₋₄haloalkenyloxy is to be construed accordingly.

As used herein, the term “C₁₋₆alkylsulfanyl” refers to a radical of the formula —SRₐ where Rₐ is a C₁₋₆alkyl radical as generally defined above. C₁₋₄alkylsulfanyl is to be construed accordingly.

As used herein, the term “C₁₋₆alkylsulfonyl” refers to a radical of the formula —S(O)₂Rₐ where Rₐ is a C₁₋₆alkyl radical as generally defined above. C₁₋₄alkylsulfonyl is to be construed accordingly.

As used herein, the term “C₁₋₆alkylsulfonylamino” refers to a radical of the formula —HNS(O)₂Rₐ where Rₐ is a C₁₋₆alkyl radical as generally defined above. C₁₋₄alkylsulfonylamino is to be construed accordingly.

As used herein, the term “C₁₋₄alkoxycarbonyl” refers to a radical of the formula —C(O)ORₐ where Rₐ is a C₁₋₄alkyl radical as generally defined above.

As used herein, the term “C₁₋₆alkoxycarbonylamino” refers to a radical of the formula —HNC(O)ORₐ where Rₐ is a C₁₋₆alkyl radical as generally defined above. C₁₋₄alkoxycarbonylamino is to be construed accordingly.

As used herein, the term “C₁₋₆alkylcarbonyloxy” refers to a radical of the formula —OC(O)Rₐ where Rₐ is a C₁₋₆alkyl radical as generally defined above.

As used herein, the term “N—C₁₋₄alkoxyamino” refers to a radical of the formula —NH—Rₐ where Rₐ is a C₁₋₄alkoxy radical as defined above.

As used herein, the term “N—C₁₋₄alkylamino” refers to a radical of the formula —NH—Rₐ where Rₐ is a C₁₋₄alkyl radical as defined above.

As used herein, the term “N,N-diC₁₋₄alkylamino” refers to a radical of the formula —N(Rₐ)—Rₐ where each Rₐ is a C₁₋₄alkyl radical, which may be the same or different, as defined above.

As used herein, the term “N—C₁₋₄alkylaminocarbonyl” refers to a radical of the formula —C(O)NHRₐ where Rₐ is a C₁₋₄alkyl radical as generally defined above.

As used herein, the term “N,N-diC₁₋₄alkylaminocarbonyl” refers to a radical of the formula —C(O)NRₐ(Rₐ) where each Rₐ is a C₁₋₄alkyl radical as generally defined above.

As used herein, the term “heteroaryl” refers to a 5- or 6-membered monocyclic aromatic ring radical which comprises 1, 2, 3 or 4 heteroatoms individually selected from nitrogen, oxygen and sulfur. The heteroaryl radical may be bonded via a carbon atom or heteroatom. Examples of heteroaryl include, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl or pyridyl.

As used herein, the term “C₃₋₈cycloalkyl” refers to a stable, monocyclic ring radical which is saturated or unsaturated and contains 3 to 8 carbon atoms. C₃₋₆cycloalkyl is to be construed accordingly. Examples of C₃₋₈cycloalkyl include, but are not limited to, cyclopropyl, cyclobutyl, cyclopentyl and cyclohexyl.

As used herein, the term “heterocyclyl” or “heterocyclic” refers to a stable, 5- or 6-membered non-aromatic monocyclic ring radical which comprises 1, 2, or 3, heteroatoms individually selected from nitrogen, oxygen and sulfur. The heterocyclyl radical may be bonded to the rest of the molecule via a carbon atom or heteroatom. Examples of heterocyclyl include, but are not limited to, pyrrolinyl, pyrrolidyl, tetrahydrofuryl, tetrahydrothienyl, tetrahydrothiopyranyl, piperidyl, piperazinyl, tetrahydropyranyl, dioxolanyl, morpholinyl or perhydroazepinyl.

As used herein, the term “phenylC₁₋₄alkyl” refers to a phenyl ring attached to the rest of the molecule by a C₁₋₄alkylene radical as defined above. The term “phenylC₁₋₂alkyl” should be construed accordingly. Examples of phenylC₁₋₄alkyl include, but are not limited to, benzyl.

As used herein, the term “heteroarylC₁₋₄alkyl” refers to a heteroaryl ring as defined above which is attached to the rest of the molecule by a C₁₋₄alkylene radical as defined above. Likewise, the terms “heteroarylC₁₋₂alkyl” is to be construed accordingly.

As used herein, the term “C₃₋₈cycloalkylC₁₋₄alkyl” refers to a C₃₋₈cycloalkyl ring as defined above attached to the rest of the molecule by a C₁₋₄alkylene radical as defined above. The terms “C₃₋₆cycloalkylC₁₋₂alkyl” and “C₃₋₄cycloalkylC₁₋₂alkyl” are to be construed accordingly. Examples of C₃₋₈cycloalkylC₁₋₄alkyl include, but are not limited to cyclopropyl-methyl, cyclobutyl-ethyl, cyclopentyl-propyl.

As used herein, the term “heterocyclylC₁₋₄alkyl” refers to a heterocyclic ring as defined above which is attached to the rest of the molecule by a C₁₋₄alkylene radical as defined above. The term “heterocyclylC₁₋₂alkyl” should be construed accordingly.

As used herein, the term “phenylC₁₋₆alkoxy” refers to a phenyl ring attached to the rest of the molecule by a C₁₋₆alkoxy radical as defined above. The term “phenylC₁₋₂alkoxy” should be construed accordingly.

As used herein, the term “heteroarylC₁₋₆alkoxy” refers to a heteroaryl ring as defined above which is attached to the rest of the molecule by a C₁₋₆alkoxy radical as defined above. Likewise, the term “heteroarylC₁₋₂alkoxy” is to be construed accordingly.

As used herein, the term “C₃₋₈cycloalkylC₁₋₆alkoxy” refers to a C₃₋₈cycloalkyl ring as defined above attached to the rest of the molecule by a C₁₋₆alkoxy radical as defined above. The terms “C₃₋₆cycloalkylC₁₋₂alkoxy” and “C₃₋₄cycloalkylC₁₋₂alkoxy” are to be construed accordingly.

As used herein, the term “heterocyclylC₁₋₆alkoxy” refers to a heterocyclic ring as defined above which is attached to the rest of the molecule by a C₁₋₆alkoxy radical as defined above. The term “heterocyclylC₁₋₂alkoxy” should be construed accordingly.

The presence of one or more possible asymmetric carbon atoms in a compound of formula (I) means that the compounds may occur in chiral isomeric forms, i.e., enantiomeric or diastereomeric forms. Also atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers (including lactam-lactim tautomerism and keto-enol tautomerism) where present. The present invention includes all possible tautomeric forms for a compound of formula (I).

In each case, the compounds of formula (I) according to the invention are in free form, in oxidized form as an N-oxide, in covalently hydrated form, or in salt form, e.g., an agronomically usable or agrochemically acceptable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The following list provides definitions, including preferred definitions, for substituents n, A¹, A², A³, A⁴, R¹, R², R³, R⁴, R⁵, R⁶, R⁷, R⁸, R⁹, R¹⁰, R¹¹, R¹², R¹³, R¹⁴, R¹⁵, R¹⁶ and R¹⁷ with reference to the compounds of formula (I). For any one of these substituents, any of the definitions given below may be combined with any definition of any other substituent given below or elsewhere in this document.

n represents 0, 1 or 2. In some embodiments of the invention, n is 0. In other embodiments of the invention, n is 1. In other embodiments of the invention, n is 2. Preferably, n is 0 or 1, and more preferably 1.

A¹ represents N or CR¹, wherein R¹ represents hydrogen, halogen, methyl, ethyl, trifluoromethyl, methoxy, ethoxy, or difluoromethoxy. Preferably, A¹ represents N or CR¹, wherein R¹ is selected from hydrogen, chloro, fluoro, methyl, methoxy, or trifluoromethyl.

A² represents N or CR², wherein R² represents hydrogen, halogen, methyl, ethyl, trifluoromethyl, methoxy, ethoxy, or difluoromethoxy. Preferably, A² represents CR² and R² is selected from hydrogen, chloro, fluoro, methyl, methoxy, or trifluoromethyl.

A³ represents N or CR³, wherein R³ represents hydrogen or halogen. Preferably, A³ represents CR³ and R³ is hydrogen.

A⁴ represents N or CR⁴, wherein R⁴ represents hydrogen or halogen. Preferably, A⁴ represents CR⁴ and R⁴ is hydrogen.

In some embodiments, A³ represents CR³ and R³ is hydrogen and A⁴ represents CR⁴ and R⁴ is hydrogen.

In the compounds according to Formula (I) of the invention, no more than two of A¹ to A⁴ are N (nitrogen). Preferably, one or none of A¹ to A⁴ are N, in particular, A¹ may be N and A² to A⁴ are all C—H. Most preferably, none of A¹ to A⁴ are N, ie, all of A¹ to A⁴ correspond to CR¹, CR², CR³, CR⁴, respectively. Even more preferably, none of A¹ to A⁴ are N, and A¹ to A⁴ are all C—H.

In some embodiments of the invention, the 6-membered ring comprising A¹ to A⁴ is a phenyl (where A¹, A², A³ and A⁴ are C—H), a pyridinyl (where A¹ is N and A², A³ and A⁴ are C—H, or A³ is N and A¹, A² and A⁴ are C—H), a fluorophenyl (where A¹ is C—F and A², A³ and A⁴ are C—H, or A³ is C—F and A¹, A² and A⁴ are C—H) or a difluorophenyl (eg, where A¹ and A² are C—F and A³ and A⁴ are C—H, or A and A³ are C—F and A² and A⁴ are C—H) group.

R⁵ and R⁶ independently represent hydrogen, C₁₋₄alkyl, halogen, cyano, trifluoromethyl and difluoromethyl, or R⁵ and R⁶ together with the carbon atom they share form a cyclopropyl. Preferably, R⁵ and R⁶ are independently selected from hydrogen and C₁₋₄alkyl. More preferably, R⁵ and R⁶ are independently selected from hydrogen and methyl, or R⁵ and R⁶ are hydrogen.

R⁷ represents hydrogen, hydroxy, C₁₋₄alkyl, C₁₋₄haloalkyl, C₁₋₄alkoxy, C₁₋₄haloalkoxy, hydroxyC₁₋₄alkyl, C₁₋₂alkoxyC₁₋₄alkyl, C₁₋₂haloalkoxyC₁₋₄alkyl, cyanoC₁₋₄alkyl, C₃₋₆alkenyl, C₃₋₆alkynyl, C₃₋₆alkenyloxy, C₃₋₆alkynyloxy, C₃₋₆haloalkenyl, C₃₋₆haloalkenyloxy, or C₃₋₆cycloalkyl, C₃₋₆cycloalkylC₁₋₂alkyl, C₃₋₆cycloalkylC₁₋₂alkoxy, phenyl, phenylC₁₋₂alkyl, phenylC₁₋₂alkoxy, heteroaryl, heteroarylC₁₋₂alkyl, heteroarylC₁₋₂alkoxy, heterocyclyl, heterocyclylC₁₋₂alkyl, or heterocyclylC₁₋₂alkoxy, wherein the heteroaryl moiety is a 5- or 6-membered monocyclic aromatic ring which comprises 1, 2, 3 or 4 heteroatoms individually selected from N, O and S, the heterocyclyl moiety is a 4- to 6-membered non-aromatic ring which comprises 1 or 2 heteroatoms individually selected from N, O and S, and wherein any of cycloalkyl, phenyl, heteroaryl and heterocyclyl moieties are optionally substituted by 1 or 2 substituents selected from cyano, fluoro, chloro, bromo, methyl, difluoromethyl, trifluoromethyl, methoxy, ethoxy, or difluoromethoxy.

Preferably, R⁷ represents hydrogen, C₁₋₄alkyl, C₁₋₄alkoxy, C₁₋₂alkoxyC₁₋₄alkyl, C₃₋₄alkenyl, C₃₋₆alkynyl, C₃₋₆alkenyloxy, C₃₋₆alkynyloxy, C₃₋₆haloalkenyloxy, or C₃₋₆cycloalkyl, C₃₋₆cycloalkylC₁₋₂alkyl, phenyl, phenylC₁₋₂alkyl, heteroaryl or heteroarylC₁₋₂alkyl wherein the heteroaryl moiety is a 5- or 6-membered monocyclic aromatic ring which comprises 1 or 2 heteroatoms individually selected from N and O, heterocyclyl or heterocyclylC₁₋₂alkyl wherein the heterocyclyl moiety is a 4- to 6-membered non-aromatic ring which comprises 1 or 2 heteroatoms individually selected from N and O, and wherein any of cycloalkyl, phenyl, heteroaryl and heterocyclyl moieties are optionally substituted by 1 or 2 substituents selected from cyano, fluoro, chloro, bromo, methyl, difluoromethyl, trifluoromethyl, methoxy, ethoxy, or difluoromethoxy. More preferably, R⁷ represents hydrogen, C₁₋₄alkyl, C₁₋₄alkoxy, C₁₋₂alkoxyC₁₋₄alkyl, C₃₋₄alkenyl, C₃₋₄alkynyl, C₃₋₆cycloalkyl or C₃₋₆cycloalkylC₁₋₂alkyl. Even more preferably, R⁷ represents hydrogen, C₁₋₄alkyl, methoxy, ethoxy or cyclopropyl. Still more preferably, R⁷ represents hydrogen, methyl or methoxy.

R⁸ represents —C(═R⁹)—R¹⁰, wherein R⁹ represents O or N—C₁₋₄alkoxy.

In one embodiment of the invention, R⁸ represents —C(═O)—R¹⁰. In another embodiment, R⁸ represents —C(═N—C₁₋₄alkoxy)-R¹⁰, and preferably, R⁹ represents methoxy.

R¹⁰ represents hydrogen, cyano, C₁₋₆alkyl, C₂₋₆alkenyl, C₃₋₆alkenyloxy, C₂₋₆alkynyl, cyanoC₁₋₆alkyl, C₁₋₆haloalkyl, C₃₋₆haloalkenyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkylcarbonylC₂₋₆alkenyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₆alkylsulfanylC₁₋₆alkyl, C₁₋₆alkylsulfonylC₁₋₆alkyl, C₁₋₆alkylsulfonylaminoC₁₋₆alkyl, or R¹⁰ represents C₃₋₈cycloalkyl, C₃₋₈cycloalkylC₁₋₆alkyl, C₃₋₈cycloalkylC₁₋₆alkoxy wherein the cycloalkyl moiety is optionally partially unsaturated, phenyl, phenylC₁₋₆alkyl, phenylC₁₋₆alkoxy, heteroaryl, heteroarylC₁₋₆alkyl, heteroarylC₁₋₆alkoxy, wherein the heteroaryl moiety is a 5- or 6-membered monocyclic aromatic ring which comprises 1, 2, 3 or 4 heteroatoms individually selected from N, O and S, heterocyclyl, heterocyclylC₁₋₆alkyl, heterocyclylC₁₋₆alkoxy wherein the heterocyclyl moiety is a 4- to 6-membered non-aromatic ring which comprises 1, 2 or 3 heteroatoms individually selected from N, O and S; wherein for R¹⁰, any of C₃₋₈cycloalkyl, C₃₋₈cycloalkylC₁₋₆alkyl, C₃₋₈cycloalkylC₁₋₆alkoxy, phenyl, phenylC₁₋₆alkyl, phenylC₁₋₆alkoxy, heteroaryl, heteroarylC₁₋₆alkyl, heteroarylC₁₋₆alkoxy, heterocyclyl, heterocyclylC₁₋₆alkyl and heterocyclylC₁₋₆alkoxy are optionally substituted by 1, 2 or 3 substituents, which may be the same or different, selected from R¹¹; and wherein when R¹⁰ is substituted C₃₋₈cycloalkyl, C₃₋₈cycloalkylC₁₋₆alkyl, C₃₋₈cycloalkylC₁₋₆alkoxy, heterocyclyl, heterocyclylC₁₋₆alkyl or heterocyclylC₁₋₆alkoxy, R¹¹ may also represent oxo on the C₃₋₈cycloalkyl or heterocyclyl moiety.

Preferably, R¹⁰ represents hydrogen, C₁₋₆alkyl, cyanoC₁₋₆alkyl, C₁₋₆haloalkyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₆alkylsulfanylC₁₋₆alkyl, C₁₋₆alkylsulfonylC₁₋₆alkyl, C₁₋₆alkylsulfonylaminoC₁₋₆alkyl, or phenyl, phenylC₁₋₂alkyl, heteroaryl or heteroarylC₁₋₂alkyl each which may be optionally substituted by 1 or 2 substituents, which may be the same or different, selected from R¹¹.

More preferably, R¹⁰ is hydrogen, C₁₋₆alkyl, C₃₋₆cycloalkyl, furanyl, thienyl, phenyl or phenylC₁₋₂alkyl, wherein phenyl on each phenyl or phenylC₁₋₂alkyl may optionally be substituted by 1 substituent selected from R¹¹ wherein R¹¹ is selected from cyano, halogen, hydroxy, methyl or methoxy. Even more preferably, R¹⁰ is hydrogen, methyl, ethyl, n-propyl, iso-propyl, cyclopropyl, phenyl, furan-2-yl or thien-2-yl, wherein cyclopropyl, phenyl, furan-2-yl or thien-2-yl are optionally substituted by 1 substituent selected from R¹¹, wherein R¹¹ is selected from cyano, halogen, hydroxy, methyl or methoxy.

In some embodiments, R¹⁰ is hydrogen, C₁₋₆alkyl, phenyl or phenylC₁₋₂alkyl, wherein phenyl on each phenyl or phenylC₁₋₂alkyl may be optionally substituted by 1 substituent selected from R¹¹.

R¹¹ represents cyano, halogen, hydroxy, C₁₋₄alkyl, C₂₋₄alkenyl, C₂₋₄alkynyl, C₁₋₄haloalkyl, C₂₋₄haloalkenyl, C₁₋₄alkoxy, C₁₋₄haloalkoxy, C₃₋₄alkenyloxy, C₃₋₄alkynyloxy, N—C₁₋₄alkylamino, N,N-diC₁₋₄alkylamino, C₁₋₄alkylcarbonyl, C₁₋₄alkoxycarbonyl, carbonylamino, N—C₁₋₄alkylaminocarbonyl, N,N-diC₁₋₄alkylaminocarbonyl or C₁₋₄alkoxycarbonylamino. Preferably, R¹¹ represents cyano, halogen, hydroxy, C₁₋₄alkyl or C₁₋₄alkoxy. More preferably, R¹¹ represents cyano, halogen, hydroxy, methyl or methoxy.

R⁸ also represents —C(═O)—OR¹².

R¹² represents hydrogen, C₁₋₄alkyl, C₃₋₆alkenyl, C₃₋₆alkynyl, cyanoC₁₋₆alkyl, C₁₋₆haloalkyl, C₃₋₆haloalkenyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkylcarbonylC₂₋₆alkenyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₄alkylsulfanylC₁₋₆alkyl, C₁₋₆alkylsulfonylC₁₋₆alkyl, C₁₋₆alkylsulfonylaminoC₁₋₆alkyl, or R¹² represents C₃₋₈cycloalkyl or C₃₋₈cycloalkylC₁₋₆alkyl wherein the cycloalkyl moiety is optionally partially unsaturated, phenyl or phenylC₁₋₆alkyl heteroaryl or heteroarylC₁₋₆alkyl wherein the heteroaryl moiety is a 5- or 6-membered monocyclic aromatic ring which comprises 1, 2, 3 or 4 heteroatoms individually selected from N, O and S, heterocyclyl or heterocyclylC₁₋₆alkyl wherein the heterocyclyl moiety is a 4- to 6-membered non-aromatic ring which comprises 1, 2 or 3 heteroatoms individually selected from N, O and S, wherein for R¹², any of C₃₋₈cycloalkyl, C₃₋₈cycloalkylC₁₋₆alkyl, phenyl, phenylC₁₋₆alkyl, heteroaryl, heteroarylC₁₋₆alkyl, heterocyclyl and heterocyclylC₁₋₆alkyl are optionally substituted by 1, 2 or 3 substituents, which may be the same or different, selected from R¹³; and wherein when R¹² is substituted C₃₋₈cycloalkyl, C₃₋₈cycloalkylC₁₋₆alkyl, heterocyclyl or heterocyclylC₁₋₆alkyl, R¹³ may also represent oxo on the C₃₋₈cycloalkyl or heterocyclyl moiety.

Preferably, R¹² represents hydrogen, C₁₋₄alkyl, cyanoC₁₋₆alkyl, C₁₋₆haloalkyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₄alkylsulfanylC₁₋₆alkyl, C₁₋₆alkylsulfonylC₁₋₆alkyl, C₁₋₆alkylsulfonylaminoC₁₋₆alkyl, or C₃₋₈cycloalkyl, C₃₋₈cycloalkylC₁₋₂alkyl, heterocyclyl, heterocyclylC₁₋₂alkyl wherein each C₃₋₈cycloalkyl or heterocyclyl moiety is optionally substituted by 1 or 2 substituents, which may be the same or different, selected from R¹³. More preferably, R¹² represents hydrogen, C₁₋₆alkyl, C₁₋₄alkoxyC₁₋₆alkyl, C₄₋₆cycloalkyl or heterocyclyl wherein the heterocyclyl moiety is a 4- to 6-membered non-aromatic ring which comprises 1 heteroatom selected from N, O or S, wherein C₄₋₆cycloalkyl or heterocyclyl may each optionally be substituted by 1 or 2 substituents, which may be the same or different, selected from R¹³.

R¹³ represents cyano, halogen, hydroxy, C₁₋₄alkyl, C₂₋₄alkenyl, C₂₋₄alkynyl, C₁₋₄haloalkyl, C₂₋₄haloalkenyl, C₁₋₄alkoxy, C₁₋₄haloalkoxy, C₃₋₄alkenyloxy, C₃₋₄alkynyloxy, N—C₁₋₄alkylamino, N,N-diC₁₋₄alkylamino, C₁₋₄alkylcarbonyl, C₁₋₄alkoxycarbonyl, carbonylamino, N—C₁₋₄alkylaminocarbonyl, N,N-diC₁₋₄alkylaminocarbonyl or C₁₋₄alkoxycarbonylamino. Preferably, R¹³ represents cyano, halogen, hydroxy, C₁₋₄alkyl or C₁₋₄alkoxy. More preferably, R¹³ represents cyano, halogen, hydroxy, methyl or methoxy.

R⁸ also represents —C(═O)—NR¹⁴R¹⁴R¹⁵.

R¹⁴ represents hydrogen, amino, cyano, N,N-diC₁₋₄alkylamino, C₁₋₆alkyl, C₁₋₆alkoxy, C₂₋₆alkenyl, C₂₋₆alkynyl, cyanoC₁₋₆alkyl, C₁₋₆haloalkyl, C₃₋₆haloalkenyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkylcarbonylC₂₋₆alkenyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₄alkylsulfanylC₁₋₆alkyl, C₁₋₆alkylsulfonylC₁₋₆alkyl, C₁₋₆alkylsulfonylaminoC₁₋₆alkyl, or R¹⁴ represents C₃₋₈cycloalkyl, C₃₋₈cycloalkylC₁₋₆alkyl, C₃₋₈cycloalkylC₁₋₆alkoxy wherein the cycloalkyl moiety is optionally partially unsaturated, phenyl, phenylC₁₋₆alkyl, phenylC₁₋₆alkoxy, heteroaryl, heteroarylC₁₋₆alkyl, heteroarylC₁₋₆alkoxy, wherein the heteroaryl moiety is a 5- or 6-membered monocyclic aromatic ring which comprises 1, 2, 3 or 4 heteroatoms individually selected from N, O and S, heterocyclyl, heterocyclylC₁₋₆alkyl, heterocyclylC₁₋₆alkoxy wherein the heterocyclyl moiety is a 4- to 6-membered non-aromatic ring which comprises 1, 2 or 3 heteroatoms individually selected from N, O and S; wherein for R¹⁴, any of C₃₋₈cycloalkyl, C₃₋₈cycloalkylC₁₋₆alkyl, C₃₋₈cycloalkylC₁₋₆alkoxy, phenyl, phenylC₁₋₆alkyl, phenylC₁₋₆alkoxy, heteroaryl, heteroarylC₁₋₆alkyl, heteroarylC₁₋₆alkoxy, heterocyclyl, heterocyclylC₁₋₆alkyl and heterocyclylC₁₋₆alkoxy are optionally substituted by 1, 2 or 3 substituents, which may be the same or different, selected from R¹⁶; and wherein when R¹⁴ is substituted C₃₋₈cycloalkyl, C₃₋₈cycloalkylC₁₋₆alkyl, C₃₋₈cycloalkylC₁₋₆alkoxy, heterocyclyl, heterocyclylC₁₋₆alkyl or heterocyclylC₁₋₆alkoxy, R¹⁶ may also represent oxo on the C₃₋₈cycloalkyl or heterocyclyl moiety.

Preferably, R¹⁴ represents hydrogen, C₁₋₆alkyl, C₁₋₆alkoxy, C₂₋₆alkenyl, C₂₋₆alkynyl, cyanoC₁₋₆alkyl, C₁₋₄alkoxyC₁₋₆alkyl, C₁₋₆alkylsulfonylC₁₋₆alkyl, or C₃₋₈cycloalkyl, C₃₋₈cycloalkylC₁₋₆alkyl, phenyl, phenylC₁₋₆alkyl, heteroaryl or heteroarylC₁₋₆alkyl wherein the heteroaryl moiety is a 5- or 6-membered monocyclic aromatic ring which comprises 1 or 2 heteroatoms individually selected from N, O and S, heterocyclyl or heterocyclylC₁₋₆alkyl wherein the heterocyclyl moiety is a 4- to 6-membered non-aromatic ring which comprises 1 or 2 heteroatoms individually selected from N and O, wherein C₃₋₈cycloalkyl, C₃₋₈cycloalkylC₁₋₆alkyl, phenyl, phenylC₁₋₆alkyl, heteroaryl, heteroarylC₁₋₆alkyl, heterocyclyl or heterocyclylC₁₋₆alkyl are optionally substituted by 1 or 2 substituents, which may be the same or different, selected from R¹⁶. More preferably, R¹⁴ represents hydrogen, C₁₋₆alkyl, C₁₋₄alkoxy, C₂₋₄alkenyl, C₂₋₄alkynyl, cyanoC₁₋₄alkyl, C₁₋₄alkoxyC₁₋₄alkyl, C₁₋₄alkylsulfonylC₁₋₄alkyl, or C₃₋₆cycloalkyl, C₃₋₆cycloalkylC₁₋₂alkyl, phenyl, phenylC₁₋₂alkyl, heteroaryl or heteroarylC₁₋₂alkyl wherein the heteroaryl moiety is a 5- or 6-membered monocyclic aromatic ring which comprises 1 or 2 heteroatoms individually selected from N and O, heterocyclyl or heterocyclylC₁₋₂alkyl wherein the heterocyclyl moiety is a 4- to 6-membered non-aromatic ring which comprises 1 or 2 heteroatoms individually selected from N and O, wherein C₃₋₆cycloalkyl, C₃₋₆cycloalkylC₁₋₂alkyl, phenyl, phenylC₁₋₂alkyl, heteroaryl, heteroarylC₁₋₂alkyl, heterocyclyl or heterocyclylC₁₋₂alkyl are optionally substituted by 1 or 2 substituents, which may be the same or different, selected from R¹⁶. Even more preferably, R¹⁴ represents hydrogen, C₁₋₄alkyl, methoxy, ethoxy, methoxyethyl, cyclopropyl, cyclopropylmethyl, 1,4-dioxanyl (including 1,4-dioxan-2-yl), tetrahydrofuranyl (including tetrahydrofuran-3-yl), wherein cyclopropyl, 1,4-dioxanyl or tetrahydrofuranyl are optionally substituted by 1 or 2 substituents, which may be the same or different, selected from R¹⁶. Still more preferably, R¹⁴ represents hydrogen or C₁₋₄alkyl, in particular, hydrogen or methyl.

R¹⁵ represents hydrogen, C₁₋₄alkyl, C₁₋₄alkoxyC₁₋₄alkyl, cyanoC₁₋₄alkyl, N-diC₁₋₄alkylamino. Preferably, R¹⁵ represents hydrogen, methyl, ethyl, C₁₋₂alkoxyC₁₋₂alkyl or cyanoC₁₋₂alkyl. More preferably, R¹⁵ represents hydrogen, methyl or ethyl.

R¹⁴ and R¹⁵ together with the nitrogen atom to which they are bonded form a 4-, 5- or 6-membered cycle optionally containing a further heteroatom comprising O or S, or NR¹⁷. Preferably, R¹⁴ and R¹⁵ together with the nitrogen atom to which they are bonded form a 4-, 5- or 6-membered cycle optionally containing a further hetetroatom which is O.

R¹⁶ represents cyano, halogen, hydroxy, C₁₋₄alkyl, C₂₋₄alkenyl, C₂₋₄alkynyl, C₁₋₄haloalkyl, C₂₋₄haloalkenyl, C₁₋₄alkoxy, C₁₋₄haloalkoxy, C₃₋₄alkenyloxy, C₃₋₄alkynyloxy, N—C₁₋₄alkylamino, N,N-diC₁₋₄alkylamino, C₁₋₄alkylcarbonyl, C₁₋₄alkoxycarbonyl, carbonylamino, N—C₁₋₄alkylaminocarbonyl, N,N-diC₁₋₄alkylaminocarbonyl or C₁₋₄alkoxycarbonylamino. Preferably, R¹⁶ represents cyano, halogen, hydroxy, C₁₋₄alkyl, C₁₋₄haloalkyl, C₁₋₄alkoxy, C₁₋₄haloalkoxy, C₃₋₄alkenyloxy, C₃₋₄alkynyloxy, C₁₋₄alkylcarbonyl, C₁₋₄alkoxycarbonyl, N—C₁₋₄alkylaminocarbonyl or N,N-diC₁₋₄alkylaminocarbonyl. More preferably, R¹⁶ represents C₁₋₄alkoxy or C₁₋₄alkoxycarbonyl.

R¹⁷ represents hydrogen, methyl, methoxy, formyl or acyl.

In a compound according to formula (I) of the invention, n can represent 0 and R⁷ can represent hydrogen, C₁₋₄alkyl, or C₁₋₄alkoxy.

In a compound according to formula (I) of the invention, n can represent 2 and R⁵ and R⁶ can represent hydrogen.

In a compound according to formula (I) of the invention, A¹ can represent N or CR¹, where R¹ is selected from hydrogen, fluoro, chloro, methyl, methoxy, or trifluoromethyl, A² can represent CR² and R² is hydrogen or fluorine, A³ can represent CR³ and R³ is hydrogen or fluorine, and A⁴ can represent CR⁴ and R⁴ is hydrogen. In a compound according to formula (I) of the invention, A¹ to A⁴ can be C—H.

Preferably, the compound according to Formula (I) is selected from a compound 1.1 to 1.100 listed in Table T1 (below), or a compound 2.1 to 2.15 listed in Table T2 (below), or a compound 3.1 to 3.10 listed in Table T3 (below), or a compound 4.1 to 4.111 listed in Table T4 (below).

Preferably, in a compound according to formula (I) of the invention, n is 0 or 1;
A¹ represents N or CR¹, wherein R¹ is selected from hydrogen, chloro, fluoro, methyl, methoxy, or trifluoromethyl;
A² to A⁴ all represent C—H;
R⁵ and R⁶ are independently selected from hydrogen and C₁₋₄alkyl;
R⁷ is selected from hydrogen, C₁₋₄alkyl, C₁₋₄alkoxy, C₁₋₂alkoxyC₁₋₄alkyl, C₃₋₄alkenyl, C₃₋₄alkynyl, C₃₋₆cycloalkyl or C₃₋₆cycloalkylC₁₋₂alkyl;
R⁸ is —C(═R⁹)—R¹⁰;
R¹⁰ is selected from hydrogen, C₁₋₆alkyl, cyanoC₁₋₆alkyl, C₁₋₆haloalkyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₆alkylsulfanylC₁₋₆alkyl, C₁₋₆alkylsulfonylC₁₋₆alkyl, C₁₋₆alkylsulfonylaminoC₁₋₆alkyl, or phenyl, phenylC₁₋₂alkyl, heteroaryl or heteroarylC₁₋₂alkyl wherein each phenyl or heteroaryl moiety may be optionally substituted by 1 or 2 substituents, which may be the same or different, selected from R¹¹; and
R¹¹ is selected from cyano, halogen, hydroxy, methyl or methoxy.

Preferably, in a compound according to formula (I) of the invention, n is 0 or 1;
A¹ represents N or CR¹, wherein R¹ is selected from hydrogen, chloro, fluoro, methyl, methoxy, or trifluoromethyl;
A² to A⁴ all represent C—H;
R⁵ and R⁶ are independently selected from hydrogen and C₁₋₄alkyl;
R⁷ is selected from hydrogen, C₁₋₄alkyl, C₁₋₄alkoxy, C₁₋₂alkoxyC₁₋₄alkyl, C₃₋₄alkenyl, C₃₋₄alkynyl, C₃₋₆cycloalkyl or C₃₋₆cycloalkylC₁₋₂alkyl;
R⁸ is —C(═O)—OR¹²;
R¹² is selected from hydrogen, C₁₋₄alkyl, cyanoC₁₋₆alkyl, C₁₋₆haloalkyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₄alkylsulfanylC₁₋₆alkyl, C₁₋₆alkylsulfonylC₁₋₆alkyl, C₁₋₆alkylsulfonylaminoC₁₋₆alkyl, or C₃₋₈cycloalkyl, C₃₋₈cycloalkylC₁₋₂alkyl, heterocyclyl, heterocyclylC₁₋₂alkyl wherein each C₃₋₈cycloalkyl or heterocyclyl moiety is optionally substituted by 1 or 2 substituents, which may be the same or different, selected from R¹³; and
R¹³ represents cyano, halogen, hydroxy, methyl and methoxy.

Preferably, in a compound according to formula (I) of the invention, n is 0 or 1;
A¹ represents N or CR¹, wherein R¹ is selected from hydrogen, chloro, fluoro, methyl, methoxy, or trifluoromethyl;
A² to A⁴ all represent C—H;
R⁵ and R⁶ are independently selected from hydrogen and C₁₋₄alkyl;
R⁷ is selected from hydrogen, C₁₋₄alkyl, C₁₋₄alkoxy, C₁₋₂alkoxyC₁₋₄alkyl, C₃₋₄alkenyl, C₃₋₄alkynyl, C₃₋₆cycloalkyl or C₃₋₆cycloalkylC₁₋₂alkyl;
R⁸ is —C(═O)—NR¹⁴R¹⁵;
R¹⁴ is selected from hydrogen, C₁₋₆alkyl, C₁₋₄alkoxy, C₂₋₄alkenyl, C₂₋₄alkynyl, cyanoC₁₋₄alkyl, C₁₋₄alkoxyC₁₋₄alkyl, C₁₋₄alkylsulfonylC₁₋₄alkyl, or C₃₋₆cycloalkyl, C₃₋₆cycloalkylC₁₋₂alkyl, phenyl, phenylC₁₋₂alkyl, heteroaryl or heteroarylC₁₋₂alkyl wherein the heteroaryl moiety is a 5- or 6-membered monocyclic aromatic ring which comprises 1 or 2 heteroatoms individually selected from N and O, heterocyclyl or heterocyclylC₁₋₂alkyl wherein the heterocyclyl moiety is a 4- to 6-membered non-aromatic ring which comprises 1 or 2 heteroatoms individually selected from N and O, wherein C₃₋₆cycloalkyl, C₃₋₆cycloalkylC₁₋₂alkyl, phenyl, phenylC₁₋₂alkyl, heteroaryl, heteroarylC₁₋₂alkyl, heterocyclyl or heterocyclylC₁₋₂alkyl are optionally substituted by 1 or 2 substituents, which may be the same or different, selected from R¹⁶;
R¹⁶ represents cyano, halogen, hydroxy, C₁₋₄alkyl, C₁₋₄haloalkyl, C₁₋₄alkoxy, C₁₋₄haloalkoxy, C₃₋₄alkenyloxy, C₃₋₄alkynyloxy, C₁₋₄alkylcarbonyl, C₁₋₄alkoxycarbonyl, N—C₁₋₄alkylaminocarbonyl and N,N-diC₁₋₄alkylaminocarbonyl; and
R¹⁵ is selected from hydrogen, methyl, ethyl, C₁₋₂alkoxyC₁₋₂alkyl or cyanoC₁₋₂alkyl.

More preferably, in a compound according to formula (I) of the invention, n is 0 or 1;
A¹ represents N or CR¹, wherein R¹ is selected from hydrogen or fluorine;
A² to A⁴ all represent C—H;
R⁵ and R⁶ are independently selected from hydrogen and methyl;
R⁷ represents hydrogen, methyl or methoxy;
R⁸ is —C(═R⁹)—R¹⁰;
R¹⁰ is selected from hydrogen, C₁₋₆alkyl, phenyl or phenylC₁₋₂alkyl, wherein phenyl on each of phenyl or phenylC₁₋₂alkyl may be optionally substituted by 1 substituent selected from R¹¹; and
R¹¹ is selected from cyano, halogen, hydroxy, methyl or methoxy.

More preferably, in a compound according to formula (I) of the invention, n is 0 or 1;
A¹ represents N or CR¹, wherein R¹ is selected from hydrogen or fluorine;
A² to A⁴ all represent C—H;
R⁵ and R⁶ are independently selected from hydrogen and methyl;
R⁷ represents hydrogen, methyl or methoxy;
R⁸ is —C(═O)—OR¹²;
R¹² represents hydrogen, C₁₋₆alkyl, C₁₋₄alkoxyC₁₋₆alkyl, C₄₋₆cycloalkyl or heterocyclyl wherein the heterocyclyl moiety is a 4- to 6-membered non-aromatic ring which comprises 1 heteroatom selected from N, O or S, wherein C₄₋₆cycloalkyl or heterocyclyl may each optionally be substituted by 1 or 2 substituents, which may be the same or different, selected from R¹³;
R¹³ represents cyano, halogen, hydroxy, methyl or methoxy.

More preferably, in a compound according to formula (I) of the invention, n is 0 or 1;
A¹ represents N or CR¹, wherein R¹ is selected from hydrogen or fluorine;
A² to A⁴ all represent C—H;
R⁵ and R⁶ are independently selected from hydrogen and methyl;
R⁷ is selected from hydrogen, C₁₋₄alkyl, C₁₋₄alkoxy, C₁₋₂alkoxyC₁₋₄alkyl, C₃₋₄alkenyl, C₃₋₄alkynyl, C₃₋₆cycloalkyl or C₃₋₆cycloalkylC₁₋₂alkyl;
R⁸ is —C(═O)—NR¹⁴R¹⁵;
R¹⁴ is selected from hydrogen or C₁₋₄alkyl; and
R¹⁵ is selected from hydrogen or methyl.

The compounds of the present invention (when n=1) may be enantiomers of the compound of Formula (I) as represented by a Formula (Ia) or a Formula (Ib), wherein R⁵ and R⁶ are different.

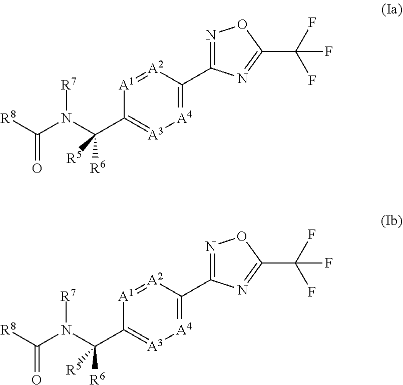

Likewise, the compounds of the present invention may be enantiomers (when n=2) when at one of the two carbon positions bound to R⁵ and R⁶, R⁵ and R⁶ are different substituents and at the other carbon position, R⁵ and R⁶ are the same. Alternatively, the compounds of Formula (I) may be diastereomers (when n=2) when at each of the two carbon positions bound to R⁵ and R⁶, R⁵ and R⁶ are different.

It is understood that when in aqueous media, the compounds of formula (I) according to the invention may be present in a reversible equilibrium with the corresponding covalently hydrated forms (ie, the compounds of formula (I-I) and formula (I-II) as shown below) at the CF₃-oxadiazole motif. This dynamic equilibrium may be important for the biological activity of the compounds of Formula (I). The designations of n, A¹, A², A³, A⁴, R¹, R², R³, R⁴, R⁵, R⁶, R⁷, R⁸, R⁹, R¹⁰, R¹¹, R¹², R¹³, R¹⁴, R¹⁵, R¹⁶ and R¹⁷ with reference to the compounds of formula (I) of the present invention apply generally to the compounds of Formula (I-I) and Formula (I-II), as well as to the specific disclosures of combinations of n, A¹, A², A³, A⁴, R¹, R², R³, R⁴, R⁵, R⁶, R⁷, R⁸, R⁹, R¹⁰, R¹¹, R¹², R¹³, R¹⁴, R¹⁵, R¹⁶ and R¹⁷ as represented in Tables 1.1 to 1.34, 2.1 to 2.33 and 3.1 to 3.31 (below), or the compounds 1.1 to 1.100 described in Table T1 (below), the compounds 2.1 to 2.15 described in Table T2 (below), the compounds 3.1 to 3.10 described in Table T3 (below), or the compounds 4.1 to 4.111 described in Table T4 (below).

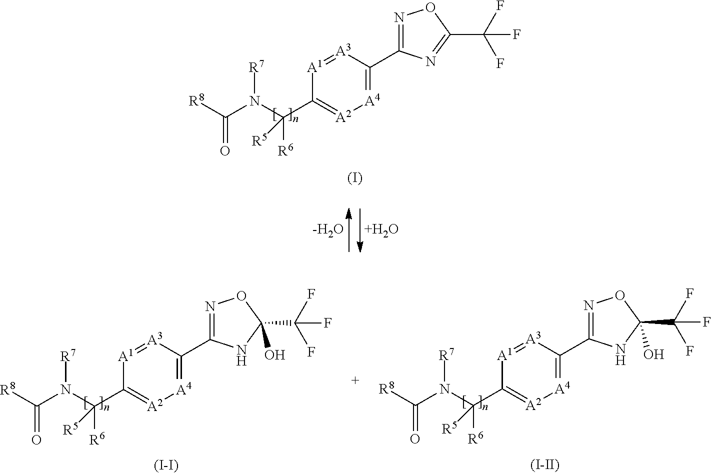

Compounds of the present invention can be made as shown in the following schemes 1 to 17, in which, unless otherwise stated, the definition of each variable is as defined above for a compound of formula (I).

The compounds of formula (I) can be obtained by an amide coupling transformation with compounds of formula (II) and compounds of formula (III) by activating the carboxylic acid function of the compounds of formula (III), a process that usually takes place by converting the —OH of the carboxylic acid into a good leaving group, such as a chloride group, for example by using (COCl)₂ or SOCl₂, prior to treatment with the compounds of formula (II), preferably in a suitable solvent (eg, dimethylformamide, dichloromethane or tetrahydrofuran), preferably at a temperature of between 22° C. and 100° C., and optionally in the presence of a base such as triethylamine or N,N-diisopropylethylamine, or under conditions described in the literature for an amide coupling. For examples, see WO 2003/028729. Compounds of formula (III) are commercially available or prepared using known methods. For related examples, see: Nelson, T. D et al Tetrahedron Lett. (2004), 45, 8917; Senthil, K. et al Pest. Res. Journal (2009), 21, 133; and Crich, D., Zou, Y. J. Org. Chem. (2005), 70, 3309. This reaction is shown in Scheme 1.

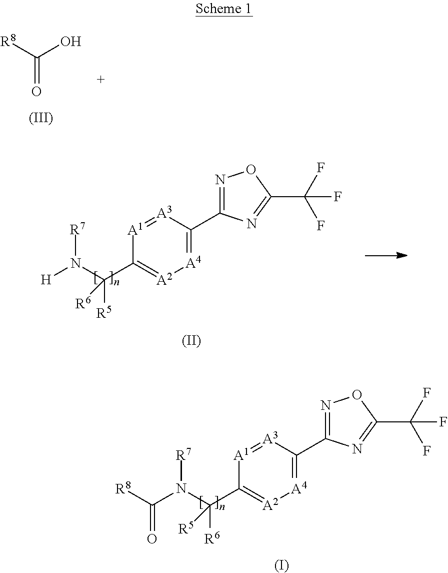

Alternatively, the compounds of formula (I) can be obtained by an amide coupling transformation with compounds of formula (IV), wherein R^{N}—Nu is R¹¹—OH, its alkoxide conjugate, or R^{N}—Nu is R¹³—N(H)—R¹⁴, and compounds of formula (V) by activating the carboxylic acid function of the compounds of formula (V), a process that usually takes place by converting the —OH of the carboxylic acid into a good leaving group, such as a chloride group, for example by using (COCl)₂ or SOCl₂, prior to treatment with the compounds of formula (IV), preferably in a suitable solvent (eg, dimethylformamide, dichloromethane or tetrahydrofuran), preferably at a temperature of between 22° C. and 100° C., and optionally in the presence of a base such as triethylamine or N,N-diisopropylethylamine, or under conditions described in the literature for an amide coupling. For examples, see WO 2003/028729. Compounds of formula (IV) are commercially available or prepared using known methods. For related examples, see Charalambides, Y. C., Moratti, S. C. Synth. Commun. (2007), 37, 1037; Schaefer, G. et al Angew. Chem., Int. Ed. (2012) 51, 9173; Lengyel, I. et al Heterocycles (2007), 73, 349; and Benalil, A et al Synthesis (1991), 9, 787. This reaction is shown in Scheme 2.

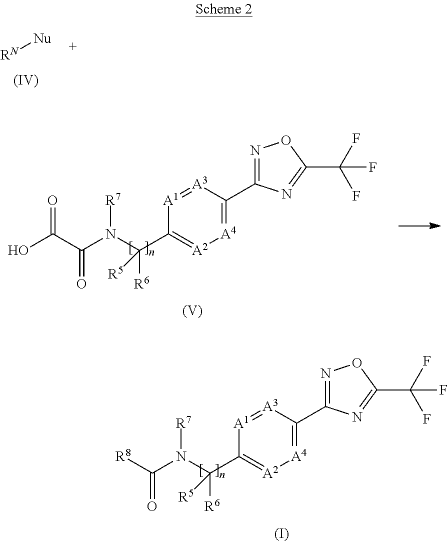

Compounds of formula (V) can be prepared from compounds of formula (Ia), wherein R^{alk} is methyl or ethyl, by treatment with lithium hydroxide in a suitable solvent, such as a tetrahydrofuran and water mixture, at a temperature of 22° C. For related examples, see WO 2003/028729 and WO 2010/045251. This reaction is shown in Scheme 3.

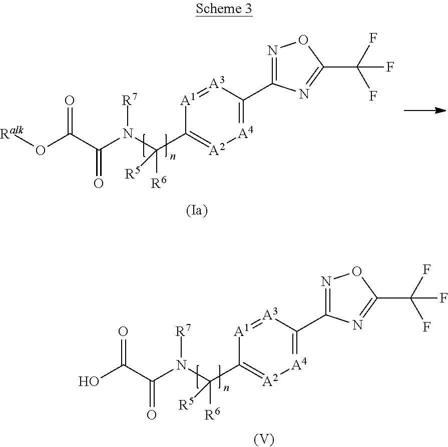

Additionally, compounds of formula (I) can be prepared from compounds of formula (VI) by treatment with trifluoroacetic anhydride in the presence of a base (eg, pyridine or 4-dimethylaminopyridine) in a suitable solvent, such as tetrahydrofuran or ethanol, at a temperature between 25° C. and 75° C. For related examples, see WO 2003/028729 and WO 2010/045251. This reaction is shown in Scheme 4.

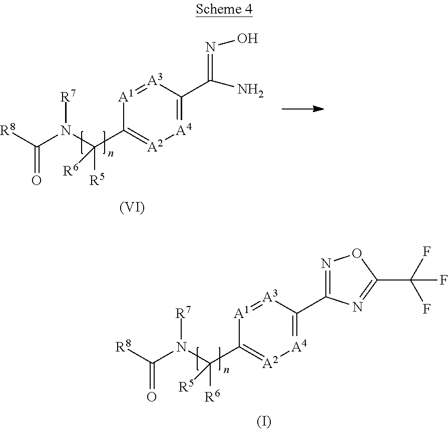

Compounds of formula (VI) can be prepared from compounds of formula (VII) by treatment with a hydroxylamine hydrochloride salt in the presence of a base, such as triethylamine, in a suitable solvent, such as methanol, at a temperature between 0° C. and 100° C. For related examples, see Kitamura, S. et al Chem. Pharm. Bull. (2001), 49, 268 and WO 2013/066838. This reaction is shown in Scheme 5.

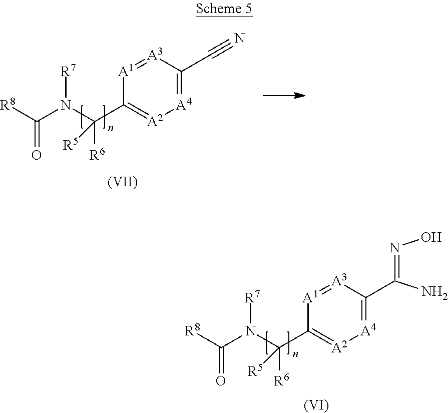

Compounds of formula (VII) can be prepared from compounds of formula (VIII), wherein Z is Br or I, via metal-promoted reaction with a suitable cyanide reagent, such as Pd(0)/Zn(CN)₂ or CuCN, in a suitable solvent (eg, dimethylformamide or N-methylpyrrolidone) at elevated temperature between 100° C. and 120° C. For related examples, see US 2007/0155739 and WO 2009/022746. This reaction is shown in Scheme 6.

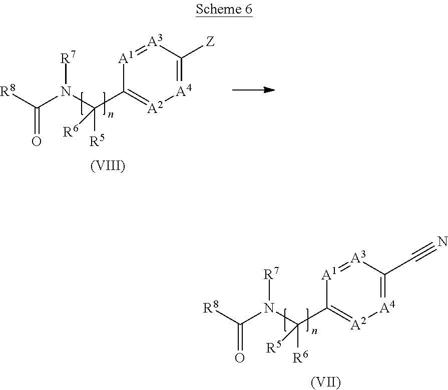

Compounds of formula (II), wherein n is preferably 0 or 1, and R⁶ is hydrogen, can be prepared from carbonyl compounds of formula (IX), starting with treatment by compounds of formula (X), wherein R^{PG} is tert-butylsulfinamide, optionally in the presence of an activating reagent (eg, Ti(OEt)₄), in a suitable solvent, (eg, tetrahydrofuran) at a temperature between 60° C. and 75° C. and followed by the addition of a reagent of formula (XI), such as an alkyl Grignard reagent (eg. alkylMgBr), Me₃SiCN, or a metal hydride (eg, NaBH₄, NaBH₃CN, or LiAlH₄), in a suitable solvent, (eg, tetrahydrofuran or ethanol) at temperatures between 0° C. and 25° C. Removal of the tert-butanesulfinyl group with concomitant liberation of amine compounds of formula (II) occurs upon treatment with methanolic HCl. For related examples, see Cogan, D., Ellman J. A. J. Am. Chem. Soc. (1999), 121, 268. This reaction is shown in Scheme 7.

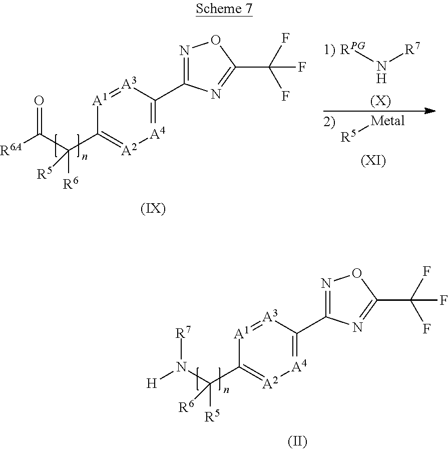

Alternatively, compounds of formula (II), wherein n is preferably 1, can be prepared from compounds of formula (XIII), wherein X is Cl or Br, by treatment with amines of formula (XII), wherein Y is tert-butylcarboxylate, in a suitable solvent (eg, tetrahydrofuran) at a temperature between 25° C. and 60° C. Removal of the tert-butylcarboxylate groups with concomitant liberation of benzylamines of formula (II) occurs upon treatment with HCl or trifluoroacetic acid in a suitable solvent (eg, dioxane or MeOH). For related examples, see Miyawaki, K. et al Heterocycles (2001), 54, 887, WO 2003/028729, and WO 2013/066839. This reaction is shown in Scheme 8.

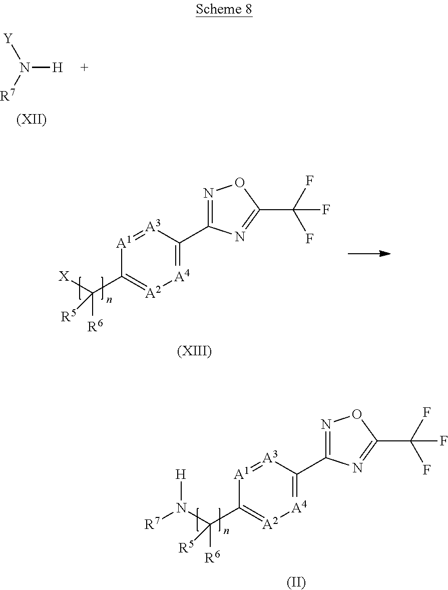

Compounds of formula (XIII), wherein n is 1, can be prepared from compounds of formula (XIV), wherein X is Cl or Br, by treatment with a halogen source (eg, N-bromosuccinimide (NBS) or N-chlorosuccinimide (NCS)) and a radical initiator (eg, (PhCO₂)₂ or azobisisobutyronitrile (AIBN)) in a suitable solvent, such as tetrachloromethane, at temperatures between 55° and 100° C. in the presence of ultraviolet light. For related examples, see Liu, S. et al Synthesis (2001), 14, 2078 and Kompella, A. et al Org. Proc. Res. Dev. (2012), 16, 1794. This reaction is shown in Scheme 9.

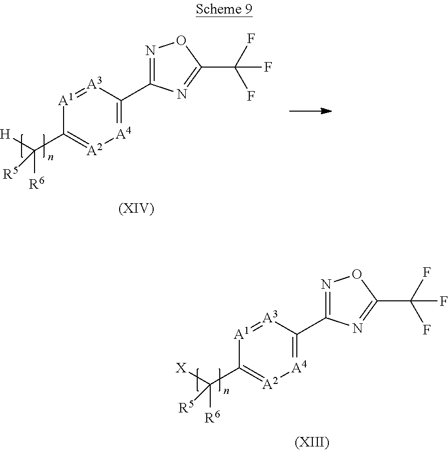

The compounds of formula (VIII) can be obtained by an amide coupling transformation with compounds of formula (XV) and compounds of formula (III) by activating the carboxylic acid function of the compounds of formula (III), a process that usually takes place by converting the —OH of the carboxylic acid into a good leaving group, such as a chloride group, for example by using (COCl)₂ or SOCl₂, prior to treatment with the compounds of formula (XV), preferably in a suitable solvent (eg, dimethylformamide, dichloromethane or tetrahydrofuran), preferably at a temperature of between 25° C. and 100° C., and optionally in the presence of a base such as triethylamine or N,N-diisopropylethylamine, or under conditions described in the literature for an amide coupling. For examples, see WO 2003/028729. Compounds of formula (III) are commercially available or prepared using known methods. For related examples, see: Nelson, T. D et al Tetrahedron Lett. (2004), 45, 8917; Senthil, K. et al Pest. Res. Journal (2009), 21, 133; and Crich, D., Zou, Y. J. Org. Chem. (2005), 70, 3309. This reaction is shown in Scheme 10.

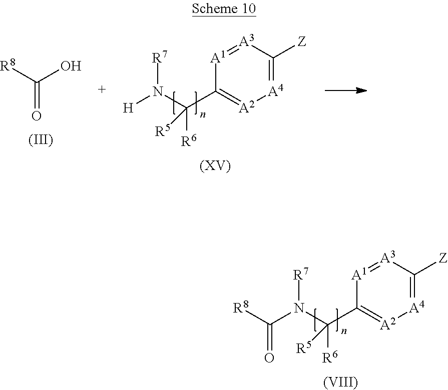

Alternatively, the compounds of formula (VIII) can be obtained by an amide coupling transformation with compounds of formula (IV), wherein R^{N}—Nu is R¹¹—OH, its alkoxide conjugate, or R^{N}-Nu is R¹³—N(H)—R¹⁴, and compounds of formula (XVI) by activating the carboxylic acid function of the compounds of formula (XVI), a process that usually takes place by converting the —OH of the carboxylic acid into a good leaving group, such as a chloride group, for example by using (COCl)₂ or SOCl₂, prior to treatment with the compounds of formula (IV), preferably in a suitable solvent (eg, dimethylformamide, dichloromethane or tetrahydrofuran), preferably at a temperature of between 22° C. and 100° C., and optionally in the presence of a base such as triethylamine or N,N-diisopropylethylamine, or under conditions described in the literature for an amide coupling. For examples, see WO 2003/028729. Compounds of formula (IV) are commercially available or prepared using known methods. For related examples, see Charalambides, Y. C., Moratti, S. C. Synth. Commun. (2007), 37, 1037; Schaefer, G. et al Angew. Chem., Int. Ed. (2012) 51, 9173; Lengyel, I. et al Heterocycles (2007), 73, 349; and Benalil, A et al Synthesis (1991), 9, 787. This reaction is shown in Scheme 11.

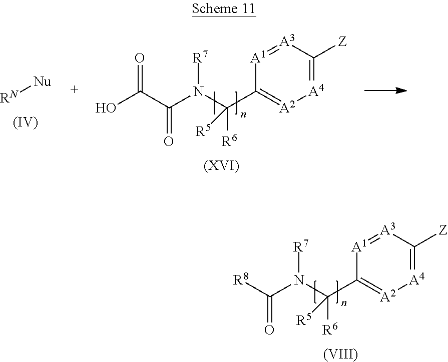

Compounds of formula (XVI) can be prepared from compounds of formula (Villa), wherein R^{alk} is methyl or ethyl, by treatment with lithium hydroxide in a suitable solvent, such as a tetrahydrofuran and water mixture, at a temperature of 22° C. For related examples, see WO 2003/028729 and WO 2010/045251. This reaction is shown in Scheme 12.

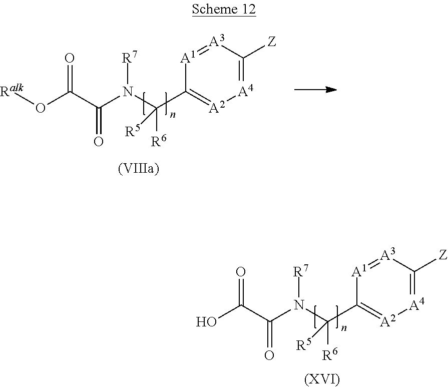

Furthermore, compounds of formula (VIII), wherein Z is Br, I, or CN and R⁸ is R⁹, OR¹¹ or NR¹³R¹⁴ and R¹⁴ is not hydrogen and n is preferably 1, can be prepared from compounds of formula (XVIII), wherein X is Cl, Br or I, via treatment with amides of formula (XVII), wherein Y is tert-butylcarboxylate, in the presence of a suitable base, such as NaH, in a suitable solvent, such as dimethylformamide, at a temperature between 0° C. and 100° C. In some cases, a better reaction performance may be gained from the use of a catalyst (eg, Nal or 4-dimethylaminopyridine) and with microwave irradiation. Removal of the tert-butylcarboxylate groups with concomitant liberation of benzylamides of formula (VIII) occurs upon treatment with HCl or trifluoroacetic acid in a suitable solvent (eg, dioxane or MeOH). For related examples, see Miyawaki, K. et al Heterocycles (2001), 54, 887. This reaction is shown in Scheme 13.

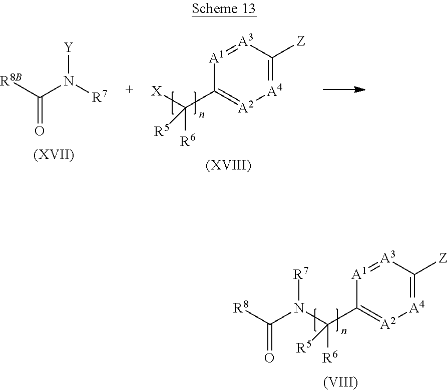

Compounds of formula (XVIII), wherein Z is Br, I, or CN and X is Cl or Br and n is preferably 1, are either commercially available or can be prepared from compounds of formula (XIX), by treatment with a halogen source, (eg, N-bromosuccinimide (NBS) or N-chlorosuccinimide (NCS)) and a radical initiator, such as (PhCO₂)₂ or azobisisobutyronitrile (AIBN), in the presence of ultraviolet light, in a suitable solvent, such as tetrachloromethane, at temperatures between 55° C. and 100° C. For related examples, see Liu, S. et al Syntheis (2001), 14, 2078 and Kompella, A. et al Org. Proc. Res. Dev. (2012), 16, 1794. This reaction is shown in Scheme 14.

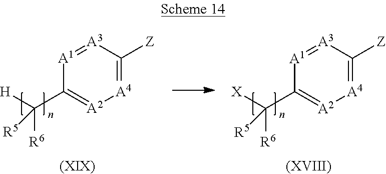

Alternatively, compounds of formula (XVIII), wherein X is Cl, Br, I, or OSO₂Me and Z is Br, I and n is preferably 1, or CN are either commercially available or can be prepared from compounds of formula (XX), by treatment with a halogen source (eg, CBr₄, CCl₄ or I₂) in the presence of triphenylphosphine, or with methanesulfonyl chloride (ClSO₂Me), in a suitable solvent, (eg, dichloromethane) at a temperature between 0° C. and 100° C. For related examples, see Liu, H. et al Bioorg. Med. Chem. (2008), 16, 10013, WO 2014/020350 and Kompella, A. et al Bioorg. Med. Chem. Lett. (2001), 1, 3161. Compounds of formula (XIX) are commercially available. This reaction is shown in Scheme 15.

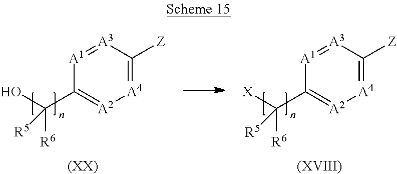

Compounds of formula (XV), wherein n is preferably 0 and R⁶ is hydrogen, can be prepared from carbonyl compounds of formula (XXI), starting with treatment by compounds of formula (X), wherein R^{PG} is tert-butylsulfinamide, optionally in the presence of an activating reagent (eg, Ti(OEt)₄), in a suitable solvent, (eg, tetrahydrofuran) at a temperature between 60° C. and 75° C. and followed by the addition of a reagent of formula (XI), such as an alkyl Grignard reagent (eg. alkylMgBr), Me₃SiCN, or a metal hydride (eg, NaBH₄, NaBH₃CN, or LiAlH₄), in a suitable solvent, (eg, tetrahydrofuran or ethanol) at temperatures between 0° C. and 25° C. Removal of the tert-butanesulfinyl group with concomitant liberation of amine compounds of formula (XV) occurs upon treatment with methanolic HCl. For related examples, see Cogan, D., Ellman J. A. J. Am. Chem. Soc. (1999), 121, 268. This reaction is shown in Scheme 16.

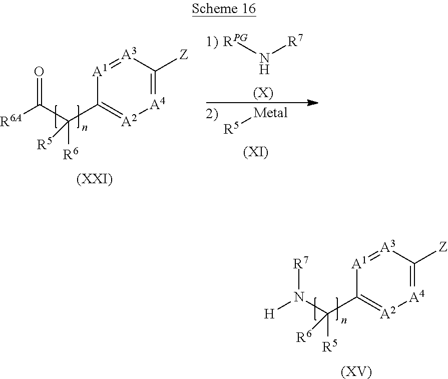

Compounds of formula (XV), wherein Z is Br, I, or CN and X is Cl or Br and n is 2, are either commercially available or can be prepared from compounds of formula (XXII), by reduction using BH₃ in a suitable solvent, such as methonol, at temperatures between 55° C. and 100° C. Compound of formula (XXII) are commercially available. This reaction is shown in Scheme 17.

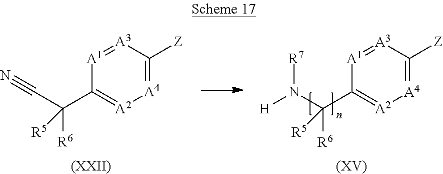

As already indicated, surprisingly, it has now been found that the novel compounds of formula (I) of the present invention have, for practical purposes, a very advantageous level of biological activity for protecting plants against diseases that are caused by fungi.

The compounds of formula (I) can be used in the agricultural sector and related fields of use, e.g., as active ingredients for controlling plant pests or on non-living materials for the control of spoilage microorganisms or organisms potentially harmful to man. The novel compounds are distinguished by excellent activity at low rates of application, by being well tolerated by plants and by being environmentally safe. They have very useful curative, preventive and systemic properties and can be used for protecting numerous cultivated plants. The compounds of formula I can be used to inhibit or destroy the pests that occur on plants or parts of plants (fruit, blossoms, leaves, stems, tubers, roots) of different crops of useful plants, while at the same time protecting also those parts of the plants that grow later, e.g., from phytopathogenic microorganisms.

The present invention further relates to a method for controlling or preventing infestation of plants or plant propagation material and/or harvested food crops susceptible to microbial attack by treating plants or plant propagation material and/or harvested food crops wherein an effective amount a compound of formula (I) is applied to the plants, to parts thereof or the locus thereof.

It is also possible to use compounds of formula (I) as fungicide. The term “fungicide” as used herein means a compound that controls, modifies, or prevents the growth of fungi. The term “fungicidally effective amount” where used means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

It may also be possible to use compounds of formula (I) as dressing agents for the treatment of plant propagation material, e.g., seed, such as fruits, tubers or grains, or plant cuttings, for the protection against fungal infections as well as against phytopathogenic fungi occurring in the soil. The propagation material can be treated with a composition comprising a compound of formula (I) before planting: seed, for example, can be dressed before being sown. The active compounds of formula (I) can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore, the compounds of formula (I) can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

The compounds of formula (I) are for example, effective against fungi and fungal vectors of disease as well as phytopathogenic bacteria and viruses. These fungi and fungal vectors of disease as well as phytopathogenic bacteria and viruses are for example:

Absidia corymbifera, Alternaria spp, Aphanomyces spp, Ascochyta spp, Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. inclusing B. cinerea, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, Cercosporidium personatum, Cladosporium spp, Claviceps purpurea, Coccidioides immitis, Cochliobolus spp, Colletotrichum spp. including C. musae, Cryptococcus neoformans, Diaporthe spp, Didymella spp, Drechslera spp, Elsinoe spp, Epidermophyton spp, Erwinia amylovora, Erysiphe spp. including E. cichoracearum, Eutypa lata, Fusarium spp. including F. culmorum, F. graminearum, F. langsethiae, F. moniliforme, F. oxysporum, F. proliferatum, F. subglutinans, F. solani, Gaeumannomyces graminis, Gibberella fujikuroi, Gloeodes pomigena, Gloeosporium musarum, Glomerella cingulate, Guignardia bidwellii, Gymnosporangium juniperi-virginianae, Helminthosporium spp, Hemileia spp, Histoplasma spp. including H. capsulatum, Laetisaria fuciformis, Leptographium lindbergi, Leveillula taurica, Lophodermium seditiosum, Microdochium nivale, Microsporum spp, Monilinia spp, Mucor spp, Mycosphaerella spp. including M. graminicola, M. pomi, Oncobasidium theobromaeon, Ophiostoma piceae, Paracoccidioides spp, Penicillium spp. including P. digitatum, P. italicum, Petriellidium spp, Peronosclerospora spp. Including P. maydis, P. philippinensis and P. sorghi, Peronospora spp, Phaeosphaeria nodorum, Phakopsora pachyrhizi, Phellinus igniarus, Phialophora spp, Phoma spp, Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudocercosporella herpotrichoides, Pseudomonas spp, Pseudoperonospora spp. including P. cubensis, P. humuli, Pseudopeziza tracheiphila, Puccinia Spp. including P. hordei, P. recondita, P. striiformis, P. triticina, Pyrenopeziza spp, Pyrenophora spp, Pyricularia spp. including P. oryzae, Pythium spp. including P. ultimum, Ramularia spp, Rhizoctonia spp, Rhizomucor pusillus, Rhizopus arrhizus, Rhynchosporium spp, Scedosporium spp. including S. apiospermum and S. prolificans, Schizothyrium pomi, Sclerotinia spp, Sclerotium spp, Septoria spp, including S. nodorum, S. tritici, Sphaerotheca macularis, Sphaerotheca fusca (Sphaerotheca fuliginea), Sporothorix spp, Stagonospora nodorum, Stemphylium spp. Stereum hirsutum, Thanatephorus cucumeris, Thielaviopsis basicola, Tilletia spp, Trichoderma spp. including T. harzianum, T. pseudokoningii, T. viride, Trichophyton spp, Typhula spp, Uncinula necator, Urocystis spp, Ustilago spp, Venturia spp. including V. inaequalis, Verticillium spp, and Xanthomonas spp.

The compounds of formula (I) may be used for example on turf, ornamentals, such as flowers, shrubs, broad-leaved trees or evergreens, for example conifers, as well as for tree injection, pest management and the like.

Within the scope of present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The term “useful plants” is to be understood as also including useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® Agrisure® GT Advantage (GA21 glyphosate-tolerant trait), Agrisure® CB Advantage (Bt11 corn borer (CB) trait), Agrisure® RW (corn rootworm trait) and Protecta®.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins from Bacillus cereus or Bacillus popilliae; or insecticidal proteins from Bacillus thuringiensis, such as 8-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by δ-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO93/07278, WO95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Btl 1 maize also transgenically expresses the enzyme PAT to achieve tolerance to the herbicide glufosinate ammonium.
2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified Cry3A toxin. This toxin is Cry3A155 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein CryIF for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There can be mentioned for example seeds (in the strict sense), roots, fruits, tubers, bulbs, rhizomes and parts of plants. Germinated plants and young plants which are to be transplanted after germination or after emergence from the soil, may also be mentioned. These young plants can be protected before transplantation by a total or partial treatment by immersion. Preferably “plant propagation material” is understood to denote seeds.

The compounds of formula I may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and carriers that are useful in formulating the compositions of the invention in the formulation types described above are well known to those skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc., ethylene glycol, propylene glycol, glycerine and N-methyl-2-pyrrolidinone. Water is generally the carrier of choice for the dilution of concentrates.

Suitable solid carriers include, for example, talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants and sticking agents.

In addition, further, other biocidally active ingredients or compositions may be combined with the compositions of the invention and used in the methods of the invention and applied simultaneously or sequentially with the compositions of the invention. When applied simultaneously, these further active ingredients may be formulated together with the compositions of the invention or mixed in, for example, the spray tank. These further biocidally active ingredients may be fungicides, herbicides, insecticides, bactericides, acaricides, nematicides and/or plant growth regulators.

Pesticidal agents are referred to herein using their common name are known, for example, from “The Pesticide Manual”, 15th Ed., British Crop Protection Council 2009.

In addition, the compositions of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-S-methyl.

The compounds of formula (I) are normally used in the form of agrochemical compositions and can be applied to the crop area or plant to be treated, simultaneously or in succession with further compounds. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula (I) may be used in the form of (fungicidal) compositions for controlling or protecting against phytopathogenic microorganisms, comprising as active ingredient at least one compound of formula (I) or of at least one preferred individual compound as defined herein, in free form or in agrochemically usable salt form, and at least one of the above-mentioned adjuvants.

The invention therefore provides a composition, preferably a fungicidal composition, comprising at least one compound formula (I) an agriculturally acceptable carrier and optionally an adjuvant. An agricultural acceptable carrier is for example a carrier that is suitable for agricultural use. Agricultural carriers are well known in the art. Preferably said composition may comprise at least one or more pesticidally-active compounds, for example an additional fungicidal active ingredient in addition to the compound of formula (I).

The compound of formula (I) may be the sole active ingredient of a composition or it may be admixed with one or more additional active ingredients such as a pesticide, fungicide, synergist, herbicide or plant growth regulator where appropriate. An additional active ingredient may, in some cases, result in unexpected synergistic activities.

Examples of suitable additional active ingredients include the following: acycloamino acid fungicides, aliphatic nitrogen fungicides, amide fungicides, anilide fungicides, antibiotic fungicides, aromatic fungicides, arsenical fungicides, aryl phenyl ketone fungicides, benzamide fungicides, benzanilide fungicides, benzimidazole fungicides, benzothiazole fungicides, botanical fungicides, bridged diphenyl fungicides, carbamate fungicides, carbanilate fungicides, conazole fungicides, copper fungicides, dicarboximide fungicides, dinitrophenol fungicides, dithiocarbamate fungicides, dithiolane fungicides, furamide fungicides, furanilide fungicides, hydrazide fungicides, imidazole fungicides, mercury fungicides, morpholine fungicides, organophosphorous fungicides, organotin fungicides, oxathiin fungicides, oxazole fungicides, phenylsulfamide fungicides, polysulfide fungicides, pyrazole fungicides, pyridine fungicides, pyrimidine fungicides, pyrrole fungicides, quaternary ammonium fungicides, quinoline fungicides, quinone fungicides, quinoxaline fungicides, strobilurin fungicides, sulfonanilide fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, valinamide fungicides, and zinc fungicides.

Examples of suitable additional active ingredients also include the following: 3-difluoromethyl-1-methyl-1H-pyrazole-4-carboxylic acid (9-dichloromethylene-1,2,3,4-tetrahydro-1,4-methano-naphthalen-5-yl)-amide, 3-difluoromethyl-1-methyl-1H-pyrazole-4-carboxylic acid methoxy-[1-methyl-2-(2,4,6-trichlorophenyl)-ethyl]-amide, 1-methyl-3-difluoromethyl-1H-pyrazole-4-carboxylic acid (2-dichloromethylene-3-ethyl-1-methyl-indan-4-yl)-amide (1072957-71-1), 1-methyl-3-difluoromethyl-1H-pyrazole-4-carboxylic acid (4′-methylsulfanyl-biphenyl-2-yl)-amide, 1-methyl-3-difluoromethyl-4H-pyrazole-4-carboxylic acid [2-(2,4-dichloro-phenyl)-2-methoxy-1-methyl-ethyl]-amide, (5-Chloro-2,4-dimethyl-pyridin-3-yl)-(2,3,4-trimethoxy-6-methyl-phenyl)-methanone, (5-Bromo-4-chloro-2-methoxy-pyridin-3-yl)-(2,3,4-trimethoxy-6-methyl-phenyl)-methanone, 2-{2-[(E)-3-(2,6-Dichloro-phenyl)-1-methyl-prop-2-en-(E)-ylideneaminooxymethyl]-phenyl}-2-[(Z)-methoxyimino]-N-methyl-acetamide, 3-[5-(4-Chloro-phenyl)-2,3-dimethyl-isoxazolid in-3-yl]-pyridine, (E)-N-methyl-2-[2-(2, 5-dimethylphenoxymethyl) phenyl]-2-methoxy-iminoacetamide, 4-bromo-2-cyano-N, N-dimethyl-6-trifluoromethylbenzimidazole-1-sulphonamide, a-[N-(3-chloro-2, 6-xylyl)-2-methoxyacetamido]-y-butyrolactone, 4-chloro-2-cyano-N,-dimethyl-5-p-tolylimidazole-1-sulfonamide, N-allyl-4, 5,-dimethyl-2-trimethylsilylthiophene-3-carboxamide, N— (I-cyano-1, 2-dimethylpropyl)-2-(2, 4-dichlorophenoxy) propionamide, N-(2-m ethoxy-5-pyridyl)-cyclopropane carboxamide, (.+-.)-cis-1-(4-chlorophenyl)-2-(1H-1,2,4-triazol-1-yl)-cycloheptanol, 2-(1-tert-butyl)-1-(2-chlorophenyl)-3-(1,2,4-triazol-1-yl)-propan-2-ol, 2′,6′-dibromo-2-methyl-4-trifluoromethoxy-4′-trifluoromethyl-1,3-thiazole-5-carboxanilide, 1-imidazolyl-1-(4′-chlorophenoxy)-3,3-dimethylbutan-2-one, methyl (E)-2-[2-[6-(2-cyanophenoxy)pyrimidin-4-yloxy]phenyl]3-methoxyacrylate, methyl (E)-2-[2-[6-(2-thioamidophenoxy)pyrimidin-4-yloxy]phenyl]-3-methoxyacrylate, methyl (E)-2-[2-[6-(2-fluorophenoxy)pyrimidin-4-yloxy]phenyl]-3-methoxyacrylate, methyl (E)-2-[2-[6-(2,6-difluorophenoxy)pyrimidin-4-yloxy]phenyl]-3-methoxyacrylate, methyl (E)-2-[2-[3-(pyrimidin-2-yloxy)phenoxy]phenyl]-3-methoxyacrylate, methyl (E)-2-[2-[3-(5-methylpyrimidin-2-yloxy)-phenoxy]phenyl]-3-methoxyacrylate, methyl (E)-2-[2-[3-(phenyl-sulphonyloxy)phenoxy]phenyl-3-methoxyacrylate, methyl (E)-2-[2-[3-(4-nitrophenoxy)phenoxy]phenyl]-3-methoxyacrylate, methyl (E)-2-[2-phenoxyphenyl]-3-methoxyacrylate, methyl (E)-2-[2-(3,5-dimethyl-benzoyl)pyrrol-1-yl]-3-methoxyacrylate, methyl (E)-2-[2-(3-methoxyphenoxy)phenyl]-3-methoxyacrylate, methyl (E)-2[2-(2-phenylethen-1-yl)-phenyl]-3-methoxyacrylate, methyl (E)-2-[2-(3,5-dichlorophenoxy)pyridin-3-yl]-3-methoxyacrylate, methyl (E)-2-(2-(3-(1,1,2,2-tetrafluoroethoxy)phenoxy)phenyl)-3-methoxyacrylate, methyl (E)-2-(2-[3-(alpha-hydroxybenzyl)phenoxy]phenyl)-3-methoxyacrylate, methyl (E)-2-(2-(4-phenoxypyridin-2-yloxy)phenyl)-3-methoxyacrylate, methyl (E)-2-[2-(3-n-propyloxy-phenoxy)phenyl]3-methoxyacrylate, methyl (E)-2-[2-(3-isopropyloxyphenoxy)phenyl]-3-methoxyacrylate, methyl (E)-2-[2-[3-(2-fluorophenoxy)phenoxy]phenyl]-3-methoxyacrylate, methyl (E)-2-[2-(3-ethoxyphenoxy)phenyl]-3-methoxyacrylate, methyl (E)-2-[2-(4-tert-butyl-pyridin-2-yloxy)phenyl]-3-methoxyacrylate, methyl (E)-2-[2-[3-(3-cyanophenoxy)phenoxy]phenyl]-3-methoxyacrylate, methyl (E)-2-[2-[(3-methyl-pyridin-2-yloxymethyl)phenyl]-3-methoxyacrylate, methyl (E)-2-[2-[6-(2-methyl-phenoxy)pyrimidin-4-yloxy]phenyl]-3-methoxyacrylate, methyl (E)-2-[2-(5-bromo-pyridin-2-yloxymethyl)phenyl]-3-methoxyacrylate, methyl (E)-2-[2-(3-(3-iodopyridin-2-yloxy)phenoxy)phenyl]-3-methoxyacrylate, methyl (E)-2-[2-[6-(2-chloropyridin-3-yloxy)pyrimidin-4-yloxy]phenyl]-3-methoxyacrylate, methyl (E),(E)-2-[2-(5,6-dimethyl pyrazin-2-ylmethyloximinomethyl)phenyl]-3-methoxyacrylate, methyl (E)-2-{2-[6-(6-methylpyrid in-2-yloxy)pyrimidin-4-yloxy]phenyl}-3-methoxy-acrylate, methyl (E),(E)-2-{2-(3-m ethoxyphenyl)methyloximinomethyl]-phenyl}-3-methoxyacrylate, methyl (E)-2-{2-(6-(2-azidophenoxy)-pyrimidin-4-yloxy]phenyl}-3-methoxyacrylate, methyl (E),(E)-2-{2-[6-phenylpyrimidin-4-yl)-methyloximinomethyl]phenyl}-3-methoxyacrylate, methyl (E),(E)-2-{2-[(4-chlorophenyl)-methyloximinomethyl]-phenyl}-3-methoxyacrylate, methyl (E)-2-{2-[6-(2-n-propylphenoxy)-1,3,5-triazin-4-yloxy]phenyl}-3-methoxyacrylate, methyl (E),(E)-2-{2-[(3-nitrophenyl)methyloximinomethyl]phenyl}-3-methoxyacrylate, 3-chloro-7-(2-aza-2,7,7-trimethyl-oct-3-en-5-ine), 2,6-dichloro-N-(4-trifluoromethylbenzyl)-benzamide, 3-iodo-2-propinyl alcohol, 4-chlorophenyl-3-iodopropargyl formal, 3-bromo-2,3-diiodo-2-propenyl ethylcarbamate, 2,3,3-triiodoallyl alcohol, 3-bromo-2,3-diiodo-2-propenyl alcohol, 3-iodo-2-propinyl n-butylcarbamate, 3-iodo-2-propinyl n-hexylcarbamate, 3-iodo-2-propinyl cyclohexyl-carbamate, 3-iodo-2-propinyl phenylcarbamate; phenol derivatives, such as tribromophenol, tetrachlorophenol, 3-methyl-4-chlorophenol, 3,5-dimethyl-4-chlorophenol, phenoxyethanol, dichlorophene, o-phenylphenol, m-phenylphenol, p-phenylphenol, 2-benzyl-4-chlorophenol, 5-hydroxy-2(5H)-furanone; 4,5-dichlorodithiazolinone, 4,5-benzodithiazolinone, 4,5-trimethylenedithiazolinone, 4,5-dichloro-(3H)-1,2-dithiol-3-one, 3,5-dimethyl-tetrahydro-1,3,5-thiadiazine-2-thione, N-(2-p-chlorobenzoylethyl)-hexaminium chloride, acibenzolar, acypetacs, alanycarb, albendazole, aldimorph, allicin, allyl alcohol, ametoctradin, amisulbrom, amobam, ampropylfos, anilazine, asomate, aureofungin, azaconazole, azafendin, azithiram, azoxystrobin, barium polysulfide, benalaxyl, benalaxyl-M, benodanil, benomyl, benquinox, bentaluron, benthiavalicarb, benthiazole, benzalkonium chloride, benzamacril, benzamorf, benzohydroxamic acid, benzovindiflupyr, berberine, bethoxazin, biloxazol, binapacryl, biphenyl, bitertanol, bithionol, bixafen, blasticidin-S, boscalid, bromothalonil, bromuconazole, bupirimate, buthiobate, butylamine calcium polysulfide, captafol, captan, carbamorph, carbendazim, carbendazim chlorhydrate, carboxin, carpropamid, carvone, CGA41396, CGA41397, chinomethionate, chitosan, chlobenthiazone, chloraniformethan, chloranil, chlorfenazole, chloroneb, chloropicrin, chlorothalonil, chlorozolinate, chlozolinate, climbazole, clotrimazole, clozylacon, copper containing compounds such as copper acetate, copper carbonate, copper hydroxide, copper naphthenate, copper oleate, copper oxychloride, copper oxyquinolate, copper silicate, copper sulphate, copper tallate, copper zinc chromate and Bordeaux mixture, cresol, cufraneb, cuprobam, cuprous oxide, cyazofamid, cyclafuramid, cycloheximide, cyflufenamid, cymoxanil, cypendazole, cyproconazole, cyprodinil, dazomet, debacarb, decafentin, dehydroacetic acid, di-2-pyridyl disulphide 1, 1′-dioxide, dichlofluanid, diclomezine, dichlone, dicloran, dichlorophen, dichlozoline, diclobutrazol, diclocymet, diethofencarb, difenoconazole, difenzoquat, diflumetorim, 0, O-di-iso-propyl-S-benzyl thiophosphate, dimefluazole, dimetachlone, dimetconazole, dimethomorph, dimethirimol, diniconazole, diniconazole-M, dinobuton, dinocap, dinocton, dinopenton, dinosulfon, dinoterbon, diphenylamine, dipyrithione, disulfiram, ditalimfos, dithianon, dithioether, dodecyl dimethyl ammonium chloride, dodemorph, dodicin, dodine, doguadine, drazoxolon, edifenphos, enestroburin, epoxiconazole, etaconazole, etem, ethaboxam, ethirimol, ethoxyquin, ethilicin, ethyl (Z)-N-benzyl-N([methyl (methyl-thioethylideneamino-oxycarbonyl) amino] thio)-β-alaninate, etridiazole, famoxadone, fenamidone, fenaminosulf, fenapanil, fenarimol, fenbuconazole, fenfuram, fenhexamid, fenitropan, fenoxanil, fenpiclonil, fenpicoxamid, fenpropidin, fenpropimorph, fenpyrazamine, fentin acetate, fentin hydroxide, ferbam, ferimzone, fluazinam, fludioxonil, flumetover, flumorph, flupicolide, fluopyram, fluoroimide, fluotrimazole, fluoxastrobin, fluquinconazole, flusilazole, flusulfamide, flutanil, flutolanil, flutriafol, fluxapyroxad, folpet, formaldehyde, fosetyl, fuberidazole, furalaxyl, furametpyr, furcarbanil, furconazole, furfural, furmecyclox, furophanate, glyodin, griseofulvin, guazatine, halacrinate, hexa chlorobenzene, hexachlorobutadiene, hexachlorophene, hexaconazole, hexylthiofos, hydrargaphen, hydroxyisoxazole, hymexazole, imazalil, imazalil sulphate, imibenconazole, iminoctadine, iminoctadine triacetate, inezin, iodocarb, ipconazole, ipfentrifluconazole, iprobenfos, iprodione, iprovalicarb, isopropanyl butyl carbamate, isoprothiolane, isopyrazam, isotianil, isovaledione, izopamfos, kasugamycin, kresoxim-methyl, LY186054, LY211795, LY248908, mancozeb, mandipropamid, maneb, mebenil, mecarbinzid, mefenoxam, mefentrifluconazole, mepanipyrim, mepronil, mercuric chloride, mercurous chloride, meptyldinocap, metalaxyl, metalaxyl-M, metam, metazoxolon, metconazole, methasulfocarb, methfuroxam, methyl bromide, methyl iodide, methyl isothiocyanate, metiram, metiram-zinc, metominostrobin, metrafenone, metsulfovax, milneb, moroxydine, myclobutanil, myclozolin, nabam, natamycin, neoasozin, nickel dimethyldithiocarbamate, nitrostyrene, nitrothal-iso-propyl, nuarimol, octhilinone, ofurace, organomercury compounds, orysastrobin, osthol, oxadixyl, oxasulfuron, oxine-copper, oxolinic acid, oxpoconazole, oxycarboxin, parinol, pefurazoate, penconazole, pencycuron, penflufen, pentachlorophenol, penthiopyrad, phenamacril, phenazin oxide, phosdiphen, phosetyl-Al, phosphorus acids, phthalide, picoxystrobin, piperalin, polycarbamate, polyoxin D, polyoxrim, polyram, probenazole, prochloraz, procymidone, propamidine, propamocarb, propiconazole, propineb, propionic acid, proquinazid, prothiocarb, prothioconazole, pydiflumetofen, pyracarbolid, pyraclostrobin, pyrametrostrobin, pyraoxystrobin, pyrazophos, pyribencarb, pyridinitril, pyrifenox, pyrimethanil, pyriofenone, pyroquilon, pyroxychlor, pyroxyfur, pyrrolnitrin, quaternary ammonium compounds, quinacetol, quinazamid, quinconazole, quinomethionate, quinoxyfen, quintozene, rabenzazole, santonin, sedaxane, silthiofam, simeconazole, sipconazole, sodium pentachlorophenate, solatenol, spiroxamine, streptomycin, sulphur, sultropen, tebuconazole, tebfloquin, tecloftalam, tecnazene, tecoram, tetraconazole, thiabendazole, thiadifluor, thicyofen, thifluzamide, 2-(thiocyanomethylthio) benzothiazole, thiophanate-methyl, thioquinox, thiram, tiadinil, timibenconazole, tioxymid, tolclofos-methyl, tolylfluanid, triadimefon, triadimenol, triamiphos, triarimol, triazbutil, triazoxide, tricyclazole, tridemorph, trifloxystrobin, triflumazole, triforine, triflumizole, triticonazole, uniconazole, urbacide, validamycin, valifenalate, vapam, vinclozolin, zarilamid, zineb, ziram, and zoxamide.

The compounds of the invention may also be used in combination with anthelmintic agents. Such anthelmintic agents include, compounds selected from the macrocyclic lactone class of compounds such as ivermectin, avermectin, abamectin, emamectin, eprinomectin, doramectin, selamectin, moxidectin, nemadectin and milbemycin derivatives as described in EP-357460, EP-444964 and EP-594291. Additional anthelmintic agents include semisynthetic and biosynthetic avermectin/milbemycin derivatives such as those described in U.S. Pat. No. 5,015,630, WO-9415944 and WO-9522552. Additional anthelmintic agents include the benzimidazoles such as albendazole, cambendazole, fenbendazole, flubendazole, mebendazole, oxfendazole, oxibendazole, parbendazole, and other members of the class. Additional anthelmintic agents include imidazothiazoles and tetrahydropyrimidines such as tetramisole, levamisole, pyrantel pamoate, oxantel or morantel. Additional anthelmintic agents include flukicides, such as triclabendazole and clorsulon and the cestocides, such as praziquantel and epsiprantel.

The compounds of the invention may be used in combination with derivatives and analogues of the paraherquamide/marcfortine class of anthelmintic agents, as well as the antiparasitic oxazolines such as those disclosed in U.S. Pat. No. 5,478,855, U.S. Pat. No. 4,639,771 and DE-19520936.

The compounds of the invention may be used in combination with derivatives and analogues of the general class of dioxomorpholine antiparasitic agents as described in WO 96/15121 and also with anthelmintic active cyclic depsipeptides such as those described in WO 96/11945, WO 93/19053, WO 93/25543, EP 0 626 375, EP 0 382 173, WO 94/19334, EP 0 382 173, and EP 0 503 538.

The compounds of the invention may be used in combination with other ectoparasiticides; for example, fipronil; pyrethroids; organophosphates; insect growth regulators such as lufenuron; ecdysone agonists such as tebufenozide and the like; neonicotinoids such as imidacloprid and the like.

The compounds of the invention may be used in combination with terpene alkaloids, for example those described in International Patent Application Publication Numbers WO 95/19363 or WO 04/72086, particularly the compounds disclosed therein.

Other examples of such biologically active compounds that the compounds of the invention may be used in combination with include but are not restricted to the following: Organophosphates: acephate, azamethiphos, azinphos-ethyl, azinphos-methyl, bromophos, bromophos-ethyl, cadusafos, chlorethoxyphos, chlorpyrifos, chlorfenvinphos, chlormephos, demeton, demeton-S-methyl, demeton-S-methyl sulphone, dialifos, diazinon, dichlorvos, dicrotophos, dimethoate, disulfoton, ethion, ethoprophos, etrimfos, famphur, fenamiphos, fenitrothion, fensulfothion, fenthion, flupyrazofos, fonofos, formothion, fosthiazate, heptenophos, isazophos, isothioate, isoxathion, malathion, methacriphos, methamidophos, methidathion, methyl-parathion, mevinphos, monocrotophos, naled, omethoate, oxydemeton-methyl, paraoxon, parathion, parathion-methyl, phenthoate, phosalone, phosfolan, phosphocarb, phosmet, phosphamidon, phorate, phoxim, pirimiphos, pirimiphos-methyl, profenofos, propaphos, proetamphos, prothiofos, pyraclofos, pyridapenthion, quinalphos, sulprophos, temephos, terbufos, tebupirimfos, tetrachlorvinphos, thimeton, triazophos, trichlorfon, vamidothion.

Carbamates: alanycarb, aldicarb, 2-sec-butylphenyl methylcarbamate, benfuracarb, carbaryl, carbofuran, carbosulfan, cloethocarb, ethiofencarb, fenoxycarb, fenthiocarb, furathiocarb, HCN-801, isoprocarb, indoxacarb, methiocarb, methomyl, 5-methyl-m-cumenylbutyryl(methyl)carbamate, oxamyl, pirimicarb, propoxur, thiodicarb, thiofanox, triazamate, UC-51717.

Pyrethroids: acrinathin, allethrin, alphametrin, 5-benzyl-3-furylmethyl (E)-(1R)-cis-2,2-dimethyl-3-(2-oxothiolan-3-ylidenemethyl)cyclopropanecarboxylate, bifenthrin, beta-cyfluthrin, cyfluthrin, a-cypermethrin, beta-cypermethrin, bioallethrin, bioallethrin((S)-cyclopentylisomer), bioresmethrin, bifenthrin, NCI-85193, cycloprothrin, cyhalothrin, cythithrin, cyphenothrin, deltamethrin, empenthrin, esfenvalerate, ethofenprox, fenfluthrin, fenpropathrin, fenvalerate, flucythrinate, flumethrin, fluvalinate (D isomer), imiprothrin, cyhalothrin, lambda-cyhalothrin, permethrin, phenothrin, prallethrin, pyrethrins (natural products), resmethrin, tetramethrin, transfluthrin, theta-cypermethrin, silafluofen, t-fluvalinate, tefluthrin, tralomethrin, Zeta-cypermethrin.

Arthropod growth regulators: a) chitin synthesis inhibitors: benzoylureas: chlorfluazuron, diflubenzuron, fluazuron, flucycloxuron, flufenoxuron, hexaflumuron, lufenuron, novaluron, teflubenzuron, triflumuron, buprofezin, diofenolan, hexythiazox, etoxazole, chlorfentazine; b) ecdysone antagonists: halofenozide, methoxyfenozide, tebufenozide; c) juvenoids: pyriproxyfen, methoprene (including S-methoprene), fenoxycarb; d) lipid biosynthesis inhibitors: spirodiclofen.

Other antiparasitics: acequinocyl, amitraz, AKD-1022, ANS-118, azadirachtin, Bacillus thuringiensis, bensultap, bifenazate, binapacryl, bromopropylate, BTG-504, BTG-505, camphechlor, cartap, chlorobenzilate, chlordimeform, chlorfenapyr, chromafenozide, clothianidine, cyromazine, diacloden, diafenthiuron, DBI-3204, dinactin, dihydroxymethyldihydroxypyrrolidine, dinobuton, dinocap, endosulfan, ethiprole, ethofenprox, fenazaquin, flumite, MTI-800, fenpyroximate, fluacrypyrim, flubenzimine, flubrocythrinate, flufenzine, flufenprox, fluproxyfen, halofenprox, hydramethylnon, IKI-220, kanemite, NC-196, neem guard, nidinorterfuran, nitenpyram, SD-35651, WL-108477, pirydaryl, propargite, protrifenbute, pymethrozine, pyridaben, pyrimidifen, NC-1111, R-195, RH-0345, RH-2485, RYI-210, S-1283, S-1833, SI-8601, silafluofen, silomadine, spinosad, tebufenpyrad, tetradifon, tetranactin, thiacloprid, thiocyclam, thiamethoxam, tolfenpyrad, triazamate, triethoxyspinosyn, trinactin, verbutin, vertalec, YI-5301.

Biological agents: Bacillus thuringiensis ssp aizawai, kurstaki, Bacillus thuringiensis delta endotoxin, baculovirus, entomopathogenic bacteria, virus and fungi.

Bactericides: chlortetracycline, oxytetracycline, streptomycin.

Other biological agents: enrofloxacin, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, carprofen, metaflumizone, praziquarantel, triclabendazole.

The following mixtures of the compounds of formula (I) with active ingredients are preferred.

The abbreviation “TX” means one compound selected from the group consisting of the compounds described in Tables 1.1 to 1.34, 2.1 to 2.33 and 3.1 to 3.31 (below) or Tables T1, T2, T3 and T4 (below).

an adjuvant selected from the group of substances consisting of petroleum oils (628)+TX,

an acaricide selected from the group of substances consisting of 1,1-bis(4-chlorophenyl)-2-ethoxyethanol (IUPAC name) (910)+TX, 2,4-dichlorophenyl benzenesulfonate (IUPAC/Chemical Abstracts name) (1059)+TX, 2-fluoro-N-methyl-N-1-naphthylacetamide (IUPAC name) (1295)+TX, 4-chlorophenyl phenyl sulfone (IUPAC name) (981)+TX, abamectin (1)+TX, acequinocyl (3)+TX, acetoprole [CCN]+TX, acrinathrin (9)+TX, aldicarb (16)+TX, aldoxycarb (863)+TX, alpha-cypermethrin (202)+TX, amidithion (870)+TX, amidoflumet [CCN]+TX, amidothioate (872)+TX, amiton (875)+TX, amiton hydrogen oxalate (875)+TX, amitraz (24)+TX, aramite (881)+TX, arsenous oxide (882)+TX, AVI 382 (compound code)+TX, AZ 60541 (compound code)+TX, azinphos-ethyl (44)+TX, azinphos-methyl (45)+TX, azobenzene (IUPAC name) (888)+TX, azocyclotin (46)+TX, azothoate (889)+TX, benomyl (62)+TX, benoxafos [CCN]+TX, benzoximate (71)+TX, benzyl benzoate (IUPAC name) [CCN]+TX, bifenazate (74)+TX, bifenthrin (76)+TX, binapacryl (907)+TX, brofenvalerate+TX, bromocyclen (918)+TX, bromophos (920)+TX, bromophos-ethyl (921)+TX, bromopropylate (94)+TX, buprofezin (99)+TX, butocarboxim (103)+TX, butoxycarboxim (104)+TX, butylpyridaben+TX, calcium polysulfide (IUPAC name) (111)+TX, camphechlor (941)+TX, carbanolate (943)+TX, carbaryl (115)+TX, carbofuran (118)+TX, carbophenothion (947)+TX, CGA 50′439 (development code) (125)+TX, chinomethionat (126)+TX, chlorbenside (959)+TX, chlordimeform (964)+TX, chlordimeform hydrochloride (964)+TX, chlorfenapyr (130)+TX, chlorfenethol (968)+TX, chlorfenson (970)+TX, chlorfensulfide (971)+TX, chlorfenvinphos (131)+TX, chlorobenzilate (975)+TX, chloromebuform (977)+TX, chloromethiuron (978)+TX, chloropropylate (983)+TX, chlorpyrifos (145)+TX, chlorpyrifos-methyl (146)+TX, chlorthiophos (994)+TX, cinerin 1 (696)+TX, cinerin II (696)+TX, cinerins (696)+TX, clofentezine (158)+TX, closantel [CCN]+TX, coumaphos (174)+TX, crotamiton [CCN]+TX, crotoxyphos (1010)+TX, cufraneb (1013)+TX, cyanthoate (1020)+TX, cyflumetofen (CAS Reg. No.: 400882-07-7)+TX, cyhalothrin (196)+TX, cyhexatin (199)+TX, cypermethrin (201)+TX, DCPM (1032)+TX, DDT (219)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulfon (1039)+TX, diafenthiuron (226)+TX, dialifos (1042)+TX, diazinon (227)+TX, dichlofluanid (230)+TX, dichlorvos (236)+TX, dicliphos+TX, dicofol (242)+TX, dicrotophos (243)+TX, dienochlor (1071)+TX, dimefox (1081)+TX, dimethoate (262)+TX, dinactin (653)+TX, dinex (1089)+TX, dinex-diclexine (1089)+TX, dinobuton (269)+TX, dinocap (270)+TX, dinocap-4 [CCN]+TX, dinocap-6 [CCN]+TX, dinocton (1090)+TX, dinopenton (1092)+TX, dinosulfon (1097)+TX, dinoterbon (1098)+TX, dioxathion (1102)+TX, diphenyl sulfone (IUPAC name) (1103)+TX, disulfiram [CCN]+TX, disulfoton (278)+TX, DNOC (282)+TX, dofenapyn (1113)+TX, doramectin [CCN]+TX, endosulfan (294)+TX, endothion (1121)+TX, EPN (297)+TX, eprinomectin [CCN]+TX, ethion (309)+TX, ethoate-methyl (1134)+TX, etoxazole (320)+TX, etrimfos (1142)+TX, fenazaflor (1147)+TX, fenazaquin (328)+TX, fenbutatin oxide (330)+TX, fenothiocarb (337)+TX, fenpropathrin (342)+TX, fenpyrad+TX, fenpyroximate (345)+TX, fenson (1157)+TX, fentrifanil (1161)+TX, fenvalerate (349)+TX, fipronil (354)+TX, fluacrypyrim (360)+TX, fluazuron (1166)+TX, flubenzimine (1167)+TX, flucycloxuron (366)+TX, flucythrinate (367)+TX, fluenetil (1169)+TX, flufenoxuron (370)+TX, flumethrin (372)+TX, fluorbenside (1174)+TX, fluvalinate (1184)+TX, FMC 1137 (development code) (1185)+TX, formetanate (405)+TX, formetanate hydrochloride (405)+TX, formothion (1192)+TX, formparanate (1193)+TX, gamma-HCH (430)+TX, glyodin (1205)+TX, halfenprox (424)+TX, heptenophos (432)+TX, hexadecyl cyclopropanecarboxylate (IUPAC/Chemical Abstracts name) (1216)+TX, hexythiazox (441)+TX, iodomethane (IUPAC name) (542)+TX, isocarbophos (473)+TX, isopropyl O-(methoxyaminothiophosphoryl)salicylate (IUPAC name) (473)+TX, ivermectin [CCN]+TX, jasmolin I (696)+TX, jasmolin II (696)+TX, jodfenphos (1248)+TX, lindane (430)+TX, lufenuron (490)+TX, malathion (492)+TX, malonoben (1254)+TX, mecarbam (502)+TX, mephosfolan (1261)+TX, mesulfen [CCN]+TX, methacrifos (1266)+TX, methamidophos (527)+TX, methidathion (529)+TX, methiocarb (530)+TX, methomyl (531)+TX, methyl bromide (537)+TX, metolcarb (550)+TX, mevinphos (556)+TX, mexacarbate (1290)+TX, milbemectin (557)+TX, milbemycin oxime [CCN]+TX, mipafox (1293)+TX, monocrotophos (561)+TX, morphothion (1300)+TX, moxidectin [CCN]+TX, naled (567)+TX, NC-184 (compound code)+TX, NC-512 (compound code)+TX, nifluridide (1309)+TX, nikkomycins [CCN]+TX, nitrilacarb (1313)+TX, nitrilacarb 1:1 zinc chloride complex (1313)+TX, NNI-0101 (compound code)+TX, NNI-0250 (compound code)+TX, omethoate (594)+TX, oxamyl (602)+TX, oxydeprofos (1324)+TX, oxydisulfoton (1325)+TX, pp′-DDT (219)+TX, parathion (615)+TX, permethrin (626)+TX, petroleum oils (628)+TX, phenkapton (1330)+TX, phenthoate (631)+TX, phorate (636)+TX, phosalone (637)+TX, phosfolan (1338)+TX, phosmet (638)+TX, phosphamidon (639)+TX, phoxim (642)+TX, pirimiphos-methyl (652)+TX, polychloroterpenes (traditional name) (1347)+TX, polynactins (653)+TX, proclonol (1350)+TX, profenofos (662)+TX, promacyl (1354)+TX, propargite (671)+TX, propetamphos (673)+TX, propoxur (678)+TX, prothidathion (1360)+TX, prothoate (1362)+TX, pyrethrin I (696)+TX, pyrethrin II (696)+TX, pyrethrins (696)+TX, pyridaben (699)+TX, pyridaphenthion (701)+TX, pyrimidifen (706)+TX, pyrimitate (1370)+TX, quinalphos (711)+TX, quintiofos (1381)+TX, R-1492 (development code) (1382)+TX, RA-17 (development code) (1383)+TX, rotenone (722)+TX, schradan (1389)+TX, sebufos+TX, selamectin [CCN]+TX, SI-0009 (compound code)+TX, sophamide (1402)+TX, spirodiclofen (738)+TX, spiromesifen (739)+TX, SSI-121 (development code) (1404)+TX, sulfiram [CCN]+TX, sulfluramid (750)+TX, sulfotep (753)+TX, sulfur (754)+TX, SZI-121 (development code) (757)+TX, tau-fluvalinate (398)+TX, tebufenpyrad (763)+TX, TEPP (1417)+TX, terbam+TX, tetrachlorvinphos (777)+TX, tetradifon (786)+TX, tetranactin (653)+TX, tetrasul (1425)+TX, thiafenox+TX, thiocarboxime (1431)+TX, thiofanox (800)+TX, thiometon (801)+TX, thioquinox (1436)+TX, thuringiensin [CCN]+TX, triamiphos (1441)+TX, triarathene (1443)+TX, triazophos (820)+TX, triazuron+TX, trichlorfon (824)+TX, trifenofos (1455)+TX, trinactin (653)+TX, vamidothion (847)+TX, vaniliprole [CCN] and YI-5302 (compound code)+TX,

an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX,

an anthelmintic selected from the group of substances consisting of abamectin (1)+TX, crufomate (1011)+TX, doramectin [CCN]+TX, emamectin (291)+TX, emamectin benzoate (291)+TX, eprinomectin [CCN]+TX, ivermectin [CCN]+TX, milbemycin oxime [CCN]+TX, moxidectin [CCN]+TX, piperazine [CCN]+TX, selamectin [CCN]+TX, spinosad (737) and thiophanate (1435)+TX,

an avicide selected from the group of substances consisting of chloralose (127)+TX, endrin (1122)+TX, fenthion (346)+TX, pyridin-4-amine (IUPAC name) (23) and strychnine (745)+TX, a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal [CCN]+TX,

a biological agent selected from the group of substances consisting of Adoxophyes orana GV (12)+TX, Agrobacterium radiobacter (13)+TX, Amblyseius spp. (19)+TX, Anagrapha falcifera NPV (28)+TX, Anagrus atomus (29)+TX, Aphelinus abdominalis (33)+TX, Aphidius colemani (34)+TX, Aphidoletes aphidimyza (35)+TX, Autographa californica NPV (38)+TX, Bacillus firmus (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (53)+TX, Beauveria brongniartii (54)+TX, Chrysoperla carnea (151)+TX, Cryptolaemus montrouzieri (178)+TX, Cydia pomonella GV (191)+TX, Dacnusa sibirica (212)+TX, Diglyphus isaea (254)+TX, Encarsia formosa (scientific name) (293)+TX, Eretmocerus eremicus (300)+TX, Helicoverpa zea NPV (431)+TX, Heterorhabditis bacteriophora and H. megidis (433)+TX, Hippodamia convergens (442)+TX, Leptomastix dactylopii (488)+TX, Macrolophus caliginosus (491)+TX, Mamestra brassicae NPV (494)+TX, Metaphycus helvolus (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (575)+TX, Orius spp. (596)+TX, Paecilomyces fumosoroseus (613)+TX, Phytoseiulus persimilis (644)+TX, Spodoptera exigua multicapsid nuclear polyhedrosis virus (scientific name) (741)+TX, Steinernema bibionis (742)+TX, Steinernema carpocapsae (742)+TX, Steinernema feltiae (742)+TX, Steinernema glaseri (742)+TX, Steinernema riobrave (742)+TX, Steinernema riobravis (742)+TX, Steinernema scapterisci (742)+TX, Steinernema spp. (742)+TX, Trichogramma spp. (826)+TX, Typhlodromus occidentalis (844) and Verticillium lecanii (848)+TX, a soil sterilant selected from the group of substances consisting of iodomethane (IUPAC name) (542) and methyl bromide (537)+TX,

a chemosterilant selected from the group of substances consisting of apholate [CCN]+TX, bisazir [CCN]+TX, busulfan [CCN]+TX, diflubenzuron (250)+TX, dimatif [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron [CCN]+TX, tepa [CCN]+TX, thiohempa [CCN]+TX, thiotepa [CCN]+TX, tretamine [CCN] and uredepa [CCN]+TX,

an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin [CCN]+TX, brevicomin [CCN]+TX, codlelure [CCN]+TX, codlemone (167)+TX, cuelure (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure [CCN]+TX, ethyl 4-methyloctanoate (IUPAC name) (317)+TX, eugenol [CCN]+TX, frontalin [CCN]+TX, gossyplure (420)+TX, grandlure (421)+TX, grandlure I (421)+TX, grandlure II (421)+TX, grandlure III (421)+TX, grandlure IV (421)+TX, hexalure [CCN]+TX, ipsdienol [CCN]+TX, ipsenol [CCN]+TX, japonilure (481)+TX, lineatin [CCN]+TX, litlure [CCN]+TX, looplure [CCN]+TX, medlure [CCN]+TX, megatomoic acid [CCN]+TX, methyl eugenol (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure [CCN]+TX, oryctalure (317)+TX, ostramone [CCN]+TX, siglure [CCN]+TX, sordidin (736)+TX, sulcatol [CCN]+TX, tetradec-11-en-1-yl acetate (IUPAC name) (785)+TX, trimedlure (839)+TX, trimedlure A (839)+TX, trimedlure B₁ (839)+TX, trimedlure B₂ (839)+TX, trimedlure C (839) and trunc-call [CCN]+TX,

an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX,

an insecticide selected from the group of substances consisting of 1-dichloro-1-nitroethane (IUPAC/Chemical Abstracts name) (1058)+TX, 1,1-dichloro-2,2-bis(4-ethylphenyl)ethane (IUPAC name) (1056), +TX, 1,2-dichloropropane (IUPAC/Chemical Abstracts name) (1062)+TX, 1,2-dichloropropane with 1,3-dichloropropene (IUPAC name) (1063)+TX, 1-bromo-2-chloroethane (IUPAC/Chemical Abstracts name) (916)+TX, 2,2,2-trichloro-1-(3,4-dichlorophenyl)ethyl acetate (IUPAC name) (1451)+TX, 2,2-dichlorovinyl 2-ethylsulfinylethyl methyl phosphate (IUPAC name) (1066)+TX, 2-(1,3-dithiolan-2-yl)phenyl dimethylcarbamate (IUPAC/Chemical Abstracts name) (1109)+TX, 2-(2-butoxyethoxy)ethyl thiocyanate (IUPAC/Chemical Abstracts name) (935)+TX, 2-(4,5-dimethyl-1,3-dioxolan-2-yl)phenyl methylcarbamate (IUPAC/Chemical Abstracts name) (1084)+TX, 2-(4-chloro-3,5-xylyloxy)ethanol (IUPAC name) (986)+TX, 2-chlorovinyl diethyl phosphate (IUPAC name) (984)+TX, 2-imidazolidone (IUPAC name) (1225)+TX, 2-isovalerylindan-1,3-dione (IUPAC name) (1246)+TX, 2-methyl(prop-2-ynyl)aminophenyl methylcarbamate (IUPAC name) (1284)+TX, 2-thiocyanatoethyl laurate (IUPAC name) (1433)+TX, 3-bromo-1-chloroprop-1-ene (IUPAC name) (917)+TX, 3-methyl-1-phenylpyrazol-5-yl dimethylcarbamate (IUPAC name) (1283)+TX, 4-methyl(prop-2-ynyl)amino-3,5-xylyl methylcarbamate (IUPAC name) (1285)+TX, 5,5-dimethyl-3-oxocyclohex-1-enyl dimethylcarbamate (IUPAC name) (1085)+TX, abamectin (1)+TX, acephate (2)+TX, acetamiprid (4)+TX, acethion [CCN]+TX, acetoprole [CCN]+TX, acrinathrin (9)+TX, acrylonitrile (IUPAC name) (861)+TX, alanycarb (15)+TX, aldicarb (16)+TX, aldoxycarb (863)+TX, aldrin (864)+TX, allethrin (17)+TX, allosamidin [CCN]+TX, allyxycarb (866)+TX, alpha-cypermethrin (202)+TX, alpha-ecdysone [CCN]+TX, aluminium phosphide (640)+TX, amidithion (870)+TX, amidothioate (872)+TX, aminocarb (873)+TX, amiton (875)+TX, amiton hydrogen oxalate (875)+TX, amitraz (24)+TX, anabasine (877)+TX, athidathion (883)+TX, AVI 382 (compound code)+TX, AZ 60541 (compound code)+TX, azadirachtin (41)+TX, azamethiphos (42)+TX, azinphos-ethyl (44)+TX, azinphos-methyl (45)+TX, azothoate (889)+TX, Bacillus thuringiensis delta endotoxins (52)+TX, barium hexafluorosilicate [CCN]+TX, barium polysulfide (IUPAC/Chemical Abstracts name) (892)+TX, barthrin [CCN]+TX, Bayer 22/190 (development code) (893)+TX, Bayer 22408 (development code) (894)+TX, bendiocarb (58)+TX, benfuracarb (60)+TX, bensultap (66)+TX, beta-cyfluthrin (194)+TX, beta-cypermethrin (203)+TX, bifenthrin (76)+TX, bioallethrin (78)+TX, bioallethrin S-cyclopentenyl isomer (79)+TX, bioethanomethrin [CCN]+TX, biopermethrin (908)+TX, bioresmethrin (80)+TX, bis(2-chloroethyl) ether (IUPAC name) (909)+TX, bistrifluron (83)+TX, borax (86)+TX, brofenvalerate+TX, bromfenvinfos (914)+TX, bromocyclen (918)+TX, bromo-DDT [CCN]+TX, bromophos (920)+TX, bromophos-ethyl (921)+TX, bufencarb (924)+TX, buprofezin (99)+TX, butacarb (926)+TX, butathiofos (927)+TX, butocarboxim (103)+TX, butonate (932)+TX, butoxycarboxim (104)+TX, butylpyridaben+TX, cadusafos (109)+TX, calcium arsenate [CCN]+TX, calcium cyanide (444)+TX, calcium polysulfide (IUPAC name) (111)+TX, camphechlor (941)+TX, carbanolate (943)+TX, carbaryl (115)+TX, carbofuran (118)+TX, carbon disulfide (IUPAC/Chemical Abstracts name) (945)+TX, carbon tetrachloride (IUPAC name) (946)+TX, carbophenothion (947)+TX, carbosulfan (119)+TX, cartap (123)+TX, cartap hydrochloride (123)+TX, cevadine (725)+TX, chlorbicyclen (960)+TX, chlordane (128)+TX, chlordecone (963)+TX, chlordimeform (964)+TX, chlordimeform hydrochloride (964)+TX, chlorethoxyfos (129)+TX, chlorfenapyr (130)+TX, chlorfenvinphos (131)+TX, chlorfluazuron (132)+TX, chlormephos (136)+TX, chloroform [CCN]+TX, chloropicrin (141)+TX, chlorphoxim (989)+TX, chlorprazophos (990)+TX, chlorpyrifos (145)+TX, chlorpyrifos-methyl (146)+TX, chlorthiophos (994)+TX, chromafenozide (150)+TX, cinerin 1 (696)+TX, cinerin II (696)+TX, cinerins (696)+TX, cis-resmethrin+TX, cismethrin (80)+TX, clocythrin+TX, cloethocarb (999)+TX, closantel [CCN]+TX, clothianidin (165)+TX, copper acetoarsenite [CCN]+TX, copper arsenate [CCN]+TX, copper oleate [CCN]+TX, coumaphos (174)+TX, coumithoate (1006)+TX, crotamiton [CCN]+TX, crotoxyphos (1010)+TX, crufomate (1011)+TX, cryolite (177)+TX, CS 708 (development code) (1012)+TX, cyanofenphos (1019)+TX, cyanophos (184)+TX, cyanthoate (1020)+TX, cyclethrin [CCN]+TX, cycloprothrin (188)+TX, cyfluthrin (193)+TX, cyhalothrin (196)+TX, cypermethrin (201)+TX, cyphenothrin (206)+TX, cyromazine (209)+TX, cythioate [CCN]+TX, d-limonene [CCN]+TX, d-tetramethrin (788)+TX, DAEP (1031)+TX, dazomet (216)+TX, DDT (219)+TX, decarbofuran (1034)+TX, deltamethrin (223)+TX, demephion (1037)+TX, demephion-O (1037)+TX, demephion-S(1037)+TX, demeton (1038)+TX, demeton-methyl (224)+TX, demeton-O (1038)+TX, demeton-O-methyl (224)+TX, demeton-S(1038)+TX, demeton-S-methyl (224)+TX, demeton-S-methylsulphon (1039)+TX, diafenthiuron (226)+TX, dialifos (1042)+TX, diamidafos (1044)+TX, diazinon (227)+TX, dicapthon (1050)+TX, dichlofenthion (1051)+TX, dichlorvos (236)+TX, dicliphos+TX, dicresyl [CCN]+TX, dicrotophos (243)+TX, dicyclanil (244)+TX, dieldrin (1070)+TX, diethyl 5-methylpyrazol-3-yl phosphate (IUPAC name) (1076)+TX, diflubenzuron (250)+TX, dilor [CCN]+TX, dimefluthrin [CCN]+TX, dimefox (1081)+TX, dimetan (1085)+TX, dimethoate (262)+TX, dimethrin (1083)+TX, dimethylvinphos (265)+TX, dimetilan (1086)+TX, dinex (1089)+TX, dinex-diclexine (1089)+TX, dinoprop (1093)+TX, dinosam (1094)+TX, dinoseb (1095)+TX, dinotefuran (271)+TX, diofenolan (1099)+TX, dioxabenzofos (1100)+TX, dioxacarb (1101)+TX, dioxathion (1102)+TX, disulfoton (278)+TX, dithicrofos (1108)+TX, DNOC (282)+TX, doramectin [CCN]+TX, DSP (1115)+TX, ecdysterone [CCN]+TX, El 1642 (development code) (1118)+TX, emamectin (291)+TX, emamectin benzoate (291)+TX, EMPC (1120)+TX, empenthrin (292)+TX, endosulfan (294)+TX, endothion (1121)+TX, endrin (1122)+TX, EPBP (1123)+TX, EPN (297)+TX, epofenonane (1124)+TX, eprinomectin [CCN]+TX, esfenvalerate (302)+TX, etaphos [CCN]+TX, ethiofencarb (308)+TX, ethion (309)+TX, ethiprole (310)+TX, ethoate-methyl (1134)+TX, ethoprophos (312)+TX, ethyl formate (IUPAC name) [CCN]+TX, ethyl-DDD (1056)+TX, ethylene dibromide (316)+TX, ethylene dichloride (chemical name) (1136)+TX, ethylene oxide [CCN]+TX, etofenprox (319)+TX, etrimfos (1142)+TX, EXD (1143)+TX, famphur (323)+TX, fenamiphos (326)+TX, fenazaflor (1147)+TX, fenchlorphos (1148)+TX, fenethacarb (1149)+TX, fenfluthrin (1150)+TX, fenitrothion (335)+TX, fenobucarb (336)+TX, fenoxacrim (1153)+TX, fenoxycarb (340)+TX, fenpirithrin (1155)+TX, fenpropathrin (342)+TX, fenpyrad+TX, fensulfothion (1158)+TX, fenthion (346)+TX, fenthion-ethyl [CCN]+TX, fenvalerate (349)+TX, fipronil (354)+TX, flonicamid (358)+TX, flubendiamide (CAS. Reg. No.: 272451-65-7)+TX, flucofuron (1168)+TX, flucycloxuron (366)+TX, flucythrinate (367)+TX, fluenetil (1169)+TX, flufenerim [CCN]+TX, flufenoxuron (370)+TX, flufenprox (1171)+TX, flumethrin (372)+TX, fluvalinate (1184)+TX, FMC 1137 (development code) (1185)+TX, fonofos (1191)+TX, formetanate (405)+TX, formetanate hydrochloride (405)+TX, formothion (1192)+TX, formparanate (1193)+TX, fosmethilan (1194)+TX, fospirate (1195)+TX, fosthiazate (408)+TX, fosthietan (1196)+TX, furathiocarb (412)+TX, furethrin (1200)+TX, gamma-cyhalothrin (197)+TX, gamma-HCH (430)+TX, guazatine (422)+TX, guazatine acetates (422)+TX, GY-81 (development code) (423)+TX, halfenprox (424)+TX, halofenozide (425)+TX, HCH (430)+TX, HEOD (1070)+TX, heptachlor (1211)+TX, heptenophos (432)+TX, heterophos [CCN]+TX, hexaflumuron (439)+TX, HHDN (864)+TX, hydramethylnon (443)+TX, hydrogen cyanide (444)+TX, hydroprene (445)+TX, hyquincarb (1223)+TX, imidacloprid (458)+TX, imiprothrin (460)+TX, indoxacarb (465)+TX, iodomethane (IUPAC name) (542)+TX, IPSP (1229)+TX, isazofos (1231)+TX, isobenzan (1232)+TX, isocarbophos (473)+TX, isodrin (1235)+TX, isofenphos (1236)+TX, isolane (1237)+TX, isoprocarb (472)+TX, isopropyl O-(methoxyaminothiophosphoryl)salicylate (IUPAC name) (473)+TX, isoprothiolane (474)+TX, isothioate (1244)+TX, isoxathion (480)+TX, ivermectin [CCN]+TX, jasmolin I (696)+TX, jasmolin II (696)+TX, jodfenphos (1248)+TX, juvenile hormone I [CCN]+TX, juvenile hormone II [CCN]+TX, juvenile hormone Ill [CCN]+TX, kelevan (1249)+TX, kinoprene (484)+TX, lambda-cyhalothrin (198)+TX, lead arsenate [CCN]+TX, lepimectin (CCN)+TX, leptophos (1250)+TX, lindane (430)+TX, lirimfos (1251)+TX, lufenuron (490)+TX, lythidathion (1253)+TX, m-cumenyl methylcarbamate (IUPAC name) (1014)+TX, magnesium phosphide (IUPAC name) (640)+TX, malathion (492)+TX, malonoben (1254)+TX, mazidox (1255)+TX, mecarbam (502)+TX, mecarphon (1258)+TX, menazon (1260)+TX, mephosfolan (1261)+TX, mercurous chloride (513)+TX, mesulfenfos (1263)+TX, metaflumizone (CCN)+TX, metam (519)+TX, metam-potassium (519)+TX, metam-sodium (519)+TX, methacrifos (1266)+TX, methamidophos (527)+TX, methanesulfonyl fluoride (IUPAC/Chemical Abstracts name) (1268)+TX, methidathion (529)+TX, methiocarb (530)+TX, methocrotophos (1273)+TX, methomyl (531)+TX, methoprene (532)+TX, methoquin-butyl (1276)+TX, methothrin (533)+TX, methoxychlor (534)+TX, methoxyfenozide (535)+TX, methyl bromide (537)+TX, methyl isothiocyanate (543)+TX, methylchloroform [CCN]+TX, methylene chloride [CCN]+TX, metofluthrin [CCN]+TX, metolcarb (550)+TX, metoxadiazone (1288)+TX, mevinphos (556)+TX, mexacarbate (1290)+TX, milbemectin (557)+TX, milbemycin oxime [CCN]+TX, mipafox (1293)+TX, mirex (1294)+TX, monocrotophos (561)+TX, morphothion (1300)+TX, moxidectin [CCN]+TX, naftalofos [CCN]+TX, naled (567)+TX, naphthalene (IUPAC/Chemical Abstracts name) (1303)+TX, NC-170 (development code) (1306)+TX, NC-184 (compound code)+TX, nicotine (578)+TX, nicotine sulfate (578)+TX, nifluridide (1309)+TX, nitenpyram (579)+TX, nithiazine (1311)+TX, nitrilacarb (1313)+TX, nitrilacarb 1:1 zinc chloride complex (1313)+TX, NNI-0101 (compound code)+TX, NNI-0250 (compound code)+TX, nornicotine (traditional name) (1319)+TX, novaluron (585)+TX, noviflumuron (586)+TX, O-5-dichloro-4-iodophenyl O-ethyl ethylphosphonothioate (IUPAC name) (1057)+TX, O,O-diethyl O-4-methyl-2-oxo-2H-chromen-7-yl phosphorothioate (IUPAC name) (1074)+TX, O,O-diethyl O-6-methyl-2-propylpyrimidin-4-yl phosphorothioate (IUPAC name) (1075)+TX, O,O,O′,O′-tetrapropyl dithiopyrophosphate (IUPAC name) (1424)+TX, oleic acid (IUPAC name) (593)+TX, omethoate (594)+TX, oxamyl (602)+TX, oxydemeton-methyl (609)+TX, oxydeprofos (1324)+TX, oxydisulfoton (1325)+TX, pp′-DDT (219)+TX, para-dichlorobenzene [CCN]+TX, parathion (615)+TX, parathion-methyl (616)+TX, penfluron [CCN]+TX, pentachlorophenol (623)+TX, pentachlorophenyl laurate (IUPAC name) (623)+TX, permethrin (626)+TX, petroleum oils (628)+TX, PH 60-38 (development code) (1328)+TX, phenkapton (1330)+TX, phenothrin (630)+TX, phenthoate (631)+TX, phorate (636)+TX, phosalone (637)+TX, phosfolan (1338)+TX, phosmet (638)+TX, phosnichlor (1339)+TX, phosphamidon (639)+TX, phosphine (IUPAC name) (640)+TX, phoxim (642)+TX, phoxim-methyl (1340)+TX, pirimetaphos (1344)+TX, pirimicarb (651)+TX, pirimiphos-ethyl (1345)+TX, pirimiphos-methyl (652)+TX, polychlorodicyclopentadiene isomers (IUPAC name) (1346)+TX, polychloroterpenes (traditional name) (1347)+TX, potassium arsenite [CCN]+TX, potassium thiocyanate [CCN]+TX, prallethrin (655)+TX, precocene I [CCN]+TX, precocene II [CCN]+TX, precocene III [CCN]+TX, primidophos (1349)+TX, profenofos (662)+TX, profluthrin [CCN]+TX, promacyl (1354)+TX, promecarb (1355)+TX, propaphos (1356)+TX, propetamphos (673)+TX, propoxur (678)+TX, prothidathion (1360)+TX, prothiofos (686)+TX, prothoate (1362)+TX, protrifenbute [CCN]+TX, pymetrozine (688)+TX, pyraclofos (689)+TX, pyrazophos (693)+TX, pyresmethrin (1367)+TX, pyrethrin I (696)+TX, pyrethrin II (696)+TX, pyrethrins (696)+TX, pyridaben (699)+TX, pyridalyl (700)+TX, pyridaphenthion (701)+TX, pyrimidifen (706)+TX, pyrimitate (1370)+TX, pyriproxyfen (708)+TX, quassia [CCN]+TX, quinalphos (711)+TX, quinalphos-methyl (1376)+TX, quinothion (1380)+TX, quintiofos (1381)+TX, R-1492 (development code) (1382)+TX, rafoxanide [CCN]+TX, resmethrin (719)+TX, rotenone (722)+TX, RU 15525 (development code) (723)+TX, RU 25475 (development code) (1386)+TX, ryania (1387)+TX, ryanodine (traditional name) (1387)+TX, sabadilla (725)+TX, schradan (1389)+TX, sebufos+TX, selamectin [CCN]+TX, SI-0009 (compound code)+TX, SI-0205 (compound code)+TX, SI-0404 (compound code)+TX, SI-0405 (compound code)+TX, silafluofen (728)+TX, SN 72129 (development code) (1397)+TX, sodium arsenite [CCN]+TX, sodium cyanide (444)+TX, sodium fluoride (IUPAC/Chemical Abstracts name) (1399)+TX, sodium hexafluorosilicate (1400)+TX, sodium pentachlorophenoxide (623)+TX, sodium selenate (IUPAC name) (1401)+TX, sodium thiocyanate [CCN]+TX, sophamide (1402)+TX, spinosad (737)+TX, spiromesifen (739)+TX, spirotetrmat (CCN)+TX, sulcofuron (746)+TX, sulcofuron-sodium (746)+TX, sulfluramid (750)+TX, sulfotep (753)+TX, sulfuryl fluoride (756)+TX, sulprofos (1408)+TX, tar oils (758)+TX, tau-fluvalinate (398)+TX, tazimcarb (1412)+TX, TDE (1414)+TX, tebufenozide (762)+TX, tebufenpyrad (763)+TX, tebupirimfos (764)+TX, teflubenzuron (768)+TX, tefluthrin (769)+TX, temephos (770)+TX, TEPP (1417)+TX, terallethrin (1418)+TX, terbam+TX, terbufos (773)+TX, tetrachloroethane [CCN]+TX, tetrachlorvinphos (777)+TX, tetramethrin (787)+TX, theta-cypermethrin (204)+TX, thiacloprid (791)+TX, thiafenox+TX, thiamethoxam (792)+TX, thicrofos (1428)+TX, thiocarboxime (1431)+TX, thiocyclam (798)+TX, thiocyclam hydrogen oxalate (798)+TX, thiodicarb (799)+TX, thiofanox (800)+TX, thiometon (801)+TX, thionazin (1434)+TX, thiosultap (803)+TX, thiosultap-sodium (803)+TX, thuringiensin [CCN]+TX, tolfenpyrad (809)+TX, tralomethrin (812)+TX, transfluthrin (813)+TX, transpermethrin (1440)+TX, triamiphos (1441)+TX, triazamate (818)+TX, triazophos (820)+TX, triazuron+TX, trichlorfon (824)+TX, trichlormetaphos-3 [CCN]+TX, trichloronat (1452)+TX, trifenofos (1455)+TX, triflumuron (835)+TX, trimethacarb (840)+TX, triprene (1459)+TX, vamidothion (847)+TX, vaniliprole [CCN]+TX, veratridine (725)+TX, veratrine (725)+TX, XMC (853)+TX, xylylcarb (854)+TX, YI-5302 (compound code)+TX, zeta-cypermethrin (205)+TX, zetamethrin+TX, zinc phosphide (640)+TX, zolaprofos (1469) and ZXI 8901 (development code) (858)+TX, cyantraniliprole [736994-63-19+TX, chlorantraniliprole [500008-45-7]+TX, cyenopyrafen [560121-52-0]+TX, cyflumetofen [400882-07-7]+TX, pyrifluquinazon [337458-27-2]+TX, spinetoram [187166-40-1+187166-15-0]+TX, spirotetramat [203313-25-1]+TX, sulfoxaflor [946578-00-3]+TX, flufiprole [704886-18-0]+TX, meperfluthrin [915288-13-0]+TX, tetramethylfluthrin [84937-88-2]+TX, triflumezopyrim (disclosed in WO 2012/092115)+TX, fluxametamide (WO 2007/026965)+TX, epsilon-metofluthrin [240494-71-7]+TX, epsilon-momfluorothrin [1065124-65-3]+TX, fluazaindolizine [1254304-22-7]+TX, chloroprallethrin [399572-87-3]+TX, fluxametamide [928783-29-3]+TX, cyhalodiamide [1262605-53-7]+TX, tioxazafen [330459-31-9]+TX, broflanilide [1207727-04-5]+TX, flufiprole [704886-18-0]+TX, cyclaniliprole [1031756-98-5]+TX, tetraniliprole [1229654-66-3]+TX, guadipyr (described in WO2010/060231)+TX, cycloxaprid (described in WO2005/077934)+TX,

a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX, a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cytokinins (210)+TX, dazomet (216)+TX, DBCP (1045)+TX, DCIP (218)+TX, diamidafos (1044)+TX, dichlofenthion (1051)+TX, dicliphos+TX, dimethoate (262)+TX, doramectin [CCN]+TX, emamectin (291)+TX, emamectin benzoate (291)+TX, eprinomectin [CCN]+TX, ethoprophos (312)+TX, ethylene dibromide (316)+TX, fenamiphos (326)+TX, fenpyrad+TX, fensulfothion (1158)+TX, fosthiazate (408)+TX, fosthietan (1196)+TX, furfural [CCN]+TX, GY-81 (development code) (423)+TX, heterophos [CCN]+TX, iodomethane (IUPAC name) (542)+TX, isamidofos (1230)+TX, isazofos (1231)+TX, ivermectin [CCN]+TX, kinetin (210)+TX, mecarphon (1258)+TX, metam (519)+TX, metam-potassium (519)+TX, metam-sodium (519)+TX, methyl bromide (537)+TX, methyl isothiocyanate (543)+TX, milbemycin oxime [CCN]+TX, moxidectin [CCN]+TX, Myrothecium verrucaria composition (565)+TX, NC-184 (compound code)+TX, oxamyl (602)+TX, phorate (636)+TX, phosphamidon (639)+TX, phosphocarb [CCN]+TX, sebufos+TX, selamectin [CCN]+TX, spinosad (737)+TX, terbam+TX, terbufos (773)+TX, tetrachlorothiophene (IUPAC/Chemical Abstracts name) (1422)+TX, thiafenox+TX, thionazin (1434)+TX, triazophos (820)+TX, triazuron+TX, xylenols [CCN]+TX, YI-5302 (compound code) and zeatin (210)+TX, fluensulfone [318290-98-1]+TX,

a nitrification inhibitor selected from the group of substances consisting of potassium ethylxanthate [CCN] and nitrapyrin (580)+TX,

a plant activator selected from the group of substances consisting of acibenzolar (6)+TX, acibenzolar-S-methyl (6)+TX, probenazole (658) and Reynoutria sachalinensis extract (720)+TX, a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (91)+TX, bromethalin (92)+TX, calcium cyanide (444)+TX, chloralose (127)+TX, chlorophacinone (140)+TX, cholecalciferol (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX,

a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX,

an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX,

a virucide selected from the group of substances consisting of imanin [CCN] and ribavirin [CCN]+TX,

a wound protectant selected from the group of substances consisting of mercuric oxide (512)+TX, octhilinone (590) and thiophanate-methyl (802)+TX,

and biologically active compounds selected from the group consisting of azaconazole (60207-31-0]+TX, benzovindiflupyr [1072957-71-1]+TX, bitertanol [70585-36-3]+TX, bromuconazole [116255-48-2]+TX, cyproconazole [94361-06-5]+TX, difenoconazole [119446-68-3]+TX, diniconazole [83657-24-3]+TX, epoxiconazole [106325-08-0]+TX, fenbuconazole [114369-43-6]+TX, fluquinconazole [136426-54-5]+TX, flusilazole [85509-19-9]+TX, flutriafol [76674-21-0]+TX, hexaconazole [79983-71-4]+TX, imazalil [35554-44-0]+TX, imibenconazole [86598-92-7]+TX, ipconazole [125225-28-7]+TX, metconazole [125116-23-6]+TX, myclobutanil [88671-89-0]+TX, pefurazoate [101903-30-4]+TX, penconazole [66246-88-6]+TX, prothioconazole [178928-70-6]+TX, pyrifenox [88283-41-4]+TX, prochloraz [67747-09-5]+TX, propiconazole [60207-90-1]+TX, simeconazole [149508-90-7]+TX, tebuconazole [107534-96-3]+TX, tetraconazole [112281-77-3]+TX, triadimefon [43121-43-3]+TX, triadimenol [55219-65-3]+TX, triflumizole [99387-89-0]+TX, triticonazole [131983-72-7]+TX, ancymidol [12771-68-5]+TX, fenarimol [60168-88-9]+TX, nuarimol [63284-71-9]+TX, bupirimate [41483-43-6]+TX, dimethirimol [5221-53-4]+TX, ethirimol [23947-60-6]+TX, dodemorph [1593-77-7]+TX, fenpropidine [67306-00-7]+TX, fenpropimorph [67564-91-4]+TX, spiroxamine [118134-30-8]+TX, tridemorph [81412-43-3]+TX, cyprodinil [121552-61-2]+TX, mepanipyrim [110235-47-7]+TX, pyrimethanil [53112-28-0]+TX, fenpiclonil [74738-17-3]+TX, fludioxonil [131341-86-1]+TX, benalaxyl [71626-11-4]+TX, furalaxyl [57646-30-7]+TX, metalaxyl [57837-19-1]+TX, R-metalaxyl [70630-17-0]+TX, ofurace [58810-48-3]+TX, oxadixyl [77732-09-3]+TX, benomyl [17804-35-2]+TX, carbendazim [10605-21-7]+TX, debacarb [62732-91-6]+TX, fuberidazole [3878-19-1]+TX, thiabendazole [148-79-8]+TX, chlozolinate [84332-86-5]+TX, dichlozoline [24201-58-9]+TX, iprodione [36734-19-7]+TX, myclozoline [54864-61-8]+TX, procymidone [32809-16-8]+TX, vinclozoline [50471-44-8]+TX, boscalid [188425-85-6]+TX, carboxin [5234-68-4]+TX, fenfuram [24691-80-3]+TX, fenpicoxamid [517875-34-2]+TX, flutolanil [66332-96-5]+TX, mepronil [55814-41-0]+TX, oxycarboxin [5259-88-1]+TX, penthiopyrad [183675-82-3]+TX, thifluzamide [130000-40-7]+TX, guazatine [108173-90-6]+TX, dodine [2439-10-3] [112-65-2] (free base)+TX, iminoctadine [13516-27-3]+TX, azoxystrobin [131860-33-8]+TX, dimoxystrobin [149961-52-4]+TX, enestroburin {Proc. BCPC, Int. Congr., Glasgow, 2003, 1, 93}+TX, fluoxastrobin [361377-29-9]+TX, kresoxim-methyl [143390-89-0]+TX, metominostrobin [133408-50-1]+TX, trifloxystrobin [141517-21-7]+TX, orysastrobin [248593-16-0]+TX, picoxystrobin [117428-22-5]+TX, pyraclostrobin [175013-18-0]+TX, ferbam [14484-64-1]+TX, mancozeb [8018-01-7]+TX, maneb [12427-38-2]+TX, metiram [9006-42-2]+TX, propineb [12071-83-9]+TX, thiram [137-26-8]+TX, zineb [12122-67-7]+TX, ziram [137-30-4]+TX, captafol [2425-06-1]+TX, captan [133-06-2]+TX, dichlofluanid [1085-98-9]+TX, fluoroimide [41205-21-4]+TX, folpet [133-07-3]+TX, tolylfluanid [731-27-1]+TX, bordeaux mixture [8011-63-0]+TX, copperhydroxid [20427-59-2]+TX, copperoxychlorid [1332-40-7]+TX, coppersulfat [7758-98-7]+TX, copperoxid [1317-39-1]+TX, mancopper [53988-93-5]+TX, oxine-copper [10380-28-6]+TX, dinocap [131-72-6]+TX, nitrothal-isopropyl [10552-74-6]+TX, edifenphos [17109-49-8]+TX, iprobenphos [26087-47-8]+TX, isoprothiolane [50512-35-1]+TX, phosdiphen [36519-00-3]+TX, pyrazophos [13457-18-6]+TX, tolclofos-methyl [57018-04-9]+TX, acibenzolar-S-methyl [135158-54-2]+TX, anilazine [101-05-3]+TX, benthiavalicarb [413615-35-7]+TX, blasticidin-S [2079-00-7]+TX, chinomethionat [2439-01-2]+TX, chloroneb [2675-77-6]+TX, chlorothalonil [1897-45-6]+TX, cyflufenamid [180409-60-3]+TX, cymoxanil [57966-95-7]+TX, dichlone [117-80-6]+TX, diclocymet [139920-32-4]+TX, diclomezine [62865-36-5]+TX, dicloran [99-30-9]+TX, diethofencarb [87130-20-9]+TX, dimethomorph [110488-70-5]+TX, SYP-LIg90 (Flumorph) [211867-47-9]+TX, dithianon [3347-22-6]+TX, ethaboxam [162650-77-3]+TX, etridiazole [2593-15-9]+TX, famoxadone [131807-57-3]+TX, fenamidone [161326-34-7]+TX, fenoxanil [115852-48-7]+TX, fentin [668-34-8]+TX, ferimzone [89269-64-7]+TX, fluazinam [79622-59-6]+TX, fluopicolide [239110-15-7]+TX, flusulfamide [106917-52-6]+TX, fenhexamid [126833-17-8]+TX, fosetyl-aluminium [39148-24-8]+TX, hymexazol [10004-44-1]+TX, iprovalicarb [140923-17-7]+TX, IKF-916 (Cyazofamid) [120116-88-3]+TX, kasugamycin [6980-18-3]+TX, methasulfocarb [66952-49-6]+TX, metrafenone [220899-03-6]+TX, oxathiapiprolin [1003318-67-9]+TX, pencycuron [66063-05-6]+TX, phthalide [27355-22-2]+TX, polyoxins [11113-80-7]+TX, probenazole [27605-76-1]+TX, propamocarb [25606-41-1]+TX, proquinazid [189278-12-4]+TX, pyroquilon [57369-32-1]+TX, quinoxyfen [124495-18-7]+TX, quintozene [82-68-8]+TX, sulfur [7704-34-9]+TX, tiadinil [223580-51-6]+TX, triazoxide [72459-58-6]+TX, tricyclazole [41814-78-2]+TX, triforine [26644-46-2]+TX, validamycin [37248-47-8]+TX, zoxamide (RH7281) [156052-68-5]+TX, mandipropamid [374726-62-2]+TX, isopyrazam [881685-58-1]+TX, sedaxane [874967-67-6]+TX, 3-difluoromethyl-1-methyl-1H-pyrazole-4-carboxylic acid (9-dichloromethylene-1,2,3,4-tetrahydro-1,4-methano-naphthalen-5-yl)-amide (dislosed in WO 2007/048556)+TX, 3-difluoromethyl-1-methyl-1H-pyrazole-4-carboxylic acid (3′,4′,5′-trifluoro-biphenyl-2-yl)-amide (disclosed in WO 2006/087343)+TX, [(3S,4R,4aR,6S,6aS,12R, 12aS, 12bS)-3-[(cyclopropylcarbonyl)oxy]-1,3,4,4a,5,6,6a,12,12a,12b-decahydro-6,12-dihydroxy-4,6a, 12b-trimethyl-11-oxo-9-(3-pyridinyl)-2H,11 Hnaphtho[2,1-b]pyrano[3,4-e]pyran-4-yl]methyl-cyclopropanecarboxylate [915972-17-7]+TX and 1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide [926914-55-8]+TX; lancotrione [1486617-21-3]+TX, florpyrauxifen [943832-81-3]]+TX, ipfentrifluconazole [1417782-08-1]+TX, mefentrifluconazole [1417782-03-6]+TX, quinofumelin [861647-84-9]]+TX, chloroprallethrin [399572-87-3]]+TX, cyhalodiamide [1262605-53-7]]+TX, fluazaindolizine [1254304-22-7]+TX, fluxametamide [928783-29-3]+TX, epsilon-metofluthrin [240494-71-7]]+TX, epsilon-momfluorothrin [1065124-65-3]+TX, pydiflumetofen [1228284-64-7]+TX, kappa-bifenthrin [439680-76-9]+TX, broflanilide [1207727-04-5]+TX, dicloromezotiaz [1263629-39-5]+TX, dipymetitrone [16114-35-5]+TX, pyraziflumid [942515-63-1] and kappa-tefluthrin [391634-71-2]+TX; and

microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD#32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomonas fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HC®+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus; and

Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame pepermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®); and

pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC-LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (E+TX,Z+TX,Z)-3+TX,8+TX,11 Tetradecatrienyl acetate+TX, (Z+TX,Z+TX,E)-7+TX,11+TX,13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate; and

Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline Cucumeris®)+TX, Amblyseius fallacis (Fallacis®)+TX, Amblyseius swirskii (Bugline Swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretlinee®)+TX, Eretmocerus eremicus (Bemimix®)+TX, Eretmocerus hayati+TX, Eretmocerus mundus (Bemipar®+TX, Eretline M®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline Hm®+TX, Larvanem-M®)+TX, Hippodamia convergens+TX, Hypoaspis aculeifer (Aculeifer-System®+TX, Entomite-A®)+TX, Hypoaspis miles (Hypoline M®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline C®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline I®)+TX, Orius laevigatus (Thripor-L®+TX, Oriline I®)+TX, Orius majusculus (Oriline M®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline P®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline Sf®+TX, Scia-Rid®+TX, Entonem®)+TX, Steinernema kraussei (Nemasys L®+TX, BioNem L®+TX, Exhibitline Srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine B®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator; and

other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline D®)+TX, Erwinia amylovora (Harpin) (ProAct®+TX, Ni-HIBIT Gold CST®)+TX, Ferri-phosphate (Ferramol®)+TX, Funnel traps (Trapline Y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline F®)+TX, Microctonus hyperodae+TX, Mycoleptodiscus terrestris (Des-X®)+TX, BioGain®+TX, Aminomite®+TX, Zenox®+TX, Pheromone trap (Thripline Ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+B®)+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright® 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “develoment code” is used or, if neither one of those designations nor a “common name” is used, an “alternative name” is employed. “CAS Reg. No” means the Chemical Abstracts Registry Number.

The active ingredient mixture of the compounds of formula (I) selected from a compound described in one of Tables 1.1 to 1.34, 2.1 to 2.33 or 3.1 to 3.31 ((below) or one of Tables T1, T2, T3 or T4 (below), and an active ingredient as described above are preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula (I) selected from one of Tables 1.1 to 1.34, 2.1 to 2.33 and 3.1 to 3.31 (below), or Table T1, Table T2, Table T3 or Table T4 (below), and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula (I) selected from Tables 1.1 to 1.34, 2.1 to 2.33 and 3.1 to 3.31 (below), or Table T1, Table T2, Table T3 or Table T4 (below), and the active ingredient(s) as described above, is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds (I) for the preparation of these compositions are also a subject of the invention.

Another aspect of invention is related to the use of a compound of formula (I) or of a preferred individual compound as defined herein, of a composition comprising at least one compound of formula (I) or at least one preferred individual compound as above-defined, or of a fungicidal or insecticidal mixture comprising at least one compound of formula (I) or at least one preferred individual compound as above-defined, in admixture with other fungicides or insecticides as described above, for controlling or preventing infestation of plants, e.g. useful plants such as crop plants, propagation material thereof, e.g. seeds, harvested crops, e.g. harvested food crops, or non-living materials by insects or by phytopathogenic microorganisms, preferably fungal organisms.

A further aspect of invention is related to a method of controlling or preventing an infestation of plants, e.g., useful plants such as crop plants, propagation material thereof, e.g. seeds, harvested crops, e.g., harvested food crops, or of non-living materials by insects or by phytopathogenic or spoilage microorganisms or organisms potentially harmful to man, especially fungal organisms, which comprises the application of a compound of formula (I) or of a preferred individual compound as above-defined as active ingredient to the plants, to parts of the plants or to the locus thereof, to the propagation material thereof, or to any part of the non-living materials.

Controlling or preventing means reducing infestation by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, or insects which comprises the application of a compound of formula (I), or an agrochemical composition which contains at least one of said compounds, is foliar application. The frequency of application and the rate of application will depend on the risk of infestation by the corresponding pathogen or insect. However, the compounds of formula (I) can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula I may also be applied to seeds (coating) by impregnating the seeds or tubers either with a liquid formulation of the fungicide or coating them with a solid formulation.

A formulation, e.g. a composition containing the compound of formula (I), and, if desired, a solid or liquid adjuvant or monomers for encapsulating the compound of formula (I), may be prepared in a known manner, typically by intimately mixing and/or grinding the compound with extenders, for example solvents, solid carriers and, optionally, surface active compounds (surfactants).

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

When the combinations of the present invention are used for treating seed, rates of 0.001 to 50 g of a compound of formula I per kg of seed, preferably from 0.01 to 10 g per kg of seed are generally sufficient.

Suitably, a composition comprising a compound of formula (I) according to the present invention is applied either preventative, meaning prior to disease development or curative, meaning after disease development.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the ondensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula (I) optionally together with other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

Whereas it is preferred to formulate commercial products as concentrates, the end user will normally use dilute formulations.

### Table 1.1:

This table discloses 64 specific compounds of the formula (T-1):

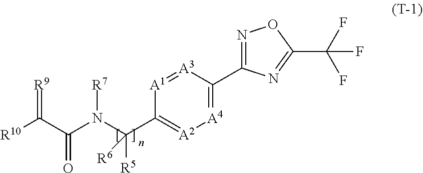

wherein n is 1, A¹ is C—R¹, A² is C—R², A³ is C—R³, A⁴ is C—R⁴ and R¹, R², R³, R⁴, R⁵, R⁶, and R⁷ are hydrogen, R⁹ is O, and R¹⁰ is as defined below in Table 1.

Each of Tables 1.2 to 1.34 (which follow Table 1.1) make available 64 individual compounds of the formula (T-1) in which n, A¹, A², A³, A⁴, R¹, R², R³, R⁴, R⁵, R⁶, R⁷ and R⁹ are as specifically defined in Tables 1.2 to 1.34, which refer to Table 1 wherein R¹⁰ is specifically defined.

TABLE 1

| Compound | |
|---|---|
| no. | R¹⁰ |
| 1.001 | methyl |
| 1.002 | ethyl |
| 1.003 | propyl |
| 1.004 | isopropyl |
| 1.005 | butyl |
| 1.006 | sec-butyl |
| 1.007 | tert-butyl |
| 1.008 | isobutyl |
| 1.009 | allyl |
| 1.010 | 2-methylallyl |
| 1.011 | 1,1-dimethylallyl |
| 1.012 | prop-2-ynyl |
| 1.013 | 1-acetoxymethyl |
| 1.014 | 2-acetoxyethyl |
| 1.015 | hydroxymethyl |
| 1.016 | 2-hydroxyethyl |
| 1.017 | 2-hydroxypropyl |
| 1.018 | 3-hydroxypropyl |
| 1.019 | fluoromethyl |
| 1.020 | difluoromethyl |
| 1.021 | trifluoromethyl |
| 1.022 | 2,2,2-trifluoroethyl |
| 1.023 | 3,3,3-trifluoropropyl |
| 1.024 | 4,4,4-trifluorobutyl |
| 1.025 | but-3-ynyl |
| 1.026 | but-2-ynyl |
| 1.027 | cyanomethyl |
| 1.028 | 2-cyanoethyl |
| 1.029 | 1-methoxymethyl |
| 1.030 | 2-methoxyethyl |
| 1.031 | 3-methoxypropyl |
| 1.032 | 2-ethoxyethyl |
| 1.033 | 1-methoxyethyl |
| 1.034 | 2-methoxypropyl |
| 1.035 | 2-methylsulfanylethyl |
| 1.036 | 2-methylsulfonylethyl |
| 1.037 | difluoromethoxymethyl |
| 1.038 | 1-difluoromethoxyethyl |
| 1.039 | 2-difluoromethoxyethyl |
| 1.040 | phenyl |
| 1.041 | pyrid-2-yl |
| 1.042 | pyrid-3-yl |
| 1.043 | pyrid-4-yl |
| 1.044 | phenylmethyl |
| 1.045 | pyrid-2-ylmethyl |
| 1.046 | cyclopropyl |
| 1.047 | cyclobutyl |
| 1.048 | cyclopentyl |
| 1.049 | cyclohexyl |
| 1.050 | cyclopropylmethyl |
| 1.051 | cyclobutylmethyl |
| 1.052 | cyclopentylmethyl |
| 1.053 | cyclohexylmethyl |
| 1.054 | tetrahydrofuran-2-yl |
| 1.055 | tetrahydrofuran-3-yl |
| 1.056 | tetrahydropyran-3-yl |
| 1.057 | tetrahydropyran-4-yl |
| 1.058 | tetrahydrofuran-2-ylmethyl |
| 1.059 | tetrahydrofuran-3-ylmethyl |
| 1.060 | tetrahydropyran-2-ylmethyl |
| 1.061 | tetrahydropyran-3-ylmethyl |
| 1.061 | 2-thienyl |
| 1.063 | 2-furyl |
| 1.064 | 3-fluorophenyl |

### Table 1.2:

This table discloses 64 specific compounds of formula (T-1) wherein n is 1, A¹ is C—R¹, A² is C—R², A³ is C—R³, A⁴ is C—R⁴ and R², R³, R⁴, R⁵, R⁶, and R⁷ are hydrogen, R¹ is fluorine, R⁹ is O, and R¹⁰ is as defined above in Table 1.

### Table 1.3:

This table discloses 64 specific compounds of formula (T-1) wherein n is 1, A¹ is C—R¹, A² is C—R², A³ is C—R³, A⁴ is C—R⁴ and R², R³, R⁴, R⁵, R⁶, and R⁷ are hydrogen, R¹ is chlorine, R⁹ is O, and R¹⁰ is as defined above in Table 1.

Table 1.4: This table discloses 64 specific compounds of formula (T-1) wherein n is 1, A¹ is C—R¹, A² is C—R², A³ is C—R³, A⁴ is C—R⁴ and R², R³, R⁴, R⁵, R⁶, and R⁷ are hydrogen, R¹ is methyl, R⁹ is O, and R¹⁰ is as defined above in Table 1.

### Table 1.5:

This table discloses 64 specific compounds of formula (T-1) wherein n is 1, A¹ is C—R¹, A² is C—R², A³ is C—R³, A⁴ is C—R⁴ and R², R³, R⁴, R⁵, R⁶, and R⁷ are hydrogen, R¹ is trifluoromethyl, R⁹ is O, and R¹⁰ is as defined above in Table 1.

### Table 1.6:

This table discloses 64 specific compounds of formula (T-1) wherein n is 1, A¹ is C—R¹, A² is C—R², A³ is C—R³, A⁴ is C—R⁴ and R², R³, R⁴, R⁵, R⁶, and R⁷ are hydrogen, R¹ is methoxy, R⁹ is O, and R¹⁰ is as defined above in Table 1.

### Table 1.7:

This table discloses 64 specific compounds of formula (T-1) wherein n is 1, A¹ is N, A² is C—R², A³ is C—R³, A⁴ is C—R⁴ and R¹, R², R³, R⁴, R⁵, R⁶, and R⁷ are hydrogen, R⁹ is O, and R¹⁰ is as defined above in Table 1.

### Table 1.8:

This table discloses 64 specific compounds of formula (T-1) wherein n is 1, A¹ is C—R¹, A² is C—R², A³ is C—R³, A⁴ is C—R⁴ and R¹, R², R⁴, R⁵, R⁶, and R⁷ are hydrogen, R³ is fluorine, R⁹ is O, and R¹⁰ is as defined above in Table 1.

### Table 1.9:

This table discloses 64 specific compounds of formula (T-1) wherein n is 1, A¹ is C—R¹, A² is C—R², A³ is C—R³, A⁴ is C—R⁴ and R², R⁴, R⁵, R⁶, and R⁷ are hydrogen, R¹ and R³ are fluorine, R⁹ is O, and R¹⁰ is as defined above in Table 1.

### Table 1.10:

This table discloses 64 specific compounds of formula (T-1) wherein n is 1, A¹ is C—R¹, A² is C—R², A³ is C—R³, A⁴ is C—R⁴ and R³, R⁴, R⁵, R⁶, and R⁷ are hydrogen, R¹ and R² are fluorine, R⁹ is O, and R¹⁰ is as defined above in Table 1.

### Table 1.11:

This table discloses 64 specific compounds of formula (T-1) wherein n is 2, A¹ is C—R¹, A² is C—R², A³ is C—R³, A⁴ is C—R⁴ and R¹, R², R³, R⁴, R⁵, R⁶, and R⁷ are hydrogen, R⁹ is O, and R¹⁰ is as defined above in Table 1.

### Table 1.12:

This table discloses 64 specific compounds of formula (T-1) wherein n is 2, A¹ is N, A² is C—R², A³ is C—R³, A⁴ is C—R⁴ and R², R³, R⁴, R⁵, R⁶, and R⁷ are hydrogen, R⁹ is O, and R¹⁰ is as defined above in Table 1.

### Table 1.13:

This table discloses 64 specific compounds of formula (T-1) wherein n is 1, A¹ is N, A² is N, A³ is C—R³, A⁴ is C—R⁴ and R³, R⁴, R⁵, R⁶, and R⁷ are hydrogen, R⁹ is O, and R¹⁰ is as defined above in Table 1.

### Table 1.14:

This table discloses 64 specific compounds of formula (T-1) wherein n is 1, A¹ is C—R¹, A² is C—R², A³ is N, A⁴ is C—R⁴ and R¹, R², R⁴, R⁵, R⁶, and R⁷ are hydrogen, R⁹ is O, and R¹⁰ is as defined above in Table 1.

### Table 1.15:

This table discloses 64 specific compounds of formula (T-1) wherein n is 1, A¹ is C—R¹, A² is C—R², A³ is C—R³, A⁴ is C—R⁴ and R¹, R², R³ R⁴, R⁵, and R⁷ are hydrogen, R⁶ is methyl, R⁹ is O, and R¹⁰ is as defined above in Table 1.

### Table 1.16:

This table discloses 64 specific compounds of formula (T-1) wherein n is 0, A¹ is C—R¹, A² is C—R², A³ is C—R³, A⁴ is C—R⁴ and R¹, R², R³, R⁴, and R⁷ are hydrogen, R⁹ is O, and R¹⁰ is as defined above in Table 1.

### Table 1.17:

This table discloses 64 specific compounds of formula (T-1) wherein n is 0, A¹ is C—R¹, A² is C—R², A³ is C—R³, A⁴ is C—R⁴ and R¹, R², R³, and R⁴ are hydrogen, and R⁷ is methyl, R⁹ is O, and R¹⁰ is as defined above in Table 1.

### Table 1.18:

This table discloses 64 specific compounds of formula (T-1) wherein n is 0, A¹ is N, A² is C—R², A³ is C—R³, A⁴ is C—R⁴ and R², R³, R⁴, and R⁷ are hydrogen, R¹ is fluorine, R⁹ is O, and R¹⁰ is as defined above in Table 1.

### Table 1.19:

This table discloses 64 specific compounds of formula (T-1) wherein n is 0, A¹ is C—R¹, A² is C—R², A³ is C—R³, A⁴ is C—R⁴ and R², R³, R⁴, and R⁷ are hydrogen, R⁹ is O, and R¹⁰ is as defined above in Table 1.

### Table 1.20:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is methyl, R⁹ is O, and R¹⁰ is as defined above in Table 1.

### Table 1.21:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is methoxy, R⁹ is O, and R¹⁰ is as defined above in Table 1.

### Table 1.22:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is ethyl, R⁹ is O, and R¹⁰ is as defined above in Table 1.

### Table 1.23:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is allyl, R⁹ is O, and R¹⁰ is as defined above in Table 1.

### Table 1.24:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is isobutyl, R⁹ is O, and R¹⁰ is as defined above in Table 1.

### Table 1.25:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is 2-methoxyethyl, R⁹ is O, and R¹⁰ is as defined above in Table 1.

### Table 1.26:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is propyloxy, R⁹ is O, and R¹⁰ is as defined above in Table 1.

### Table 1.27:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is propyn-2-yl, R⁹ is O, and R¹⁰ is as defined above in Table 1.

### Table 1.28:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is 4-chlorophenyl)methoxy, and R⁹ is as defined above in Table 1.

### Table 1.29:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is 3,3-dichloroallyloxy, R⁹ is O, and R¹⁰ is as defined above in Table 1.

### Table 1.30:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is 2-furylmethyl, R⁹ is O, and R¹⁰ is as defined above in Table 1.

### Table 1.31:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is tetrahydrofuran-2-ylmethyl, R⁹ is O, and R¹⁰ is as defined above in Table 1.

### Table 1.32:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is cyclopropylmethyl, R⁹ is O, and R¹⁰ is as defined above in Table 1.

### Table 1.33:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, and R⁶ are hydrogen, R⁷ is cyclopropyl, R⁹ is O, and R¹⁰ is as defined above in Table 1.

### Table 1.34:

This table discloses 64 specific compounds of formula (T-1) wherein n is 1, A¹ is C—R¹, A² is C—R², A³ is C—R³, A⁴ is C—R⁴ and R¹, R², R³, R⁴, R⁵, R⁶, and R⁷ are hydrogen, R⁹ is N—(OMe), and R¹⁰ is as defined above in Table 1.

### Table 2.1:

This table discloses 53 specific compounds of the formula (T-2):

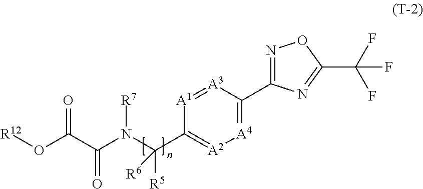

wherein n is 1, A¹ is C—R¹, A² is C—R², A³ is C—R³, A⁴ is C—R⁴ and R¹, R², R³, R⁴, R⁵, R⁶, and R⁷ are hydrogen and R¹² is as defined below in Table 2.

Each of Tables 2.1 to 2.33 (which follow Table 2.1) make available 53 individual compounds of the formula (T-2) in which n, A¹, A², A³, A⁴, R¹, R², R³, R⁴, R⁵, R⁶, and R⁷ are as specifically defined in Tables 2.1 to 2.33, which refer to Table 2 wherein R¹² is specifically defined.

TABLE 2

| Compound | |
|---|---|
| no. | R¹² |
| 2.001 | methyl |
| 2.002 | ethyl |
| 2.003 | propyl |
| 2.004 | isopropyl |
| 2.005 | butyl |
| 2.006 | sec-butyl |
| 2.007 | tert-butyl |
| 2.008 | isobutyl |
| 2.009 | pentyl |
| 2.010 | allyl |
| 2.011 | 2-methylallyl |
| 2.012 | 1,1-dimethylallyl |
| 2.013 | prop-2-ynyl |
| 2.014 | 2-acetoxyethyl |
| 2.015 | 2-hydroxyethyl |
| 2.016 | 2-hydroxypropyl |
| 2.017 | 3-hydroxypropyl |
| 2.018 | 2,2-difluoroethyl |
| 2.019 | 2,2,2-trifluoroethyl |
| 2.020 | 3,3,3-trifluoropropyl |
| 2.021 | 4,4,4-trifluorobutyl |
| 2.022 | but-3-ynyl |
| 2.023 | but-2-ynyl |
| 2.024 | 2-cyanoethyl |
| 2.025 | 2-methoxyethyl |
| 2.026 | 2-ethoxyethyl |
| 2.027 | 2-methoxypropyl |
| 2.028 | 1-methoxy-4-piperidyl |
| 2.029 | oxetan-3-yl |
| 2.030 | phenyl |
| 2.031 | pyrid-2-yl |
| 2.032 | pyrid-3-yl |
| 2.033 | pyrid-4-yl |
| 2.034 | phenylmethyl |
| 2.035 | pyrid-2-ylmethyl |
| 2.036 | cyclopropyl |
| 2.037 | 1-cyanocycloprop-1-yl |
| 2.038 | 1-fluorocycloprop-1-yl |
| 2.039 | 1-methylcycloprop-1-yl |
| 2.040 | cyclobutyl |
| 2.041 | cyclopentyl |
| 2.042 | cyclohexyl |
| 2.043 | cyclopropylmethyl |
| 2.044 | cyclobutylmethyl |
| 2.045 | cyclopentylmethyl |
| 2.046 | cyclohexylmethyl |
| 2.047 | tetrahydrofuran-3-yl |
| 2.048 | tetrahydropyran-3-yl |
| 2.049 | tetrahydropyran-4-yl |
| 2.050 | tetrahydrofuran-2-ylmethyl |
| 2.051 | tetrahydrofuran-3-ylmethyl |
| 2.052 | tetrahydropyran-2-ylmethyl |
| 2.053 | tetrahydropyran-3-ylmethyl |

### Table 2.2:

This table discloses 53 specific compounds of formula (T-2) wherein n is 1, A¹ is C—R¹, A² is C—R², A³ is C—R³, A⁴ is C—R⁴ and R², R³, R⁴, R⁵, R⁶, and R⁷ are hydrogen, R¹ is fluorine, and R¹² is as defined above in Table 2.

### Table 2.3:

This table discloses 53 specific compounds of formula (T-2) wherein n is 1, A¹ is C—R¹, A² is C—R², A³ is C—R³, A⁴ is C—R⁴ and R², R³, R⁴, R⁵, R⁶, and R⁷ are hydrogen, R¹ is chlorine, and R¹² is as defined above in Table 2.

### Table 2.4:

This table discloses 53 specific compounds of formula (T-2) wherein n is 1, A¹ is C—R¹, A² is C—R², A³ is C—R³, A⁴ is C—R⁴ and R², R³, R⁴, R⁵, R⁶, and R⁷ are hydrogen, R¹ is methyl, and R¹² is as defined above in Table 2.

### Table 2.5:

This table discloses 53 specific compounds of formula (T-2) wherein n is 1, A¹ is C—R¹, A² is C—R², A³ is C—R³, A⁴ is C—R⁴ and R², R³, R⁴, R⁵, R⁶, and R⁷ are hydrogen, R¹ is trifluoromethyl, and R¹² is as defined above in Table 2.

### Table 2.6:

This table discloses 53 specific compounds of formula (T-2) wherein n is 1, A¹ is C—R¹, A² is C—R², A³ is C—R³, A⁴ is C—R⁴ and R², R³, R⁴, R⁵, R⁶, and R⁷ are hydrogen, R¹ is methoxy, and R¹² is as defined above in Table 2.

### Table 2.7:

This table discloses 53 specific compounds of formula (T-2) wherein n is 1, A¹ is N, A² is C—R², A³ is C—R³, A⁴ is C—R⁴ and R¹, R², R³, R⁴, R⁵, R⁶, and R⁷ are hydrogen, and R¹² is as defined above in Table 2.

### Table 2.8:

This table discloses 53 specific compounds of formula (T-2) wherein n is 1, A¹ is C—R¹, A² is C—R², A³ is C—R³, A⁴ is C—R⁴ and R¹, R², R⁴, R⁵, R⁶, and R⁷ are hydrogen, R³ is fluorine, and R¹² is as defined above in Table 2.

### Table 2.9:

This table discloses 53 specific compounds of formula (T-2) wherein n is 1, A¹ is C—R¹, A² is C—R², A³ is C—R³, A⁴ is C—R⁴ and R², R⁴, R⁵, R⁶, and R⁷ are hydrogen, R¹ and R³ are fluorine, and R¹² is as defined above in Table 2.

### Table 2.10:

This table discloses 53 specific compounds of formula (T-2) wherein n is 1, A¹ is C—R¹, A² is C—R², A³ is C—R³, A⁴ is C—R⁴ and R³, R⁴, R⁵, R⁶, and R⁷ are hydrogen, R¹ and R² are fluorine, and R¹² is as defined above in Table 2.

### Table 2.11:

This table discloses 53 specific compounds of formula (T-2) wherein n is 2, A¹ is C—R¹, A² is C—R², A³ is C—R³, A⁴ is C—R⁴ and R¹, R², R³, R⁴, R⁵, R⁶, and R⁷ are hydrogen, and R¹² is as defined above in Table 2.

### Table 2.12:

This table discloses 53 specific compounds of formula (T-2) wherein n is 2, A¹ is N, A² is C—R², A³ is C—R³, A⁴ is C—R⁴ and R², R³, R⁴, R⁵, R⁶, and R⁷ are hydrogen, and R¹² is as defined above in Table 2.

### Table 2.13:

This table discloses 53 specific compounds of formula (T-2) wherein n is 1, A¹ is N, A² is N, A³ is C—R³, A⁴ is C—R⁴ and R³, R⁴, R⁵, R⁶, and R⁷ are hydrogen, and R¹² is as defined above in Table 2.

### Table 2.14:

This table discloses 53 specific compounds of formula (T-2) wherein n is 1, A¹ is C—R¹, A² is C—R², A³ is N, A⁴ is C—R⁴ and R¹, R², R⁴, R⁵, R⁶, and R⁷ are hydrogen, and R¹² is as defined above in Table 2.

### Table 2.15:

This table discloses 53 specific compounds of formula (T-2) wherein n is 1, A¹ is C—R¹, A² is C—R², A³ is C—R³, A⁴ is C—R⁴ and R¹, R², R³ R⁴, R⁵, and R⁷ are hydrogen, R⁶ is methyl, and R¹² is as defined above in Table 2.

### Table 2.16:

This table discloses 53 specific compounds of formula (T-2) wherein n is 0, A¹ is C—R¹, A² is C—R², A³ is C—R³, A⁴ is C—R⁴ and R¹, R², R³, R⁴, and R⁷ are hydrogen, and R¹² is as defined above in Table 2.

### Table 2.17:

This table discloses 53 specific compounds of formula (T-2) wherein n is 0, A¹ is C—R¹, A² is C—R², A³ is C—R³, A⁴ is C—R⁴ and R¹, R², R³, and R⁴ are hydrogen, and R⁷ is methyl, and R¹² is as defined above in Table 2.

### Table 2.18:

This table discloses 53 specific compounds of formula (T-2) wherein n is 0, A¹ is N, A² is C—R², A³ is C—R³, A⁴ is C—R⁴ and R², R³, R⁴, and R⁷ are hydrogen, R¹ is fluorine, and R¹² is as defined above in Table 2.

### Table 2.19:

This table discloses 53 specific compounds of formula (T-2) wherein n is 0, A¹ is C—R¹, A² is C—R², A³ is C—R³, A⁴ is C—R⁴ and R², R³, R⁴, and R⁷ are hydrogen, and R¹² is as defined above in Table 2.

### Table 2.20:

This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is methyl, and R¹² is as defined above in Table 2.

### Table 2.21:

This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is methoxy, and R¹² is as defined above in Table 2.

### Table 2.22:

This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is ethyl, and R¹² is as defined above in Table 2.

### Table 2.23:

This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is allyl, and R¹² is as defined above in Table 2.

### Table 2.24:

This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is isobutyl, and R¹² is as defined above in Table 2.

### Table 2.25:

This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is 2-methoxyethyl, and R¹² is as defined above in Table 2.

### Table 2.26:

This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is propyloxy, and R¹² is as defined above in Table 2.

### Table 2.27:

This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is propyn-2-yl, n is 1, and R¹² is as defined above in Table 2.

### Table 2.28:

This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is 4-chlorophenyl)methoxy, and R¹² is as defined above in Table 2.

### Table 2.29:

This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is 3,3-dichloroallyloxy, and R¹² is as defined above in Table 2.

### Table 2.30:

This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is 2-furylmethyl, and R¹² is as defined above in Table 2.

Table 2.31: This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is tetrahydrofuran-2-ylmethyl, and R¹² is as defined above in Table 2.

### Table 2.32:

This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is cyclopropylmethyl, and R¹² is as defined above in Table 2.

### Table 2.33:

This table discloses 53 specific compounds of formula (T-2) wherein n is 1, A¹ is C—R¹, A² is C—R², A³ is C—R³, A⁴ is C—R⁴ and R¹, R², R³, R⁴, R⁵, and R⁶ are hydrogen, R⁷ is cyclopropyl, and R¹² is as defined above in Table 2.

### Table 3.1:

This table discloses 130 specific compounds of the formula (T-3):

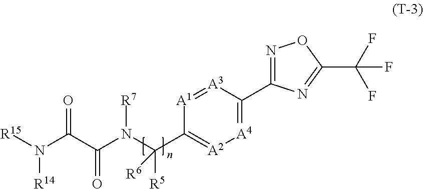

wherein A¹ is C—R¹, A² is C—R², A³ is C—R³, A⁴ is C—R⁴ and R¹, R², R³, R⁴, R⁵, R⁶, and R⁷ are hydrogen, n is 1, and R¹⁴ and R¹⁵ are as defined below in Table 3.

Each of Tables 3.2 to 3.31 (which follow Table 3.1) make available 130 individual compounds of the formula (T-3) in which n, A¹, A², A³, A⁴, R¹, R², R³, R⁴, R⁵, R⁶, and R⁷ are as specifically defined in Tables 3.2 to 3.31, which refer to Table 3 wherein R¹⁴ and R¹⁵ are specifically defined.

TABLE 3

| Compound | | |
|---|---|---|
| no. | R¹⁴ | R¹⁵ |
| 3.001 | H | H |
| 3.002 | methyl | H |
| 3.003 | ethyl | H |
| 3.004 | propyl | H |
| 3.005 | isopropyl | H |
| 3.006 | butyl | H |
| 3.007 | sec-butyl | H |
| 3.008 | tert-butyl | H |
| 3.009 | isobutyl | H |
| 3.010 | neopentyl | H |
| 3.011 | pentyl | H |
| 3.012 | allyl | H |
| 3.013 | 2-methylallyl | H |
| 3.014 | 1,1-dimethylallyl | H |
| 3.015 | prop-2-ynyl | H |
| 3.016 | 2,2-difluoroethyl | H |
| 3.017 | 2,2,2-trifluoroethyl | H |
| 3.018 | 2-cyanoethyl | H |
| 3.019 | 2-methoxyethyl | H |
| 3.020 | 1-methoxy-4-piperidyl | H |
| 3.021 | oxetan-3-yl | H |
| 3.022 | phenyl | H |
| 3.023 | pyrid-2-yl | H |
| 3.024 | pyrid-3-yl | H |
| 3.025 | pyrid-4-yl | H |
| 3.026 | phenylmethyl | H |
| 3.027 | pyrid-2-ylmethyl | H |
| 3.028 | cyclopropyl | H |
| 3.029 | 1-cyanocycloprop-1-yl | H |
| 3.030 | 1-fluorocycloprop-1-yl | H |
| 3.031 | 1-methylcycloprop-1-yl | H |
| 3.032 | cyclobutyl | H |
| 3.033 | cyclopentyl | H |
| 3.034 | cyclohexyl | H |
| 3.035 | cyclopropylmethyl | H |
| 3.036 | cyclobutylmethyl | H |
| 3.037 | cyclopentylmethyl | H |
| 3.038 | cyclohexylmethyl | H |
| 3.039 | tetrahydrofuran-3-yl | H |
| 3.040 | tetrahydropyran-3-yl | H |
| 3.041 | tetrahydropyran-4-yl | H |
| 3.042 | tetrahydrofuran-2-ylmethyl | H |
| 3.043 | tetrahydrofuran-3-ylmethyl | H |
| 3.044 | tetrahydropyran-2-ylmethyl | H |
| 3.045 | tetrahydropyran-3-ylmethyl | H |
| 3.046 | methoxy | H |
| 3.047 | ethoxy | H |
| 3.048 | propoxy | H |
| 3.049 | isopropoxy | H |
| 3.050 | butoxy | H |
| 3.051 | 2-furylmethyl | H |
| 3.052 | methyl | methyl |
| 3.060 | ethyl | methyl |
| 3.054 | propyl | methyl |
| 3.055 | isopropyl | methyl |
| 3.056 | butyl | methyl |
| 3.057 | sec-butyl | methyl |
| 3.058 | tert-butyl | methyl |
| 3.059 | isobutyl | methyl |
| 3.060 | neopentyl | methyl |
| 3.061 | pentyl | methyl |
| 3.062 | allyl | methyl |
| 3.063 | 2-methylallyl | methyl |
| 3.064 | 1,1-dimethylallyl | methyl |
| 3.065 | prop-2-ynyl | methyl |
| 3.066 | 2,2-difluoroethyl | methyl |
| 3.067 | 2,2,2-trifluoroethyl | methyl |
| 3.068 | 2-cyanoethyl | methyl |
| 3.069 | 2-methoxyethyl | methyl |
| 3.070 | 1-methoxy-4-piperidyl | methyl |
| 3.071 | oxetan-3-yl | methyl |
| 3.072 | phenyl | methyl |
| 3.073 | phenylmethyl | methyl |
| 3.074 | cyclopropyl | methyl |
| 3.075 | cyclobutyl | methyl |
| 3.076 | cyclopentyl | methyl |
| 3.077 | cyclohexyl | methyl |
| 3.078 | tetrahydrofuran-3-yl | methyl |
| 3.079 | tetrahydropyran-3-yl | methyl |
| 3.080 | tetrahydropyran-4-yl | methyl |
| 3.081 | tetrahydrofuran-2-ylmethyl | methyl |
| 3.082 | tetrahydrofuran-3-ylmethyl | methyl |
| 3.083 | tetrahydropyran-2-ylmethyl | methyl |
| 3.084 | tetrahydropyran-3-ylmethyl | methyl |
| 3.085 | methoxy | methyl |
| 3.086 | ethoxy | methyl |
| 3.087 | propoxy | methyl |
| 3.088 | isopropoxy | methyl |
| 3.089 | butoxy | methyl |
| 3.09 | methyl | methoxy |
| 3.091 | ethyl | methoxy |
| 3.092 | propyl | methoxy |
| 3.093 | isopropyl | methoxy |
| 3.094 | butyl | methoxy |
| 3.095 | sec-butyl | methoxy |
| 3.096 | tert-butyl | methoxy |
| 3.097 | isobutyl | methoxy |
| 3.098 | neopentyl | methoxy |
| 3.099 | pentyl | methoxy |
| 3.100 | allyl | methoxy |
| 3.101 | 2-methylallyl | methoxy |
| 3.102 | 1,1-dimethylallyl | methoxy |
| 3.103 | prop-2-ynyl | methoxy |
| 3.104 | 2,2-difluoroethyl | methoxy |
| 3.105 | 2,2,2-trifluoroethyl | methoxy |
| 3.106 | 2-cyanoethyl | methoxy |
| 3.107 | 2-methoxyethyl | methoxy |
| 3.108 | 1-methoxy-4-piperidyl | methoxy |
| 3.109 | oxetan-3-yl | methoxy |
| 3.110 | phenyl | methoxy |
| 3.111 | phenylmethyl | methoxy |
| 3.112 | cyclopropyl | methoxy |
| 3.113 | cyclobutyl | methoxy |
| 3.114 | cyclopentyl | methoxy |
| 3.115 | cyclohexyl | methoxy |
| 3.116 | tetrahydrofuran-3-yl | methoxy |
| 3.117 | tetrahydropyran-3-yl | methoxy |
| 3.118 | tetrahydropyran-4-yl | methoxy |
| 3.119 | tetrahydrofuran-2-ylmethyl | methoxy |
| 3.120 | tetrahydrofuran-3-ylmethyl | methoxy |
| 3.121 | tetrahydropyran-2-ylmethyl | methoxy |
| 3.122 | tetrahydropyran-3-ylmethyl | methoxy |
| 3.123 | methoxy | methoxy |
| 3.124 | ethoxy | methoxy |
| 3.125 | propoxy | methoxy |
| 3.126 | isopropoxy | methoxy |
| 3.127 | butoxy | methoxy |
| 3.128 | dimethylamino | H |

| | |
|---|---|
| 3.129 | —CH₂CH₂—O—CH₂CH₂— |
| 3.130 | —CH₂CH₂CH₂CH₂CH₂— |

### Table 3.2:

This table discloses 130 specific compounds of formula (T-3) wherein wherein A¹ is C—R¹, A² is C—R², A³ is C—R³, A⁴ is C—R⁴ and R², R³, R⁴, R⁵, R⁶, and R⁷ are hydrogen, R¹ is fluorine, n is 1, and R¹⁴ and R¹⁵ are as defined above in Table 3.

### Table 3.3:

This table discloses 130 specific compounds of formula (T-3) wherein A¹ is C—R¹, A² is C—R², A³ is C—R³, A⁴ is C—R⁴ and R², R³, R⁴, R⁵, R⁶, and R⁷ are hydrogen, R¹ is chlorine, n is 1, and R¹⁴ and R¹⁵ are as defined above in Table 3.

### Table 3.4:

This table discloses 130 specific compounds of formula (T-3) wherein A¹ is C—R¹, A² is C—R², A³ is C—R³, A⁴ is C—R⁴ and R², R³, R⁴, R⁵, R⁶, and R⁷ are hydrogen, R¹ is methyl, n is 1, and R¹⁴ and R¹⁵ are as defined above in Table 3.

### Table 3.5:

This table discloses 130 specific compounds of formula (T-3) wherein A¹ is C—R¹, A² is C—R², A³ is C—R³, A⁴ is C—R⁴ and R², R³, R⁴, R⁵, R⁶, and R⁷ are hydrogen, R¹ is trifluoromethyl, n is 1, and R¹⁴ and R¹⁵ are as defined above in Table 3.

### Table 3.6:

This table discloses 130 specific compounds of formula (T-3) wherein A¹ is C—R¹, A² is C—R², A³ is C—R³, A⁴ is C—R⁴ and R², R³, R⁴, R⁵, R⁶, and R⁷ are hydrogen, R¹ is methoxy, n is 1, and R¹⁴ and R¹⁵ are as defined above in Table 3.

### Table 3.7:

This table discloses 130 specific compounds of formula (T-3) wherein A¹ is N, A² is C—R², A³ is C—R³, A⁴ is C—R⁴ and R¹, R², R³, R⁴, R⁵, R⁶, and R⁷ are hydrogen, n is 1, and R¹⁴ and R¹⁵ are as defined above in Table 3.

### Table 3.8:

This table discloses 130 specific compounds of formula (T-3) wherein A¹ is C—R¹, A² is C—R², A³ is C—R³, A⁴ is C—R⁴ and R¹, R², R⁴, R⁵, R⁶, and R⁷ are hydrogen, R³ is fluorine, n is 1, and R¹⁴ and R¹⁵ are as defined above in Table 3.

### Table 3.9:

This table discloses 130 specific compounds of formula (T-3) wherein A¹ is C—R¹, A² is C—R², A³ is C—R³, A⁴ is C—R⁴ and R², R⁴, R⁵, R⁶, and R⁷ are hydrogen, R¹ and R³ are fluorine, n is 1, and R¹⁴ and R¹⁵ are as defined above in Table 3.

### Table 3.10:

This table discloses 130 specific compounds of formula (T-3) wherein A¹ is C—R¹, A² is C—R², A³ is C—R³, A⁴ is C—R⁴ and R³, R⁴, R⁵, R⁶, and R⁷ are hydrogen, R¹ and R² are fluorine, n is 1, and R¹⁴ and R¹⁵ are as defined above in Table 3.

### Table 3.11:

This table discloses 130 specific compounds of formula (T-3) wherein A¹ is C—R¹, A² is C—R², A³ is C—R³, A⁴ is C—R⁴ and R¹, R², R³, R⁴, R⁵, R⁶, and R⁷ are hydrogen, n is 2, and R¹⁴ and R¹⁵ are as defined above in Table 3.

### Table 3.12:

This table discloses 130 specific compounds of formula (T-3) wherein A¹ is N, A² is C—R², A³ is C—R³, A⁴ is C—R⁴ and R², R³, R⁴, R⁵, R⁶, and R⁷ are hydrogen, n is 2, and R¹⁴ and R¹⁵ are as defined above in Table 3.

### Table 3.13:

This table discloses 130 specific compounds of formula (T-3) wherein A¹ is N, A² is N, A³ is C—R³, A⁴ is C—R⁴ and R³, R⁴, R⁵, R⁶, and R⁷ are hydrogen, n is 1, and R¹⁴ and R¹⁵ are as defined above in Table 3.

### Table 3.14:

This table discloses 130 specific compounds of formula (T-3) wherein A¹ is C—R¹, A² is C—R², A³ is N, A⁴ is C—R⁴ and R¹, R², R⁴, R⁵, R⁶, and R⁷ are hydrogen, n is 1, and R¹⁴ and R¹⁵ are as defined above in Table 3.

### Table 3.15:

This table discloses 130 specific compounds of formula (T-3) wherein A¹ is C—R¹, A² is C—R², A³ is C—R³, A⁴ is C—R⁴ and R¹, R², R³ R⁴, R⁵, and R⁷ are hydrogen, R⁶ is methyl, n is 1, and R¹⁴ and R¹⁵ are as defined above in Table 3.

### Table 3.16:

This table discloses 130 specific compounds of formula (T-3) wherein wherein A¹ is C—R¹, A² is C—R², A³ is C—R³, A⁴ is C—R⁴ and R¹, R², R³, R⁴, and R⁷ are hydrogen, n is 0, and R¹⁴ and R¹⁵ are as defined above in Table 3.

### Table 3.17:

This table discloses 130 specific compounds of formula (T-3) wherein A¹ is C—R¹, A² is C—R², A³ is C—R³, A⁴ is C—R⁴ and R¹, R², R³, and R⁴ are hydrogen, R⁷ is methyl, n is 0, and R¹⁴ and R¹⁵ are as defined above in Table 3.

### Table 3.18:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is methyl, n is 1, and R¹⁴ and R¹⁵ are as defined above in Table 3.

### Table 3.19:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is methoxy, n is 1, and R¹⁴ and R¹⁵ are as defined above in Table 3.

### Table 3.20:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is ethyl, n is 1, and R¹⁴ and R¹⁵ are as defined above in Table 3.

### Table 3.21:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is allyl, n is 1, and R¹⁴ and R¹⁵ are as defined above in Table 3.

### Table 3.22:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is isobutyl, n is 1, and R¹⁴ and R¹⁵ are as defined above in Table 3.

### Table 3.23:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is 2-methoxyethyl, n is 1, R¹⁴ and R¹⁵ are as defined above in Table 3.

### Table 3.24:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is propyloxy, n is 1, and R¹⁴ and R¹⁵ are as defined above in Table 3.

### Table 3.25:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is propyn-2-yl, n is 1, and R¹⁴ and R¹⁵ are as defined above in Table 3.

### Table 3.26:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is 4-chlorophenyl)methoxy, n is 1, and R¹⁴ and R¹⁵ are as defined above in Table 3.

### Table 3.27:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is 3,3-dichloroallyloxy, n is 1, and R¹⁴ and R¹⁵ are as defined above in Table 3.

### Table 3.28:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is 2-furylmethyl, n is 1, and R¹⁴ and R¹⁵ are as defined above in Table 3.

### Table 3.29:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is tetrahydrofuran-2-ylmethyl, n is 1, and R¹⁴ and R¹⁵ are as defined above in Table 3.

### Table 3.30:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is cyclopropylmethyl, n is 1, and R¹⁴ and R¹⁵ are as defined above in Table 3.

### Table 3.31:

This table discloses 130 specific compounds of formula (T-3) wherein A¹ is C—R¹, A² is C—R², A³ is C—R³, A⁴ is C—R⁴ and R¹, R², R³, R⁴, R⁵, and R⁶ are hydrogen, R⁷ is cyclopropyl, n is 1, and R¹⁴ and R¹⁵ are as defined above in Table 3.

### EXAMPLES

The Examples which follow serve to illustrate the invention. The compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm or 0.2 ppm.

Compounds of Formula (I) may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile (including improved crop tolerance), improved physico-chemical properties, or increased biodegradability).

Throughout this description, temperatures are given in degrees Celsius (° C.) and “mp.” means melting point.

LC/MS means Liquid Chromatography Mass Spectrometry and the description of the apparatus and the method (Methods A and B) is as follows:

The description of the LC/MS apparatus and the method A is:
SQ Detector 2 from Waters
Ionisation method: Electrospray
Polarity: positive and negative ions

### Capillary (kV) 3.0, Cone (V) 30.00, Extractor (V) 2.00, Source Temperature (° C.) 150, Desolvation

### Temperature (° C.) 350, Cone Gas Flow (L/Hr) 0, Desolvation Gas Flow (L/Hr) 650

Mass range: 100 to 900 Da
DAD Wavelength range (nm): 210 to 500
Method Waters ACQUITY UPLC with the following HPLC gradient conditions:
(Solvent A: Water/Methanol 20:1+0.05% formic acid and Solvent B: Acetonitrile+0.05% formic acid)

| Time (minutes) | A (%) | B (%) | Flow rate (ml/min) |
|---|---|---|---|
| 0 | 100 | 0 | 0.85 |
| 1.2 | 0 | 100 | 0.85 |
| 1.5 | 0 | 100 | 0.85 |

Type of column: Waters ACQUITY UPLC HSS T3; Column length: 30 mm; Internal diameter of column: 2.1 mm; Particle Size: 1.8 micron; Temperature: 60° C.
The description of the LC/MS apparatus and the method B is:
SQ Detector 2 from Waters
lonisation method: Electrospray
Polarity: positive ions

### Capillary (kV) 3.5, Cone (V) 30.00, Extractor (V) 3.00, Source Temperature (° C.) 150, Desolvation

### Temperature (° C.) 400, Cone Gas Flow (L/Hr) 60, Desolvation Gas Flow (L/Hr) 700

Mass range: 140 to 800 Da
DAD Wavelength range (nm): 210 to 400
Method Waters ACQUITY UPLC with the following HPLC gradient conditions
(Solvent A: Water/Methanol 9:1+0.1% formic acid and Solvent B: Acetonitrile+0.1% formic acid)

| Time (minutes) | A (%) | B (%) | Flow rate (ml/min) |
|---|---|---|---|
| 0 | 100 | 0 | 0.75 |
| 2.5 | 0 | 100 | 0.75 |
| 2.8 | 0 | 100 | 0.75 |
| 3.0 | 100 | 0 | 0.75 |

Type of column: Waters ACQUITY UPLC HSS T3; Column length: 30 mm; Internal diameter of column: 2.1 mm; Particle Size: 1.8 micron; Temperature: 60° C.

Where necessary, enantiomerically pure final compounds may be obtained from racemic materials as appropriate via standard physical separation techniques, such as reverse phase chiral chromatography, or through stereoselective synthetic techniques, eg, by using chiral starting materials.

### FORMULATION EXAMPLES

| Wettable powders | a) | b) | c) |
|---|---|---|---|
| active ingredient [compound of formula (I)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredient [compound of formula (I)] | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The active ingredient is thoroughly mixed with the adjuvants and the mixture is thoroughly ground in a suitable mill, affording powders that can be used directly for seed treatment.

| Emulsifiable concentrate | |
|---|---|
| active ingredient [compound of formula (I)] | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredient [compound of formula (I)] | 5% | 6% | 4% |
| Talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

| Extruder granules | |
|---|---|
| Active ingredient [compound of formula (I)] | 15% |
| sodium lignosulfonate | 2% |
| Carboxymethylcellulose | 1% |
| Kaolin | 82% |

The active ingredient is mixed and ground with the adjuvants, and the mixture is moistened with water. The mixture is extruded and then dried in a stream of air.

| Coated granules | |
|---|---|
| Active ingredient [compound of formula (I)] | 8% |
| polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

Suspension concentrate

| | |
|---|---|
| active ingredient [compound of formula (I)] | 40% |
| propylene glycol | 10% |
| nonylphenol polyethylene glycol ether (15 mol of ethylene oxide) | 6% |
| Sodium lignosulfonate | 10% |
| Carboxymethylcellulose | 1% |
| silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredient [compound of formula (I)] | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% solution in | 0.5% |
| water) | |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow-Release Capsule Suspension

28 parts of a combination of the compound of formula I are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### LIST OF ABBREVIATIONS

AIBN=azobisisobutyronitrile
BOP—Cl=phosphoric acid bis(2-oxooxazolidide) chloride
DIBAL-H=diisobutylaluminium hydride
DIEA=N-ethyl-N-isopropyl-propan-2-amine
DIPEA=N,N-diisopropylethylamine
   DMA=dimethylacetamide
   DMAP=dimethylaminopyridine
   DMF=dimethylformamide
   EdCl=3-(ethyliminomethyleneamino)-N,N-dimethylpropan-1-amine
EtOAc=ethyl acetate
EtOH=ethyl alcohol
HCl=hydrochloric acid
HOAt=1-hydroxy-7-azabenzotriazole
HATU=1-[bis(dimethylamino)methylene]-1H-1,2,3-triazolo[4,5-b]pyridinium 3-oxid-hexafluorophosphate
mp=melting point
MeOH=methyl alcohol
NaOH=sodium hydroxide
NBS=N-bromosuccinimide
rt=room temperature
TBME=tert-butyl methyl ether
TFAA=trifluoroacetic acid anhydride
THF=tetrahydrofuran

### PREPARATION EXAMPLES

### Example 1: Preparation of N-methoxy-N-[4-(5-trifluoromethyl-[1,2,4]oxadiazol-3-yl)-benzyl]-oxalamic acid ethyl ester (Compound 3.10 of Table T3)

### Step 1: Preparation of 4-[5-(trifluoromethyl)-1,2,4-oxadiazol-3-yl]benzoyl chloride

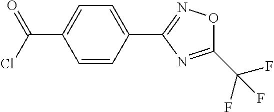 [SMILES: O=C(Cl)c1ccc(-c2noc(C(F)(F)F)n2)cc1]

4-[5-(trifluoromethyl)-1,2,4-oxadiazol-3-yl]benzoic acid (4.00 g, 15.0 mmol) was suspended in dichloromethane (90 mL) DMF (0.01 mL, 0.150 mmol) was added followed by oxalyl chloride (1.46 mL, 16.5 mmol). The mixture was heated at reflux for 2 hours. The mixture was evaporated under reduced pressure to afford 4.15 g of 4-[5-(trifluoromethyl)-1,2,4-oxadiazol-3-yl]benzoyl chloride as a yellow solid.

### Step 2: Preparation of N-methoxy-N-methyl-4-[5-(trifluoromethyl)-1,2,4-oxadiazol-3-yl]benzamide

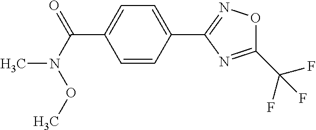 [SMILES: CON(C)C(=O)c1ccc(-c2noc(C(F)(F)F)n2)cc1]

A solution of 4-[5-(trifluoromethyl)-1,2,4-oxadiazol-3-yl]benzoyl chloride from Step 1 (4.15 g, 14.6 mmol) in dichloromethane (20 mL) was added drop wise at room temperature to a stirred solution of N-methoxymethanamine (1.10 g, 17.5 mmol) and triethylamine (3.10 mL, 21.8 mmol) in dichloromethane (80 mL). The mixture was stirred at room temperature for 18 hours. The reaction mixture was poured into water extracted twice with dichloromethane. The combined organic layers were washed with brine, dried over sodium sulfate, and filtered. The solvent was removed under reduced pressure and the resultant crude residue was subjected to flash chromatography over silica gel (heptane:EtOAc eluent gradient 9:1 to 65:35) to afford 4.12 g of N-methoxy-N-methyl-4-[5-(trifluoromethyl)-1,2,4-oxadiazol-3-yl]benzamide as a solid. LC/MS (Method A) retention time=0.97 minutes, 302 (M+H).

¹H NMR (400 MHz, CDCl₃) δ ppm: 8.18 (d, 2H), 7.84 (d, 2H), 3.56 (s, 3H), 3.40 (s, 3H).

### Step 3: Preparation of 4-[5-(trifluoromethyl)-1,2,4-oxadiazol-3-yl]benzaldehyde

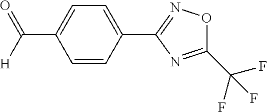 [SMILES: O=Cc1ccc(-c2noc(C(F)(F)F)n2)cc1]

In a 75-mL multi neck flask equipped with stirrer, thermometer at −78° C. under argon, DIBAL-H, 1.0M in toluene (16 mL, 16.0 mmol) was added drop-wise to a solution of N-methoxy-N-methyl-4-[5-(trifluoromethyl)-1,2,4-oxadiazol-3-yl]benzamide (4.10 g, 13.3 mmol) in 2-methyltetrahydrofuran (90 mL). The mixture was stirred for two hours at −78° C. and for one hour temperature was let increase to 0° C. Complete conversion observed by LC-MS. The mixture was quenched by drop wise addition of sat. ammonium chloride solution. Precipitation of a white solid occurred and 4M HCl was added until full solubilisation. The mixture was extracted thrice with ethyl acetate. Combined organics were dried over magnesium sulfate and evaporated to afford the crude as beige solid. The crude was subject to flash chromatography over silicagel (heptane:EtOAc eluent gradient 99:1 to 90:10) to afford 2.93 g of 4-[5-(trifluoromethyl)-1,2,4-oxadiazol-3-yl]benzaldehyde as a white solid mp: 40-50° C.

¹H NMR (400 MHz, CDCl₃) δ ppm: 10.12 (s, 1H), 8.31 (d, 2H), 8.05 (d, 2H).

¹⁹F NMR (400 MHz, CDCl₃) δ ppm: −65.29 (s).

### Step 4: Preparation of N-methoxy-1-[4-[5-(trifluoromethyl)-1,2,4-oxadiazol-3-yl]phenyl]methanimine

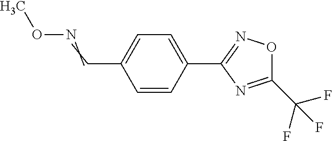 [SMILES: CON=Cc1ccc(-c2noc(C(F)(F)F)n2)cc1]

A solution of 4-[5-(trifluoromethyl)-1,2,4-oxadiazol-3-yl]benzaldehyde (0.30 g, 1.1 mmol) in methanol (11 mL) was treated at room temperature with pyridine (0.15 mL, 1.9 mmol) followed by addition of O-methylhydroxylamine hydrochloride (0.15 g, 1.8 mmol). The mixture was stirred at room temperature over the weekend, poured on water, acidified with 1M HCl and extracted with ethyl acetate. Combined organics were washed with water, dried over magnesium sulfate and evaporated to afford a resin. The crude was subject to flash chromatography over silica gel (heptane:EtOAc eluent gradient 100:0 to 95:5) to afford the title compound as a white solid mp: 55-65° C.

¹H NMR (400 MHz, CDCl₃) δ ppm: 8.14 (s, 1H), 8.11 (d, 2H), 7.73 (d, 2H).

¹⁹F NMR (400 MHz, CDCl₃) δ ppm: −65.40 (s).

### Step 5: Preparation of N-methoxy-1-[4-[5-(trifluoromethyl)-1,2,4-oxadiazol-3-yl]phenyl]methanamine

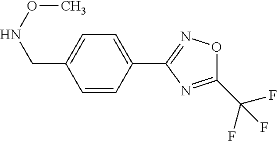 [SMILES: CONCc1ccc(-c2noc(C(F)(F)F)n2)cc1]

A solution of N-methoxy-1-[4-[5-(trifluoromethyl)-1,2,4-oxadiazol-3-yl]phenyl]methanimine (0.81 g, 2.89 mmol) in acetic acid (11 mL) was treated at 15° C. with sodium cyano borohydride (0.4 g, 6.1 mmol). The mixture was stirred at 22° C. for 18 hours until complete consumption of starting material was observed. The reaction mixture was carefully poured in 0.1M NaOH solution. By addition of 1M NaOH pH was adjusted to 12-13. The mixture was extracted four times with TBME. Combined organics were washed twice with water and once with brine then dried over magnesium sulfate and concentrated under reduced pressure. The resultant green oil was purified by flash chromatography over silica gel (heptane:EtOAc eluent gradient 99:1 to 70:30) to give 0.60 g of the title compound as an oil.

¹H NMR (400 MHz, CDCl₃) δ ppm: 8.09 (d, 2H), 7.53 (d, 2H), 5.33 (Sbr, 1H), 4.12 (s, 2H), 3.50 (s, 3H).

¹⁹F NMR (400 MHz, CDCl₃) δ ppm: −65.32 (s).

### Step 6: Preparation N-methoxy-N-[4-(5-trifluoromethyl-[1,2,4]oxadiazol-3-yl)-benzyl]-oxalamic acid ethyl ester

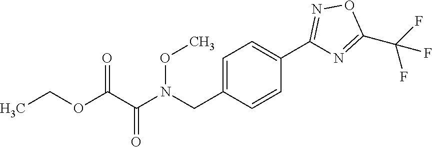 [SMILES: CCOC(=O)C(=O)N(Cc1ccc(-c2noc(C(F)(F)F)n2)cc1)OC]

To a stirred solution of O-methyl-N-[4-(5-trifluoromethyl-[1,2,4]oxadiazol-3-yl)-benzyl]-hydroxylamine (2.43 g, 8.89 mmol) in dry dichloromethane at 0° C. were added triethylamine (2.5 mL, 17.8 mmol) and ethyl oxalyl chloride (1.19 mL, 10.7 mmol). The reaction mixture was stirred at 22° C. for 30 minutes and then quenched with water. The resultant mixture was shaken and the layers were separated. The aqueous layer was extracted thrice with dichloromethane and the combined organic layers were washed with brine, dried over sodium sulfate, filtered and evaporated to dryness. The crude oil was purified by flash chromatography over silica gel (eluent: cyclohexane/ethyl acetate) to give (2.80 g, 7.5 mmol, 84%) of the title compound as an oil.

LC/MS (Method A) retention time=1.08 minutes, 374 (M+H).

¹H NMR (400 MHz, CDCl₃) δ ppm: 8.10 (d, 2H), 7.50 (d, 2H), 4.87 (s, 2H), 4.36 (q, 2H), 3.71 (s, 3H), 1.36 (t, 3H).

### Example 2: This example illustrates the preparation of intermediate N-methoxy-N-[4-(5-trifluoromethyl-[1,2,4]oxadiazol-3-yl)-benzyl]-oxalamic acid (Compound 3.7 of Table T3)

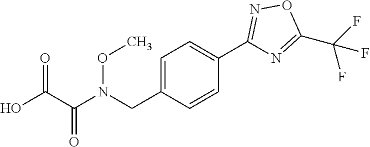 [SMILES: CON(Cc1ccc(-c2noc(C(F)(F)F)n2)cc1)C(=O)C(=O)O]

To a stirred solution of N-methoxy-N-[4-(5-trifluoromethyl-[1,2,4]oxadiazol-3-yl)-benzyl]-oxalamic acid ethyl ester (2.80 g, 7.50 mmol) in tetrahydrofurane/water (1/1) was added lithium hydroxide hydrate (0.359 g, 15.0 mmol). The reaction mixture was stirred at 22° C. for 16 hours and then acidified to pH=1 with diluted HCl. The resultant mixture was shaken and the layers were separated. The aqueous layer was extracted thrice with ethyl acetate and the combined organic layers were washed with brine, dried over sodium sulfate, filtered and evaporated to dryness. The crude oil was purified by flash chromatography over silica gel (reversed phase, eluent: acetonitrile/water) to give (0.584 g, 1.69 mmol, 23%) of the title compound as an oil.

¹H NMR (400 MHz, CD₃OD) δ ppm: 8.12 (d, 2H), 7.58 (d, 2H), 4.93 (s, 2H), 3.80 (s, 3H).

¹⁹F NMR (400 MHz, CD₃OD) δ ppm: −67.36 (s).

### Example 3: This example illustrates the preparation of N-[4-(5-trifluoromethyl-[1,2,4]oxadiazol-3-yl)-benzyl]-oxalamic acid ethyl ester (Compound 3.9 of Table T3)

### Step 1: Preparation of N′-hydroxy-4-methyl-benzamidine

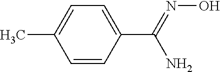

To a stirred suspension of 4-methylbenzonitrile (35 g, 0.29 mol) in ethanol (220 mL) and water (440 mL) was added at 22° C. hydroxylamine hydrochloride (41.1 g, 0.58 mol), potassium carbonate (65.4 g, 0.47 mol) and 8-hydroxyquinoline (0.22 g, 1.5 mmol). The reaction mixture was heated at 80° C. for 4 hours. The mixture was cooled to 22° C. and diluted with 2N HCl until pH 8. Ethanol was evaporated under reduced pressure. The mixture was filtered, washed with water and dried under vacuum to afford 39.1 g of N′-hydroxy-4-methyl-benzamidine. LC/MS (Method A) retention time=0.23 minutes, 151.0 (M+H).

### Step 2: Preparation of 3-(p-tolyl)-5-(trifluoromethyl)-1,2,4-oxadiazole

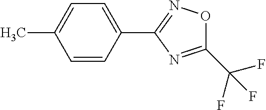 [SMILES: Cc1ccc(-c2noc(C(F)(F)F)n2)cc1]

To a stirred solution of N′-hydroxy-4-methyl-benzamidine (38.7 g, 0.25 mol) in 2-methyltetrahydrofuran (750 mL) was added TFAA at 0° C. The reaction mixture was stirred at 15° C. for two hours and diluted with water. The organic layer was separated, washed successively with sodium bicarbonate solution, ammonium chloride solution and water, dried over sodium sulfate, filtered and evaporated to dryness. The crude residue was subject to flash chromatography over silica gel (heptane:EtOAc eluent gradient 99:1 to 90:10) to afford 54.1 g of 3-(p-tolyl)-5-(trifluoromethyl)-1,2,4-oxadiazole as clear oil, which solidified after storage.

LC/MS (Method A) retention time=1.15 minutes, mass not detected.

¹H NMR (400 MHz, CDCl₃) δ ppm: 8.00 (d, 2H), 7.32 (d, 2H), 2.45 (s, 3H). ¹⁹F NMR (400 MHz, CDCl₃) δ ppm: −65.41 (s).

### Step 3a: Preparation of 3-[4-(bromomethyl)phenyl]-5-(trifluoromethyl)-1,2,4-oxadiazole

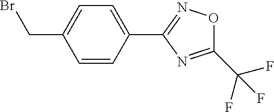 [SMILES: FC(F)(F)c1nc(-c2ccc(CBr)cc2)no1]

A stirred mixture of 3-(p-tolyl)-5-(trifluoromethyl)-1,2,4-oxadiazole (56.0 g, 0.24 mol) and NBS (45.4 g, 0.25 mol) in tetrachloromethane (480 mL) under argon was heated to 70° C. AIBN (4.03 g, 24 mmol) was added and the reaction mixture was stirred at 65° C. for 18 hours. The mixture was cooled to 22° C. and diluted with dichloromethane and water. The organic layer was washed with sodium bicarbonate solution, dried over sodium sulfate, filtered and evaporated to dryness. The crude residue was subject to flash chromatography over silica gel (cyclohehane:EtOAc eluent gradient 100:0 to 95:5) to afford 44.7 g of 3-[4-(bromomethyl)phenyl]-5-(trifluoromethyl)-1,2,4-oxadiazole as a white solid mp: 58-63° C.

¹H NMR (400 MHz, CDCl₃) δ ppm: 8.11 (d, 2H), 7.55 (d, 2H), 4.53 (s, 2H).

¹⁹F NMR (400 MHz, CDCl₃) δ ppm: −65.32 (s).

3-[4-(dibromomethyl)phenyl]-5-(trifluoromethyl)-1,2,4-oxadiazole (see below) was isolated as by-product as white solid (mp 61-66° C.).

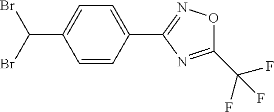 [SMILES: FC(F)(F)c1nc(-c2ccc(C(Br)Br)cc2)no1]

¹H NMR (400 MHz, CDCl₃) δ ppm: 8.15 (d, 2H), 7.73 (d, 2H), 6.68 (s, 1H).

¹⁹F NMR (400 MHz, CDCl₃) δ ppm: −65.34 (s).

### Step 3b: Preparation of 3-[4-(bromomethyl)phenyl]-5-(trifluoromethyl)-1,2,4-oxadiazole

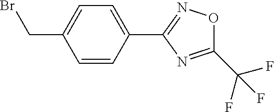 [SMILES: FC(F)(F)c1nc(-c2ccc(CBr)cc2)no1]

To a stirred 1:9 ratio mixture of 3-[4-(bromomethyl)phenyl]-5-(trifluoromethyl)-1,2,4-oxadiazole and 3-[4-(dibromomethyl)phenyl]-5-(trifluoromethyl)-1,2,4-oxadiazole (10.2 g) in acetonitrile (95 mL), water (1.9 mL) and DIEA (6.20 mL, 35.7 mmol) was added diethylphosphite (4.7 mL, 35.7 mmol) at 5° C. The mixture was stirred at 5-10° C. for two hours, water and 1M HCl were added, and acetonitrile was evaporated under reduced pressure. The white slurry was extracted with dichloromethane and the combined organic layers were dried over sodium sulfate, and filtered. The solvent was removed under reduced pressure and the resultant crude residue was subject to flash chromatography over silica gel (cyclohexane:EtOAc eluent gradient 99:1 to 9:1) to afford 7.10 g of 3-[4-(bromomethyl)phenyl]-5-(trifluoromethyl)-1,2,4-oxadiazole.

¹H NMR (400 MHz, CDCl₃) δ ppm: 8.11 (d, 2H), 7.55 (d, 2H), 4.53 (s, 2H).

¹⁹F NMR (400 MHz, CDCl₃) δ ppm: −65.32 (s).

### Step 4: Preparation of [4-[5-(trifluoromethyl)-1,2,4-oxadiazol-3-yl]phenyl]methanamine hydrochloride

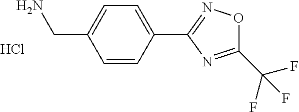 [SMILES: Cl ; NCc1ccc(-c2noc(C(F)(F)F)n2)cc1]

A dry flask equipped with stirrer under argon was charged with sodium hydride (2 equiv., 3.13 mmol, 60 mass % NaH) and tetrahydrofuran (25 mL). To this white suspension was added tert-butyl N-tert-butoxycarbonylcarbamate (1.1 equiv, 1.72 mmol) and while stirring for 5 min gas evolution was observed. 3-[4-(bromomethyl)phenyl]-5-(trifluoromethyl)-1,2,4-oxadiazole (0.500 g, 1.56 mmol) was then introduced and the contents were stirred for 12 hours. Upon reaction completion, the solution was poured into water and extracted with ethyl acetate (2×30 mL). The organic layers were combined and dried over sodium sulfate, filtered, and concentrated at reduced pressure to produce a pale yellow oil which partially crystalize upon sitting. The yellow material was dissolved in dioxane (5 mL) and hydrogen chloride (15 equiv., 24.7 mmol, 4M in dioxane) was introduced dropwise. After stirring overnight at 22° C. the reaction solution was diluted with ether and provided a white precipitate (70% yield) whose analytics matched the reported values and which was used without further purification. mp: >200° C.

LC/MS (Method A) retention time=0.61 minutes, 244 (M+H).

¹H NMR (400 MHz, DMSO) δ ppm: 8.56 (s_{br}, 2H), 8.13 (d, 2H), 7.75 (d, 2H), 4.15 (s, 2H).

¹⁹F NMR (400 MHz, DMSO) δ ppm: −64.69 (s).

Alternatively, the titled compound can be prepared using an analagous procedure as described in WO 2013/066839.

To a stirred solution of tert-butyl N-[[4-[5-(trifluoromethyl)-1,2,4-oxadiazol-3-yl]phenyl]methyl]-carbamate, (23.1 g, 65.4 mmol) in 1,4-dioxane (196 mL), was added drop wise at 70° C. a solution of 4 M HCl in 1,4-dioxane (41 mL, 163 mmol). Precipitation of a white solid and gas liberation started 5 minutes after addition. The mixture was stirred for 6 hours at 70° C. The white suspension was cooled down to 23° C., filtered over sinter no 4 funnel, washed with 1,4-dioxane and dried under reduced pressure at 40° C. to yield 17.3 g of [4-[5-(trifluoromethyl)-1,2,4-oxadiazol-3-yl]phenyl]methanamine hydrochloride as a yellow solid.

### Step 5: Preparation of N-[4-(5-trifluoromethyl-[1,2,4]oxadiazol-3-yl)-benzyl]-oxalamic acid ethyl ester

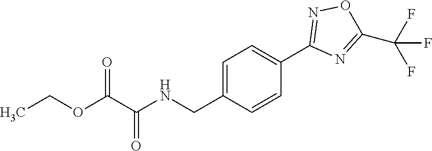 [SMILES: CCOC(=O)C(=O)NCc1ccc(-c2noc(C(F)(F)F)n2)cc1]

To a stirred solution of 4-(5-trifluoromethyl-[1,2,4]oxadiazol-3-yl)-benzylamine hydrochloride (2.50 g, 8.94 mmol) in dry dichloromethane at 0° C. were added triethylamine (3.78 mL, 26.8 mmol) and ethyl oxalyl chloride (1.20 mL, 10.7 mmol). The reaction mixture was stirred at 22° C. for 30 minutes and then quenched with water. The resultant mixture was shaken and the layers were separated. The aqueous layer was extracted thrice with dichloromethane and the combined organic layers were washed with brine, dried over sodium sulfate, filtered and evaporated to dryness. The crude residue was purified by flash chromatography over silica gel (eluent: cyclohexane/ethyl acetate) to give 2.74 g of N-[4-(5-trifluoromethyl-[1,2,4]oxadiazol-3-yl)-benzyl]-oxalamic acid ethyl ester as a white solid.

LC/MS (Method A) retention time=0.98 minutes, 344 (M+H).

¹H NMR (400 MHz, CDCl₃) δ ppm: 8.10 (d, 2H), 7.46 (d, 2H), 4.60 (d, 2H), 4.38 (q, 2H), 1.38 (t, 3H).

### Example 4: This example illustrates the preparation of intermediate N-[4-(5-trifluoromethyl-[1,2,4]oxadiazol-3-yl)-benzyl]-oxalamic acid

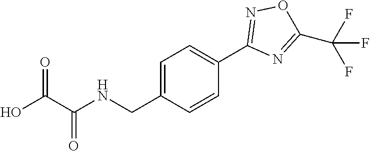 [SMILES: O=C(O)C(=O)NCc1ccc(-c2noc(C(F)(F)F)n2)cc1]

To a stirred solution of N-[4-(5-trifluoromethyl-[1,2,4]oxadiazol-3-yl)-benzyl]-oxalamic acid ethyl ester (2.74 g, 7.98 mmol) in tetrahydrofurane/water (1/1) was added lithium hydroxide hydrate (0.382 g, 16.0 mmol). The reaction mixture was stirred at 22° C. for 16 hrs and then acidified to pH=1 with diluted HCl. The resultant mixture was shaken and the layers were separated. The aqueous layer was extracted thrice with ethyl acetate and the combined organic layers were washed with brine, dried over sodium sulfate, filtered and evaporated to dryness to give 1.94 g of N-[4-(5-trifluoromethyl-[1,2,4]oxadiazol-3-yl)-benzyl]-oxalamic acid as a white solid.

LC/MS (Method A) retention time=0.78 minutes, 314 (M−H).

¹H NMR (400 MHz, CD₃OD) δ ppm: 8.10 (d, 2H), 7.52 (d, 2H), 4.55 (s, 2H).

¹⁹F NMR (400 MHz, CD₃OD) δ ppm: −67.39 (s).

### Example 5: This example illustrates the preparation of intermediate N-propoxy-1-[4-[5-(trifluoromethyl)-1,2,4-oxadiazol-3-yl]phenyl]methanamine

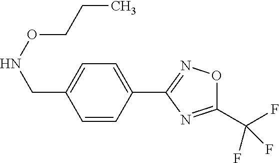 [SMILES: CCCONCc1ccc(-c2noc(C(F)(F)F)n2)cc1]

A solution of 3-[4-(bromomethyl)phenyl]-5-(trifluoromethyl)-1,2,4-oxadiazole (1.30 g, 4.06 mmol) in dichloromethane (10 mL) was added drop wise at 22° C. to a stirred solution of O-propylhydroxylamine hydrochloride (3.74 g, 32.5 mmol) and DIEA (6.40 mL, 36.6 mmol) in dichloromethane (6 mL). The mixture was stirred at 22° C. for 24 hours. The reaction mixture was poured onto water and the layers were separated. The aqueous layer was extracted trice with dichloromethane. The combined organic layers were washed with brine, dried over sodium sulfate, and filtered. The solvent was removed under reduced pressure and the resultant crude was purified by flash chromatography over silica gel (cyclohexane:EtOAc eluent gradient 1:0 to 1:1) to give 0.92 g of N-propoxy-1-[4-[5-(trifluoromethyl)-1,2,4-oxadiazol-3-yl]phenyl]methanamine as a clear oil.

LC/MS (Method A) retention time=1.12 minutes, 302 (M+H).

¹H NMR (400 MHz, CDCl₃) δ ppm: 8.09 (d, 2H), 7.02 (d, 2H), 5.70 (s_{br}, 1H), 4.11 (s, 2H), 3.59 (m, 2H), 1.52 (m, 2H), 0.86 (s, 3H).

¹⁹F NMR (400 MHz, CDCl₃) δ ppm: −65.33 (s).

### Example 6: This example illustrates the preparation of intermediate N-[[4-[5-(trifluoromethyl)-1,2,4-oxadiazol-3-yl]phenyl]methyl]ethanamine

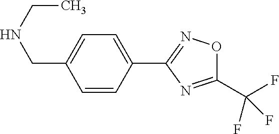 [SMILES: CCNCc1ccc(-c2noc(C(F)(F)F)n2)cc1]

A solution of 3-[4-(bromomethyl)phenyl]-5-(trifluoromethyl)-1,2,4-oxadiazole (1.50 g, 4.69 mmol) in dichloromethane (9.4 mL) was added drop-wise at rt to a stirred solution of ethylamine 2 M in MeOH (12 mL, 24.0 mmol). The mixture was stirred at room temperature for 24 hours. The reaction mixture was poured into water and the layers were separated. The aqueous layer was extracted thrice with dichloromethane. The combined organic layers were washed with brine, dried over sodium sulfate and filtered. The solvent was removed under reduced pressure and the resultant crude was purified by flash chromatography over silica gel (cyclohexane:EtOAc eluent gradient 1:0 to 1:1) to give 0.92 g of N-[[4-[5-(trifluoromethyl)-1,2,4-oxadiazol-3-yl]phenyl]methyl]ethanamine as a white solid. mp: 102-112° C.

LC/MS (Method A) retention time=0.66 minutes, 272 (M+H).

¹H NMR (400 MHz, DMSO) δ ppm: 8.01 (d, 2H), 7.57 (d, 2H), 3.86 (q, 2H), 3.29 (s_{br}, 1H), 2.53 (q, 2H), 1.05 (t, 3H).

¹⁹F NMR (400 MHz, CDCl₃) δ ppm: −64.77 (s).

### Example 7: This example illustrates the preparation of the intermediate N-[[4-[5-(trifluoromethyl)-1,2,4-oxadiazol-3-yl]phenyl]methyl]butan-2-amine

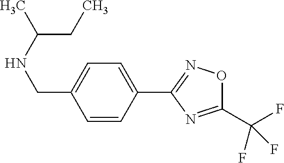 [SMILES: CCC(C)NCc1ccc(-c2noc(C(F)(F)F)n2)cc1]

A solution of 3-[4-(bromomethyl)phenyl]-5-(trifluoromethyl)-1,2,4-oxadiazole (1.50 g, 4.69 mmol) in dichloromethane (9.4 mL) was added drop wise at 22° C. to a stirred solution of sec-butylamine (2.4 mL, 23.4 mmol) in dichloromethane (5 mL). The mixture was stirred at 22° C. for 24 hours. The reaction mixture was poured into water and extracted with dichloromethane. The combined organic layers were washed with brine, dried over sodium sulfate, and filtered. The solvent was removed under reduced pressure and the resultant crude residue was purified by flash chromatography over silica gel (cyclohexane:EtOAc eluent gradient 1:0 to 1:1) to give 1.18 g of N-[[4-[5-(trifluoromethyl)-1,2,4-oxadiazol-3-yl]phenyl]methyl]ethanamine as a clear oil.

LC/MS (Method A) retention time=0.71 minutes, 300 (M+H).

¹H NMR (400 MHz, CDCl₃) δ ppm: 8.06 (d, 2H), 7.49 (d, 2H), 3.86 (q, 2H), 2.62 (m, 1H), 1.53 (m, 1H), 1.49 (m, 1H), 1.09 (d, 3H), 0.91 (m, 3H).

¹⁹F NMR (400 MHz, CDCl₃) δ ppm: −65.39 (s).

### Example 8: This example illustrates the preparation of the intermediate N-methoxy-1-[4-[5-(trifluoromethyl)-1,2,4-oxadiazol-3-yl]phenyl]ethanamine

### Step 1: Preparation of 4-[(E)-N-methoxy-C-methyl-carbonimidoyl]benzonitrile

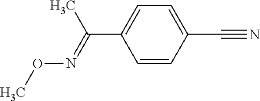

A solution of 4-acetyl benzonitrile (5.0 g, 33.8 mmol) in EtOH (270 mL) was treated at room temperature with O-methylhydroxylamine hydrochloride (4.32 g, 50.6 mmol). The mixture was stirred at room temperature for 70 hours, poured on water and extracted thrice with dichloromethane. Combined organics were washed with water, dried over magnesium sulfate and evaporated to afford 5.95 g of 4-[(E)-N-methoxy-C-methyl-carbonimidoyl]benzonitrile as clear oil. No further purification was required.

LC/MS (Method A) retention time=0.93 minutes, 175 (M+H).

¹H NMR (400 MHz, CDCl₃) δ ppm: 7.78 (d, 2H), 7.65 (d, 2H), 4.03 (s, 3H), 2.22 (s, 3H).

### Step 2: Preparation of N′-hydroxy-4-[(E)-N-methoxy-C-methyl-carbonimidoyl]benzamidine

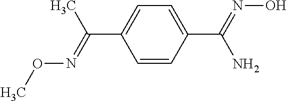

To a stirred suspension of 4-[(E)-N-methoxy-C-methyl-carbonimidoyl]benzonitrile (5.95 g, 33.5 mmol) in ethanol (50 mL) and water (100 mL) was added at 22° C. hydroxylamine hydrochloride (4.7 g, 66.9 mmol), potassium carbonate (7.48 g, 53.6 mmol) and 8-hydroxyquinoline (0.025 g, 0.17 mmol). The reaction mixture was heated at 80° C. for 3 hours. The mixture was cooled to 22° C. and 1M HCl was added until pH 8-9. Ethanol was removed under reduced pressure at 50° C. The mixture was stirred 30 minutes at 5° C., filtered, washed with water and dried under vacuum to afford 6.6 g of N′-hydroxy-4-[(E)-N-methoxy-C-methyl-carbonimidoyl]benzamidine as a white solid mp: 134-139° C.

LC/MS (Method A) retention time=0.43 minutes, 208 (M+H).

¹H NMR (400 MHz, CDCl₃) δ ppm: 8.23 (s_{br}, 1H), 7.69 (d, 2H), 7.62 (d, 2H), 4.88 (s_{br}, 2H), 4.00 (s, 3H), 2.23 (s, 3H).

### Step 3: Preparation of N-methoxy-1-[4-[5-(trifluoromethyl)-1,2,4-oxadiazol-3-yl]phenyl]ethanimine

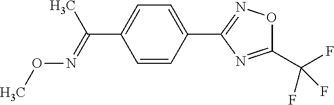 [SMILES: CO/N=C(\C)c1ccc(-c2noc(C(F)(F)F)n2)cc1]

To a stirred solution of N′-hydroxy-4-[(E)-N-methoxy-C-methyl-carbonimidoyl]benzamidine (6.46 g, 31.2 mmol) in 2-methyltetrahydrofuran (93 mL) was added TFAA (6.24 mL, 43.7 mmol) at 15° C. The reaction mixture was stirred at 15° C. for two hours and diluted with water. The organic layer was separated and washed successively with sodium bicarbonate solution, ammonium chloride solution and water, dried over sodium sulfate, filtered and evaporated to dryness to afford 8.9 g of N-methoxy-1-[4-[5-(trifluoromethyl)-1,2,4-oxadiazol-3-yl]phenyl]ethanimine as beige solid mp: 56-61° C., LC/MS (Method A) retention time=1.19 minutes, 286 (M+H).

¹H NMR (400 MHz, CDCl₃) δ ppm: 8.13 (d, 2H), 7.81 (d, 2H), 4.03 (s, 3H), 2.25 (s, 3H).

¹⁹F NMR (400 MHz, CDCl₃) δ ppm: −65.34 (s).

### Step 4: Preparation of N-methoxy-1-[4-[5-(trifluoromethyl)-1,2,4-oxadiazol-3-yl]phenyl]ethanamine

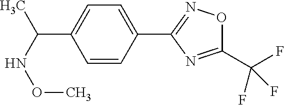 [SMILES: CONC(C)c1ccc(-c2noc(C(F)(F)F)n2)cc1]

A solution of N-methoxy-1-[4-[5-(trifluoromethyl)-1,2,4-oxadiazol-3-yl]phenyl]ethanimine (8.9 g, 29.6 mmol) in acetic acid (119 mL) was treated at 15° C. portion wise with sodium cyano borohydride (4.12 g, 62.3 mmol). The mixture was stirred at 23° C. for 18 hours. Partial consumption of starting material was observed. Additional sodium cyano borohydride (4.12 g, 62.3 mmol) was added portion wise. The reaction mixture was carefully poured in 0.1M sodium hydroxide solution. By addition of 4 M NaOH pH was adjusted to 9-12. The mixture was extracted four times with TBME. Combined organics were washed twice with water and once with brine then dried over magnesium sulfate and concentrated under reduced. The resultant green oil was purified by flash chromatography over silica gel (heptane:EtOAc eluent gradient 99:1 to 70:30) to give 5.0 g of of N-methoxy-1-[4-[5-(trifluoromethyl)-1,2,4-oxadiazol-3-yl]phenyl]ethanamine as an oil. LC/MS retention time=1.05 minutes, 288 (M+H).

¹H NMR (400 MHz, CDCl₃) δ ppm: 8.10 (d, 2H), 7.51 (d, 2H), 5.65 (s_{br}, 1H), 4.22 (q, 1H), 3.47 (s, 3H), 1.38 (d, 3H).

¹⁹F NMR (400 MHz, CDCl₃) δ ppm: −65.37 (s).

### Example 9: This example illustrates the preparation of intermediate of 2-[4-[5-(trifluoromethyl)-1,2,4-oxadiazol-3-yl]phenyl]ethylammonium chloride

### Step 1: Preparation of tert-butyl-[2-(4-cyanophenyl)ethyl]carbamate

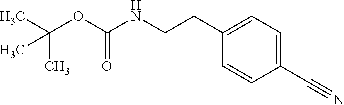 [SMILES: CC(C)(C)OC(=O)NCCc1ccc(C#N)cc1]

To a solution of 2-(4-cyanophenyl)ethylammonium chloride (3.0 g, 16 mmol) in THF (70 mL) was added triethylamine (6.9 mL, 49 mmol) and DMAP (200 mg, 1.6 mmol). The resulting beige solution was cooled using an ice bath and tert-butoxycarbonyl tert-butyl carbonate (5.4 g, 25 mmol) was introduced dropwise as a THF solution (12 mL). The ice bath was removed and stirring continued overnight. Ice and water were added and extraction was carried out with Et₂O (2×40 mL). The combined organic layers were washed with brine, dried over Na₂SO₄, filtered, and concentrated under reduced pressure to afford a light yellow solid. The resulting crude residue was absorbed on isolute and purified via combiflash column chromatography using a cyclohexane/ethylacetate eluent gradient to afford 1.56 g of tert-butyl N-[2-(4-cyanophenyl)ethyl]carbamate as a white solid. mp. 70-74° C.

LC/MS (Method A) retention time=0.94 minutes, mass not detected.

¹H NMR (400 MHz, CDCl₃) δ ppm: 7.60 (d, 2H), 7.30 (d, 2H), 4.55 (s_{br}, 1H), 3.37 (m, 2H), 2.85 (m, 2H), 1.40 (s, 9H).

### Step 2: Preparation of tert-butyl N-[2-[4-[5-(trifluoromethyl)-1,2,4-oxadiazol-3-yl]phenyl]ethyl]carbamate

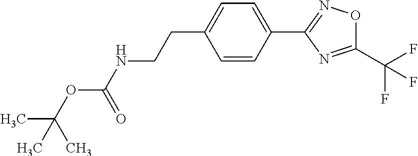 [SMILES: CC(C)(C)OC(=O)NCCc1ccc(-c2noc(C(F)(F)F)n2)cc1]

To a solution of tert-butyl N-[2-(4-cyanophenyl)ethyl]carbamate (912 mg, 3.7 mmol) in ethanol (18.5 mL) was added triethylamine (1.04 mL, 7.4 mmol) followed by the portion-wise introduction hydroxylamine hydrochloride (520 mg, 7.4 mmol). The reaction mixture was then heated to 80° C. for 3.5 hours. After the reaction mixture cooled to 22° C., the ethanol was removed under reduced pressure, and the resulting crude tert-butyl N-[2-[4-[N′-hydroxycarbamimidoyl]phenyl]ethyl]carbamate residue was suspended in THF (37 mL). Pyridine (1.2 mL, 14.8 mL) was introduced and the reaction contents were cooled using an ice bath. Trifluoroacetic anhydride (1.57 mL, 11.1 mmol) was then added dropwise. The ice bath was removed and stirring was continued overnight. The reaction contents were concentrated under reduced pressure and diethyl acetate and water were introduced. The layers were separated and the organic fraction was washed sequentially with an aqueous 1M NaOH solution, water, and brine then dried over sodium sulfate, filtered, and concentrated to give a yellow crude solid that was absorbed on isolute and purified via combiflash column chromatography using a cyclohexane/ethyl acetate eluent gradient to afford 826 mg of tert-butyl N-[2-[4-[5-(trifluoromethyl)-1,2,4-oxadiazol-3-yl]phenyl]ethyl]carbamate as a white solid. mp: 81-83° C.

LC/MS (Method A) retention time=1.17 minutes, mass not detected.

¹H NMR (400 MHz, CDCl₃) δ ppm: 8.05 (d, 2H), 7.85 (d, 2H), 4.55 (s_{br}, 1H), 3.48 (m, 2H), 2.88 (m 2H), 1.42 (s, 9H).

### Step 3: Preparation of 2-[4-[5-(trifluoromethyl)-1,2,4-oxadiazol-3-yl]phenyl]ethylammonium chloride

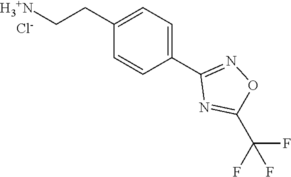

To a solution of tert-butyl N-[2-[4-[5-(trifluoromethyl)-1,2,4-oxadiazol-3-yl]phenyl]ethyl]carbamate (500 mg, 1.4 mmol) in ethyl acetate (10 mL) cooled with an ice bath was introduced dropwise a 4M HCl 1,4-dioxane solution (2.8 mL, 11.2 mmol). The ice bath was removed and stirring was continued overnight. A fine white suspension slowly formed and was collected via filtration, washed twice with ethyl acetate, and dried in a vacuum oven to afford 378 mg of 2-[4-[5-(trifluoromethyl)-1,2,4-oxadiazol-3-yl]phenyl]ethylammonium chloride as a white solid. mp>225° C.

LC/MS (Method A) retention time=0.67 minutes, 258 [M-Cl]⁺.

¹H NMR (400 MHz, DMSO) δ ppm: 8.05 (d, 2H), 7.52 (d, 2H), 3.10 (m, 2H), 3.00 (m, 2H).

The following general procedure was used in a combinatorial fashion using appropriate building blocks (compounds (II) and (III)) to provide the compounds of Formula (I). The compounds prepared via the following combinatorial protocol were analyzed using LC/MS Method B.

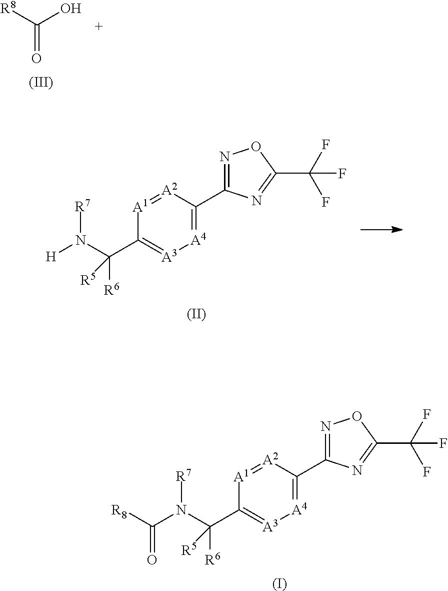

By way of exemplification, acid derivatives of formula (III) (0.0375 mmol in 375 μl DMA) were transferred to a 96 slot deep well plate (DWP96) containing the [4-[5-(trifluoromethyl)-1,2,4-oxadiazol-3-yl]aryl]methanamine derivative of formula (II) (0.03 mmol) and DIPEA (0.09 mmol) in 250 μl DMA, followed by the addition of BOP—Cl (0.06 mmol) dissolved in DMA (250 μl). The DWP was sealed and stirred at 50° C. for 18 hours. The solvent was removed under a stream of nitrogen. The resultant crude residues were solubilized in a mixture of MeOH (250 μl) and DMA (500 μl) and directly submitted for preparative LC/MS purification which provided the compounds of formula (I) in 10-85% yields.

Alternatively, the following general procedure was used in a combinatorial fashion using appropriate building blocks (compounds (IV) and (V)) to provide the compounds of Formula (I). The compounds prepared via the following combinatorial protocol were analyzed using LC/MS Method B.

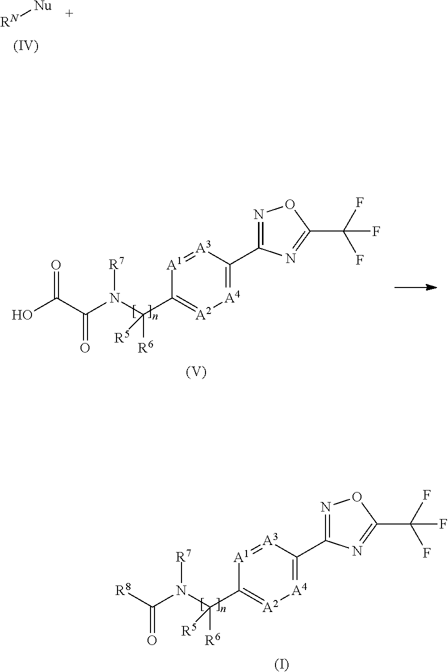

By way of exemplification, nucleophile derivatives of formula (IV) (0.0375 mmol in 375 μl DMA) were transferred to a 96 slot deep well plate (DWP96) containing the N-[4-(5-trifluoromethyl-[1,2,4]oxadiazol-3-yl)-aryl]oxalamic acid derivative of formula (V) (0.03 mmol) and DIPEA (0.09 mmol) in 250 μl DMA, followed by the addition of BOP—Cl (0.06 mmol) dissolved in DMA (250 μl). The DWP was sealed and stirred at 50° C. for 18 hours. The solvent was removed under a stream of nitrogen. The resultant crude residues were solubilized in a mixture of MeOH (250 μl) and DMA (500 μl) and directly submitted for preparative LC/MS purification which provided the compounds of formula (I) in 10-85% yields.

Where necessary, enantiomerically pure final compounds may be obtained from racemic materials as appropriate via standard physical separation techniques, such as reverse phase chiral chromatography, or through stereoselective synthetic techniques, (eg, by using chiral starting materials).

TABLE T1
Melting point (mp) data and/or retention times (RT) for compounds according to Formula (I):

| | | | | Mass | | |
|---|---|---|---|---|---|---|
| | | | | charge | | |
| | | | RT | [M + | Meth- | MP |
| Entry | Compound name | Structure | (min) | H] | od | (° C.) |
| 1.1 | 2-oxo-2- phenyl-N-[4-[5- (trifluoromethyl)- 1,2,4- oxadiazol-3- yl]phenyl] acetamide | 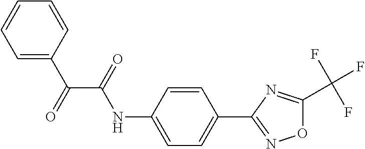 [SMILES: O=C(Nc1ccc(-c2noc(C(F)(F)F)n2)cc1)C(=O)c1ccccc1] | 1.85 | 362.3 | B | |
| 1.2 | 2-(3- fluorophenyl)-2- oxo-N-[4-[5- (trifluoromethyl)- 1,2,4- oxadiazol-3- yl]phenyl] acetamide | 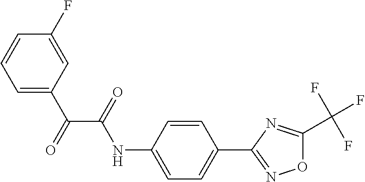 [SMILES: O=C(Nc1ccc(-c2noc(C(F)(F)F)n2)cc1)C(=O)c1cccc(F)c1] | 1.89 | 380.2 | B | |
| 1.3 | N-methoxy- 2-oxo-N-[[4- [5-(trifluoromethyl)- 1,2,4-oxadiazol-3- yl]phenyl]methyl] pentanamide | 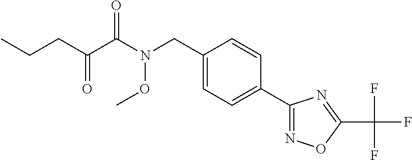 [SMILES: CCCC(=O)C(=O)N(Cc1ccc(-c2noc(C(F)(F)F)n2)cc1)OC] | 1.88 | 372.2 | B | |
| 1.4 | N-cyclopropyl- 2-oxo-N- [[4-[5- (trifluoromethyl)- 1,2,4-oxadiazol-3- yl]phenyl]methyl] pentanamide | 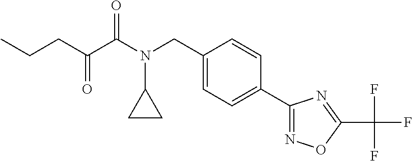 [SMILES: CCCC(=O)C(=O)N(Cc1ccc(-c2noc(C(F)(F)F)n2)cc1)C1CC1] | 1.94 | 382.2 | B | |
| 1.5 | 2-oxo-N-prop- 2-ynyl-N- [[4-[5- (trifluoromethyl)- 1,2,4-oxadiazol-3- yl]phenyl]methyl] pentanamide | 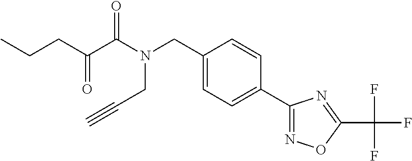 [SMILES: C#CCN(Cc1ccc(-c2noc(C(F)(F)F)n2)cc1)C(=O)C(=O)CCC] | 1.91 | 380.2 | B | |
| 1.6 | N-(cyclopropyl- methyl)-2- oxo-N-[[4-[5- (trifluoromethyl)- 1,2,4- oxadiazol-3- yl]phenyl]methyl] pentanamide | 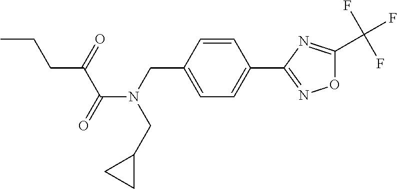 [SMILES: CCCC(=O)C(=O)N(Cc1ccc(-c2noc(C(F)(F)F)n2)cc1)CC1CC1] | 2.03 | 396.2 | B | |
| 1.7 | 2-oxo-N- propoxy-N-[[4- [5-(trifluoromethyl)- 1,2,4-oxadiazol-3- yl]phenyl]methyl] pentanamide | 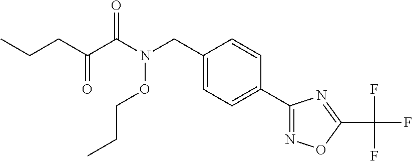 [SMILES: CCCON(Cc1ccc(-c2noc(C(F)(F)F)n2)cc1)C(=O)C(=O)CCC] | 2.02 | 400.4 | B | |
| 1.8 | 2-oxo-N- propoxy-N-[[4- [5-(trifluoromethyl)- 1,2,4-oxadiazol-3- yl]phenyl]methyl] propanamide | 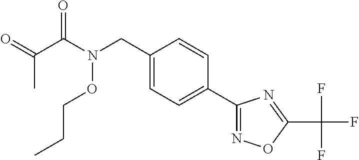 [SMILES: CCCON(Cc1ccc(-c2noc(C(F)(F)F)n2)cc1)C(=O)C(C)=O] | 1.82 | 372.3 | | |
| 1.9 | N-(2- methoxyethyl)-2- oxo-N-[[4-[5- (trifluoromethyl)- 1,2,4- oxadiazol-3- yl]phenyl]methyl] pentanamide | 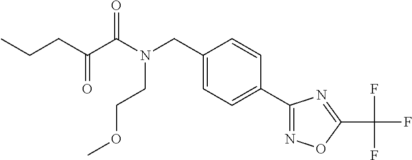 [SMILES: CCCC(=O)C(=O)N(CCOC)Cc1ccc(-c2noc(C(F)(F)F)n2)cc1] | 1.87 | 400.4 | B | |
| 1.10 | N-(2- methoxyethyl)-2- oxo-N-[[4-[5- (trifluoromethyl)- 1,2,4- oxadiazol-3- yl]phenyl]methyl] propanamide | 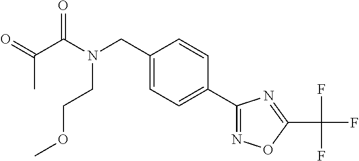 [SMILES: COCCN(Cc1ccc(-c2noc(C(F)(F)F)n2)cc1)C(=O)C(C)=O] | 1.65 | 372.3 | B | |
| 1.11 | 2-oxo-N-sec- butyl-N-[[4- [5-(trifluoromethyl)- 1,2,4-oxadiazol-3- yl]phenyl]methyl] pentanamide | 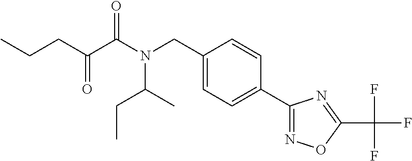 [SMILES: CCCC(=O)C(=O)N(Cc1ccc(-c2noc(C(F)(F)F)n2)cc1)C(C)CC] | 2.02 | 398.3 | B | |
| 1.12 | 2-oxo-N-sec- butyl-N-[[4- [5-(trifluoromethyl)- 1,2,4-oxadiazol-3- yl]phenyl]methyl] propanamide | 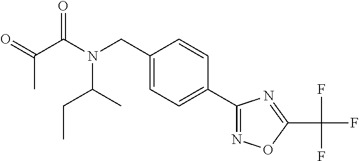 [SMILES: CCC(C)N(Cc1ccc(-c2noc(C(F)(F)F)n2)cc1)C(=O)C(C)=O] | 1.82 | 370.3 | B | |
| 1.13 | 2-oxo-N- (tetrahydrofuran-2- ylmethyl)-N-[[4-[5- (trifluoromethyl)- 1,2,4- oxadiazol-3- yl]phenyl]methyl] pentanamide | 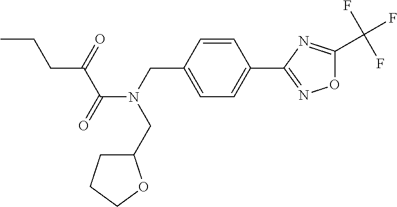 [SMILES: CCCC(=O)C(=O)N(Cc1ccc(-c2noc(C(F)(F)F)n2)cc1)CC1CCCO1] | 1.94 | 426.4 | B | |
| 1.14 | 2-oxo-N- (tetrahydrofuran-2- ylmethyl)-N-[[4-[5- (trifluoromethyl)- 1,2,4- oxadiazol-3- yl]phenyl]methyl] propanamide | 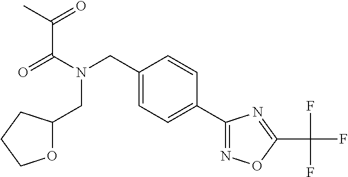 [SMILES: CC(=O)C(=O)N(Cc1ccc(-c2noc(C(F)(F)F)n2)cc1)CC1CCCO1] | 1.74 | 398.3 | B | |
| 1.15 | N-(2-furylmethyl)- 2-oxo- N-[[4-[5- (trifluoromethyl)- 1,2,4- oxadiazol-3- yl]phenyl]methyl] pentanamide | 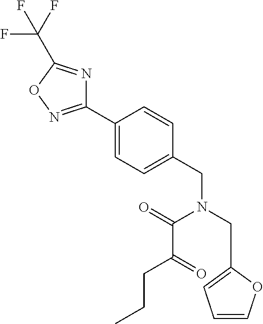 [SMILES: CCCC(=O)C(=O)N(Cc1ccc(-c2noc(C(F)(F)F)n2)cc1)Cc1ccco1] | 1.97 | 422.3 | B | |
| 1.16 | N-allyl-2- oxo-N-[[4-[5- (trifluoromethyl)- 1,2,4- oxadiazol-3- yl]phenyl]methyl] propanamide | 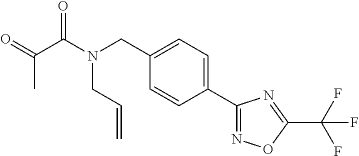 [SMILES: C=CCN(Cc1ccc(-c2noc(C(F)(F)F)n2)cc1)C(=O)C(C)=O] | 1.73 | 354.2 | B | |
| 1.17 | N-allyl-2- oxo-N-[[4-[5- (trifluoromethyl)- 1,2,4- oxadiazol-3- yl]phenyl]methyl] pentanamide | 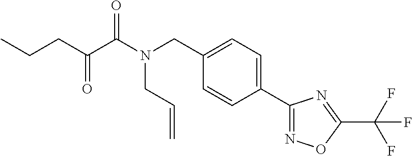 [SMILES: C=CCN(Cc1ccc(-c2noc(C(F)(F)F)n2)cc1)C(=O)C(=O)CCC] | 1.94 | 382.3 | B | |
| 1.18 | N-ethyl-2- oxo-N-[[4-[5- (trifluoromethyl)- 1,2,4- oxadiazol-3- yl]phenyl]methyl] propanamide | 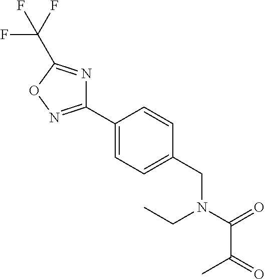 [SMILES: CCN(Cc1ccc(-c2noc(C(F)(F)F)n2)cc1)C(=O)C(C)=O] | 1.66 | 342.3 | B | |
| 1.19 | N-ethyl-2- oxo-N-[[4-[5- (trifluoromethyl)- 1,2,4- oxadiazol-3- yl]phenyl]methyl] pentanamide | 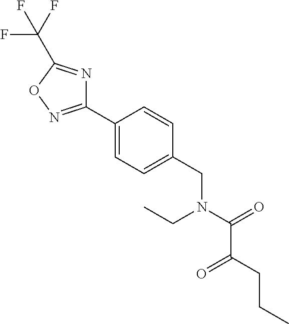 [SMILES: CCCC(=O)C(=O)N(CC)Cc1ccc(-c2noc(C(F)(F)F)n2)cc1] | 1.88 | 370.3 | B | |
| 1.20 | N-methyl-2- oxo-N-[[4-[5- (trifluoromethyl)- 1,2,4- oxadiazol-3- yl]phenyl]methyl] propanamide | 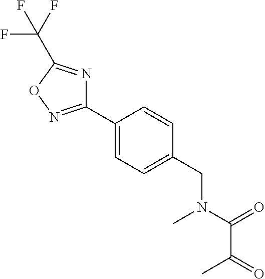 [SMILES: CC(=O)C(=O)N(C)Cc1ccc(-c2noc(C(F)(F)F)n2)cc1] | 1.55 | 328.2 | B | |
| 1.21 | N-methyl-2- oxo-N-[[4-[5- (trifluoromethyl)- 1,2,4- oxadiazol-3- yl]phenyl]methyl] pentanamide | 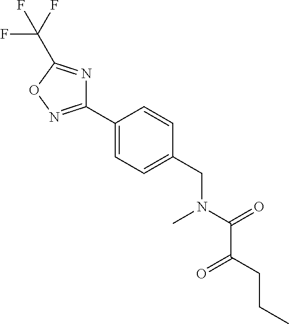 [SMILES: CCCC(=O)C(=O)N(C)Cc1ccc(-c2noc(C(F)(F)F)n2)cc1] | 1.78 | 356.3 | B | |
| 1.22 | N-(3,3-dichloro- allyloxy)- 2-oxo-N-[[4-[5- (trifluoromethyl)- 1,2,4- oxadiazol-3- yl]phenyl]methyl] propanamide | 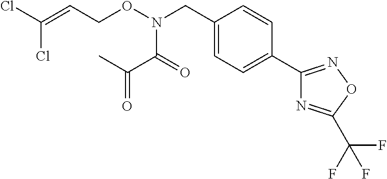 [SMILES: CC(=O)C(=O)N(Cc1ccc(-c2noc(C(F)(F)F)n2)cc1)OCC=C(Cl)Cl] | 1.94 | 438.2 | B | |
| 1.23 | N-(3,3-dichloro- allyloxy)- 2-oxo-N-[[4-[5- (trifluoromethyl)- 1,2,4- oxadiazol-3- yl]phenyl]methyl] pentanamide | 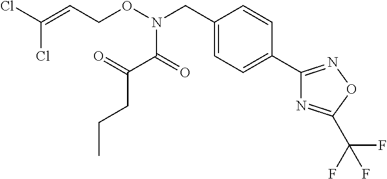 [SMILES: CCCC(=O)C(=O)N(Cc1ccc(-c2noc(C(F)(F)F)n2)cc1)OCC=C(Cl)Cl] | 2.12 | 466.3 | B | |
| 1.24 | N-[(4- chlorophenyl) methoxy]- 2-oxo-N-[[4-[5- (trifluoromethyl)- 1,2,4- oxadiazol-3- yl]phenyl]methyl] pentanamide | 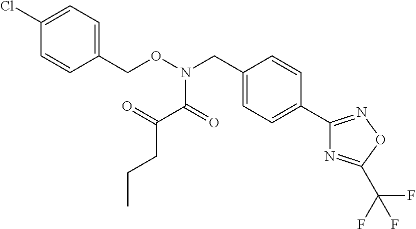 [SMILES: CCCC(=O)C(=O)N(Cc1ccc(-c2noc(C(F)(F)F)n2)cc1)OCc1ccc(Cl)cc1] | 2.17 | 482.3 | B | |
| 1.25 | N-[[3-fluoro-4-[5- (trifluoromethyl)- 1,2,4- oxadiazol-3- yl]phenyl] methyl]-2-oxo- propanamide | 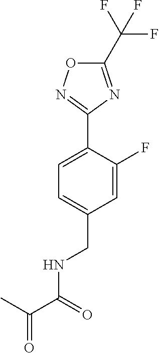 [SMILES: CC(=O)C(=O)NCc1ccc(-c2noc(C(F)(F)F)n2)c(F)c1] | 1.52 | 332.05 | B | |
| 1.26 | N-[[2-fluoro-4-[5- (trifluoromethyl)- 1,2,4- oxadiazol-3- yl]phenyl]methyl]- 2-oxo- propanamide | 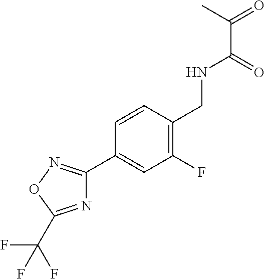 [SMILES: CC(=O)C(=O)NCc1ccc(-c2noc(C(F)(F)F)n2)cc1F] | 1.52 | 332.04 | B | |
| 1.27 | N-[[2,3- difluoro-4-[5- (trifluoromethyl)- 1,2,4- oxadiazol-3- yl]phenyl]methyl]- 2-oxo- propanamide | 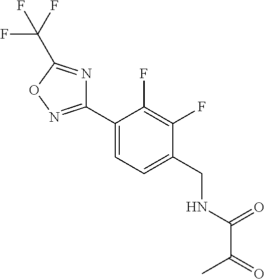 [SMILES: CC(=O)C(=O)NCc1ccc(-c2noc(C(F)(F)F)n2)c(F)c1F] | 1.50 | 350.05 | B | |
| 1.28 | 2-oxo-N-[[5-[5- (trifluoromethyl)- 1,2,4- oxadiazol-3-yl]-2- pyridyl]methyl] propanamide | 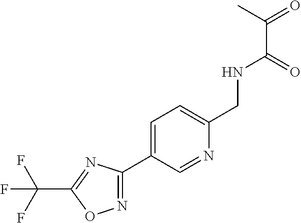 [SMILES: CC(=O)C(=O)NCc1ccc(-c2noc(C(F)(F)F)n2)cn1] | 1.22 | 315.02 | B | |
| 1.29 | N-ethoxy-2- oxo-N-[[4-[5- (trifluoromethyl)- 1,2,4- oxadiazol-3- yl]phenyl]methyl] butanamide | 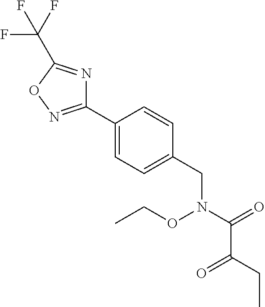 [SMILES: CCON(Cc1ccc(-c2noc(C(F)(F)F)n2)cc1)C(=O)C(=O)CC] | 1.82 | 372.16 | B | |
| 1.30 | N-butoxy-2- oxo-N-[[4-[5- (trifluoromethyl)- 1,2,4- oxadiazol-3- yl]phenyl]methyl] butanamide | 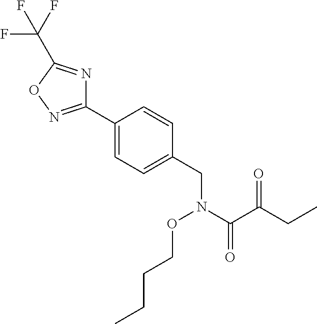 [SMILES: CCCCON(Cc1ccc(-c2noc(C(F)(F)F)n2)cc1)C(=O)C(=O)CC] | 2.01 | 400.19 | B | |
| 1.31 | 2-oxo-N- propyl-N-[[4-[5- (trifluoromethyl)- 1,2,4- oxadiazol-3- yl]phenyl]methyl] butanamide | 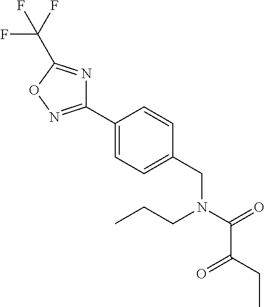 [SMILES: CCCN(Cc1ccc(-c2noc(C(F)(F)F)n2)cc1)C(=O)C(=O)CC] | 1.86 | 370.19 | B | |
| 1.32 | N-isopropyl- 2-oxo-[[4- [5-(trifluoromethyl)- 1,2,4-oxadiazol-3- yl]phenyl]methyl] butanamide | 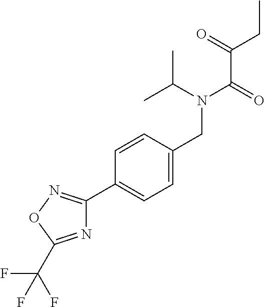 [SMILES: CCC(=O)C(=O)N(Cc1ccc(-c2noc(C(F)(F)F)n2)cc1)C(C)C] | 1.81 | 370.19 | B | |
| 1.33 | N-(2- hydroxyethyl)-2- oxo-N-[[4-[5- (trifluoromethyl)- 1,2,4- oxadiazol-3- yl]phenyl]methyl] butanamide | 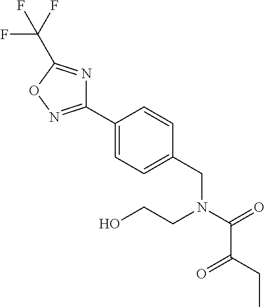 [SMILES: CCC(=O)C(=O)N(CCO)Cc1ccc(-c2noc(C(F)(F)F)n2)cc1] | 1.40 | 372.16 | B | |
| 1.34 | N-methyl- 2-oxo-[4-[5- (trifluoromethyl)- 1,2,4- oxadiazol-3- yl]phenyl] butanamide | 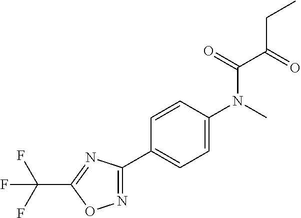 [SMILES: CCC(=O)C(=O)N(C)c1ccc(-c2noc(C(F)(F)F)n2)cc1] | 1.59 | 328.1 | B | |
| 1.35 | 2-oxo-N-[[4-[5- (trifluoromethyl)- 1,2,4- oxadiazol-3- yl]phenyl]methyl] propanamide | 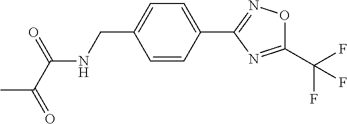 [SMILES: CC(=O)C(=O)NCc1ccc(-c2noc(C(F)(F)F)n2)cc1] | | | | 75.8- 77.5 |
| 1.36 | N-methoxy- 2-oxo-N-[[4- [5-(trifluoromethyl)- 1,2,4-oxadiazol-3- yl]phenyl]methyl] propanamide | 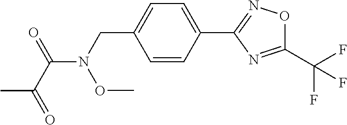 [SMILES: CON(Cc1ccc(-c2noc(C(F)(F)F)n2)cc1)C(=O)C(C)=O] | 1.61 | 344.07 | B | |
| 1.37 | N-[[2-fluoro-4-[5- (trifluoromethyl)- 1,2,4- oxadiazol-3- yl]phenyl] methyl]-2-oxo- 2-phenyl-acetamide | 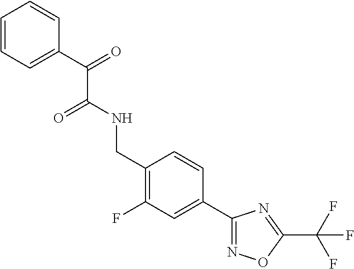 [SMILES: O=C(NCc1ccc(-c2noc(C(F)(F)F)n2)cc1F)C(=O)c1ccccc1] | 1.77 | 394.06 | B | |
| 1.38 | N-[[2-fluoro-4-[5- (trifluoromethyl)- 1,2,4- oxadiazol-3- yl]phenyl]methyl]- 4-methyl-2-oxo- pentanamide | 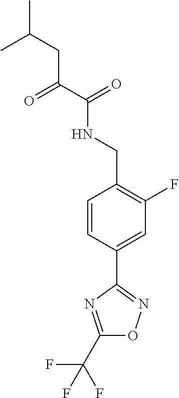 [SMILES: CC(C)CC(=O)C(=O)NCc1ccc(-c2noc(C(F)(F)F)n2)cc1F] | 1.85 | 374.08 | B | |
| 1.39 | N-[[2-fluoro-4-[5- (trifluoromethyl)- 1,2,4- oxadiazol-3- yl]phenyl] methyl]-2-oxo- 2-(2-thienyl) acetamide | 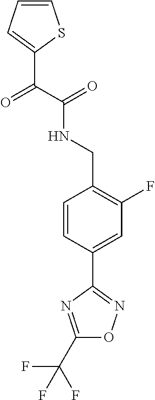 [SMILES: O=C(NCc1ccc(-c2noc(C(F)(F)F)n2)cc1F)C(=O)c1cccs1] | 1.78 | 400 | B | |
| 1.40 | N-[[2-fluoro-4-[5- (trifluoromethyl)- 1,2,4- oxadiazol-3- yl]phenyl] methyl]-3,3- dimethyl-2-oxo- butanamide | 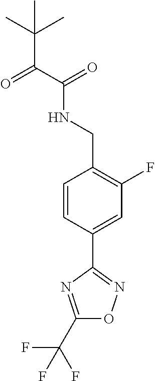 [SMILES: CC(C)(C)C(=O)C(=O)NCc1ccc(-c2noc(C(F)(F)F)n2)cc1F] | 1.85 | 374.07 | B | |
| 1.41 | N-[[2-fluoro-4-[5- (trifluoromethyl)- 1,2,4- oxadiazol-3- yl]phenyl] methyl]-2-oxo- butanamide | 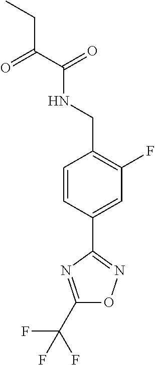 [SMILES: CCC(=O)C(=O)NCc1ccc(-c2noc(C(F)(F)F)n2)cc1F] | 1.63 | 346.06 | B | |
| 1.42 | N-[[3-fluoro-4-[5- (trifluoromethyl)- 1,2,4- oxadiazol-3- yl]phenyl] methyl]-2-oxo- 2-phenyl- acetamide | 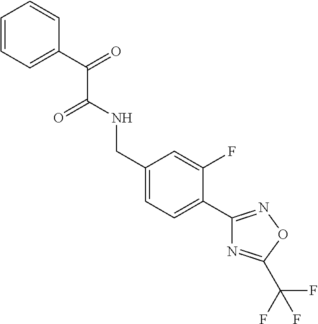 [SMILES: O=C(NCc1ccc(-c2noc(C(F)(F)F)n2)c(F)c1)C(=O)c1ccccc1] | 1.77 | 394.06 | B | |
| 1.43 | N-[[3-fluoro-4-[5- (trifluoromethyl)- 1,2,4- oxadiazol-3- yl]phenyl]methyl]- 4-methyl-2-oxo- pentanamide | 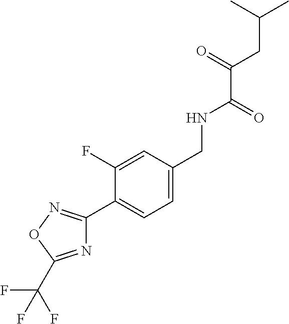 [SMILES: CC(C)CC(=O)C(=O)NCc1ccc(-c2noc(C(F)(F)F)n2)c(F)c1] | 1.85 | 374.07 | B | |
| 1.44 | N-[[3-fluoro-4-[5- (trifluoromethyl)- 1,2,4- oxadiazol-3- yl]phenyl] methyl]-2-oxo- 2-(2-thienyl) acetamide | 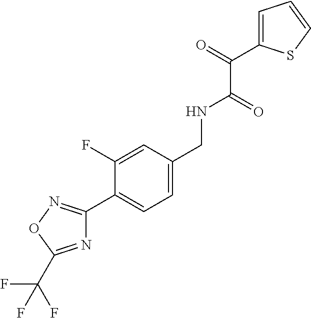 [SMILES: O=C(NCc1ccc(-c2noc(C(F)(F)F)n2)c(F)c1)C(=O)c1cccs1] | 1.79 | 400 | B | |
| 1.45 | N-[[3-fluoro-4-[5- (trifluoromethyl)- 1,2,4- oxadiazol-3- yl]phenyl] methyl]-3,3- dimethyl-2-oxo- butanamide | 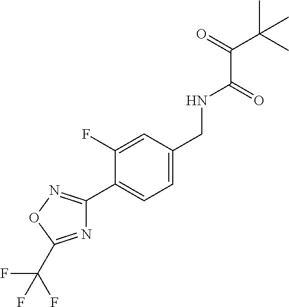 [SMILES: CC(C)(C)C(=O)C(=O)NCc1ccc(-c2noc(C(F)(F)F)n2)c(F)c1] | 1.85 | 374.07 | B | |
| 1.46 | N-[[2,3- difluoro-4-[5- (trifluoromethyl)- 1,2,4- oxadiazol-3- yl]phenyl] methyl]-2-oxo- 2-phenyl- acetamide | 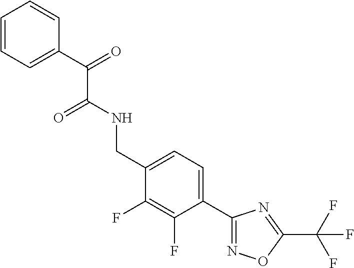 [SMILES: O=C(NCc1ccc(-c2noc(C(F)(F)F)n2)c(F)c1F)C(=O)c1ccccc1] | 1.75 | 412.03 | B | |
| 1.47 | N-[[2,3- difluoro-4-[5- (trifluoromethyl)- 1,2,4- oxadiazol-3- yl]phenyl]methyl]- 4-methyl-2-oxo- pentanamide | 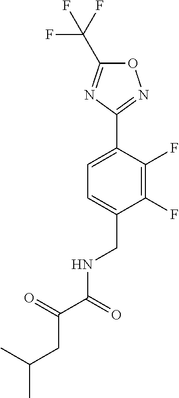 [SMILES: CC(C)CC(=O)C(=O)NCc1ccc(-c2noc(C(F)(F)F)n2)c(F)c1F] | 1.83 | 392.07 | B | |
| 1.48 | N-[[2,3- difluoro-4-[5- (trifluoromethyl)- 1,2,4- oxadiazol-3- yl]phenyl] methyl]-2-oxo- 2-(2-thienyl) acetamide | 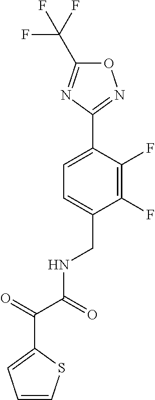 [SMILES: O=C(NCc1ccc(-c2noc(C(F)(F)F)n2)c(F)c1F)C(=O)c1cccs1] | 1.76 | 417.98 | B | |
| 1.49 | N-[[2,3- difluoro-4-[5- (trifluoromethyl)- 1,2,4- oxadiazol-3- yl]phenyl] methyl]-3,3- dimethyl-2-oxo- butanamide | 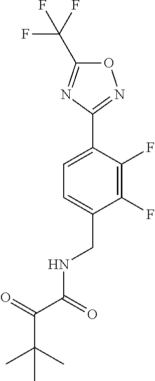 [SMILES: CC(C)(C)C(=O)C(=O)NCc1ccc(-c2noc(C(F)(F)F)n2)c(F)c1F] | 1.74 | 392.08 | B | |
| 1.50 | 4-methyl- 2-oxo-N-[[4-[5- (trifluoromethyl)- 1,2,4- oxadiazol-3- yl]phenyl]methyl] pentanamide | 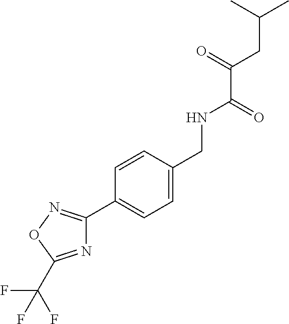 [SMILES: CC(C)CC(=O)C(=O)NCc1ccc(-c2noc(C(F)(F)F)n2)cc1] | 1.82 | 356.09 | B | |
| 1.51 | 2-oxo-2- (2-thienyl)-N- [[4-[5- (trifluoromethyl)- 1,2,4-oxadiazol-3- yl]phenyl]methyl] acetamide | 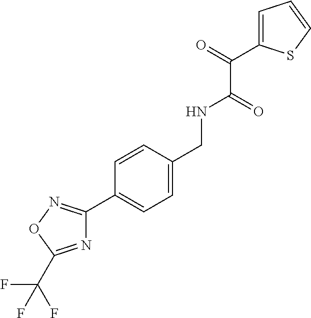 [SMILES: O=C(NCc1ccc(-c2noc(C(F)(F)F)n2)cc1)C(=O)c1cccs1] | 1.74 | 382.01 | B | |
| 1.52 | 3,3-dimethyl- 2-oxo-N- [[4-[5- (trifluoromethyl)- 1,2,4-oxadiazol-3- yl]phenyl]methyl] butanamide | 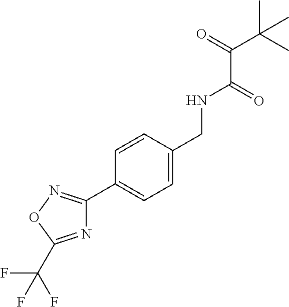 [SMILES: CC(C)(C)C(=O)C(=O)NCc1ccc(-c2noc(C(F)(F)F)n2)cc1] | 1.81 | 356.09 | B | |
| 1.53 | 2-oxo-N-[[4-[5- (trifluoromethyl)- 1,2,4- oxadiazol-3- yl]phenyl]methyl] butanamide | 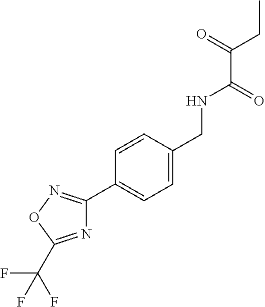 [SMILES: CCC(=O)C(=O)NCc1ccc(-c2noc(C(F)(F)F)n2)cc1] | 1.58 | 328.06 | B | |
| 1.54 | N-[[2- fluoro-4-[5- (trifluoromethyl)- 1,2,4- oxadiazol-3- yl]phenyl] methyl]-N- methoxy-2-oxo-2- phenyl-acetamide | 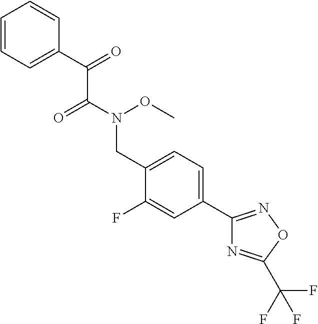 [SMILES: CON(Cc1ccc(-c2noc(C(F)(F)F)n2)cc1F)C(=O)C(=O)c1ccccc1] | 1.88 | 424.04 | B | |
| 1.55 | N-[[2- fluoro-4-[5- (trifluoromethyl)- 1,2,4- oxadiazol-3- yl]phenyl] methyl]-N- methoxy-4- methyl-2-oxo- pentanamide | 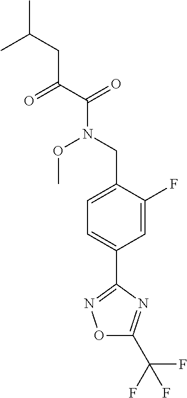 [SMILES: CON(Cc1ccc(-c2noc(C(F)(F)F)n2)cc1F)C(=O)C(=O)CC(C)C] | 1.97 | 404.08 | B | |
| 1.56 | N-[[2-fluoro-4-[5- (trifluoromethyl)- 1,2,4- oxadiazol-3- yl]phenyl] methyl]-N- methoxy-2- oxo-2-(2- thienyl)acetamide | 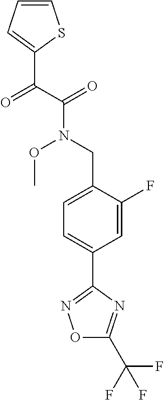 [SMILES: CON(Cc1ccc(-c2noc(C(F)(F)F)n2)cc1F)C(=O)C(=O)c1cccs1] | 1.82 | 429.99 | B | |
| 1.57 | N-[[2-fluoro-4-[5- (trifluoromethyl)- 1,2,4- oxadiazol-3- yl]phenyl]methyl]- N-methoxy-3,3- dimethyl-2- oxo-butanamide | 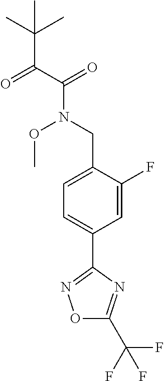 [SMILES: CON(Cc1ccc(-c2noc(C(F)(F)F)n2)cc1F)C(=O)C(=O)C(C)(C)C] | 1.98 | 404.09 | B | |
| 1.58 | N-[[2-fluoro-4-[5- (trifluoromethyl)- 1,2,4- oxadiazol-3- yl]phenyl] methyl]-N- methoxy-2-oxo- butanamide | 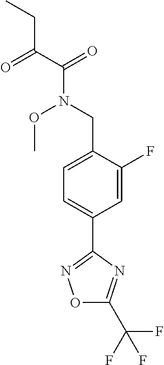 [SMILES: CCC(=O)C(=O)N(Cc1ccc(-c2noc(C(F)(F)F)n2)cc1F)OC] | 1.77 | 376.07 | B | |
| 1.59 | N-[[2-chloro-4-[5- (trifluoromethyl)- 1,2,4- oxadiazol-3- yl]phenyl] methyl]-2-oxo- 2-phenyl- acetamide | 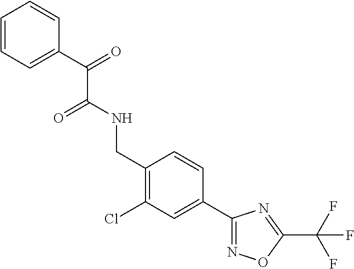 [SMILES: O=C(NCc1ccc(-c2noc(C(F)(F)F)n2)cc1Cl)C(=O)c1ccccc1] | 1.87 | 410.01 | B | |
| 1.60 | N-[[2-chloro-4-[5- (trifluoromethyl)- 1,2,4- oxadiazol-3- yl]phenyl]methyl]- 2-oxo- propanamide | 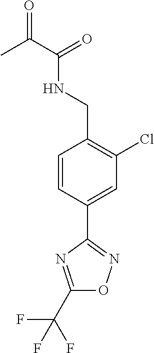 [SMILES: CC(=O)C(=O)NCc1ccc(-c2noc(C(F)(F)F)n2)cc1Cl] | 1.61 | 348.03 | B | |
| 1.61 | N-[[2-chloro-4-[5- (trifluoromethyl)- 1,2,4- oxadiazol-3- yl]phenyl]methyl]- 4-methyl-2-oxo- pentanamide | 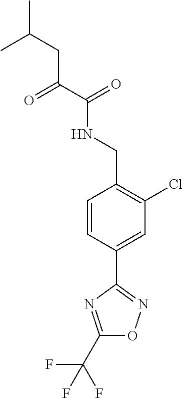 [SMILES: CC(C)CC(=O)C(=O)NCc1ccc(-c2noc(C(F)(F)F)n2)cc1Cl] | 1.96 | 390.04 | B | |
| 1.62 | N-[[2-chloro-4-[5- (trifluoromethyl)- 1,2,4- oxadiazol-3- yl]phenyl] methyl]-2-oxo- 2-(2-thienyl) acetamide | 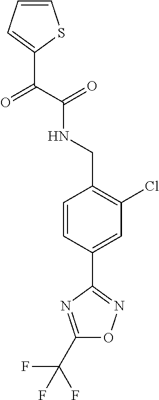 [SMILES: O=C(NCc1ccc(-c2noc(C(F)(F)F)n2)cc1Cl)C(=O)c1cccs1] | 1.89 | 415.96 | B | |
| 1.63 | N-[[2-chloro-4-[5- (trifluoromethyl)- 1,2,4- oxadiazol-3- yl]phenyl] methyl]-3,3- dimethyl-2-oxo- butanamide | 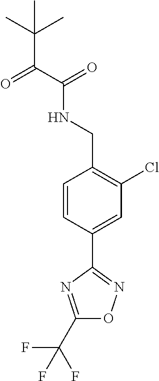 [SMILES: CC(C)(C)C(=O)C(=O)NCc1ccc(-c2noc(C(F)(F)F)n2)cc1Cl] | 1.96 | 390.05 | B | |
| 1.64 | N-[[2-chloro-4-[5- (trifluoromethyl)- 1,2,4- oxadiazol-3- yl]phenyl] methyl]-2-oxo- butanamide | 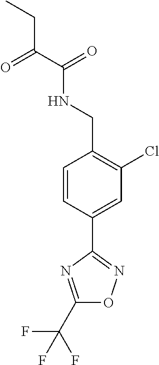 [SMILES: CCC(=O)C(=O)NCc1ccc(-c2noc(C(F)(F)F)n2)cc1Cl] | 1.78 | 362.02 | B | |
| 1.65 | N-[[2-methyl-4-[5- (trifluoromethyl)- 1,2,4- oxadiazol-3- yl]phenyl] methyl]-2-oxo-2- phenyl-acetamide | 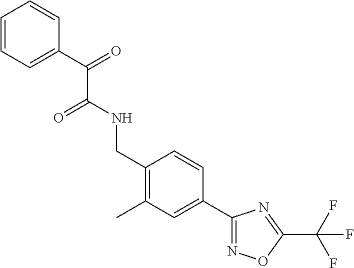 [SMILES: Cc1cc(-c2noc(C(F)(F)F)n2)ccc1CNC(=O)C(=O)c1ccccc1] | 1.82 | 390.07 | B | |
| 1.66 | N-[[2-methyl-4-[5- (trifluoromethyl)- 1,2,4- oxadiazol-3- yl]phenyl] methyl]-2-oxo- 2-(2-thienyl) acetamide | 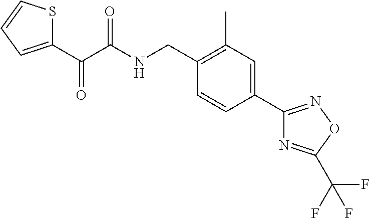 [SMILES: Cc1cc(-c2noc(C(F)(F)F)n2)ccc1CNC(=O)C(=O)c1cccs1] | 1.83 | 396.03 | B | |
| 1.67 | 3,3-dimethyl-N- [[2-methyl-4-[5- (trifluoromethyl)- 1,2,4- oxadiazol-3- yl]phenyl] methyl]-2-oxo- butanamide | 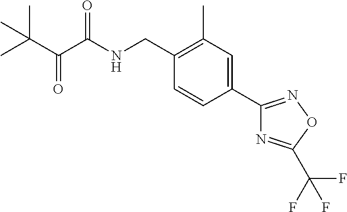 [SMILES: Cc1cc(-c2noc(C(F)(F)F)n2)ccc1CNC(=O)C(=O)C(C)(C)C] | 1.9 | 370.1 | B | |
| 1.68 | N-[[2,6- difluoro-4-[5- (trifluoromethyl)- 1,2,4- oxadiazol-3- yl]phenyl] methyl]-2-oxo- 2-phenyl- acetamide | 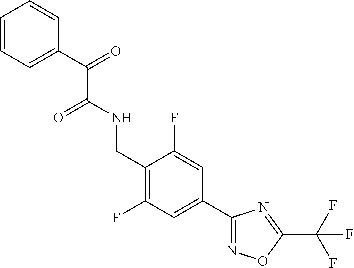 [SMILES: O=C(NCc1c(F)cc(-c2noc(C(F)(F)F)n2)cc1F)C(=O)c1ccccc1] | 1.79 | 412.04 | B | |
| 1.69 | N-[[2,6- difluoro-4-[5- (trifluoromethyl)- 1,2,4- oxadiazol-3- yl]phenyl]methyl]- 4-methyl-2-oxo- pentanamide | 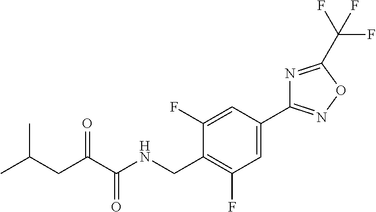 [SMILES: CC(C)CC(=O)C(=O)NCc1c(F)cc(-c2noc(C(F)(F)F)n2)cc1F] | 1.87 | 392.08 | B | |
| 1.70 | N-[[2,6- difluoro-4-[5- (trifluoromethyl)- 1,2,4- oxadiazol-3- yl]phenyl] methyl]-2-oxo- 2-(2-thienyl) acetamide | 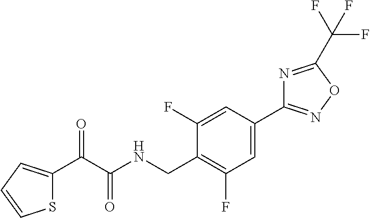 [SMILES: O=C(NCc1c(F)cc(-c2noc(C(F)(F)F)n2)cc1F)C(=O)c1cccs1] | 1.79 | 417.98 | B | |
| 1.71 | N-[[2,6- difluoro-4-[5- (trifluoromethyl)- 1,2,4- oxadiazol-3- yl]phenyl] methyl]-3,3- dimethyl-2-oxo- butanamide | 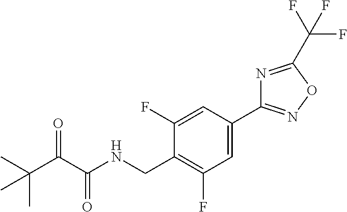 [SMILES: CC(C)(C)C(=O)C(=O)NCc1c(F)cc(-c2noc(C(F)(F)F)n2)cc1F] | 1.87 | 392.08 | B | |
| 1.72 | N-methoxy- 4-methyl-2- oxo-N-[[4-[5- (trifluoromethyl)- 1,2,4- oxadiazol-3- yl]phenyl]methyl] pentanamide | 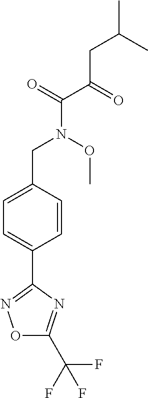 [SMILES: CON(Cc1ccc(-c2noc(C(F)(F)F)n2)cc1)C(=O)C(=O)CC(C)C] | 1.93 | 386.11 | B | |
| 1.73 | N-methoxy- 2-oxo-2-(2- thienyl)-N-[[4-[5- (trifluoromethyl)- 1,2,4- oxadiazol-3- yl]phenyl]methyl] acetamide | 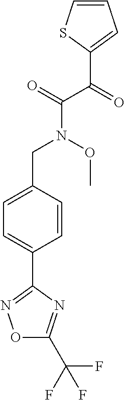 [SMILES: CON(Cc1ccc(-c2noc(C(F)(F)F)n2)cc1)C(=O)C(=O)c1cccs1] | 1.78 | 412.02 | B | |
| 1.74 | N-methoxy-3,3- dimethyl-2- oxo-N-[[4-[5- (trifluoromethyl)- 1,2,4- oxadiazol-3- yl]phenyl]methyl] butanamide | 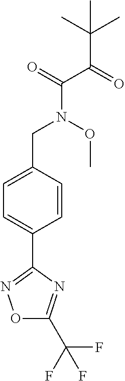 [SMILES: CON(Cc1ccc(-c2noc(C(F)(F)F)n2)cc1)C(=O)C(=O)C(C)(C)C] | 1.94 | 386.11 | B | |
| 1.75 | 2-oxo-2-phenyl- N-[[4-[5- (trifluoromethyl)- 1,2,4- oxadiazol-3- yl]phenyl]methyl] acetamide | 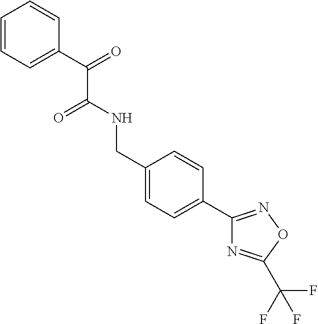 [SMILES: O=C(NCc1ccc(-c2noc(C(F)(F)F)n2)cc1)C(=O)c1ccccc1] | 1.74 | 376.04 | B | |
| 1.76 | N-methoxy-2-oxo- phenyl-N-[[4-[5- (trifluoromethyl)- 1,2,4- oxadiazol-3- yl]phenyl]methyl] acetamide | 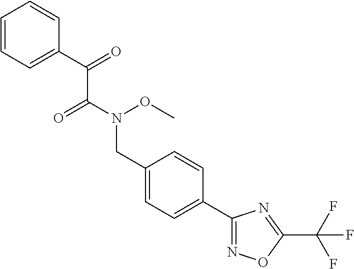 [SMILES: CON(Cc1ccc(-c2noc(C(F)(F)F)n2)cc1)C(=O)C(=O)c1ccccc1] | 1.85 | 406.07 | B | |
| 1.77 | N-[[2-fluoro-4-[5- (trifluoromethyl)- 1,2,4- oxadiazol-3- yl]phenyl] methyl]-2-(2- furyl)-2-oxo- acetamide | 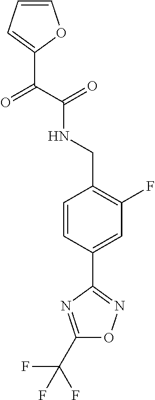 [SMILES: O=C(NCc1ccc(-c2noc(C(F)(F)F)n2)cc1F)C(=O)c1ccco1] | 1.65 | 384.03 | B | |
| 1.78 | N-[[3-fluoro-4-[5- (trifluoromethyl)- 1,2,4- oxadiazol-3- yl]phenyl] methyl]-2-oxo- butanamide | 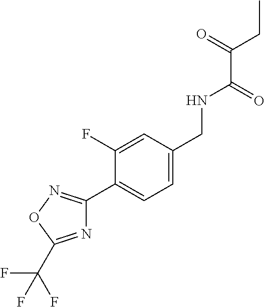 [SMILES: CCC(=O)C(=O)NCc1ccc(-c2noc(C(F)(F)F)n2)c(F)c1] | 1.63 | 346.06 | B | |
| 1.79 | N-[[3-fluoro-4-[5- (trifluoromethyl)- 1,2,4- oxadiazol-3- yl]phenyl] methyl]-2-(2- furyl)-2-oxo- acetamide | 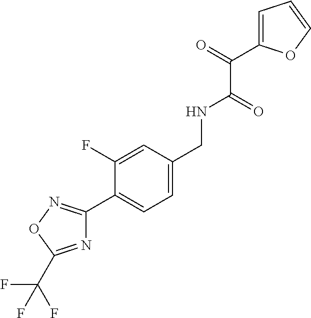 [SMILES: O=C(NCc1ccc(-c2noc(C(F)(F)F)n2)c(F)c1)C(=O)c1ccco1] | 1.65 | 384.03 | B | |
| 1.80 | N-[[2,3- difluoro-4-[5- (trifluoromethyl)- 1,2,4- oxadiazol-3- yl]phenyl] methyl]-2-oxo- butanamide | 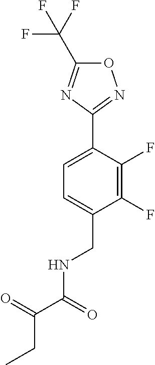 [SMILES: CCC(=O)C(=O)NCc1ccc(-c2noc(C(F)(F)F)n2)c(F)c1F] | 1.61 | 364.05 | B | |
| 1.81 | N-[[2,3- difluoro-4-[5- (trifluoromethyl)- 1,2,4- oxadiazol-3- yl]phenyl] methyl]-2-(2- furyl)-2-oxo- acetamide | 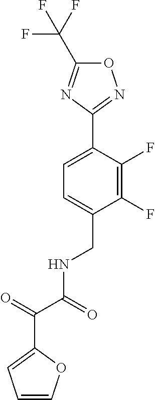 [SMILES: O=C(NCc1ccc(-c2noc(C(F)(F)F)n2)c(F)c1F)C(=O)c1ccco1] | 1.63 | 402.01 | B | |
| 1.82 | 2-(2-furyl)- 2-oxo-N-[[4-[5- (trifluoromethyl)- 1,2,4-oxadiazol-3- yl]phenyl]methyl] acetamide | 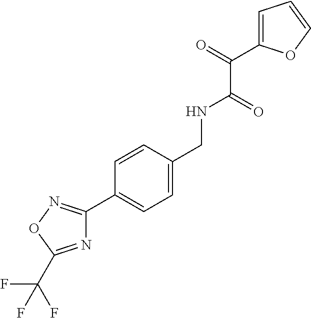 [SMILES: O=C(NCc1ccc(-c2noc(C(F)(F)F)n2)cc1)C(=O)c1ccco1] | 1.40 | 366.00 | B | |
| 1.83 | N-[[2-fluoro-4-[5- (trifluoromethyl)- 1,2,4- oxadiazol-3- yl]phenyl] methyl]-2-(2- furyl)-N-methoxy- 2-oxo- acetamide | 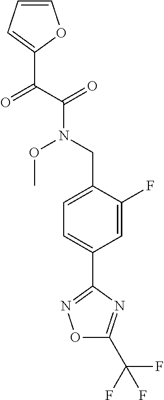 [SMILES: CON(Cc1ccc(-c2noc(C(F)(F)F)n2)cc1F)C(=O)C(=O)c1ccco1] | 1.71 | 414.03 | B | |
| 1.84 | N-[[2-chloro-4-[5- (trifluoromethyl)- 1,2,4- oxadiazol-3- yl]phenyl] methyl]-2-(2- furyl)-2-oxo- acetamide | 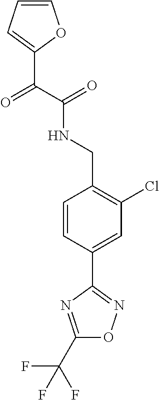 [SMILES: O=C(NCc1ccc(-c2noc(C(F)(F)F)n2)cc1Cl)C(=O)c1ccco1] | 1.76 | 400.00 | B | |
| 1.85 | N-[[2-methyl-4-[5- (trifluoromethyl)- 1,2,4- oxadiazol-3- yl]phenyl] methyl]-2-oxo- butanamide | 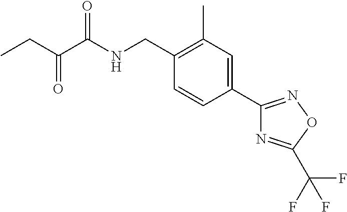 [SMILES: CCC(=O)C(=O)NCc1ccc(-c2noc(C(F)(F)F)n2)cc1C] | 1.73 | 342.09 | B | |
| 1.86 | 2-(2-furyl)-N- [[2-methyl-4- [5-(trifluoromethyl)- 1,2,4-oxadiazol-3- yl]phenyl] methyl]-2-oxo- butanamide | 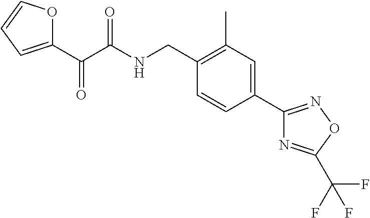 [SMILES: Cc1cc(-c2noc(C(F)(F)F)n2)ccc1CNC(=O)C(=O)c1ccco1] | 1.70 | 380.04 | B | |
| 1.87 | N-[[2,6- difluoro-4-[5- (trifluoromethyl)- 1,2,4- oxadiazol-3- yl]phenyl] methyl]-2-oxo- propanamide | 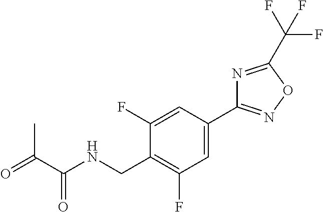 [SMILES: CC(=O)C(=O)NCc1c(F)cc(-c2noc(C(F)(F)F)n2)cc1F] | 1.53 | 350.04 | B | |
| 1.88 | N-[[2,6- difluoro-4-[5- (trifluoromethyl)- 1,2,4- oxadiazol-3- yl]phenyl] methyl]-2-(2- furyl)-2-oxo- acetamide | 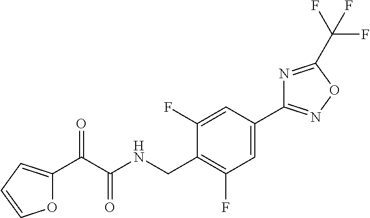 [SMILES: O=C(NCc1c(F)cc(-c2noc(C(F)(F)F)n2)cc1F)C(=O)c1ccco1] | 1.66 | 402.02 | B | |
| 1.89 | N-methoxy- 2-oxo-N-[[4-[5- (trifluoromethyl)- 1,2,4-oxadiazol-3- yl]phenyl]methyl] butanamide | 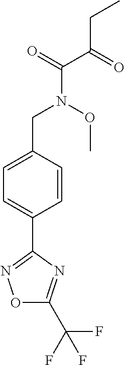 [SMILES: CCC(=O)C(=O)N(Cc1ccc(-c2noc(C(F)(F)F)n2)cc1)OC] | 1.73 | 358.07 | B | |
| 1.90 | 2-(2-furyl)-N- methoxy-2- oxo-N-[[4-[5- (trifluoromethyl)- 1,2,4- oxadiazol-3- yl]phenyl]methyl] acetamide | 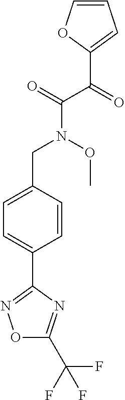 [SMILES: CON(Cc1ccc(-c2noc(C(F)(F)F)n2)cc1)C(=O)C(=O)c1ccco1] | 1.67 | 396.06 | B | |
| 1.91 | N-cyclopropyl- 2-oxo-N- [[4-[5- (trifluoromethyl)- 1,2,4-oxadiazol-3- yl]phenyl]methyl] propanamide | 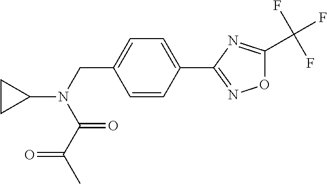 [SMILES: CC(=O)C(=O)N(Cc1ccc(-c2noc(C(F)(F)F)n2)cc1)C1CC1] | 1.07 | 354 | A | |
| 1.92 | N,2-dicyclopropyl- 2-oxo- N-[[4-[5- (trifluoromethyl)- 1,2,4- oxadiazol-3- yl]phenyl]methyl] acetamide | 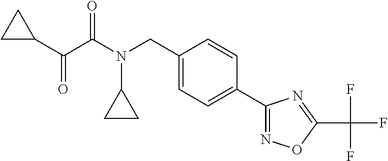 [SMILES: O=C(C(=O)N(Cc1ccc(-c2noc(C(F)(F)F)n2)cc1)C1CC1)C1CC1] | 1.12 | 380 | A | |
| 1.93 | 2-cyclopropyl- N-ethyl-2- oxo-N-[[4-[5- (trifluoromethyl)- 1,2,4- oxadiazol-3- yl]phenyl]methyl] acetamide | 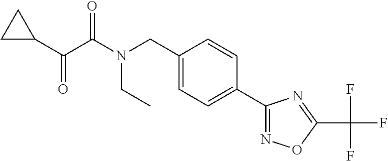 [SMILES: CCN(Cc1ccc(-c2noc(C(F)(F)F)n2)cc1)C(=O)C(=O)C1CC1] | 1.11 | 368 | A | |
| 1.94 | 2-cyclopropyl- N-methyl- 2-oxo-N-[[4-[5- (trifluoromethyl)- 1,2,4- oxadiazol-3- yl]phenyl]methyl] acetamide | 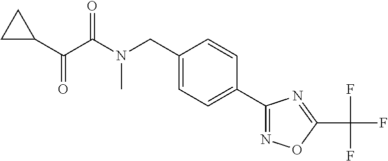 [SMILES: CN(Cc1ccc(-c2noc(C(F)(F)F)n2)cc1)C(=O)C(=O)C1CC1] | 1.06 | 354 | A | |
| 1.95 | N,3-dimethyl- 2-oxo-N- [[4-[5- (trifluoromethyl)- 1,2,4-oxadiazol-3- yl]phenyl]methyl] butanamide | 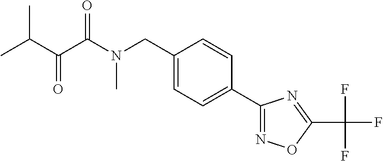 [SMILES: CC(C)C(=O)C(=O)N(C)Cc1ccc(-c2noc(C(F)(F)F)n2)cc1] | 1.12 | 356 | A | |
| 1.96 | N-ethyl-3- methyl-2-oxo- N-[[4-[5- (trifluoromethyl)- 1,2,4- oxadiazol-3- yl]phenyl]methyl] butanamide | 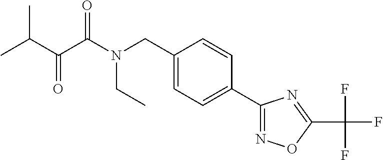 [SMILES: CCN(Cc1ccc(-c2noc(C(F)(F)F)n2)cc1)C(=O)C(=O)C(C)C] | 1.16 | 370 | A | |
| 1.97 | N-cyclopropyl- 3-methyl- 2-oxo-N-[[4-[5- (trifluoromethyl)- 1,2,4- oxadiazol-3- yl]phenyl]methyl] butanamide | 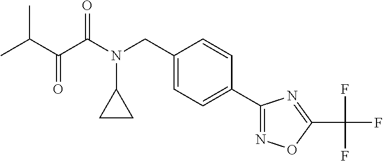 [SMILES: CC(C)C(=O)C(=O)N(Cc1ccc(-c2noc(C(F)(F)F)n2)cc1)C1CC1] | 1.17 | 382 | A | |
| 1.98 | N-methyl-2- oxo-N-[[4-[5- (trifluoromethyl)- 1,2,4- oxadiazol-3- yl]phenyl]methyl] butanamide | 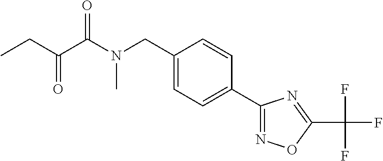 [SMILES: CCC(=O)C(=O)N(C)Cc1ccc(-c2noc(C(F)(F)F)n2)cc1] | 1.07 | 342 | A | |
| 1.99 | N-ethyl-2- oxo-N-[[4-[5- (trifluoromethyl)- 1,2,4- oxadiazol-3- yl]phenyl]methyl] butanamide | 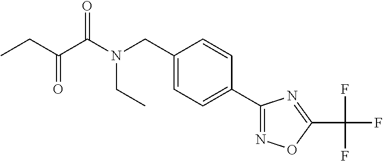 [SMILES: CCC(=O)C(=O)N(CC)Cc1ccc(-c2noc(C(F)(F)F)n2)cc1] | 1.11 | 356 | A | |
| 1.100 | N-cyclopropyl- 2-oxo-N- [[4-[5- (trifluoromethyl)- 1,2,4-oxadiazol-3- yl]phenyl]methyl] butanamide | 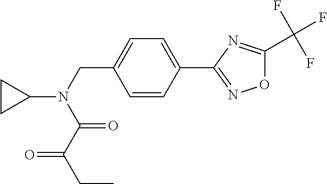 [SMILES: CCC(=O)C(=O)N(Cc1ccc(-c2noc(C(F)(F)F)n2)cc1)C1CC1] | 1.12 | 368 | A | |

TABLE T2
Melting point (mp) data and/or retention times (RT) for compounds according to Formula (I):

| | | | | Mass | | |
|---|---|---|---|---|---|---|
| | | | RT | charge | | MP |
| Entry | Compound name | Structure | (min) | [M + H] | Method | (° C.) |
| 2.1 | (2E)-2-methoxyimino-2- phenyl-N-[4-[5- (trifluoromethyl)-1,2,4- oxadiazol-3- yl]phenyl]acetamide | 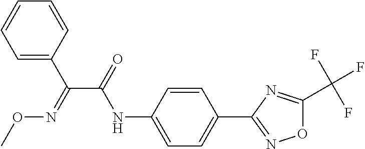 [SMILES: CO/N=C(/C(=O)Nc1ccc(-c2noc(C(F)(F)F)n2)cc1)c1ccccc1] | 1.93 | 391.24 | B | |
| 2.2 | 2-methoxyimino-2-phenyl- N-[[4-[5-(trifluoromethyl)- 1,2,4-oxadiazol-3- yl]phenyl]methyl]acetamide | 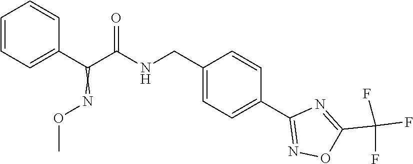 [SMILES: CON=C(C(=O)NCc1ccc(-c2noc(C(F)(F)F)n2)cc1)c1ccccc1] | 1.80 | 405.0 | B | |
| 2.3 | ethyl 2-oxo-2-[4-[5- (trifluoromethyl)-1,2,4- oxadiazol-3- yl]anilino]acetate | 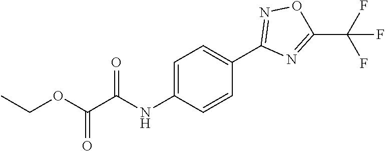 [SMILES: CCOC(=O)C(=O)Nc1ccc(-c2noc(C(F)(F)F)n2)cc1] | | | | 164-166 |
| 2.4 | N-methoxy-2- methoxyimino-2-phenyl-N- [[4-[5-(trifluoromethyl)- 1,2,4-oxadiazol-3- yl]phenyl]methyl]acetamide | 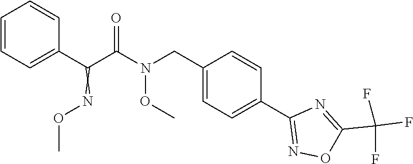 [SMILES: CON=C(C(=O)N(Cc1ccc(-c2noc(C(F)(F)F)n2)cc1)OC)c1ccccc1] | 1.95 | 435.2 | | |
| 2.5 | N-cyclopropyl-2- methoxyimino-2-phenyl-N- [[4-[5-(trifluoromethyl)- 1,2,4-oxadiazol-3- yl]phenyl]methyl]acetamide | 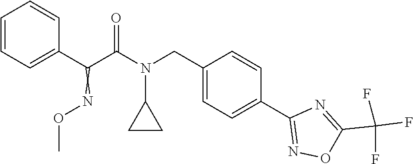 [SMILES: CON=C(C(=O)N(Cc1ccc(-c2noc(C(F)(F)F)n2)cc1)C1CC1)c1ccccc1] | 2.06 | 445.2 | B | |
| 2.6 | 2-methoxyimino-2-phenyl- N-prop-2-ynyl-N-[[4-[5- (trifluoromethyl)-1,2,4- oxadiazol-3- yl]phenyl]methyl]acetamide | 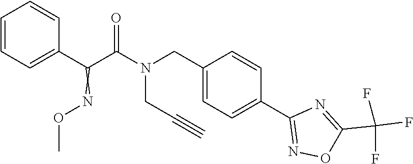 [SMILES: C#CCN(Cc1ccc(-c2noc(C(F)(F)F)n2)cc1)C(=O)C(=NOC)c1ccccc1] | 1.99 | 443.2 | B | |
| 2.7 | N-(cyclopropylmethyl)-2- methoxyimino-2-phenyl-N- [[4-[5-(trifluoromethyl)- 1,2,4-oxadiazol-3- yl]phenyl]methyl]acetamide | 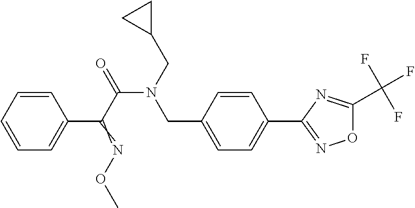 [SMILES: CON=C(C(=O)N(Cc1ccc(-c2noc(C(F)(F)F)n2)cc1)CC1CC1)c1ccccc1] | 2.12 | 459.2 | B | |
| 2.8 | 2-methoxyimino-2-phenyl- N-[6-[5-(trifluoromethyl)- 1,2,4-oxadiazol-3-yl]-3- pyridyl]acetamide | 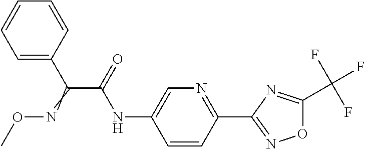 [SMILES: CON=C(C(=O)Nc1ccc(-c2noc(C(F)(F)F)n2)nc1)c1ccccc1] | 1.7 | 392.16 | B | |
| 2.9 | (2E)-2-methoxyimino-N- [[4-[5-(trifluoromethyl)- 1,2,4-oxadiazol-3- yl]phenyl]methyl] propanamide | 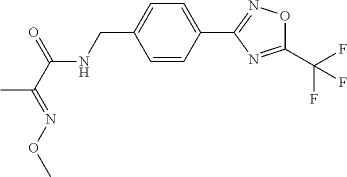 [SMILES: CO/N=C(\C)C(=O)NCc1ccc(-c2noc(C(F)(F)F)n2)cc1] | | | | 75.7-76.2 |
| 2.10 | (2E)-N-methoxy-2- methoxyimino-N-[[4-[5- (trifluoromethyl)-1,2,4- oxadiazol-3- yl]phenyl]methyl] propanamide | 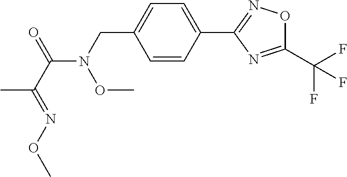 [SMILES: CO/N=C(\C)C(=O)N(Cc1ccc(-c2noc(C(F)(F)F)n2)cc1)OC] | 1.13 | 373 | A | |
| 2.11 | (2Z)-N-methoxy-2- methoxyimino-N-[[4-[5- (trifluoromethyl)-1,2,4- oxadiazol-3- yl]phenyl]methyl] propanamide | 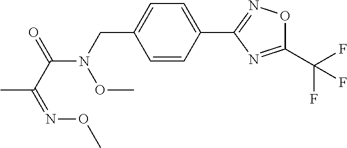 [SMILES: CO/N=C(/C)C(=O)N(Cc1ccc(-c2noc(C(F)(F)F)n2)cc1)OC] | 1.08 | 373 | A | |
| 2.12 | 2-ethoxyimino-N-methoxy- N-[[4-[5-(trifluoromethyl)- 1,2,4-oxadiazol-3- yl]phenyl]methyl] propanamide | 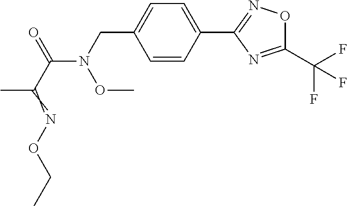 [SMILES: CCON=C(C)C(=O)N(Cc1ccc(-c2noc(C(F)(F)F)n2)cc1)OC] | 1.12 | 387 | A | |
| 2.13 | (1E)-N-methoxy-2-oxo-2- [[4-[5-(trifluoromethyl)- 1,2,4-oxadiazol-3- yl]phenyl]methylamino] acetimidoyl cyanide | 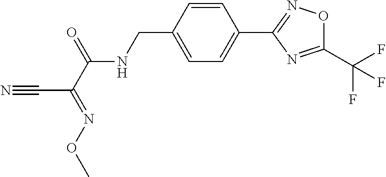 [SMILES: CO/N=C(\C#N)C(=O)NCc1ccc(-c2noc(C(F)(F)F)n2)cc1] | | | | 130-131 |
| 2.14 | (1E)-N-methoxy-2- [methoxy-[[4-[5- (trifluoromethyl)-1,2,4- oxadiazol-3- yl]phenyl]methyl]amino]- 2-oxo-acetimidoyl cyanide | 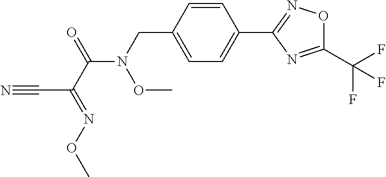 [SMILES: CO/N=C(\C#N)C(=O)N(Cc1ccc(-c2noc(C(F)(F)F)n2)cc1)OC] | | | | 67-68 |
| 2.15 | (1E)-2-[cyclopropyl-[[4-[5- (trifluoromethyl)-1,2,4- oxadiazol-3- yl]phenyl]methyl]amino]- N-methoxy-2-oxo- acetimidoyl cyanide | 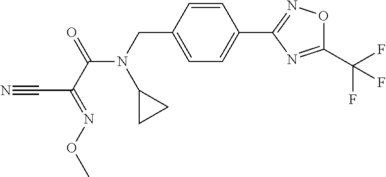 [SMILES: CO/N=C(\C#N)C(=O)N(Cc1ccc(-c2noc(C(F)(F)F)n2)cc1)C1CC1] | 1.14 | 394 | A | |

TABLE T3
Melting point (mp) data and/or retention times (RT) for compounds according to Formula (I):

| | | | | Mass | | |
|---|---|---|---|---|---|---|
| | | | RT | charge | | MP |
| Entry | Compound name | Structure | (min) | [M + H] | Method | (° C.) |
| 3.1 | 2-oxo-2-[4-[5- (trifluoromethyl)- 1,2,4-oxadiazol-3- yl]anilino]acetic acid | 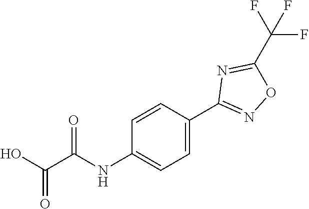 [SMILES: O=C(O)C(=O)Nc1ccc(-c2noc(C(F)(F)F)n2)cc1] | | | | 184- 186 |
| 3.2 | methyl 2- [methoxy-[[4[5- (trifluoromethyl)- 1,2,4-oxadiazol-3- yl]phenyl]methyl] amino]-2-oxo-acetate | 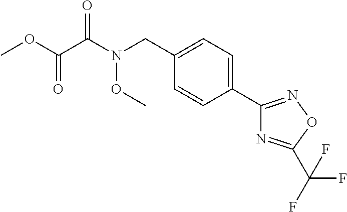 [SMILES: COC(=O)C(=O)N(Cc1ccc(-c2noc(C(F)(F)F)n2)cc1)OC] | 1.03 | 360 | A | |
| 3.3 | oxetan-3-yl 2- [methoxy-[[4- [5-(trifluoromethyl)- 1,2,4-oxadiazol-3- yl]phenyl]methyl] amino]-2-oxo-acetate | 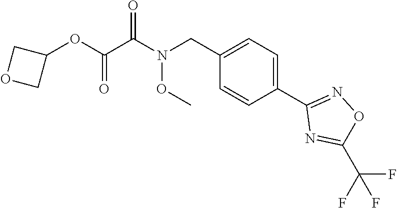 [SMILES: CON(Cc1ccc(-c2noc(C(F)(F)F)n2)cc1)C(=O)C(=O)OC1COC1] | 1.05 | 402 | A | |
| 3.4 | 2-ethoxyethyl 2- oxo-2-[[4-[5- (trifluoromethyl)- 1,2,4-oxadiazol-3- yl]phenyl] methylamino]acetate | 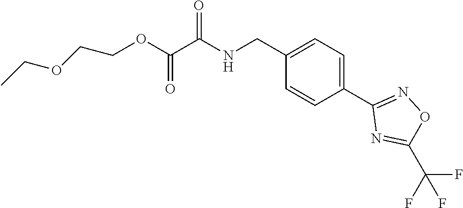 [SMILES: CCOCCOC(=O)C(=O)NCc1ccc(-c2noc(C(F)(F)F)n2)cc1] | | | | 112- 114 |
| 3.5 | (3-methylcyclopentyl) 2-oxo-2-[[4-[5- (trifluoromethyl)- 1,2,4-oxadiazol-3- yl]phenyl] methylamino]acetate | 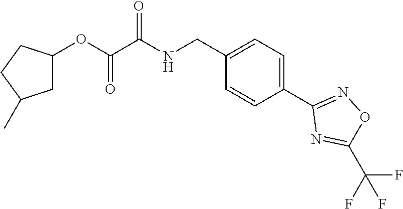 [SMILES: CC1CCC(OC(=O)C(=O)NCc2ccc(-c3noc(C(F)(F)F)n3)cc2)C1] | | | | 112- 116 |
| 3.6 | tetrahydrothiophen- 3-yl 2-oxo-2-[[4-[5- (trifluoromethyl)- 1,2,4-oxadiazol-3- yl]phenyl] methylamino]acetate | 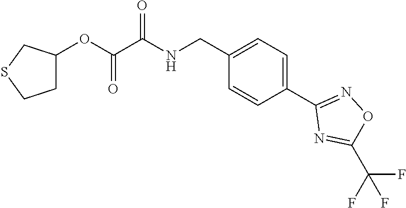 [SMILES: O=C(NCc1ccc(-c2noc(C(F)(F)F)n2)cc1)C(=O)OC1CCSC1] | | | | 102- 105 |
| 3.7 | 2-[methoxy-[[4-[5- (trifluoromethyl)- 1,2,4-oxadiazol-3- yl]phenyl]methyl] amino]-2-oxo- acetic acid | 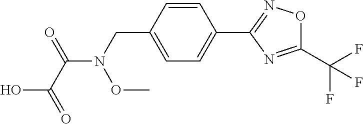 [SMILES: CON(Cc1ccc(-c2noc(C(F)(F)F)n2)cc1)C(=O)C(=O)O] | 0.75 | 346 | A | |
| 3.8 | 2-oxo-2-[[4-[5- (trifluoromethyl)- 1,2,4-oxadiazol-3- yl]phenyl] methylamino]acetic acid | 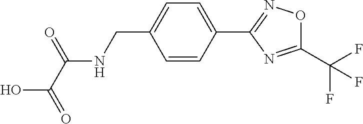 [SMILES: O=C(O)C(=O)NCc1ccc(-c2noc(C(F)(F)F)n2)cc1] | | | | 162- 173 |
| 3.9 | ethyl 2-oxo-2-[[4-[5- (trifluoromethyl)- 1,2,4-oxadiazol- 3-yl]phenyl] methylamino] acetate | 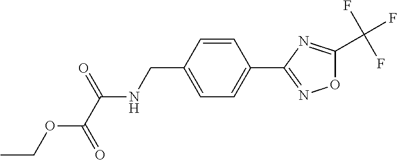 [SMILES: CCOC(=O)C(=O)NCc1ccc(-c2noc(C(F)(F)F)n2)cc1] | | | | 112- 116 |
| 3.10 | ethyl 2- [methoxy-[[4-[5- (trifluoromethyl)- 1,2,4-oxadiazol-3- yl]phenyl]methyl] amino]-2-oxo-acetate | 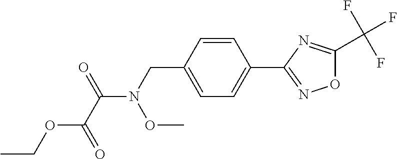 [SMILES: CCOC(=O)C(=O)N(Cc1ccc(-c2noc(C(F)(F)F)n2)cc1)OC] | 1.07 | 374 | A | |

TABLE T4
Melting point (mp) data and/or retention times (RT) for compounds according to Formula (I):

| | | | | Mass | | |
|---|---|---|---|---|---|---|
| | | | RT | charge | | MP |
| Entry | Compound name | Structure | (min) | [M + H] | Method | (° C.) |
| 4.1 | N′,N′-dimethyl-N-[4-[5- (trifluoromethyl)-1,2,4- oxadiazol-3- yl]phenyl]oxamide | 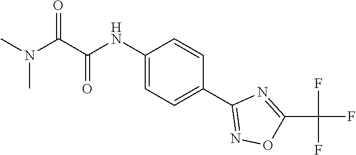 [SMILES: CN(C)C(=O)C(=O)Nc1ccc(-c2noc(C(F)(F)F)n2)cc1] | 1.43 | 329.22 | B | 127- 130 |
| 4.2 | N′,N′-dimethyl-N-[4-[5- (trifluoromethyl)-1,2,4- oxadiazol-3- yl]phenyl]methyl] oxamide | 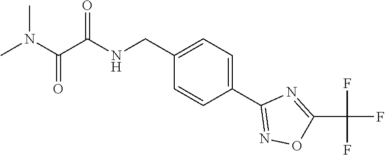 [SMILES: CN(C)C(=O)C(=O)NCc1ccc(-c2noc(C(F)(F)F)n2)cc1] | | | | 107- 112 |
| 4.3 | N-(cyclopropylmethyl)- N′-[4-[5- (trifluoromethyl)- 1,2,4-oxadiazol-3-yl] phenyl]oxamide | 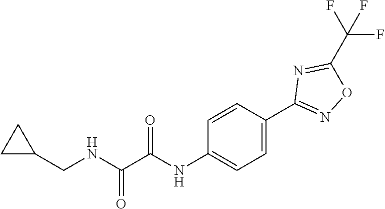 [SMILES: O=C(NCC1CC1)C(=O)Nc1ccc(-c2noc(C(F)(F)F)n2)cc1] | | | | 209- 212 |
| 4.4 | N-(2-methoxyethyl)- N′-[4-[5- (trifluoromethyl)- 1,2,4-oxadiazol-3-yl] phenyl]oxamide | 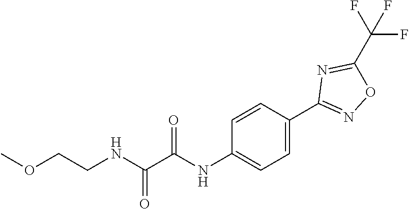 [SMILES: COCCNC(=O)C(=O)Nc1ccc(-c2noc(C(F)(F)F)n2)cc1] | | | | 204- 205 |
| 4.5 | N-prop-2-ynyl-N′-[4-[5- (trifluoromethyl)-1,2,4- oxadiazol-3- yl]phenyl]oxamide | 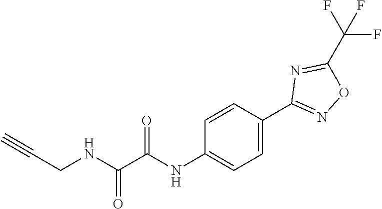 [SMILES: C#CCNC(=O)C(=O)Nc1ccc(-c2noc(C(F)(F)F)n2)cc1] | | | | 212- 214 |
| 4.6 | N′-cyclopropyl-N-[4-[5- (trifluoromethyl)-1,2,4- oxadiazol-3- yl]phenyl]oxamide | 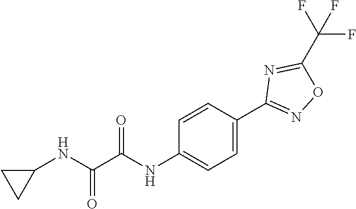 [SMILES: O=C(Nc1ccc(-c2noc(C(F)(F)F)n2)cc1)C(=O)NC1CC1] | | | | 238-241 |
| 4.7 | N-methyl-N′-[4-[5- (trifluoromethyl)-1,2,4- oxadiazol-3- yl]phenyl]oxamide | 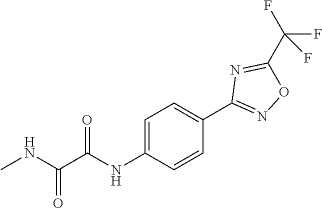 [SMILES: CNC(=O)C(=O)Nc1ccc(-c2noc(C(F)(F)F)n2)cc1] | | | | 231- 234 |
| 4.8 | 2-(2,2-dimethylhydrazino)- 2-oxo-N-[4-[5- (trifluoromethyl)-1,2,4- oxadiazol-3- yl]phenyl]acetamide | 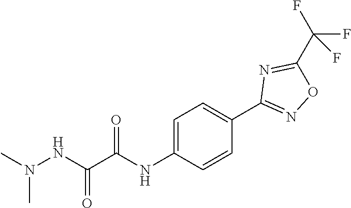 [SMILES: CN(C)NC(=O)C(=O)Nc1ccc(-c2noc(C(F)(F)F)n2)cc1] | | | | 232- 234 |
| 4.9 | N′-methoxy-N′-methyl-N- [4-[5-(trifluoromethyl)- 1,2,4-oxadiazol-3- yl]phenyl]oxamide | 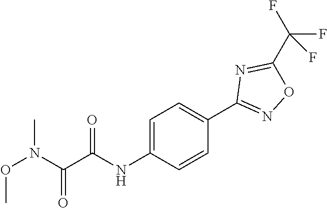 [SMILES: CON(C)C(=O)C(=O)Nc1ccc(-c2noc(C(F)(F)F)n2)cc1] | | | | 163- 165 |
| 4.10 | N′,N′-dimethyl-N-[5-[5- (trifluoromethyl)-1,2,4- oxadiazol- 3-yl]-2-pyridyl] oxamide | 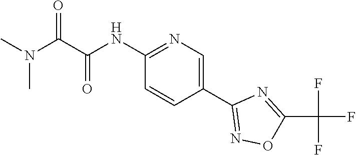 [SMILES: CN(C)C(=O)C(=O)Nc1ccc(-c2noc(C(F)(F)F)n2)cn1] | 1.34 | 329.9 | B | |
| 4.11 | N′-propoxy-N′-[[4-[5- (trifluoromethyl)-1,2,4- oxadiazol-3- yl]phenyl]methyl] oxamide | 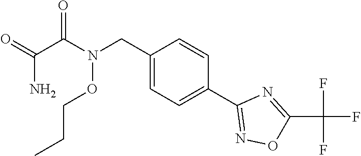 [SMILES: CCCON(Cc1ccc(-c2noc(C(F)(F)F)n2)cc1)C(=O)C(N)=O] | 1.5 | 373.3 | B | |
| 4.12 | N′-(2-methoxyethyl)-N′- [[4-[5-(trifluoromethyl)- 1,2,4-oxadiazol-3- yl]phenyl]methyl] oxamide | 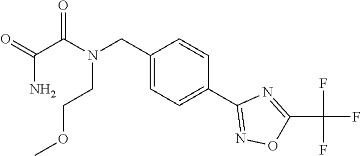 [SMILES: COCCN(Cc1ccc(-c2noc(C(F)(F)F)n2)cc1)C(=O)C(N)=O] | 1.35 | 373.3 | B | |
| 4.13 | N′-sec-butyl-N′-[[4-[5- (trifluoromethyl)-1,2,4- oxadiazol-3- yl]phenyl]methyl] oxamide | 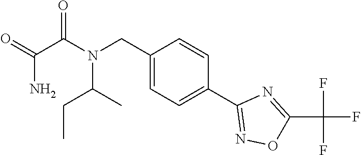 [SMILES: CCC(C)N(Cc1ccc(-c2noc(C(F)(F)F)n2)cc1)C(=O)C(N)=O] | 1.53 | 371.3 | B | |
| 4.14 | N′-(tetrahydrofuran-2- ylmethyl)-N′-[[4-[5- (trifluoromethyl)-1,2,4- oxadiazol-3- yl]phenyl]methyl] oxamide | 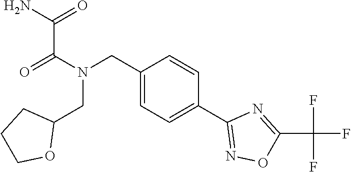 [SMILES: NC(=O)C(=O)N(Cc1ccc(-c2noc(C(F)(F)F)n2)cc1)CC1CCCO1] | 1.44 | 399.3 | B | |
| 4.15 | N′-benzyl-N′-(2- methoxyethyl)-N-[[4-[5- (trifluoromethyl)-1,2,4- oxadiazol-3- yl]phenyl]oxamide | 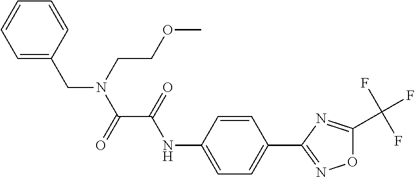 [SMILES: COCCN(Cc1ccccc1)C(=O)C(=O)Nc1ccc(-c2noc(C(F)(F)F)n2)cc1] | 1.84 | 447.16 | B | |
| 4.16 | N′-(1,3-benzodioxol-5- ylmethyl)-N′-(1,3- dioxolan-2-ylmethyl)- N-[4-[5- (trifluoromethyl)- 1,2,4-oxadiazol-3- yl]phenyl]oxamide | 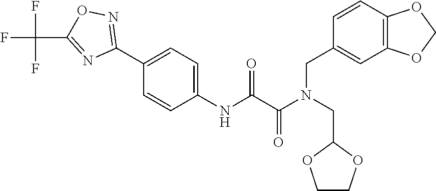 [SMILES: O=C(Nc1ccc(-c2noc(C(F)(F)F)n2)cc1)C(=O)N(Cc1ccc2c(c1)OCO2)CC1OCCO1] | 1.8 | 519.22 | B | |
| 4.17 | N′-(1,1-dimethyl-2- methylsulfonyl-ethyl)- N-[[4-[5- (trifluoromethyl)-1,2,4- oxadiazol-3- yl]phenyl]oxamide | 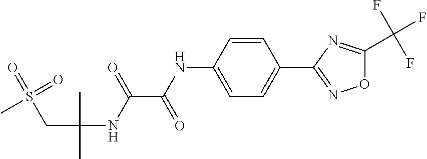 [SMILES: CC(C)(CS(C)(=O)=O)NC(=O)C(=O)Nc1ccc(-c2noc(C(F)(F)F)n2)cc1] | 1.54 | 433.12 | B | |
| 4.18 | N′,N′-bis(2-cyanoethyl)-N- [4-[5-(trifluoromethyl)- 1,2,4-oxadiazol-3- yl]phenyl]oxamide | 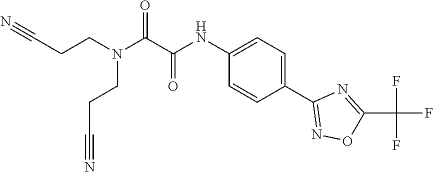 [SMILES: N#CCCN(CCC#N)C(=O)C(=O)Nc1ccc(-c2noc(C(F)(F)F)n2)cc1] | 1.48 | 405.12 | B | |
| 4.19 | N-(1,4-dioxan-2-ylmethyl)- N′-[4-[5-(trifluoromethyl)- 1,2,4-oxadiazol-3- yl]phenyl]oxamide | 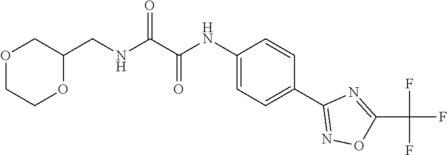 [SMILES: O=C(NCC1COCCO1)C(=O)Nc1ccc(-c2noc(C(F)(F)F)n2)cc1] | 1.48 | 399.1 | B | |
| 4.20 | N′-(1-cyclopropylethyl)-N- [4-[5-(trifluoromethyl)- 1,2,4-oxadiazol-3- yl]phenyl] oxamide | 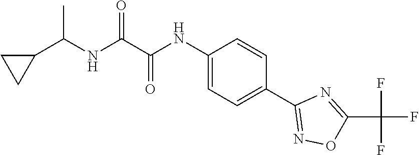 [SMILES: CC(NC(=O)C(=O)Nc1ccc(-c2noc(C(F)(F)F)n2)cc1)C1CC1] | 1.81 | 367.09 | B | |
| 4.21 | N′-(2,2-dimethoxyethyl)- N'-methyl-N-[4-[5- (trifluoromethyl)-1,2,4- oxadiazol-3- yl]phenyl] oxamide | 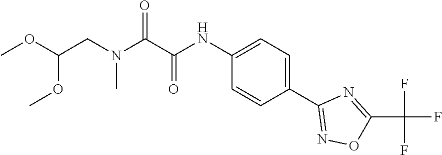 [SMILES: COC(CN(C)C(=O)C(=O)Nc1ccc(-c2noc(C(F)(F)F)n2)cc1)OC] | 1.56 | 401.1 | B | |
| 4.22 | tert-butyl 3-[methyl-[2- oxo-2-[4-[5- (trifluoromethyl)-1,2,4- oxadiazol-3- yl]anilino]acetyl]amino] pyrrolidine-1-carboxylate | 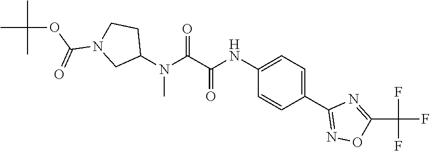 [SMILES: CN(C(=O)C(=O)Nc1ccc(-c2noc(C(F)(F)F)n2)cc1)C1CCN(C(=O)OC(C)(C)C)C1] | 1.78 | 482.25 | B | |
| 4.23 | N′-(2-furylmethyl)-N,N- dimethyl-N′-[[4-[5- (trifluoromethyl)- 1,2,4-oxadiazol-3- yl]phenyl]methyl] oxamide | 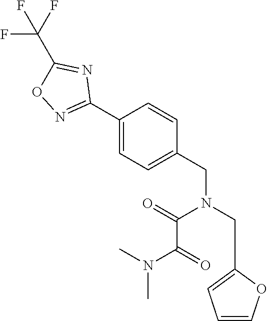 [SMILES: CN(C)C(=O)C(=O)N(Cc1ccc(-c2noc(C(F)(F)F)n2)cc1)Cc1ccco1] | 1.63 | 423.3 | B | |
| 4.24 | N′-(2-furylmethyl)- N′-[[4-[5- (trifluoromethyl)- 1,2,4-oxadiazol-3- yl]phenyl]methyl] oxamide | 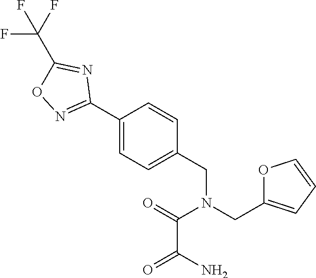 [SMILES: NC(=O)C(=O)N(Cc1ccc(-c2noc(C(F)(F)F)n2)cc1)Cc1ccco1] | 1.51 | 395.2 | B | |
| 4.25 | N′-allyl-N,N-dimethyl-N′- [[4-[5-(trifluoromethyl)- 1,2,4-oxadiazol-3- yl]phenyl]methyl] oxamide | 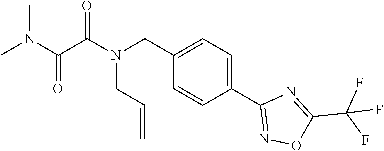 [SMILES: C=CCN(Cc1ccc(-c2noc(C(F)(F)F)n2)cc1)C(=O)C(=O)N(C)C] | 1.57 | 383.3 | B | |
| 4.26 | N′-allyl-N′-[[4-[5- (trifluoromethyl)-1,2,4- oxadiazol-3- yl]phenyl]methyl] oxamide | 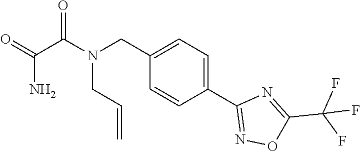 [SMILES: C=CCN(Cc1ccc(-c2noc(C(F)(F)F)n2)cc1)C(=O)C(N)=O] | 1.45 | 355.2 | B | |
| 4.27 | N′-ethyl-N,N-dimethyl-N′- [[4-[5-(trifluoromethyl)- 1,2,4-oxadiazol-3- yl]phenyl]methyl] oxamide | 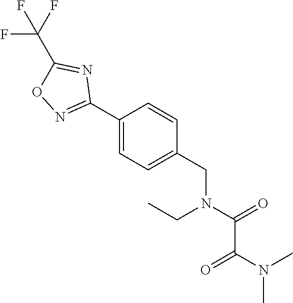 [SMILES: CCN(Cc1ccc(-c2noc(C(F)(F)F)n2)cc1)C(=O)C(=O)N(C)C] | 1.49 | 371.3 | B | |
| 4.28 | N′-ethyl-N′- [[4-[5- (trifluoromethyl)- 1,2,4-oxadiazol-3- yl]phenyl]methyl] oxamide | 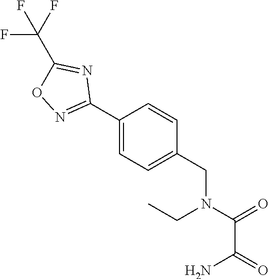 [SMILES: CCN(Cc1ccc(-c2noc(C(F)(F)F)n2)cc1)C(=O)C(N)=O] | 1.37 | 343.2 | B | |
| 4.29 | N,N,N′-trimethyl- N'-[[4-[5- (trifluoromethyl)- 1,2,4-oxadiazol-3- yl]phenyl]methyl] oxamide | 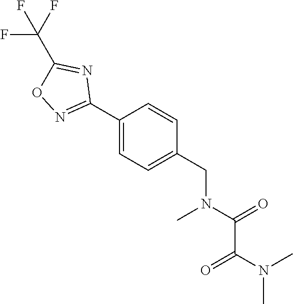 [SMILES: CN(C)C(=O)C(=O)N(C)Cc1ccc(-c2noc(C(F)(F)F)n2)cc1] | 1.38 | 357.3 | B | |
| 4.30 | N′-methyl-N′-[[4-[5- (trifluoromethyl)-1,2,4- oxadiazol-3- yl]phenyl]methyl] oxamide | 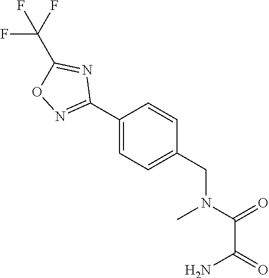 [SMILES: CN(Cc1ccc(-c2noc(C(F)(F)F)n2)cc1)C(=O)C(N)=O] | 1.27 | 329.2 | B | 120- 125 |
| 4.31 | N′-(3,3-dichloroallyloxy)- N,N-dimethyl-N′- [[4-[5-(trifluoromethyl)- 1,2,4-oxadiazol-3- yl]phenyl]methyl] oxamide | 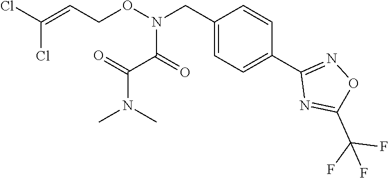 [SMILES: CN(C)C(=O)C(=O)N(Cc1ccc(-c2noc(C(F)(F)F)n2)cc1)OCC=C(Cl)Cl] | 1.8 | 467.2 | B | |
| 4.32 | N′-(3,3-dichloroallyloxy)- N′-[[4-[5-(trifluoromethyl)- 1,2,4-oxadiazol-3- yl]phenyl]methyl] oxamide | 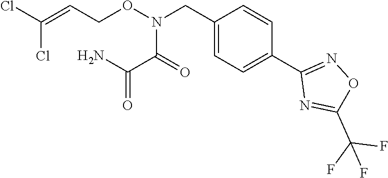 [SMILES: NC(=O)C(=O)N(Cc1ccc(-c2noc(C(F)(F)F)n2)cc1)OCC=C(Cl)Cl] | 1.65 | 439.2 | B | |
| 4.33 | N′-[(4- chlorophenyl)methoxy]- N,N-dimethyl-N′-[[4-[5- (trifluoromethyl)-1,2,4- oxadiazol-3- yl]phenyl]methyl] oxamide | 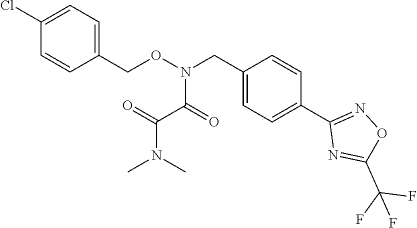 [SMILES: CN(C)C(=O)C(=O)N(Cc1ccc(-c2noc(C(F)(F)F)n2)cc1)OCc1ccc(Cl)cc1] | 1.87 | 483.3 | B | |
| 4.34 | N′-[(4- chlorophenyl)methoxy]-N′- [[4-[5-(trifluoromethyl)- 1,2,4-oxadiazol-3- yl]phenyl]methyl] oxamide | 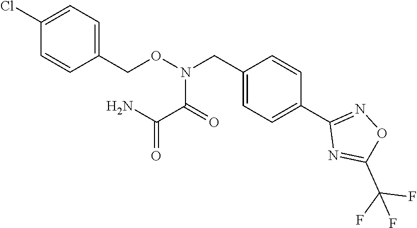 [SMILES: NC(=O)C(=O)N(Cc1ccc(-c2noc(C(F)(F)F)n2)cc1)OCc1ccc(Cl)cc1] | 1.73 | 455.5 | B | |
| 4.35 | N′-methoxy-N-methyl-N′- [[4-[5-(trifluoromethyl)- 1,2,4-oxadiazol-3- yl]phenyl]methyl] oxamide | 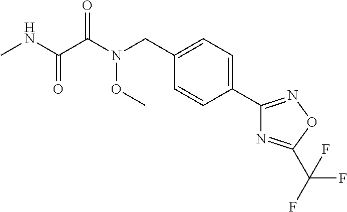 [SMILES: CNC(=O)C(=O)N(Cc1ccc(-c2noc(C(F)(F)F)n2)cc1)OC] | | | | 87.7- 88.8 |
| 4.36 | N-methyl-N′-[2-methyl-4- [5-(trifluoromethyl)-1,2,4- oxadiazol-3- yl]phenyl]methyl] | 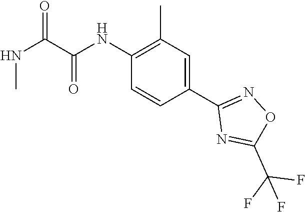 [SMILES: CNC(=O)C(=O)Nc1ccc(-c2noc(C(F)(F)F)n2)cc1C] | | | | 172- 184 |
| 4.37 | N′,N′-dimethyl-N-[2- methyl-4-[5- (trifluoromethyl)-1,2,4- oxadiazol-3- yl]phenyl]oxamide | 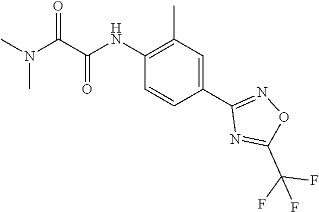 [SMILES: Cc1cc(-c2noc(C(F)(F)F)n2)ccc1NC(=O)C(=O)N(C)C] | | | | 79- 89 |
| 4.38 | N-(2-methoxyethyl)-N′-[2- methyl-4-[5- (trifluoromethyl)-1,2,4- oxadiazol-3- yl]phenyl] oxamide | 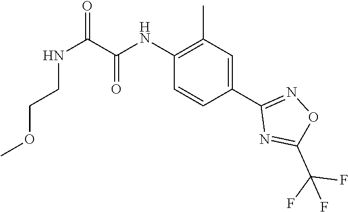 [SMILES: COCCNC(=O)C(=O)Nc1ccc(-c2noc(C(F)(F)F)n2)cc1C] | | | | 126- 130 |
| 4.39 | N′-methoxy- N,N-dimethyl- N′-[[4-[5- (trifluoromethyl)- 1,2,4-oxadiazol-3- yl]phenyl]methyl] oxamide | 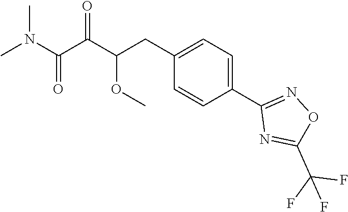 [SMILES: COC(Cc1ccc(-c2noc(C(F)(F)F)n2)cc1)C(=O)C(=O)N(C)C] | 1.46 | 373.08 | B | |
| 4.40 | 2-oxo-2-(1-piperidyl)- N-[[4-[5- (trifluoromethyl)- 1,2,4-oxadiazol-3- yl]phenyl]methyl] acetamide | 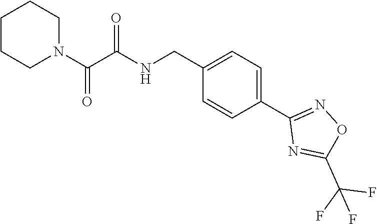 [SMILES: O=C(NCc1ccc(-c2noc(C(F)(F)F)n2)cc1)C(=O)N1CCCCC1] | | | | 104- 108 |
| 4.41 | N′-methyl-N′- tetrahydrofuran- 3-yl-N-[[4-[5- (trifluoromethyl)- 1,2,4- oxadiazol-3- yl]phenyl]methyl] oxamide | 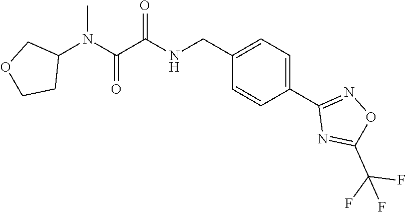 [SMILES: CN(C(=O)C(=O)NCc1ccc(-c2noc(C(F)(F)F)n2)cc1)C1CCOC1] | 0.92 | 399 | A | |
| 4.42 | N′-propoxy-N-[[4-[5- (trifluoromethyl)-1,2,4- oxadiazol-3- yl]phenyl]methyl] oxamide | 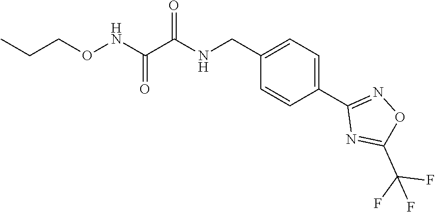 [SMILES: CCCONC(=O)C(=O)NCc1ccc(-c2noc(C(F)(F)F)n2)cc1] | 0.99 | 373 | A | |
| 4.43 | N′-ethoxy-N′- methyl-N-[[4-[5- (trifluoromethyl)- 1,2,4- oxadiazol-3- yl]phenyl]methyl] oxamide | 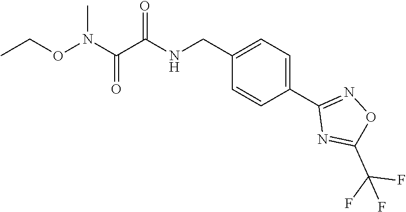 [SMILES: CCON(C)C(=O)C(=O)NCc1ccc(-c2noc(C(F)(F)F)n2)cc1] | 0.97 | 373 | A | |
| 4.44 | N′-(1-methoxy-4- piperidyl)-N′- methyl-N-[[4-[5- (trifluoromethyl)- 1,2,4-oxadiazol-3- yl]phenyl]methyl] oxamide | 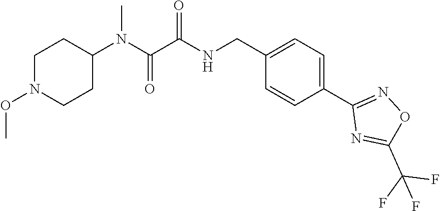 [SMILES: CON1CCC(N(C)C(=O)C(=O)NCc2ccc(-c3noc(C(F)(F)F)n3)cc2)CC1] | | | | 123- 125 |
| 4.45 | N′-(2,2-dimethylpropyl)- N-[[4-[5- (trifluoromethyl)- 1,2,4-oxadiazol-3- yl]phenyl]methyl] oxamide | 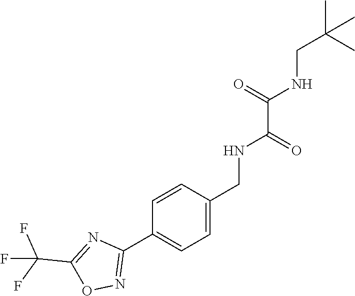 [SMILES: CC(C)(C)CNC(=O)C(=O)NCc1ccc(-c2noc(C(F)(F)F)n2)cc1] | 1.76 | 385.13 | B | |
| 4.46 | N′-isopropyl-N- [[4-[5- (trifluoromethyl)- 1,2,4- oxadiazol-3- yl]phenyl]methyl] oxamide | 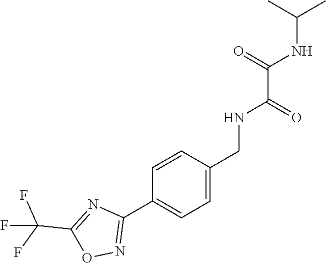 [SMILES: CC(C)NC(=O)C(=O)NCc1ccc(-c2noc(C(F)(F)F)n2)cc1] | 1.55 | 357.09 | B | |
| 4.47 | N-methyl-N′- [[4-[5- (trifluoromethyl)- 1,2,4-oxadiazol- 3-yl]phenyl]methyl] oxamide | 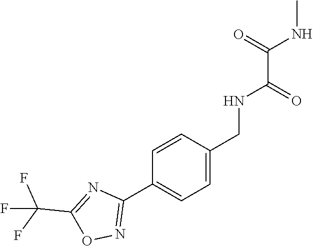 [SMILES: CNC(=O)C(=O)NCc1ccc(-c2noc(C(F)(F)F)n2)cc1] | 1.33 | 329.2 | B | |
| 4.48 | N′-methoxy-N′- methyl-N- [[4-[5- (trifluoromethyl)- 1,2,4-oxadiazol-3- yl]phenyl]methyl] oxamide | 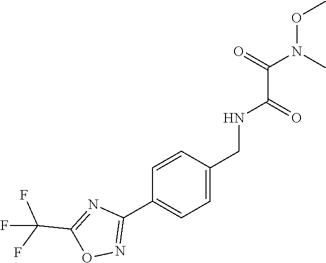 [SMILES: CON(C)C(=O)C(=O)NCc1ccc(-c2noc(C(F)(F)F)n2)cc1] | 1.36 | 359.07 | B | |
| 4.49 | N-ethyl-N′-[[4-[5- (trifluoromethyl)-1,2,4- oxadiazol-3- yl]phenyl]methyl] oxamide | 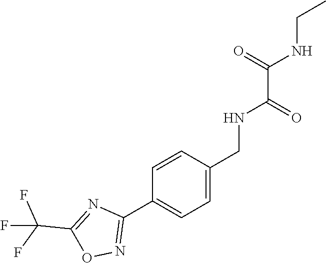 [SMILES: CCNC(=O)C(=O)NCc1ccc(-c2noc(C(F)(F)F)n2)cc1] | 1.44 | 343.09 | B | |
| 4.50 | N′-isobutyl-N- [[4-[5- (trifluoromethyl)- 1,2,4- oxadiazol-3- yl]phenyl]methyl] oxamide | 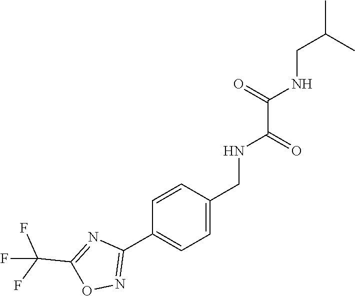 [SMILES: CC(C)CNC(=O)C(=O)NCc1ccc(-c2noc(C(F)(F)F)n2)cc1] | 1.66 | 371.11 | B | |
| 4.51 | N′-cyclopropyl- N-[[4-[5- (trifluoromethyl)- 1,2,4- oxadiazol-3- yl]phenyl]methyl] oxamide | 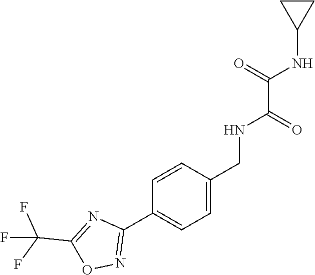 [SMILES: O=C(NCc1ccc(-c2noc(C(F)(F)F)n2)cc1)C(=O)NC1CC1] | 1.46 | 355.09 | B | |
| 4.52 | N′,N′-diethyl- N-[[4-[5- (trifluoromethyl)- 1,2,4- oxadiazol-3- yl]phenyl]methyl] oxamide | 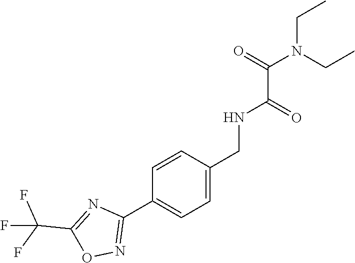 [SMILES: CCN(CC)C(=O)C(=O)NCc1ccc(-c2noc(C(F)(F)F)n2)cc1] | 1.52 | 371.1 | B | |
| 4.53 | N-benzyl-N′-[[4-[5- (trifluoromethyl)-1,2,4- oxadiazol-3- yl]phenyl]methyl] oxamide | 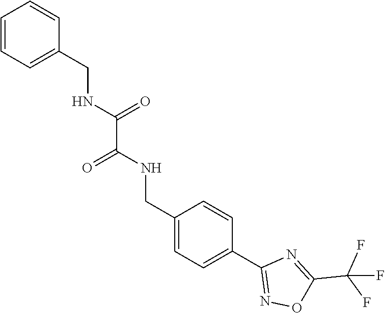 [SMILES: O=C(NCc1ccccc1)C(=O)NCc1ccc(-c2noc(C(F)(F)F)n2)cc1] | 1.68 | 404.9 | B | |
| 4.54 | N′-ethyl-N′-methyl- N-[[4-[5- (trifluoromethyl)- 1,2,4-oxadiazol-3- yl]phenyl]methyl] oxamide | 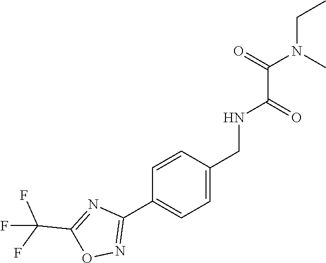 [SMILES: CCN(C)C(=O)C(=O)NCc1ccc(-c2noc(C(F)(F)F)n2)cc1] | 1.42 | 357.09 | B | |
| 4.55 | N′-ethoxy-N-[[4-[5- (trifluoromethyl)-1,2,4- oxadiazol-3- yl]phenyl]methyl] oxamide | 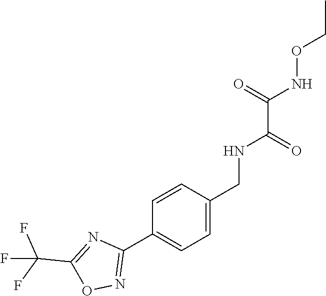 [SMILES: CCONC(=O)C(=O)NCc1ccc(-c2noc(C(F)(F)F)n2)cc1] | 1.38 | 359.06 | B | |
| 4.56 | N′-tert-butyl-N- [[4-[5- (trifluoromethyl)- 1,2,4- oxadiazol-3- yl]phenyl]methyl] oxamide | 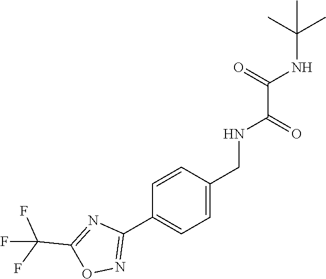 [SMILES: CC(C)(C)NC(=O)C(=O)NCc1ccc(-c2noc(C(F)(F)F)n2)cc1] | 1.72 | 371.11 | B | |
| 4.57 | N-allyl-N′-[[4-[5- (trifluoromethyl)- 1,2,4- oxadiazol-3- yl]phenyl]methyl] oxamide | 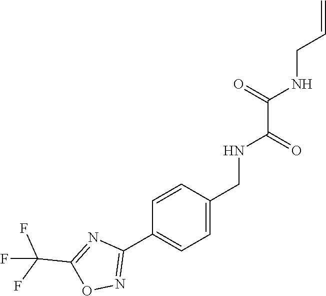 [SMILES: C=CCNC(=O)C(=O)NCc1ccc(-c2noc(C(F)(F)F)n2)cc1] | 1.5 | 355.08 | B | |
| 4.58 | N′-methoxy- N-[[4-[5- (trifluoromethyl)- 1,2,4- oxadiazol-3- yl]phenyl]methyl] oxamide | 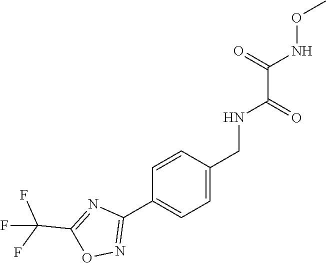 [SMILES: CONC(=O)C(=O)NCc1ccc(-c2noc(C(F)(F)F)n2)cc1] | 1.3 | 345.07 | B | |
| 4.59 | 2-morpholino- 2-oxo-N-[[4-[5- (trifluoromethyl)- 1,2,4- oxadiazol-3- yl]phenyl]methyl] acetamide | 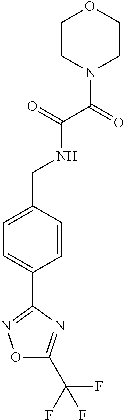 [SMILES: O=C(NCc1ccc(-c2noc(C(F)(F)F)n2)cc1)C(=O)N1CCOCC1] | 1.33 | 385.09 | B | |
| 4.60 | N′-(2-furylmethyl)- N-[[4-[5- (trifluoromethyl)- 1,2,4- oxadiazol-3- yl]phenyl]methyl] oxamide | 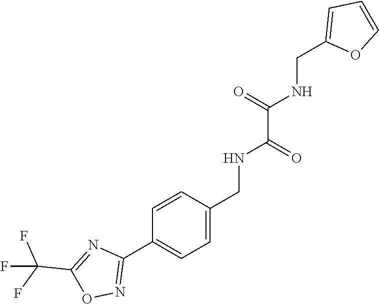 [SMILES: O=C(NCc1ccc(-c2noc(C(F)(F)F)n2)cc1)C(=O)NCc1ccco1] | 1.57 | Mass not detected | B | |
| 4.61 | N′-methoxy-N- sec-butyl- N′-[[4-[5- (trifluoromethyl)- 1,2,4-oxadiazol-3- yl]phenyl]methyl] oxamide | 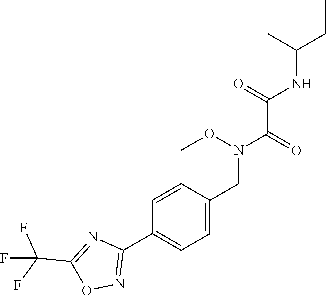 [SMILES: CCC(C)NC(=O)C(=O)N(Cc1ccc(-c2noc(C(F)(F)F)n2)cc1)OC] | 1.66 | 401.12 | B | |
| 4.62 | N-(2,2- dimethylpropyl)-N′- methoxy-N′- [[4-[5- (trifluoromethyl)- 1,2,4- oxadiazol-3- yl]phenyl]methyl] oxamide | 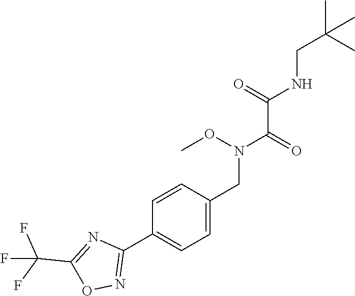 [SMILES: CON(Cc1ccc(-c2noc(C(F)(F)F)n2)cc1)C(=O)C(=O)NCC(C)(C)C] | 1.77 | 415.14 | B | |
| 4.63 | N-cyclohexyl- N′-methoxy- N′-[[4-[5- (trifluoromethyl)- 1,2,4-oxadiazol-3- yl]phenyl]methyl] oxamide | 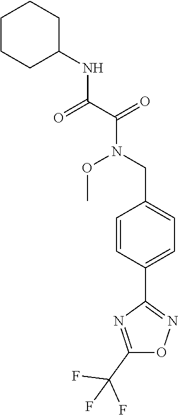 [SMILES: CON(Cc1ccc(-c2noc(C(F)(F)F)n2)cc1)C(=O)C(=O)NC1CCCCC1] | 1.79 | 427.14 | B | |
| 4.64 | N-isopropyl-N′- methoxy- N′-[[4-[5- (trifluoromethyl)- 1,2,4-oxadiazol- 3-yl]phenyl]methyl] oxamide | 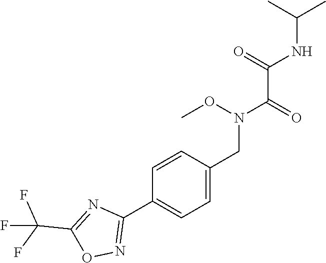 [SMILES: CON(Cc1ccc(-c2noc(C(F)(F)F)n2)cc1)C(=O)C(=O)NC(C)C] | 1.57 | 387.12 | B | |
| 4.65 | N,N′-dimethoxy- N-methyl- N′-[[4-[5- (trifluoromethyl)- 1,2,4-oxadiazol-3- yl]phenyl]methyl] oxamide | 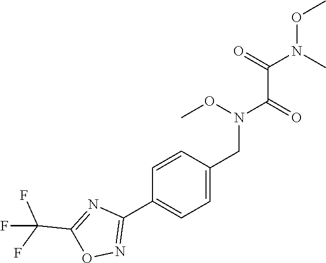 [SMILES: CON(C)C(=O)C(=O)N(Cc1ccc(-c2noc(C(F)(F)F)n2)cc1)OC] | 1.54 | 389.07 | B | |
| 4.66 | N-ethyl-N′- methoxy-N′-[[4-[5- (trifluoromethyl)- 1,2,4- oxadiazol-3- yl]phenyl]methyl] oxamide | 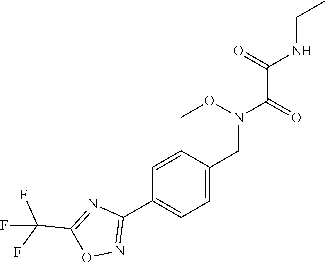 [SMILES: CCNC(=O)C(=O)N(Cc1ccc(-c2noc(C(F)(F)F)n2)cc1)OC] | 1.48 | 373.08 | B | |
| 4.67 | N-isobutyl-N′- methoxy-N′- [[4-[5- (trifluoromethyl)- 1,2,4-oxadiazol-3- yl]phenyl]methyl] oxamide | 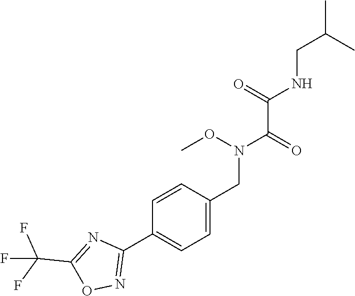 [SMILES: CON(Cc1ccc(-c2noc(C(F)(F)F)n2)cc1)C(=O)C(=O)NCC(C)C] | 1.68 | 401.12 | B | |
| 4.68 | N-cyclopropyl-N′- methoxy-N′-[[4-[5- (trifluoromethyl)-1,2,4- oxadiazol-3- yl]phenyl]methyl] oxamide | 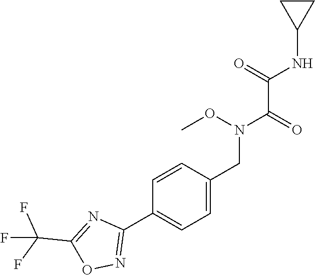 [SMILES: CON(Cc1ccc(-c2noc(C(F)(F)F)n2)cc1)C(=O)C(=O)NC1CC1] | 1.49 | 385.1 | B | |
| 4.69 | N,N-diethyl- N′-methoxy- N′-[[4-[5- (trifluoromethyl)- 1,2,4-oxadiazol-3- yl]phenyl]methyl] oxamide | 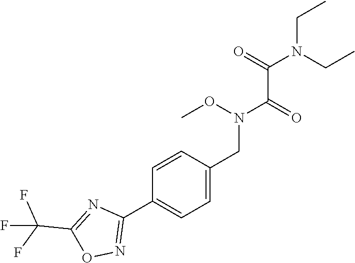 [SMILES: CCN(CC)C(=O)C(=O)N(Cc1ccc(-c2noc(C(F)(F)F)n2)cc1)OC] | 1.65 | 401.13 | B | |
| 4.70 | N′-methoxy-N- phenyl-N′- [[4-[5- (trifluoromethyl)- 1,2,4-oxadiazol-3- yl]phenyl]methyl] oxamide | 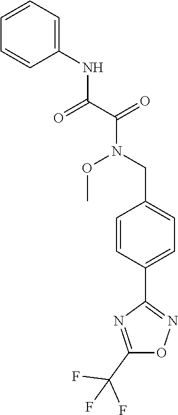 [SMILES: CON(Cc1ccc(-c2noc(C(F)(F)F)n2)cc1)C(=O)C(=O)Nc1ccccc1] | 1.75 | 421.09 | B | |
| 4.71 | N-benzyl-N′- methoxy-N'- [[4-[5- (trifluoromethyl)- 1,2,4-oxadiazol-3- yl]phenyl]methyl] oxamide | 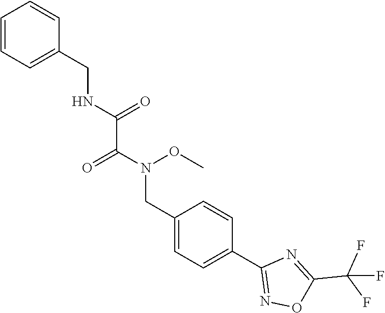 [SMILES: CON(Cc1ccc(-c2noc(C(F)(F)F)n2)cc1)C(=O)C(=O)NCc1ccccc1] | 1.71 | 435.11 | B | |
| 4.72 | N-cyclobutyl-N′- methoxy- N′-[[4-[5- (trifluoromethyl)- 1,2,4-oxadiazol-3- yl]phenyl]methyl] oxamide | 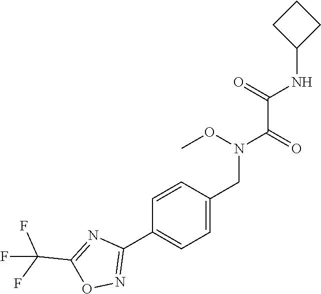 [SMILES: CON(Cc1ccc(-c2noc(C(F)(F)F)n2)cc1)C(=O)C(=O)NC1CCC1] | 1.62 | 399.12 | B | |
| 4.73 | N-ethyl-N′- methoxy-N- methyl-N′-[[4-[5- (trifluoromethyl)- 1,2,4- oxadiazol-3- yl]phenyl]methyl] oxamide | 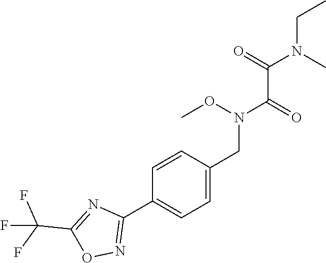 [SMILES: CCN(C)C(=O)C(=O)N(Cc1ccc(-c2noc(C(F)(F)F)n2)cc1)OC] | 1.55 | 387.11 | B | |
| 4.74 | N-ethoxy-N′- methoxy-N′- [[4-[5- (trifluoromethyl)- 1,2,4-oxadiazol-3- yl]phenyl]methyl] oxamide | 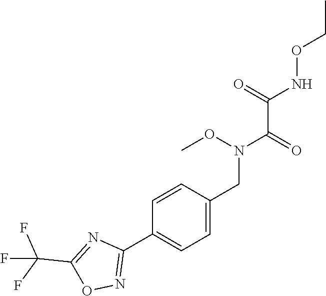 [SMILES: CCONC(=O)C(=O)N(Cc1ccc(-c2noc(C(F)(F)F)n2)cc1)OC] | 1.46 | 389.09 | B | |
| 4.75 | N-tert-butyl-N′- methoxy- N′-[[4-[5- (trifluoromethyl)- 1,2,4-oxadiazol-3- yl]phenyl]methyl] oxamide | 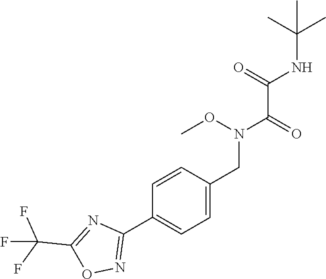 [SMILES: CON(Cc1ccc(-c2noc(C(F)(F)F)n2)cc1)C(=O)C(=O)NC(C)(C)C] | 1.7 | 401.13 | B | |
| 4.76 | N-allyl-N′-methoxy- N′-[[4-[5- (trifluoromethyl)- 1,2,4- oxadiazol-3- yl]phenyl]methyl] oxamide | 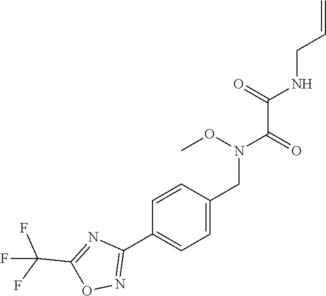 [SMILES: C=CCNC(=O)C(=O)N(Cc1ccc(-c2noc(C(F)(F)F)n2)cc1)OC] | 1.54 | 385.11 | B | |
| 4.77 | N′-methoxy-N- propyl-N′- [[4-[5- (trifluoromethyl)- 1,2,4-oxadiazol-3- yl]phenyl]methyl] oxamide | 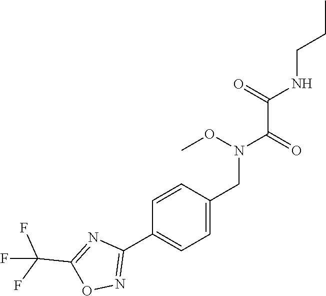 [SMILES: CCCNC(=O)C(=O)N(Cc1ccc(-c2noc(C(F)(F)F)n2)cc1)OC] | 1.58 | 387.12 | B | |
| 4.78 | N,N′-dimethoxy- N′-[[4-[5- (trifluoromethyl)- 1,2,4- oxadiazol-3- yl]phenyl]methyl] oxamide | 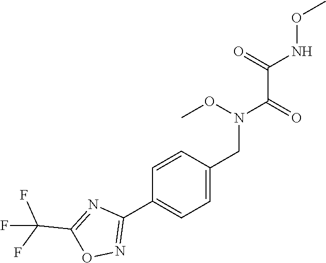 [SMILES: CONC(=O)C(=O)N(Cc1ccc(-c2noc(C(F)(F)F)n2)cc1)OC] | 1.38 | 375.06 | B | |
| 4.79 | N-cyclopentyl- N′-methoxy-N′- [[4-[5- (trifluoromethyl)- 1,2,4- oxadiazol-3- yl]phenyl]methyl] oxamide | 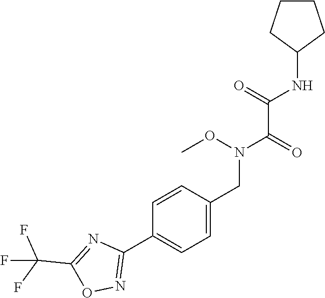 [SMILES: CON(Cc1ccc(-c2noc(C(F)(F)F)n2)cc1)C(=O)C(=O)NC1CCCC1] | 1.69 | 413.13 | B | |
| 4.80 | N-methoxy-2- morpholino- 2-oxo-N-[[4-[5- (trifluoromethyl)- 1,2,4- oxadiazol-3- yl]phenyl]methyl] acetamide | 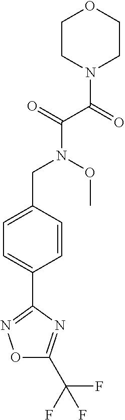 [SMILES: CON(Cc1ccc(-c2noc(C(F)(F)F)n2)cc1)C(=O)C(=O)N1CCOCC1] | 1.46 | 415.11 | B | |
| 4.81 | N-(2-furylmethyl)-N′- methoxy-N′-[[4-[5- (trifluoromethyl)- 1,2,4- oxadiazol-3- yl]phenyl]methyl] oxamide | 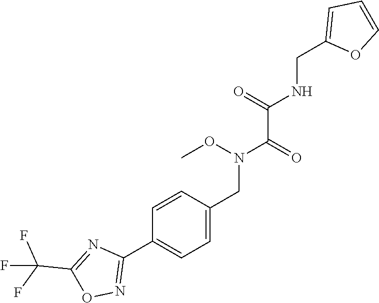 [SMILES: CON(Cc1ccc(-c2noc(C(F)(F)F)n2)cc1)C(=O)C(=O)NCc1ccco1] | 1.60 | 425.08 | B | |
| 4.82 | N′-[[2-fluoro-4-[5- (trifluoromethyl)- 1,2,4-oxadiazol-3- yl]phenyl]methyl] oxarnide | 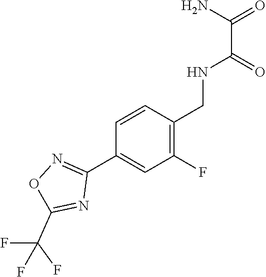 [SMILES: NC(=O)C(=O)NCc1ccc(-c2noc(C(F)(F)F)n2)cc1F] | 1.31 | 333.03 | B | |
| 4.83 | N′-[[3-fluoro-4-[5- (trifluoromethyl)- 1,2,4-oxadiazol-3- yl]phenyl]methyl] oxamide | 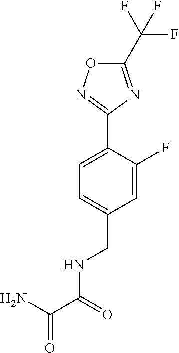 [SMILES: NC(=O)C(=O)NCc1ccc(-c2noc(C(F)(F)F)n2)c(F)c1] | 1.30 | 333.01 | B | |
| 4.84 | N′-[[2,3-difluoro- 4-[5- (trifluoromethyl)- 1,2,4-oxadiazol-3- yl]phenyl]methyl] oxamide | 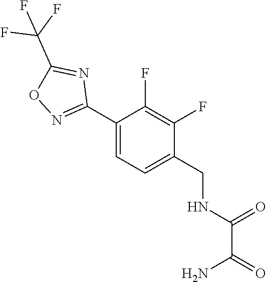 [SMILES: NC(=O)C(=O)NCc1ccc(-c2noc(C(F)(F)F)n2)c(F)c1F] | 1.29 | 351.04 | B | |
| 4.85 | N′-[2-[4-[5- (trifluoromethyl)- 1,2,4- oxadiazol-3- yl]phenyl]ethyl] oxamide | 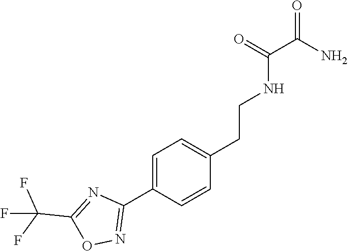 [SMILES: NC(=O)C(=O)NCCc1ccc(-c2noc(C(F)(F)F)n2)cc1] | 1.32 | 329.1 | B | |
| 4.86 | N′-ethoxy-N′-[[4-[5- (trifluoromethyl)-1,2,4- oxadiazol-3- yl]phenyl]methyl] oxarnide | 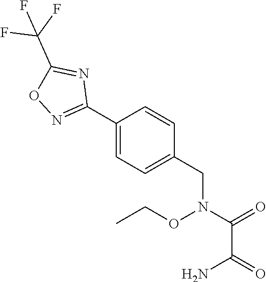 [SMILES: CCON(Cc1ccc(-c2noc(C(F)(F)F)n2)cc1)C(=O)C(N)=O] | 1.37 | 359.12 | B | |
| 4.87 | N′-butoxy-N′-[[4-[5- (trifluoromethyl)-1,2,4- oxadiazol-3- yl]phenyl]methyl] oxamide | 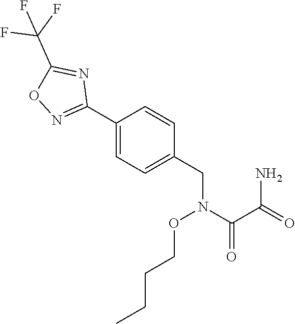 [SMILES: CCCCON(Cc1ccc(-c2noc(C(F)(F)F)n2)cc1)C(=O)C(N)=O] | 1.59 | 387.18 | B | |
| 4.88 | N′-propyl-N′-[[4-[5- (trifluoromethyl)-1,2,4- oxadiazol-3- yl]phenyl]methyl] oxamide | 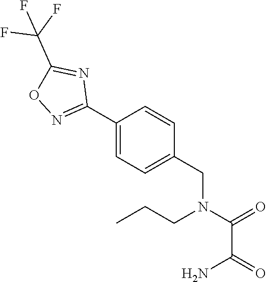 [SMILES: CCCN(Cc1ccc(-c2noc(C(F)(F)F)n2)cc1)C(=O)C(N)=O] | 1.45 | 357.16 | B | |
| 4.89 | N′-isopropyl-N′-[[4-[5- (trifluoromethyl)- 1,2,4-oxadiazol-3- yl]phenyl]methyl] oxamide | 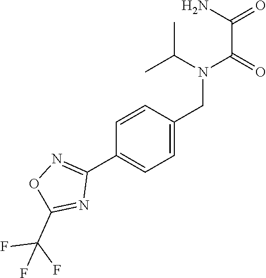 [SMILES: CC(C)N(Cc1ccc(-c2noc(C(F)(F)F)n2)cc1)C(=O)C(N)=O] | 1.41 | 357.16 | B | |
| 4.90 | N′-(oxetan-3-yl)-N′- [[4-[5- (trifluoromethyl)-1,2,4- oxadiazol-3- yl]phenyl]methyl] oxamide | 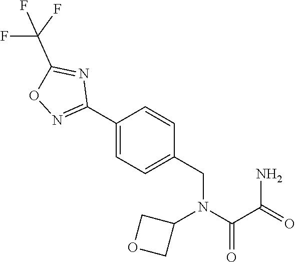 [SMILES: NC(=O)C(=O)N(Cc1ccc(-c2noc(C(F)(F)F)n2)cc1)C1COC1] | 1.19 | 371.14 | B | |
| 4.91 | N′-hydroxy-N′-[[4-[5- (trifluoromethyl)- 1,2,4- oxadiazol-3- yl]phenyl]methyl] oxamide | 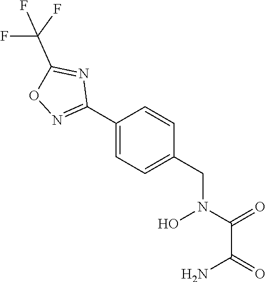 [SMILES: NC(=O)C(=O)N(O)Cc1ccc(-c2noc(C(F)(F)F)n2)cc1] | 1.15 | 331.09 | B | |
| 4.92 | N-[[2-fluoro-4-[5- (trifluoromethyl)- 1,2,4- oxadiazol-3- yl]phenyl]methyl]- N',N'- dimethyl-oxamide | 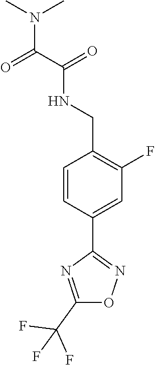 [SMILES: CN(C)C(=O)C(=O)NCc1ccc(-c2noc(C(F)(F)F)n2)cc1F] | 1.37 | 361.07 | B | |
| 4.93 | N-[[3-fluoro-4-[5- (trifluoromethyl)- 1,2,4- oxadiazol-3- yl]phenyl]methyl]- N',N'- dimethyl-oxamide | 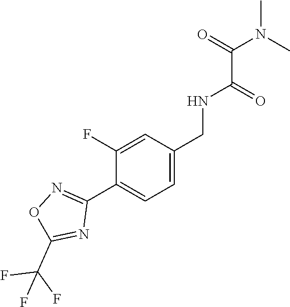 [SMILES: CN(C)C(=O)C(=O)NCc1ccc(-c2noc(C(F)(F)F)n2)c(F)c1] | 1.38 | 361.07 | B | |
| 4.94 | N-[[2,3-difluoro- 4-[5- (trifluoromethyl)- 1,2,4- oxadiazol-3- yl]phenyl]methyl]- N',N'- dimethyl-oxamide | 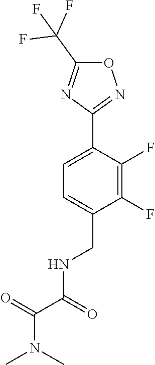 [SMILES: CN(C)C(=O)C(=O)NCc1ccc(-c2noc(C(F)(F)F)n2)c(F)c1F] | 1.37 | 379.05 | B | |
| 4.95 | N′-[[2-fluoro-4-[5- (trifluoromethyl)-1,2,4- oxadiazol-3- yl]phenyl] methyl]-N′- methoxy-N,N-dimethyl- oxamide | 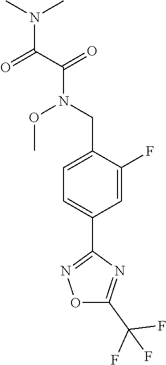 [SMILES: CON(Cc1ccc(-c2noc(C(F)(F)F)n2)cc1F)C(=O)C(=O)N(C)C] | 1.51 | 391.07 | B | |
| 4.96 | N-[[2-chloro-4-[5- (trifluoromethyl)- 1,2,4- oxadiazol-3- yl]phenyl]methyl]- N′,N′- dimethyl-oxamide | 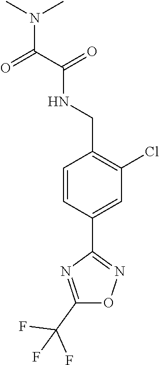 [SMILES: CN(C)C(=O)C(=O)NCc1ccc(-c2noc(C(F)(F)F)n2)cc1Cl] | 1.48 | 377.02 | B | |
| 4.97 | N′,N′-dimethyl-N-[[2- methyl-4-[5- (trifluoromethyl)- 1,2,4- oxadiazol-3- yl]phenyl]methyl] oxamide | 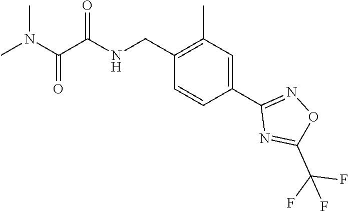 [SMILES: Cc1cc(-c2noc(C(F)(F)F)n2)ccc1CNC(=O)C(=O)N(C)C] | 1.43 | 357.09 | B | |
| 4.98 | N-[[2,6-difluoro-4-[5- (trifluoromethyl)-1,2,4- oxadiazol-3- yl]phenyl]methyl]-N′,N′- dimethyl-oxamide | 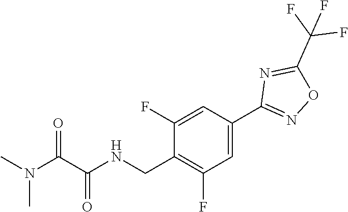 [SMILES: CN(C)C(=O)C(=O)NCc1c(F)cc(-c2noc(C(F)(F)F)n2)cc1F] | 1.39 | 379.04 | B | |
| 4.99 | N,N′-dimethyl-N′- [[4-[5- (trifluoromethyl)-1,2,4- oxadiazol-3- yl]phenyl]methyl] oxamide | 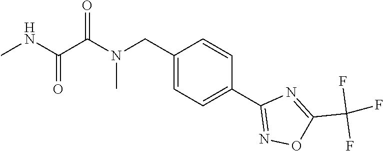 [SMILES: CNC(=O)C(=O)N(C)Cc1ccc(-c2noc(C(F)(F)F)n2)cc1] | 0.92 | 343 | A | |
| 4.100 | N′-cyclopropyl- N-methyl- N′-[[4-[5- (trifluoromethyl)- 1,2,4-oxadiazol-3- yl]phenyl]methyl] oxamide | 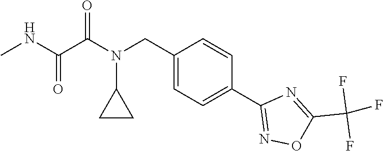 [SMILES: CNC(=O)C(=O)N(Cc1ccc(-c2noc(C(F)(F)F)n2)cc1)C1CC1] | 0.96 | 369 | A | |
| 4.101 | N′-ethyl-N- methyl-N′-[[4- [5-(trifluoromethyl)- 1,2,4- oxadiazol-3- yl]phenyl]methyl] oxamide | 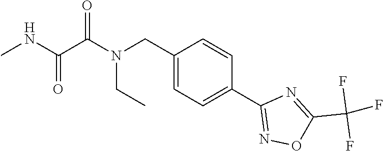 [SMILES: CCN(Cc1ccc(-c2noc(C(F)(F)F)n2)cc1)C(=O)C(=O)NC] | 0.97 | 357 | A | |
| 4.102 | N-methoxy-N′- methyl-N′- [[4-[5- (trifluoromethyl)- 1,2,4-oxadiazol-3- yl]phenyl]methyl] oxamide | 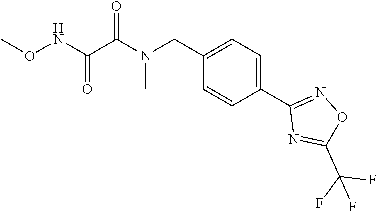 [SMILES: CONC(=O)C(=O)N(C)Cc1ccc(-c2noc(C(F)(F)F)n2)cc1] | 0.92 | 359 | A | |
| 4.103 | N′-cyclopropyl-N- methoxy-N′-[[4-[5- (trifluoromethyl)- 1,2,4-oxadiazol-3- yl]phenyl]methyl] oxamide | 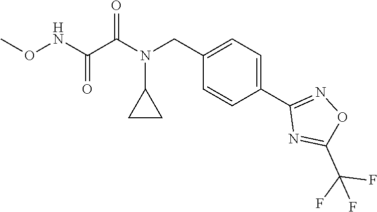 [SMILES: CONC(=O)C(=O)N(Cc1ccc(-c2noc(C(F)(F)F)n2)cc1)C1CC1] | 0.97 | 385 | A | |
| 4.104 | N′-methoxy-N,N′- dimethyl-N-[[4-[5-- (trifluoromethyl)- 1,2,4-oxadiazol-3- yl]phenyl]methyl] oxamide | 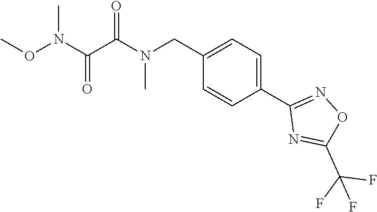 [SMILES: CON(C)C(=O)C(=O)N(C)Cc1ccc(-c2noc(C(F)(F)F)n2)cc1] | 0.99 | 373 | A | |
| 4.105 | N′-cyclopropyl-N- methoxy-N-methyl- N′-[[4-[5- (trifluoromethyl)- 1,2,4-oxadiazol-3- yl]phenyl]methyl] oxamide | 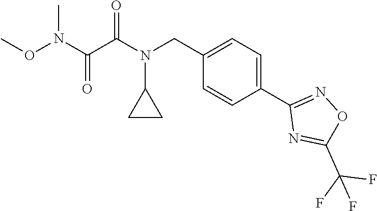 [SMILES: CON(C)C(=O)C(=O)N(Cc1ccc(-c2noc(C(F)(F)F)n2)cc1)C1CC1] | 1.05 | 399 | A | |
| 4.106 | N′-ethyl-N-methoxy-N- methyl-N′-[[4-[5- (trifluoromethyl)- 1,2,4-oxadiazol-3- yl]phenyl]methyl] oxamide | 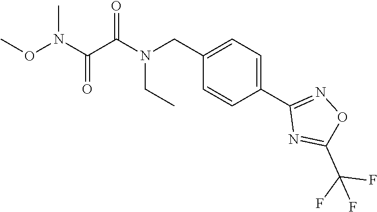 [SMILES: CCN(Cc1ccc(-c2noc(C(F)(F)F)n2)cc1)C(=O)C(=O)N(C)OC] | 1.03 | 387 | A | |
| 4.107 | N'-ethyl-N-methoxy- N′-N′-[[4-[5- (trifluoromethyl)- 1,2,4-oxadiazol-3- yl]phenyl]methyl] oxamide | 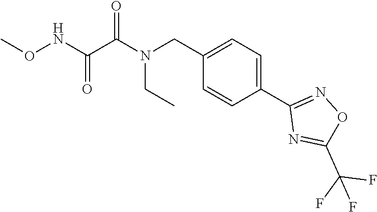 [SMILES: CCN(Cc1ccc(-c2noc(C(F)(F)F)n2)cc1)C(=O)C(=O)NOC] | 0.96 | 373 | A | |
| 4.108 | 2-(2,2-dimethylhydrazino)- | | 0.91 | 358 | A | |
| | 2-oxo-N-[[4-[5- | | | | | |
| | (trifluoromethyl)-1,2,4- | | | | | |
| | oxadiazol-3- | | | | | |
| | yl]phenyl]methyl] | | | | | |
| | acetamide | | | | | |
| 4.109 | 2-(2,2-dimethylhydrazino)- N-methyl-2-oxo-N-[[4-[5- (trifluoromethyl)-1,2,4- oxadiazol-3- yl]phenyl]methyl] acetamid | 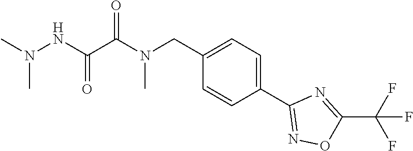 [SMILES: CN(C)NC(=O)C(=O)N(C)Cc1ccc(-c2noc(C(F)(F)F)n2)cc1] | 0.90 | 372 | A | |
| 4.110 | 2-(2,2-dimethylhydrazino)- N-ethyl-2-oxo-N-[[4-[5- (trifluoromethyl)-1,2,4- oxadiazol-3- yl]phenyl]methyl] acetamide | 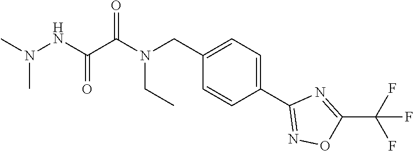 [SMILES: CCN(Cc1ccc(-c2noc(C(F)(F)F)n2)cc1)C(=O)C(=O)NN(C)C] | 0.95 | 386 | A | |
| 4.111 | N-cyclopropyl-2-(2,2- dimethylhydrazino)- 2-oxo- N-[[4-[5- 1,2,4-oxadiazol-3- yl]phenyl]methyl] acetamide | 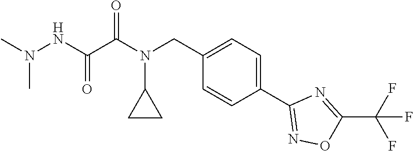 [SMILES: CN(C)NC(=O)C(=O)N(Cc1ccc(-c2noc(C(F)(F)F)n2)cc1)C1CC1] | 0.96 | 398 | A | |

### BIOLOGICAL EXAMPLES

### General Examples of Leaf Disk Tests in Well Plates:

Leaf disks or leaf segments of various plant species are cut from plants grown in a greenhouse. The cut leaf disks or segments are placed in multiwell plates (24-well format) onto water agar. The leaf disks are sprayed with a test solution before (preventative) or after (curative) inoculation. Compounds to be tested are prepared as DMSO solutions (max. 10 mg/mL) which are diluted to the appropriate concentration with 0.025% Tween20 just before spraying. The inoculated leaf disks or segments are incubated under defined conditions (temperature, relative humidity, light, etc.) according to the respective test system. A single evaluation of disease level is carried out 3 to 14 days after inoculation, depending on the pathosystem. Percent disease control relative to the untreated check leaf disks or segments is then calculated.

### General Examples of Liquid Culture Tests in Well Plates:

Mycelia fragments or conidia suspensions of a fungus prepared either freshly from liquid cultures of the fungus or from cryogenic storage, are directly mixed into nutrient broth. DMSO solutions of the test compound (max. 10 mg/ml) are diluted with 0.025% Tween20 by a factor of 50 and 10 μL of this solution is pipetted into a microtiter plate (96-well format). The nutrient broth containing the fungal spores/mycelia fragments is then added to give an end concentration of the tested compound. The test plates are incubated in the dark at 24° C. and 96% relative humidity. The inhibition of fungal growth is determined photometrically after 2 to 7 days, depending on the pathosystem, and percent antifungal activity relative to the untreated check is calculated.

### Example 1: Fungicidal Activity Against Puccinia recondita f. Sp. Tritici/Wheat/Leaf Disc Preventative (Brown Rust)

Wheat leaf segments cv. Kanzler were placed on agar in multiwell plates (24-well format) and sprayed with the formulated test compound diluted in water. The leaf disks were inoculated with a spore suspension of the fungus 1 day after application. The inoculated leaf segments were incubated at 19° C. and 75% relative humidity (rh) under a light regime of 12 hours light/12 hours darkness in a climate cabinet and the activity of a compound was assessed as percent disease control compared to untreated when an appropriate level of disease damage appears in untreated check leaf segments (7 to 9 days after application).

The following compounds at 200 ppm in the applied formulation give at least 80% disease control in this test when compared to untreated control leaf disks under the same conditions, which show extensive disease development.

Compounds (from Table T1) 1.3, 1.4, 1.5, 1.8, 1.9, 1.10, 1.12, 1.13, 1.14, 1.15, 1.17, 1.18, 1.19, 1.20, 1.21, 1.22, 1.25, 1.26, 1.27, 1.29, 1.31, 1.32, 1.33, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.61, 1.62, 1.63, 1.64, 1.65, 1.66, 1.68, 1.69, 1.70, 1.71, 1.72, 1.73, 1.74, 1.75, 1.76, 1.77, 1.78, 1.79, 1.80, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, and 1.100.

Compounds (from Table T2) 2.2, 2.3, 2.4, 2.9, 2.10, 2.11, 2.12, 2.14, and 2.15.

Compounds (from Table T3) 3.2, 3.3, 3.4, 3.5, 3.6, 3.9, and 3.10.

Compounds (from Table T4) 4.1, 4.2, 4.4, 4.7, 4.9, 4.10, 4.11, 4.12, 4.13, 4.14, 4.15, 4.17, 4.18, 4.19, 4.21, 4.22, 4.23, 4.24, 4.25, 4.26, 4.27, 4.28, 4.29, 4.30, 4.31, 4.32, 4.34, 4.35, 4.36, 4.37, 4.38, 4.39, 4.40, 4.41, 4.42, 4.43, 4.44, 4.46, 4.47, 4.48, 4.49, 4.50, 4.52, 4.53, 4.54, 4.55, 4.56, 4.57, 4.58, 4.59, 4.60, 4.61, 4.62, 4.63, 4.64, 4.65, 4.66, 4.67, 4.68, 4.69, 4.70, 4.71, 4.72, 4.73, 4.74, 4.75, 4.76, 4.77, 4.78, 4.79, 4.80, 4.81, 4.86, 4.87, 4.88, 4.89, 4.90, 4.91, 4.92, 4.93, 4.94, 4.95, 4.96, 4.97, 4.98, 4.99, 4.100, 4.101, 4.102, 4.103, 4.104, 4.105, 4.106, and 4.107.

### Example 2: Fungicidal Activity Against Puccinia recondita f. Sp. Tritici/Wheat/Leaf Disc Curative (Brown Rust)

Wheat leaf segments cv. Kanzler are placed on agar in multiwell plates (24-well format). The leaf segments are then inoculated with a spore suspension of the fungus. Plates were stored in darkness at 19° C. and 75% relative humidity. The formulated test compound diluted in water was applied 1 day after inoculation. The leaf segments were incubated at 19° C. and 75% relative humidity under a light regime of 12 hours light/12 hours darkness in a climate cabinet and the activity of a compound was assessed as percent disease control compared to untreated when an appropriate level of disease damage appears in untreated check leaf segments (6 to 8 days after application).

The following compounds at 200 ppm in the applied formulation give at least 80% disease control in this test when compared to untreated control leaf disks under the same conditions, which show extensive disease development.

Compounds (from Table T1) 1.3, 1.4, 1.5, 1.9, 1.10, 1.12, 1.14, 1.15, 1.16, 1.17, 1.18, 1.19, 1.20, 1.21, 1.25, 1.26, 1.27, 1.29, 1.31, 1.32, 1.33, 1.35, 1.36, 1.37, 1.38, 1.39, 1.40, 1.41, 1.43, 1.44, 1.45, 1.47, 1.49, 1.50, 1.51, 1.52, 1.53, 1.55, 1.58, 1.64, 1.70, 1.71, 1.72, 1.74, 1.75, 1.76, 1.77, 1.78, 1.79, 1.80, 1.82, 1.83, 1.85, 1.87, 1.88, 1.89, 1.90, 1.91, 1.92, 1.93, 1.94, 1.95, 1.96, 1.97, 1.98, 1.99, and 1.100.

Compounds (from Table T2) 2.9, 2.10, 2.11, 2.12, and 2.14.

Compounds (from Table T3) 3.2, 3.3, 3.7, and 3.10.

Compounds (from Table T4) 4.1, 4.2, 4.4, 4.7, 4.10, 4.12, 4.13, 4.14, 4.17, 4.21, 4.23, 4.25, 4.26, 4.27, 4.28, 4.29, 4.30, 4.35, 4.37, 4.38, 4.39, 4.40, 4.41, 4.42, 4.44, 4.46, 4.47, 4.48, 4.52, 4.54, 4.55, 4.56, 4.58, 4.59, 4.61, 4.62, 4.64, 4.65, 4.66, 4.67, 4.68, 4.69, 4.72, 4.73, 4.74, 4.75, 4.76, 4.77, 4.78, 4.80, 4.86, 4.88, 4.89, 4.90, 4.91, 4.92, 4.93, 4.94, 4.95, 4.96, 4.97, 4.98, 4.99, 4.100, 4.101, 4.102, 4.103, 4.104, 4.105, 4.106, and 4.107.

### Example 3: Fungicidal Activity Against Phakopsora pachyrhizi/Soybean/Leaf Disc Preventative (Asian Soybean Rust)

Soybean leaf disks are placed on water agar in multiwell plates (24-well format) and sprayed with the formulated test compound diluted in water. One day after application leaf discs are inoculated by spraying a spore suspension on the lower leaf surface. After an incubation period in a climate cabinet of 24-36 hours in darkness at 20° C. and 75% relative humidity leaf disc are kept at 20° C. with 12 hours light/day and 75% relative humidity. The activity of a compound is assessed as percent disease control compared to untreated when an appropriate level of disease damage appears in untreated check leaf disks (12 to 14 days after application).

The following compounds at 200 ppm in the applied formulation give at least 80% disease control in this test when compared to untreated control leaf disks under the same conditions, which show extensive disease development.

Compounds (from Table T1) 1.3, 1.4, 1.5, 1.6, 1.10, 1.12, 1.16, 1.17, 1.18, 1.19, 1.20, 1.21, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.61, 1.62, 1.63, 1.64, 1.65, 1.66, 1.67, 1.68, 1.70, 1.71, 1.72, 1.73, 1.74, 1.75, 1.76, 1.77, 1.78, 1.79, 1.80, 1.81, 1.82, 1.83, 1.84, 1.86, 1.87, 1.88, 1.89, 1.90, 1.91, 1.92, 1.93, 1.94, 1.95, 1.96, 1.97, 1.98, 1.99, and 1.100.

Compounds (from Table T2) 2.9, 2.10, 2.11, and 2.12.

Compounds (from Table T3) 3.8 and 3.9.

Compounds (from Table T4) 4.1, 4.2, 4.3, 4.4, 4.5, 4.6, 4.7, 4.8, 4.9, 4.19, 4.25, 4.26, 4.27, 4.28, 4.29, 4.30, 4.35, 4.36, 4.37, 4.38, 4.39, 4.40, 4.41, 4.44, 4.47, 4.52, 4.54, 4.55, 4.56, 4.58, 4.59, 4.65, 4.69, 4.73, 4.76, 4.84, 4.85, 4.86, 4.87, 4.88, 4.89, 4.90, 4.91, 4.92, 4.93, 4.94, 4.95, 4.96, 4.97, 4.98, 4.101, 4.104, 4.105, and 4.106.

### Example 4: Fungicidal Activity Against Glomerella lacenarium (Colletotrichum lacenarium) Liquid Culture/Cucumber/Preventative (Anthracnose)

Conidia of the fungus from cryogenic storage are directly mixed into nutrient broth (PDB—potato dextrose broth). After placing a (DMSO) solution of test compound into a microtiter plate (96-well format), the nutrient broth containing the fungal spores is added. The test plates are incubated at 24° C. and the inhibition of growth is measured photometrically 3 to 4 days after application.

The following compounds at 20 ppm in the applied formulation give at least 80% disease control in this test when compared to untreated control under the same conditions, which show extensive disease development.

Compounds (from Table T1) 1.2, 1.3, 1.4, 1.5, 1.6, 1.7, 1.8, 1.9, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.62, 1.63, 1.64, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.87, 1.88, 1.89, 1.90, 1.91, 1.92, 1.93, 1.94, 1.95, 1.96, 1.97, 1.98, 1.99, and 1.100.

Compounds (from Table T2) 2.2, 2.4, 2.9, 2.10, 2.11, 2.12, 2.13, 2.14, and 2.15.

Compounds (from Table T3) 3.2, 3.3, 3.4, 3.5, 3.6, 3.7, 3.9, and 3.10.

Compounds (from Table T4) 4.1, 4.2, 4.9, 4.11, 4.12, 4.13, 4.14, 4.15, 4.16, 4.21, 4.23, 4.24, 4.25, 4.26, 4.27, 4.28, 4.29, 4.30, 4.31, 4.32, 4.33, 4.34, 4.35, 4.36, 4.37, 4.38, 4.39, 4.40, 4.41, 4.42, 4.43, 4.44, 4.45, 4.46, 4.47, 4.48, 4.49, 4.52, 4.54, 4.55, 4.56, 4.57, 4.58, 4.59, 4.60, 4.61, 4.62, 4.63, 4.64, 4.65, 4.66, 4.67, 4.68, 4.69, 4.70, 4.71, 4.72, 4.73, 4.74, 4.75, 4.76, 4.77, 4.78, 4.79, 4.80, 4.81, 4.82, 4.83, 4.84, 4.85, 4.86, 4.87, 4.88, 4.89, 4.90, 4.91, 4.92, 4.93, 4.94, 4.95, 4.96, 4.97, 4.98, 4.99, 4.100, 4.101, 4.102, 4.103, 4.104, 4.105, 4.106, and 4.107.

### Example 5: Fungicidal Activity Against Uromyces viciae-Fabael Field Bean/Leaf Disc Preventative (Faba-Bean Rust)

Field bean leaf discs are placed on water agar in multiwell plates (96-well format) and 10 μL of the formulated test compound diluted in acetone and a spreader pipetted onto the leaf disc. Two hours after application leaf discs are inoculated by spraying a spore suspension on the lower leaf surface. The leaf discs are incubated in a climate cabinet at 22° C. with 18 hour days and 70% relative humidity. The activity of a compound is assessed as percent disease control compared to untreated when an appropriate level of disease damage appears in untreated check leaf disks (12 days after application).

The following compounds at 100 ppm in the applied formulation give at least 80% disease control in this test when compared to untreated control leaf discs under the same conditions, which show extensive disease development.

Compounds (from Table T1) 1.1 and 1.2.

Compound (from Table T2) 2.1, 2.2, and 2.3.

Compounds (from Table T3) 3.1.

Compounds (from Table T4) 4.1, 4.2, 4.3, 4.4, 4.5, 4.6, 4.7, 4.8, 4.9, and 4.11.

1. A compound of formula (I): 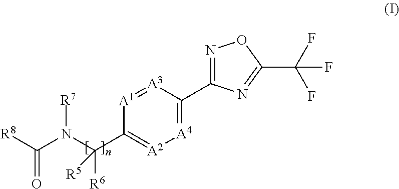
wherein
n represents 0, 1 or 2;
A¹ represents N or CR¹, wherein R¹ represents hydrogen, halogen, methyl, ethyl, difluoromethyl, trifluoromethyl, methoxy, ethoxy, or difluoromethoxy;
A² represents N or CR², wherein R² represents hydrogen, halogen, methyl, ethyl, difluoromethyl, trifluoromethyl, methoxy, ethoxy, or difluoromethoxy;
A³ represents N or CR³, wherein R³ represents hydrogen or halogen;
A⁴ represents N or CR⁴, wherein R⁴ represents hydrogen or halogen; and
wherein no more than two of A to A⁴ are N;
R⁵ and R⁶ are independently selected from hydrogen, C₁₋₄alkyl, halogen, cyano, trifluoromethyl and difluoromethyl, or R⁵ and R⁶ together with the carbon atom they share form a cyclopropyl;
R⁷ represents hydrogen, hydroxy, C₁₋₄alkyl, C₁₋₄haloalkyl, C₁₋₄alkoxy, C₁₋₄haloalkoxy, hydroxyC₁₋₄alkyl, C₁₋₂alkoxyC₁₋₄alkyl, C₁₋₂haloalkoxyC₁₋₄alkyl, cyanoC₁₋₄alkyl, C₃₋₆alkenyl, C₃₋₆alkynyl, C₃₋₆alkenyloxy, C₃₋₆alkynyloxy, C₃₋₆haloalkenyl, C₃₋₆haloalkenyloxy, or
R⁷ represents C₃₋₆cycloalkyl, C₃₋₆cycloalkylC₁₋₂alkyl, C₃₋₆cycloalkylC₁₋₂alkoxy, phenyl, phenylC₁₋₂alkyl, phenylC₁₋₂alkoxy, heteroaryl, heteroarylC₁₋₂alkyl, heteroarylC₁₋₂alkoxy, heterocyclyl, heterocyclylC₁₋₂alkyl, or heterocyclylC₁₋₂alkoxy,
wherein the heteroaryl moiety is a 5- or 6-membered monocyclic aromatic ring which comprises 1, 2, 3 or 4 heteroatoms individually selected from N, O and S, the heterocyclyl moiety is a 4- to 6-membered non-aromatic ring which comprises 1 or 2 heteroatoms individually selected from N, O and S, and wherein any of cycloalkyl, phenyl, heteroaryl and heterocyclyl moieties are optionally substituted by 1 or 2 substituents selected from cyano, fluoro, chloro, bromo, methyl, difluoromethyl, trifluoromethyl, methoxy, ethoxy, or difluoromethoxy;
R⁸ represents —C(═R⁹)—R¹⁰, wherein R⁹ represents O or N—C₁₋₄alkoxy;
R¹⁰ represents hydrogen, cyano, C₁₋₆alkyl, C₂₋₆alkenyl, C₃₋₆alkenyloxy, C₂₋₆alkynyl, cyanoC₁₋₆alkyl, C₁₋₆haloalkyl, C₃₋₆haloalkenyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkylcarbonylC₂₋₆alkenyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₆alkylsulfanylC₁₋₆alkyl, C₁₋₆alkylsulfonylC₁₋₆alkyl, C₁₋₆alkylsulfonylaminoC₁₋₆alkyl, or
R¹⁰ represents C₃₋₈cycloalkyl, C₃₋₈cycloalkylC₁₋₆alkyl, C₃₋₈cycloalkylC₁₋₆alkoxy wherein the cycloalkyl moiety is optionally partially unsaturated, phenyl, phenylC₁₋₆alkyl, phenylC₁₋₆alkoxy, heteroaryl, heteroarylC₁₋₆alkyl, heteroarylC₁₋₆alkoxy, wherein the heteroaryl moiety is a 5- or 6-membered monocyclic aromatic ring which comprises 1, 2, 3 or 4 heteroatoms individually selected from N, O and S, heterocyclyl, heterocyclylC₁₋₆alkyl, heterocyclylC₁₋₆alkoxy wherein the heterocyclyl moiety is a 4- to 6-membered non-aromatic ring which comprises 1, 2 or 3 heteroatoms individually selected from N, O and S;
wherein for R¹⁰, any C₃₋₈cycloalkyl, phenyl, heteroaryl, or heterocyclyl is optionally substituted by 1, 2 or 3 substituents, which may be the same or different, selected from R¹¹;
R¹¹ represents cyano, halogen, hydroxy, C₁₋₄alkyl, C₂₋₄alkenyl, C₂₋₄alkynyl, C₁₋₄haloalkyl, C₂₋₄haloalkenyl, C₁₋₄alkoxy, C₁₋₄haloalkoxy, C₃₋₄alkenyloxy, C₃₋₄alkynyloxy, N—C₁₋₄alkylamino, N,N-diC₁₋₄alkylamino, C₁₋₄alkylcarbonyl, C₁₋₄alkoxycarbonyl, carbonylamino, N—C₁₋₄alkylaminocarbonyl, N,N-diC₁₋₄alkylaminocarbonyl or C₁₋₄alkoxycarbonylamino;
and wherein when R¹⁰ is substituted C₃₋₈cycloalkyl, C₃₋₈cycloalkylC₁₋₆alkyl, C₃₋₈cycloalkylC₁₋₆alkoxy, heterocyclyl, heterocyclylC₁₋₆alkyl or heterocyclylC₁₋₆alkoxy, R¹¹ may also represent oxo on the C₃₋₈cycloalkyl or heterocyclyl moiety; or
R⁸ represents —C(═O)—OR²;
R¹² represents hydrogen, C₁₋₄alkyl, C₃₋₆alkenyl, C₃₋₆alkynyl, cyanoC₁₋₆alkyl, C₁₋₆haloalkyl, C₃₋₆haloalkenyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkylcarbonylC₂₋₆alkenyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₄alkylsulfanylC₁₋₆alkyl, C₁₋₆alkylsulfonylC₁₋₆alkyl, C₁₋₆alkylsulfonylaminoC₁₋₆alkyl, or
R¹² represents C₃₋₈cycloalkyl or C₃₋₈cycloalkylC₁₋₆alkyl wherein the cycloalkyl moiety is optionally partially unsaturated, phenyl, phenylC₁₋₆alkyl, heteroaryl or heteroarylC₁₋₆alkyl, wherein the heteroaryl moiety is a 5- or 6-membered monocyclic aromatic ring which comprises 1, 2, 3 or 4 heteroatoms individually selected from N, O and S, heterocyclyl or heterocyclylC₁₋₆alkyl wherein the heterocyclyl moiety is a 4- to 6-membered non-aromatic ring which comprises 1, 2 or 3 heteroatoms individually selected from N, O and S,
wherein for R¹², any C₃₋₈cycloalkyl, phenyl, heteroaryl or heterocyclyl is optionally substituted by 1, 2 or 3 substituents, which may be the same or different, selected from R¹³; wherein
R¹³ represents cyano, halogen, hydroxy, C₁₋₄alkyl, C₂₋₄alkenyl, C₂₋₄alkynyl, C₁₋₄haloalkyl, C₂₋₄haloalkenyl, C₁₋₄alkoxy, C₁₋₄haloalkoxy, C₃₋₄alkenyloxy, C₃₋₄alkynyloxy, N—C₁₋₄alkylamino, N,N-diC₁₋₄alkylamino, C₁₋₄alkylcarbonyl, C₁₋₄alkoxycarbonyl, carbonylamino, N—C₁₋₄alkylaminocarbonyl, N,N-diC₁₋₄alkylaminocarbonyl or C₁₋₄alkoxycarbonylamino;
and wherein when R¹² is substituted C₃₋₈cycloalkyl, C₃₋₈cycloalkylC₁₋₆alkyl, heterocyclyl or heterocyclylC₁₋₆alkyl, R¹³ may also represent oxo on the C₃₋₈cycloalkyl or heterocyclyl moiety; or
R⁸ represents —C(═O)—NR¹⁴R¹⁵; wherein
R¹⁴ represents hydrogen, amino, cyano, N,N-diC₁₋₄alkylamino, C₁₋₆alkyl, C₁₋₆alkoxy, C₂₋₆alkenyl, C₂₋₆alkynyl, cyanoC₁₋₆alkyl, C₁₋₆haloalkyl, C₃₋₆haloalkenyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkylcarbonylC₂₋₆alkenyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₄alkylsulfanylC₁₋₆alkyl, C₁₋₆alkylsulfonylC₁₋₆alkyl, C₁₋₆alkylsulfonylaminoC₁₋₆alkyl, or
R¹⁴ represents C₃₋₈cycloalkyl, C₃₋₈cycloalkylC₁₋₆alkyl, C₃₋₈cycloalkylC₁₋₆alkoxy wherein the cycloalkyl moiety is optionally partially unsaturated, phenyl, phenylC₁₋₆alkyl, phenylC₁₋₆alkoxy, heteroaryl, heteroarylC₁₋₆alkyl, heteroarylC₁₋₆alkoxy, wherein the heteroaryl moiety is a 5- or 6-membered monocyclic aromatic ring which comprises 1, 2, 3 or 4 heteroatoms individually selected from N, O and S, heterocyclyl, heterocyclylC₁₋₆alkyl, heterocyclylC₁₋₆alkoxy wherein the heterocyclyl moiety is a 4- to 6-membered non-aromatic ring which comprises 1, 2 or 3 heteroatoms individually selected from N, O and S;
wherein for R¹⁴, any C₃₋₈cycloalkyl, phenyl, heteroaryl or heterocyclyl is optionally substituted by 1, 2 or 3 substituents, which may be the same or different, selected from R¹⁶;
R¹⁶ represents cyano, halogen, hydroxy, C₁₋₄alkyl, C₂₋₄alkenyl, C₂₋₄alkynyl, C₁₋₄haloalkyl, C₂₋₄haloalkenyl, C₁₋₄alkoxy, C₁₋₄haloalkoxy, C₃₋₄alkenyloxy, C₃₋₄alkynyloxy, N—C₁₋₄alkylamino, N,N-diC₁₋₄alkylamino, C₁₋₄alkylcarbonyl, C₁₋₄alkoxycarbonyl, carbonylamino, N—C₁₋₄alkylaminocarbonyl, N,N-diC₁₋₄alkylaminocarbonyl or C₁₋₄alkoxycarbonylamino;
and wherein when R¹⁴ is substituted C₃₋₈cycloalkyl, C₃₋₈cycloalkylC₁₋₆alkyl, C₃₋₈cycloalkylC₁₋₆alkoxy, heterocyclyl, heterocyclylC₁₋₆alkyl or heterocyclylC₁₋₆alkoxy, R¹⁶ may also represent oxo on the C₃₋₈cycloalkyl or heterocyclyl moiety;
R¹⁵ is hydrogen, C₁₋₄alkyl, C₁₋₄alkoxyC₁₋₄alkyl, cyanoC₁₋₄alkyl, N-diC₁₋₄alkylamino; or
R¹⁴ and R¹⁵ together with the nitrogen atom to which they are bonded form a 4-, 5- or 6-membered cycle optionally containing a further heteroatom or group selected from O, S, S(O)₂, C(O) or NR¹⁷; and
R¹⁷ is hydrogen, methyl, methoxy, formyl or acyl; or
a salt or an N-oxide thereof.

2. A compound according to claim 1, wherein A¹ to A⁴ are C—H; A¹ is C—F and A² to A⁴ are C—H; or A³ is C—F and A¹, A² and A⁴ are C—H.

3. A compound according to claim 1, wherein R⁵ and R⁶ are independently selected from hydrogen and C₁₋₄alkyl, and preferably hydrogen.

4. A compound according to claim 1, wherein R⁷ is selected from hydrogen, C₁₋₄alkyl, C₁₋₄alkoxy, C₁₋₂alkoxyC₁₋₄alkyl, C₃₋₄alkenyl, C₃₋₄alkynyl, C₃₋₆cycloalkyl or C₃₋₆cycloalkylC₁₋₂alkyl.

5. A compound according to claim 1, wherein R⁸ represents —C(═O)—R¹⁰ and R¹⁰ is selected from hydrogen, C₁₋₆alkyl, cyanoC₁₋₆alkyl, C₁₋₆haloalkyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₆alkylsulfanylC₁₋₆alkyl, C₁₋₆alkylsulfonylC₁₋₆alkyl, C₁₋₆alkylsulfonylaminoC₁₋₆alkyl, or C₃₋₆cycloalkyl, phenyl, phenylC₁₋₂alkyl, heteroaryl or heteroarylC₁₋₂alkyl wherein any phenyl or heteroaryl moiety is optionally substituted by 1 or 2 substituents, which may be the same or different, selected from R¹¹, wherein R¹¹ is selected from cyano, halogen, hydroxy, methyl or methoxy.

6. A compound according to claim 5, wherein R¹⁰ is selected from hydrogen, C₁₋₆alkyl, C₃₋₆cycloalkyl, phenyl, phenylC₁₋₂alkyl, furanyl or thienyl, wherein any phenyl, furanyl or thienyl moiety is optionally substituted by 1 substituent selected from R¹¹, wherein R¹¹ is selected from cyano, halogen, hydroxy, methyl or methoxy.

7. A compound according to claim 1, wherein R⁸ represents —C(═O)—OR¹² and R¹² is selected from hydrogen, C₁₋₄alkyl, cyanoC₁₋₆alkyl, C₁₋₆haloalkyl, hydroxyC₁₋₆alkyl, C₁₋₄alkoxyC₁₋₆alkyl, C₁₋₄haloalkoxyC₁₋₆alkyl, C₁₋₄alkoxyC₁₋₄alkoxyC₁₋₆alkyl, aminoC₁₋₆alkyl, N—C₁₋₄alkylaminoC₁₋₆alkyl, N,N-diC₁₋₄alkylaminoC₁₋₆alkyl, C₁₋₆alkylcarbonylC₁₋₆alkyl, C₁₋₆alkoxycarbonylC₁₋₆alkyl, C₁₋₆alkylcarbonyloxyC₁₋₆alkyl, N—C₁₋₄alkylaminocarbonylC₁₋₆alkyl, N,N-diC₁₋₄alkylaminocarbonylC₁₋₆alkyl, C₁₋₄alkylsulfanylC₁₋₆alkyl, C₁₋₆alkylsulfonylC₁₋₆alkyl, C₁₋₆alkylsulfonylaminoC₁₋₆alkyl, or C₃₋₈cycloalkyl, C₃₋₈cycloalkylC₁₋₂alkyl, heterocyclyl, heterocyclylC₁₋₂alkyl wherein any C₃₋₈cycloalkyl or heterocyclyl moiety is optionally substituted by 1 or 2 substituents, which may be the same or different, selected from R¹³, wherein R¹³ represents cyano, halogen, hydroxy, methyl and methoxy.

8. A compound according to claim 1, wherein R⁸ represents —C(═O)—NR¹⁴R¹⁵, wherein:
R¹⁴ is selected from hydrogen, C₁₋₆alkyl, C₁₋₄alkoxy, C₂₋₄alkenyl, C₂₋₄alkynyl, cyanoC₁₋₄alkyl, C₁₋₄alkoxyC₁₋₄alkyl, C₁₋₄alkylsulfonylC₁₋₄alkyl, or C₃₋₆cycloalkyl, C₃₋₆cycloalkylC₁₋₂alkyl, phenyl, phenylC₁₋₂alkyl, heteroaryl or heteroarylC₁₋₂alkyl wherein the heteroaryl moiety is a 5- or 6-membered monocyclic aromatic ring which comprises 1 or 2 heteroatoms individually selected from N and O, heterocyclyl or heterocyclylC₁₋₂alkyl wherein the heterocyclyl moiety is a 4- to 6-membered non-aromatic ring which comprises 1 or 2 heteroatoms individually selected from N and O, wherein any C₃₋₆cycloalkyl, phenyl, heteroaryl or heterocyclyl is optionally substituted by 1 or 2 substituents, which may be the same or different, selected from R¹⁶ wherein R¹⁶ represents cyano, halogen, hydroxy, C₁₋₄alkyl, C₁₋₄haloalkyl, C₁₋₄alkoxy, C₁₋₄haloalkoxy, C₃₋₄alkenyloxy, C₃₋₄alkynyloxy, C₁₋₄alkylcarbonyl, C₁₋₄alkoxycarbonyl, N—C₁₋₄alkylaminocarbonyl and N,N-diC₁₋₄alkylaminocarbonyl; and
R¹⁵ is selected from hydrogen, methyl, ethyl, C₁₋₂alkoxyC₁₋₂alkyl or cyanoC₁₋₂alkyl.

9. A compound according to claim 8, wherein R¹⁴ represents hydrogen, C₁₋₄alkyl, methoxy, ethoxy, methoxyethyl, cyclopropyl, cyclopropylmethyl, 1,4-dioxanyl or tetrahydrofuranyl, wherein cyclopropyl, 1,4-dioxanyl or tetrahydrofuranyl are optionally substituted by 1 or 2 substituents, which may be the same or different, selected from R¹⁶.

10. A compound according to claim 9, wherein R¹⁴ is selected from hydrogen or C₁₋₄alkyl, and R¹⁵ is selected from hydrogen or methyl.

11. A compound according to claim 1, wherein n is 0 or 1.

12. An agrochemical composition comprising a fungicidally effective amount of a compound of formula (I) according to claim 1.

13. The composition according to claim 12, further comprising at least one additional active ingredient and/or an agrochemically-acceptable diluent or carrier.

14. A method of controlling or preventing infestation of useful plants by phytopathogenic microorganisms, wherein a fungicidally effective amount of a compound of formula (I) according to claim 1, or a composition comprising this compound as active ingredient, is applied to the plants, to parts thereof or the locus thereof.

15. Use of a compound of formula (I) according to claim 1 as a fungicide.
